SEQUENCE LISTING (304 sequences)

SEQ ID NO: 1-3 - skipped (3 entries)

SEQ ID NO: 4 - DNA, 894 bp, synthetic construct
  misc_feature 1..894: note = SYNTHETIC DNA SEQUENCE FOR ElxP OPEN READING FRAME
  source 1..894: mol_type = other DNA
atggataattttcttagttggcctaataaaaataaatattttgatgaaataaaagatgaagttaaaatattatatatagatagtgggtgtgatataaatcatatcgaagttaaagaaaatatattgataaatgaatctaaatcttttgtggacaatgatagtgaattatatgactatacgggacatggaacacaaattattagtgcaataacaggtaagcataatatgattggactatatcctagaagtaaaattgtaatatataaaataactaattataaaggtgaaactaaatttgaatggttatataaagcattatataaagctataaaaatggactataaaattattaacataagttattcaggatacacccaaaataattacataatatctaaattcaaaagattaatagaacaagcagttaaaaaaaatatacatattttatgtagtgctagtaatgatgaagtggaaaaaggtttttcaataccttctgattttaaaggagtctataaaattgcgagtataaatattgaagataaatattctagttatatttctaaatctaatgctgaatactttgctcctggaggagataattatttaaagacacagaatccacaatcatttattttgttagctaatagttctatttctaactttaatattggttctgattttggtatagataaaaggtatactttaaattttggtaatagtattgcatgctcctatgtttcttgttgtattgggctagtagtaacacgaagaaaaattaaatttaacaaagatacttctaaaaggtatatagattgtttatataataaatacaagcatataagtttgaatgtaatcaaaaacacaaaggagattattactaatgaacatatttaa

SEQ ID NO: 5 - protein, 297 aa, synthetic construct
  REGION 1..297: note = SYNTHETIC PEPTIDE FOR ElxP
MDNFLSWPNKNKYFDEIKDEVKILYIDSGCDINHIEVKENILINESKSFVDNDSELYDYTGHGTQIISAITGKHNMIGLYPRSKIVIYKITNYKGETKFEWLYKALYKAIKMDYKIINISYSGYTQNNYIISKFKRLIEQAVKKNIHILCSASNDEVEKGFSIPSDFKGVYKIASINIEDKYSSYISKSNAEYFAPGGDNYLKTQNPQSFILLANSSISNFNIGSDFGIDKRYTLNFGNSIACSYVSCCIGLVVTRRKIKFNKDTSKRYIDCLYNKYKHISLNVIKNTKEIITNEHI

SEQ ID NO: 6 - DNA, 2157 bp, synthetic construct
  misc_feature 1..2157: note = SYNTHETIC DNA SEQUENCE FOR His6-MBP-ElxP OPEN READING FRAME
  source 1..2157: mol_type = other DNA
atggatatcggaattaatactagtcatcatcatcatcatcacagcagcggcctggtgccgcgcggcagccatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcccaccatcaccatcaccacgcgaattcggtaccgctggttccgcgtggatctgagaacctgtacttccaatccggatccatggataattttcttagttggcctaataaaaataaatattttgatgaaataaaagatgaagttaaaatattatatatagatagtgggtgtgatataaatcatatcgaagttaaagaaaatatattgataaatgaatctaaatcttttgtggacaatgatagtgaattatatgactatacgggacatggaacacaaattattagtgcaataacaggtaagcataatatgattggactatatcctagaagtaaaattgtaatatataaaataactaattataaaggtgaaactaaatttgaatggttatataaagcattatataaagctataaaaatggactataaaattattaacataagttattcaggatacacccaaaataattacataatatctaaattcaaaagattaatagaacaagcagttaaaaaaaatatacatattttatgtagtgctagtaatgatgaagtggaaaaaggtttttcaataccttctgattttaaaggagtctataaaattgcgagtataaatattgaagataaatattctagttatatttctaaatctaatgctgaatactttgctcctggaggagataattatttaaagacacagaatccacaatcatttattttgttagctaatagttctatttctaactttaatattggttctgattttggtatagataaaaggtatactttaaattttggtaatagtattgcatgctcctatgtttcttgttgtattgggctagtagtaacacgaagaaaaattaaatttaacaaagatacttctaaaaggtatatagattgtttatataataaatacaagcatataagtttgaatgtaatcaaaaacacaaaggagattattactaatgaacatatttaa

SEQ ID NO: 7 - protein, 718 aa, synthetic construct
  REGION 1..718: note = SYNTHETIC PEPTIDE SEQUENCE FOR His6-MBP-ElxP
MDIGINTSHHHHHHSSGLVPRGSHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSHHHHHHANSVPLVPRGSENLYFQSGSMDNFLSWPNKNKYFDEIKDEVKILYIDSGCDINHIEVKENILINESKSFVDNDSELYDYTGHGTQIISAITGKHNMIGLYPRSKIVIYKITNYKGETKFEWLYKALYKAIKMDYKIINISYSGYTQNNYIISKFKRLIEQAVKKNIHILCSASNDEVEKGFSIPSDFKGVYKIASINIEDKYSSYISKSNAEYFAPGGDNYLKTQNPQSFILLANSSISNFNIGSDFGIDKRYTLNFGNSIACSYVSCCIGLVVTRRKIKFNKDTSKRYIDCLYNKYKHISLNVIKNTKEIITNEHI

SEQ ID NO: 8 - DNA, 1239 bp, synthetic construct
  misc_feature 1..1239: note = SYNTHETIC DNA SEQUENCE FOR CylA OPEN READING FRAME
  source 1..1239: mol_type = other DNA
atgaaaaaacgcggtctgacctatattctgatcagctatatctttctgattctgggcaccaccggttatgcaagcgatctgagcaacaatatcagcttctttattgataatagccagaccaccgccatcgaagaaattgaaagcgaactgagcagcgagaaagtggattacattcaagaaattggtctggtgagcttcaaaaacctggatgatagcgatcgcaaattcatcggcaaatatttcaatgtgagcgaaggtaaaaaactgccggattttaaaccggaagaagtgaatagcagcatcctgaacattaacatcctgaataaagatttcaaaagctttaattggccgtacaaaaaaatcctgagccatattgatccggtgaaagaacagctgggtaaagatattaccattgccctgattgatagcggtattgatcgtctgcatccgaatctgcaggacaataatctgcgtctgaaaaactatgtgaacgacatcgaactggatgaatatggtcatggcacccaggttgccggtgttattgataccattgcaccgcgtgttaatctgaacagctataaagttatggatggcaccgatggcaatagcattaatatgctgaaagcaattgtggatgccaccaatgatcaggtggatattatcaatgttagcctgggcagctacaaaaacatggaaattgatgatgaacgctttaccgttgaagcctttcgtaaagttgttaattacgcacgcaaaaataacatcctgattgttgcaagcgcaggtaatgaaagccgtgatattagcaccggtaacgaaaaacacattccgggtggtctggaaagcgttattaccgttggtgcaaccaaaaaaagcggtgatattgccgattacagcaattatggtagcaacgtgagcatttatggtccggcaggcggttatggtgataattacaaaatcaccggtcagattgatgcccgtgaaatgatgatgacctattatccgaccagcctggttagtccgctgggtaaagcagcagattttccggatggttataccctgagctttggcaccagcctggcaacaccggaagttagcgcagcactggcagcaattatgagcaaaaatgtggataacagcaaagacagcaatgaagttctgaacaccctgtttgaaaatgccgatagcttcatcgataaaaacagcatgctgaaatacaaagaagtgcgcattaaataa

SEQ ID NO: 9 - protein, 412 aa, synthetic construct
  REGION 1..412: note = SYNTHETIC PEPTIDE SEQUENCE FOR FULL LENGTH CylA
MKKRGLTYILISYIFLILGTTGYASDLSNNISFFIDNSQTTAIEEIESELSSEKVDYIQEIGLVSFKNLDDSDRKFIGKYFNVSEGKKLPDFKPEEVNSSILNINILNKDFKSFNWPYKKILSHIDPVKEQLGKDITIALIDSGIDRLHPNLQDNNLRLKNYVNDIELDEYGHGTQVAGVIDTIAPRVNLNSYKVMDGTDGNSINMLKAIVDATNDQVDIINVSLGSYKNMEIDDERFTVEAFRKVVNYARKNNILIVASAGNESRDISTGNEKHIPGGLESVITVGATKKSGDIADYSNYGSNVSIYGPAGGYGDNYKITGQIDAREMMMTYYPTSLVSPLGKAADFPDGYTLSFGTSLATPEVSAALAAIMSKNVDNSKDSNEVLNTLFENADSFIDKNSMLKYKEVRIK

SEQ ID NO: 10 - protein, 402 aa, synthetic construct
  REGION 1..402: note = SYNTHETIC PEPTIDE SEQUENCE FOR CylA with His6-tag, without the secretion signal peptide.
MGSSHHHHHHSQDPNSLSNNISFFIDNSQTTAIEEIESELSSEKVDYIQEIGLVSFKNLDDSDRKFIGKYFNVSEGKKLPDFKPEEVNSSILNINILNKDFKSFNWPYKKILSHIDPVKEQLGKDITIALIDSGIDRLHPNLQDNNLRLKNYVNDIELDEYGHGTQVAGVIDTIAPRVNLNSYKVMDGTDGNSINMLKAIVDATNDQVDIINVSLGSYKNMEIDDERFTVEAFRKVVNYARKNNILIVASAGNESRDISTGNEKHIPGGLESVITVGATKKSGDIADYSNYGSNVSIYGPAGGYGDNYKITGQIDAREMMMTYYPTSLVSPLGKAADFPDGYTLSFGTSLATPEVSAALAAIMSKNVDNSKDSNEVLNTLFENADSFIDKNSMLKYKEVRIK

SEQ ID NO: 11 - protein, 443 aa, synthetic construct
  REGION 1..443: note = SYNTHETIC PEPTIDE SEQUENCE FOR LicP, FULL LENGTH
MKRIYIFLLCFAVLLPVGGKTAQAKEQAGEQYLLLEHVKDKSKLLDTAEQFHIHADVIEEIGFAKVTGEKQKLAPFTKKLAEKVGADVIEKPIANTAVNETESVISGSPAWGLDGILELKEYLWFAAKQTDSYRTYQIERGHPDVKVALIDSGLDLDHPDLKASVNTNGGWNYIDGKPVSGDPTGHGTQTAGMINIIAPDVTITPYQVLDEKGGDSYNIMKAMVDAVNDGHEVINISTGSYTSLDREGKVLMKAYQRAANYAAKHQVLVFSSAGNKGVNLDEMRKTENKVHLPSALKHVVSVGSNMKSNNISPYSNQGREIEFTAPGGYLGETYDQDGMVRVTDLVLTTYPKGKDNTALDQMLNIPKGYSLSYGTSLAAPQVAGTAALVISEYRERHHRKPSAKQVHHILRKSALDLGKPGKDVIYGYGEVRAYQALKMMNKE

SEQ ID NO: 12 - protein, 432 aa, synthetic construct
  REGION 1..432: note = SYNTHETIC PEPTIDE SEQUENCE FOR LicP, with His6-tag, without the secretion signal peptide
GSSHHHHHHSQDPKEQAGEQYLLLEHVKDKSKLLDTAEQFHIHADVIEEIGFAKVTGEKQKLAPFTKKLAEKVGADVIEKPIANTAVNETESVISGSPAWGLDGILELKEYLWFAAKQTDSYRTYQIERGHPDVKVALIDSGLDLDHPDLKASVNTNGGWNYIDGKPVSGDPTGHGTQTAGMINIIAPDVTITPYQVLDEKGGDSYNIMKAMVDAVNDGHEVINISTGSYTSLDREGKVLMKAYQRAANYAAKHQVLVFSSAGNKGVNLDEMRKTENKVHLPSALKHVVSVGSNMKSNNISPYSNQGREIEFTAPGGYLGETYDQDGMVRVTDLVLTTYPKGKDNTALDQMLNIPKGYSLSYGTSLAAPQVAGTAALVISEYRERHHRKPSAKQVHHILRKSALDLGKPGKDVIYGYGEVRAYQALKMMNKE

SEQ ID NO: 13 - protein, 432 aa, synthetic construct; identical to SEQ ID NO: 12
  REGION 1..432: note = PEPTIDE SEQUENCE FOR His6-LicP-25-433

SEQ ID NO: 14 - protein, 476 aa, synthetic construct
  REGION 1..476: note = SYNTHETIC PEPTIDE SEQUENCE FOR LanP of B. licheniformis 9945A.
MYGRLGGKIMNIKRIHILLLCLLVVIIFALPGQASANDSQLDRYTIYLESPAHHDDWVKTLNERNIKLVYSVEEIGLYQIEGRQKEVQELAAEINYINSHNQSVAMQPASLHNTAPAEATIFGGTPTLWDDFQWDMRKATNNGQTYQITEGSKNTIVGIIDSGIDMNHPDLKENILSVRNFVPKGGLRGQEPYEKGDINDSTDYLGHGTFVAGQIAANGLMKGIAPETGIRSYRVFGGKSADTVWIIDAIIQAAIDDVDIINVSFGTFLVKQNRKNKQMPDTNDLAEIKAFKKAVSFAHKKGSAVVASIGNEGLDLKDKNAVFEYWRSTKQEDLSADGKVILIPAQLPNVVTVSSIGPSGMRSVFSNYGKNVVDIAADGGDLRLLNEVGEDRYYGEGLFRQEYILGAAPGVTYTFSIGTSIAAPKVSGALALLIDQKQLHDKPDKAVKILLKNADRNADKIDTGAGVLNVYRALTD

SEQ ID NO: 15 - protein, 490 aa, synthetic construct
  REGION 1..490: note = SYNTHETIC PEPTIDE SEQUENCE FOR LanP of Bacillus cereus Q1 (CerP)
MKDWLKYRNTIAKIGAGITVGLLLNLPIVGVSTISAEEMKHNQIKENSYVFTLRQGENTDEMIKEIRDKYPDLKLEVIKEIKMLNIEGDNLERVQEAKQHLLKNYKGIIEKAGREDVVELKPPTIVKNPSNKIKYAPSQLEQEEADYSKWKWDVDKVTENGESYKIEQGNHSVKIGVIDSGIDVNHPALKGNIVSQGKILVPNVESIEDNIGHGTMIAGLIAADGKIKGVAPKIGIVPYKVFQGSSADSSWIIKAIVEAANDGMDVINLSLGTYKSIKDPEEGAVYLAYKRAIEYANSKGSLLVASSGTEGFDISNPFQLAKQRGYENDLQLHMPGGLPGVVTVAGTNKEDRLSYYSNYGTNVDIAAPSGDYGSLWESEKVGDETAMVLTTYPTNLPQSQLSEWLGFDRGYELMIGTSLAVPKVSATAALVIAEYKEKFRLKPSNGFVKTRLYQGAKPALGDGKKYFGKGIVNAKGALDFRNTNFKKWER

SEQ ID NO: 16 - protein, 482 aa, synthetic construct
  REGION 1..482: note = SYNTHETIC PEPTIDE SEQUENCE FOR LanP of Bacillus cereus FRI-35.
MKRLKILMTILSVFLLSLVTQPVKAEKIETTDNYKFILLDNEIQKNNKENIINFLKENNAIEIKYTPEIQMISYKETNSNIQSDLDSKIRTKFKASIENVANSAKLTLQDPKILNPNEQFANNKLKYNLSSKRTLNSITINEPVSNVSPPWQIAKVTDNYKSHEITLGEKNVNIALVDSGVDYNHPELKNSIKYLEGKSYVPSEPDLLDQNGHGTQVAGIIAAHDKLKGIVPNITITPYKVIGKKDGESVWTIQAIIDAANNGADVINLSLGTYKAINLDDENAIIKAYERAIQYAKEKNSIVVASAGNEGINLDKPFEKVDDEKGVLYKIHVPGGLSNVLTVSGTDKNDEFVNYSNFGPEVDFSAPSGSYGTIIDNQFKFDPSYLIGTTYPTYLEPTMLDAQLNTPKGYTLNLGTSLSAPSVSGTLGAIISRYYELHSTKPSADITIKYLKDGATDLGATGKDDQYGYGLVNSYKSTSSVK

SEQ ID NO: 17 - protein, 581 aa, synthetic construct
  REGION 1..581: note = SYNTHETIC PEPTIDE SEQUENCE FOR LanP of Kyrpidia tusciae DSM 2912
MTGKGVRKRIQVVLMAGLAGGVAWGALDSVGVGRAAGLKGVDAPAVAASVDRSGTGGARTAGEPGTAGQPVRGPADPEAGNRLLVVFKSGQVPAGLVDALSAQPGVKVTPLPDIGAVAVKTATREGGDAVKRVLTARFGGDVDAVGADPILTLNTELNRPVGETLPLSAVDLTSVQVPPVPGAAPGATSPAENVSPASAKLSPAVDKAAPPATGAGSAATAVTTGSSVDVDRAVLYQQWGWDIQEVTENGKSFAVQPGNHRVKVAVIDSGIDSNHPDLRNNIVSPGRSFVPGDPSTADDFGHGTMVAGTIAADGLLLGVGPHLGIVPYRVFQHGDAQSSWVIQAIVQAVKDGADVINLSLGTYKSLKNPADRADLLAYQKAVAYALVHGVTVVASSGTDGVDIGNPAQLANQLGHPGDLEVHAPGGLPGVLTTAATNRQQARAYYSNYGRNVDLAAPGGDFGPLWDSEHIADVRNMCLVTYPTNLPQTPLSRMAGLPKGYEWMIGTSLAAPKVAAAAALVIAQERDKRGVRGVLGPWTNPANPVTVRNILERTAVDVGTPGRDPETGAGVVDAAKALQAVR

SEQ ID NO: 18 - protein, 456 aa, synthetic construct
  REGION 1..456: note = SYNTHETIC PEPTIDE SEQUENCE FOR LanP of Enterococcus caccae ATCC BAA-1240
MRRYLIHIILVSVLGAFAALSLGISAFGENQINLVEQTFLLNDKKEVTQLTQLVYDINPSIKQTVIEEIDMVHLENVTLEELETIENITQIEKLSEKSGQMSKVETESIKITDVTTNFDDVKRIDNRQALFSRKSSSLLDLLSWHVDDVTSNKQSYAIATGKNIKVAVIDSGIDTESAYFQKNLTLENAKSFVSNDESIIDENGHGTMVTGVLTQVAPDVKVTPYKVINATTGDSIWTIQAVIQAVKDKNQIINLSLGTYKKDNSKEERLTITAYERAIKFAKKNHVFVLASAGNDGLDLDALKLRERKIHLPGGMKNLFTIGATRRNDNKSSYSNYGNEIDFVAPGGDIYNDEGQLDLNEFIFTTYPVYLDNGLGALGIPQGYMFSGGTSLATPAVSGVVATIYEKYYQRYGIYPQNQVVNKLLKAGGVDIGTPGLDKYFGYGKVNGYQSLSKIN

SEQ ID NO: 19 - protein, 476 aa, synthetic construct
  REGION 1..476: note = SYNTHETIC PEPTIDE SEQUENCE FOR LanP of Bacillus cereus VPC1401
MKKWQFKLCNITLIFTTIILFTFTFNKSSNAQTEHDQLIMFTDSKISNEVNQFLEKKYPDLKKTVIPEIAVVKLEETNNKQYSKAISEIKRKFHNEIESIGPESKIINPKESSLNAIISKEEIVPSSTQNIEEKAKLYEVLGWDIKKITEDGKSFKKQKGNHNVKIALIDSGIDFNHPDLKDNIISKGKSFVPNIDNTDDNMGHGTMAAGSIAANGNMLGVGPNLGIIPYKVMDNWQDGAESAWVTQAIIAAANDGVDVINLSLGTYKSLNKPEDQVVIESYKRAVKYAHKRGVIVVASAGNESLDNTNPALIAEKRGFSGDMQVHLPGGGVPSLLTVSATDKDDLLSSYSNYGGISVAAPAGDYGPEWATQQKLDPFSMTLTTYPTNLPQPPISKALNLPEGYVLMAGTSVAAPKVSGAVGVLIAEQQKSGRKPLTLAKLEKILKNSSTDLGAYGKDPKYGYGLINVNKALEFIK

SEQ ID NO: 20 - protein, 454 aa, synthetic construct
  REGION 1..454: note = SYNTHETIC PEPTIDE SEQUENCE FOR CrnP
MRILVKNILSIISSVLIFVVLSGNRVVAFAQEPSIDLLVSEGESIEQLKEELTEVDNKLEFIEIPEINLLRIENPNEKVQEVIENSDEIDFSGKISDKLILENNTITEKNVDFPKIFQYSAEEQNIETELLNRLTWYINTLTQDKKAFEYSKGKNIKIGLIDSGVDTSHPLISPSLNLEKAKSFVKNDQSIEDSNGHGTMVAGVISQVSPEAKITPYRVMSAIDGESIWTLQAIIRAVNDKQDIINMSLGTYKYETKKDERITIEAFKRAACYAFIKKVLIISSSGNQQLDSDVNYRENKIMHLPGDIKGVITVSAINKNNQLTNYSNTGTNVQYTAPGGEIIIDENGYLDARELIYTMYPLTLPNPMKDAGIPDGYTLTYGTSFSSAGVTAVFANYYSYYLQIMQKKPNSQNAHKFIAYNSTDFGVVGKDSQYGYGLPNLIRAYELISSNKNH

SEQ ID NO: 21 - protein, 476 aa, synthetic construct
  REGION 1..476: note = SYNTHETIC PEPTIDE SEQUENCE FOR LanP of Bacillus bombysepticus
MKNWQSKLGTIIVILTTVILFTFTFNKSSNAESGNDQLFMFTDSNTSEEVNQFIKEKYPSIKSTIIPEIAMVKLEDTDNGQHIKASHEIKEKFHNKIESTGPESKIIDPKEDNLNPSISENEIIPTVIPNIEEKAKLYQELGWDIKKITEDGKSFKKQKGNHNVKVAIIDSGIDFNHPDLKGNIISKGKSFVPNVDNTDDHMGHGTMAAGSIAANGNMLGVGPELGIIPYKVMDNWQDGAESAWVTQAIIAATKDGVDVINLSLGTYKSLEKPEDRVVVESYKRAVKYAHKHGVIVVASAGNESLDNNNPTLIAEKRGFSGDKQVHLPGGGLPSLITVSATDKNDLLASYSNYGGVSVAAPAGDYGPEWATQQKLDPFSMTLTTYPTNLPQPPISKALNLPEGYVLMAGTSVAAPKVSGAVGVLIAEQQKKGRKSLKLAQLKNILKKSSTDLGEPGKDDKYGYGLINVNKALELIK

SEQ ID NO: 22 - protein, 490 aa, synthetic construct
  REGION 1..490: note = SYNTHETIC PEPTIDE SEQUENCE FOR LanP of Bacillus thuringiensis DB27
MKDWPKYRNTIAKIGAGITVGLLLNLPIVGVSTISAEEMKHNQIKENSYVFTLRQGENTDEMIKEIRDKYPDLKLEVIKEIKMLNIEGDNLERVQEAKQYLLKNYKGIIEKAGREDVVELKPPAIVKKPSNITKYAPSQLEQEEVDYSKWKWDVDKVTENGESYKIEQGNHSVKIGVIDSGIDVNHPALKGNIVSQGKILVPNVESIEDNIGHGTMIAGLIAADGKIKGVAPKIGIVPYKVFQGSSADSSWIIKAIVEAANDGMDVINLSLGTYKSIKNPEEGAVYLAYKRAIEYANSKGSLLVASSGTEGFDISNPFQLAKQRGYENDLQLHMPGGLPGVVTVAGTNKDDRLAYYSNYGTNVDIAAPSGDYGSLWESEKVGDETAMVLTTYPTNLPQSQLSEWLGFDRGYELMIGTSLAVPKVSATAALVIAEYKEKFRLKPSNGFVKTRLYQGAKPALDDGKKYFGKGIVNAKGALDFRNTNFKKWER

SEQ ID NO: 23 - protein, 484 aa, synthetic construct
  REGION 1..484: note = SYNTHETIC PEPTIDE SEQUENCE FOR LanP of Planomicrobium glaciei CHR43
MNVQHKLPKALAGILALSVSMSCLTPSAQAAGFPKLPVQAEEEAKMIYLFQEGTNLRMIAEDIQRIDPGASIDAVQEIETLTVKASNPAQSKKIKKHVQNEFGTLITEKGEDQTVKAGDVRPSSMPPVSTLTSLAAVSASQDTVNEEDSTYTKWLWDIDLVTQNGASRDIEIGNHGVKVGIVDSGLDFNHPDLKANIVSKGRSFVDGAADTQDYMGHGTMVAGSIAANGHIKGIAPEIGIVPYKVFHTGNADSSDVVEAIVAAANDDMDVINLSLGVYKSLRNKDEKAVYEAYKRALKYAEKENSFVVASSGTESAGFDIFNAKKLAAARGFSEDAQLHMPGGLDDVFTVAATNKDNALTFYSNYGENVSIGAPGGDYGPLADQGLYDVRHMTLTTYPTNLMQSITSEYAGFEKGYEFMTGTSLAAPKVSATAALVIAEYEEVHGKKPKVHEVKKMLEKGALKGDKKNFGAGIVNAYNSLTLIK

SEQ ID NO: 24 - protein, 452 aa, synthetic construct
  REGION 1..452: note = SYNTHETIC PEPTIDE SEQUENCE FOR LanP of Bacillus cereus VD045
MYRFKKYCLSIISFILIISFFPNNTNATQIAYYSILIRDNTDFNTVLDKLSKDKQEVVYSIPEVNLIQVKGEKGKIISGIGIESIEEINPSTGEFKTYTPNINDQKVLDNKAIWDVQWDIKRITNNGESYKLHSPSGKVSVALIDSGYPENHPDIKSISIQKSKNLVPKGGYKGNEENETGNIHQLTDRTGHGTSVLSQVNADGLMKGVAPGMPVNMYRVFGESSAEGSWIIKGIIEAAKDKNDVINISAGSYLLKNGTYSDGSGNNRAEIKAYEKAIHYANKKGSIVVSALGNDSINININSELLSNLNSKIKDEGKSAKGIVQDIPAQLAEVVSVASTGMDSKVSSFSNYGKNIIDFTAPGGDIKLLNKFGADVWMAEEMFKKEMILVAHPQGGYYFNYGNSLATPKVSGALALVIDKYGYKNKPNKAINHLKRNTNAENEIDLYKALQE

SEQ ID NO: 25 - protein, 452 aa, synthetic construct
  REGION 1..452: note = SYNTHETIC PEPTIDE SEQUENCE FOR B. cereus VD156 LanP (ACCESSION NO. EJR72593.1)
MYRFKKYCLSIISFILIISFFPNNTNATQIAYYSILIRDNTDFNTVLDKLSKDNQEVVYSIPEVNLIQVKGEKGKIISGIGLESIEEINPSTGEFKTYTPNINDQKVLDNKAIWDVQWDIKRITNNGESYKLHSPSGKVSVALIDSGYPENHPDIKSISIQKSKNLVPKGGYKGNEENETGNIHQLTDRTGHGTSVLSQVNADGLMKGVAPGMPVNMYRVFGESSAEGSWIIKGIIEAAKDKNDVINISAGSYLLKNGTYSDGSGNNRAEIKAYEKAIHYANKKGSIVVSALGNDSININIYSELLSILNSKIKDEGKSATGIVQDIPAQLAQVVSVASTGMDSKVSSFSNYGKNIIDFTAPGGDIKLLNKFGADVWMAEEMFKKEMILVAHPQGGYYFNYGNSLATPKVSGALALVIDKYGYKNKPNKAINHLKRNTNAENEIDLYKALQE

SEQ ID NO: 26 - DNA, 1359 bp, synthetic construct
  misc_feature 1..1359: note = SYNTHETIC DNA SEQUENCE FOR B. cereus VD156 LanP (OPTIMIZED NUCLEOTIDE SEQUENCE)
  source 1..1359: mol_type = other DNA
atgtatcgcttcaagaaatattgcctcagcattatctcgttcattctgatcatcagtttcttcccgaacaataccaacgcgactcaaatcgcctattactcaattctgatccgggataacactgacttcaacacagtactggacaaattgagtaaggataaccaggaggtggtctatagcatcccggaagtcaatctgattcaagtcaaaggcgaaaaaggtaagattattagtggtatcggtttagagagtattgaagagattaacccgtcaacaggcgaattcaaaacctacaccccgaatattaatgaccaaaaagtgctggataacaaagccatctgggacgtccagtgggatatcaaacgcattacgaacaacggcgaaagttataaattacactctccgagcgggaaagtgagtgttgcactgattgatagcggttatccggaaaaccacccagatatcaaatcaatctccatccagaaaagcaagaaccttgttccgaaggggggctataagggaaatgaagaaaacgaaaccggtaacattcaccagctgacggatcgcactggtcacgggaccagcgtgctgtctcaagtgaacgccgatggtttaatgaaaggagttgctcctggcatgcctgtgaacatgtatcgtgtcttcggcgaatcatcggcagaggggagttggatcatcaaaggaattatcgaggccgccaaggataagaatgatgttatcaacatttcagcgggcagctatttactgaagaacggcacctattctgacggaagcggcaacaatcgggcagaaatcaaagcatacgaaaaggcgatccattatgcgaacaaaaaaggaagcattgttgtgagcgctctgggcaacgacagcattaacattaatatctactccgaactgctgagcatcctgaatagcaaaattaaagatgaaggcaaaagtgccacgggcatcgtgcaagacatcccggctcagctggcacaagttgtgtctgtcgcgtcgacgggcatggatagcaaagtgtcaagcttttcgaactacggaaaaaacattattgattttaccgccccgggaggggatattaaacttctcaataaattcggcgctgatgtttggatggctgaagaaatgttcaagaaggagatgattttggtcgctcacccgcagggtggctactacttcaattacggtaactccttagctacgcccaaggtcagcggtgccctcgctctcgtgatcgataaatatggttacaaaaataaacccaacaaagctattaatcatctgaaacgtaatactaacgcggagaacgaaattgatttgtacaaagcactccaagagtaa

SEQ ID NO: 27 - protein, 65 aa, synthetic construct
  REGION 1..65: note = SYNTHETIC PEPTIDE SEQUENCE FOR B. cereus VD156 LanA3 (EJR72967.1)
MKNISEKSVGLSMKKLDTTEMEKIYGASGVDTRTHPTVVVVSRASSKFCVTVAASALLSYNMNKC

SEQ ID NO: 28 - DNA, 198 bp, synthetic construct
  misc_feature 1..198: note = SYNTHETIC DNA SEQUENCE FOR B. cereus VD156 LanA3
  source 1..198: mol_type = other DNA
atgaaaaatatttcagagaaatcagtagggcttagtatgaagaagctggataccaccgaaatggaaaaaatctatggcgcgtctggtgtagacacgcgtacgcatcccacagtcgtcgtagtgagccgtgccagctcaaaattttgcgtcaccgtagctgcatctgcactgctctcgtataacatgaataagtgttaa

SEQ ID NO: 29 - protein, 449 aa, synthetic construct
  REGION 1..449: note = SYNTHETIC PEPTIDE SEQUENCE FOR LanP from B. licheniformis 9945A (without signal sequence, with His6 tag).
MGSSHHHHHHSQDPDRYTIYLESPAHHDDWVKTLNERNIKLVYSVEEIGLYQIEGRQKEVQELAAEINYINSHNQSVAMQPASLHNTAPAEATIFGGTPTLWDDFQWDMRKATNNGQTYQITEGSKNTIVGIIDSGIDMNHPDLKENILSVRNFVPKGGLRGQEPYEKGDINDSTDYLGHGTFVAGQIAANGLMKGIAPETGIRSYRVFGGKSADTVWIIDAIIQAAIDDVDIINVSFGTFLVKQNRKNKQMPDTNDLAEIKAFKKAVSFAHKKGSAVVASIGNEGLDLKDKNAVFEYWRSTKQEDLSADGKVILIPAQLPNVVTVSSIGPSGMRSVFSNYGKNVVDIAADGGDLRLLNEVGEDRYYGEGLFRQEYILGAAPGVTYTFSIGTSIAAPKVSGALALLIDQKQLHDKPDKAVKILLKNADRNADKIDTGAGVLNVYRALTD

SEQ ID NO: 30 - protein, 439 aa, synthetic construct
  REGION 1..439: note = SYNTHETIC PEPTIDE SEQUENCE FOR LanP of B. cereus VD156 (without signal sequence, with His6 tag)
MGSSHHHHHHSQDPTQIAYYSILIRDNTDFNTVLDKLSKDNQEVVYSIPEVNLIQVKGEKGKIISGIGLESIEEINPSTGEFKTYTPNINDQKVLDNKAIWDVQWDIKRITNNGESYKLHSPSGKVSVALIDSGYPENHPDIKSISIQKSKNLVPKGGYKGNEENETGNIHQLTDRTGHGTSVLSQVNADGLMKGVAPGMPVNMYRVFGESSAEGSWIIKGIIEAAKDKNDVINISAGSYLLKNGTYSDGSGNNRAEIKAYEKAIHYANKKGSIVVSALGNDSININIYSELLSILNSKIKDEGKSATGIVQDIPAQLAQVVSVASTGMDSKVSSFSNYGKNIIDFTAPGGDIKLLNKFGADVWMAEEMFKKEMILVAHPQGGYYFNYGNSLATPKVSGALALVIDKYGYKNKPNKAINHLKRNTNAENEIDLYKALQE

SEQ ID NO: 31 - protein, 11 aa, synthetic construct
  REGION 1..11: note = SYNTHETIC SUBSTRATE SEQUENCE FROM LicP-95-105
NTAVNETESVI

SEQ ID NO: 32 - protein, 11 aa, synthetic construct
  REGION 1..11: note = SYNTHETIC PEPTIDE SUBSTRATE SEQUENCE FOR LicP-E100A-97-107.
AVNATESVISG

SEQ ID NO: 33 - protein, 11 aa, synthetic construct
  REGION 1..11: note = SYNTHETIC PEPTIDE SUBSTRATE SEQUENCE FOR Modified LicA2
  REGION 7..8: note = MISC_FEATURE - X IS Dhb.
  SITE 11: note = MISC_FEATURE - X IS Dhb.
NDVNPEXXPAX

SEQ ID NO: 34 - protein, 11 aa, synthetic construct
  REGION 1..11: note = SYNTHETIC PEPTIDE SUBSTRATE SEQUENCE FOR Linear LicA2
NDVNPETTPAT

SEQ ID NO: 35 - protein, 11 aa, synthetic construct
  REGION 1..11: note = SYNTHETIC PEPTIDE SUBSTRATE SEQUENCE FOR ProcA1.7-NDVNPE
NDVNPETIGGT

SEQ ID NO: 36 - protein, 11 aa, synthetic construct
  REGION 1..11: note = SYNTHETIC PEPTIDE SUBSTRATE SEQUENCE FOR NisA-NDVNPE
NDVNPEITSIS

SEQ ID NO: 37 - protein, 11 aa, synthetic construct
  REGION 1..11: note = SYNTHETIC PEPTIDE SUBSTRATE SEQUENCE FOR MBP-BamL
NDVNPESGSEN

SEQ ID NO: 38 - protein, 11 aa, synthetic construct
  REGION 1..11: note = SYNTHETIC PEPTIDE SUBSTRATE SEQUENCE FOR NisA-NDVNPE-I1T
NDVNPETTSIS

SEQ ID NO: 39 - protein, 11 aa, synthetic construct
  REGION 1..11: note = SYNTHETIC PEPTIDE SUBSTRATE SEQUENCE FOR NisA-NDVNPE-I1C
NDVNPECTSIS

SEQ ID NO: 40 - protein, 11 aa, synthetic construct
  REGION 1..11: note = SYNTHETIC PEPTIDE SUBSTRATE SEQUENCE FOR NisA-NDVNPE-I1G
NDVNPEGTSIS

SEQ ID NO: 41 - protein, 11 aa, synthetic construct
  REGION 1..11: note = SYNTHETIC PEPTIDE SUBSTRATE SEQUENCE FOR NisA-NDVNPE-I1W
NDVNPEWTSIS

SEQ ID NO: 42 - protein, 11 aa, synthetic construct
  REGION 1..11: note = SYNTHETIC PEPTIDE SUBSTRATE SEQUENCE FOR NisA-NDVNPE-I1L
NDVNPELTSIS

SEQ ID NO: 43 - protein, 11 aa, synthetic construct
  REGION 1..11: note = SYNTHETIC PEPTIDE SUBSTRATE SEQUENCE FOR NisA-NDVNPE-I1K
NDVNPEKTSIS

SEQ ID NO: 44 - protein, 11 aa, synthetic construct
  REGION 1..11: note = SYNTHETIC PEPTIDE SUBSTRATE SEQUENCE FOR NisA-NDVNPE-I1F
NDVNPEFTSIS

SEQ ID NO: 45 - protein, 11 aa, synthetic construct
  REGION 1..11: note = SYNTHETIC PEPTIDE SEQUENCE FOR NisA-NDVNPE-I1E
NDVNPEETSIS

SEQ ID NO: 46 - protein, 6 aa, synthetic construct
  REGION 1..6: note = SYNTHETIC CylA PEPTIDE SUBSTRATE SEQUENCE
GDVQAE

SEQ ID NO: 47 - protein, 55 aa, synthetic construct
  REGION 1..55: note = SYNTHETIC PEPTIDE SUBSTRATE SEQUENCE FOR HIS6-ElxA
MKKELFDLNLNKDIEAQKSDLNPQSASIVKTTIKASKKLCRGFTLTCGCHFTGKK

SEQ ID NO: 48 - protein, 55 aa, synthetic construct
  REGION 1..55: note = SYNTHETIC PEPTIDE SUBSTRATE SEQUENCE FOR HIS6-ElxA (Q(-1)A)
MKKELFDLNLNKDIEAQKSDLNPASASIVKTTIKASKKLCRGFTLTCGCHFTGKK

SEQ ID NO: 49 - protein, 55 aa, synthetic construct
  REGION 1..55: note = SYNTHETIC PEPTIDE SUBSTRATE SEQUENCE FOR HIS6-ElxA (P(-2)A)
MKKELFDLNLNKDIEAQKSDLNAQSASIVKTTIKASKKLCRGFTLTCGCHFTGKK

SEQ ID NO: 50 - protein, 55 aa, synthetic construct
  REGION 1..55: note = SYNTHETIC PEPTIDE SUBSTRATE SEQUENCE FOR HIS6-ElxA (N(-3)A)
MKKELFDLNLNKDIEAQKSDLAPQSASIVKTTIKASKKLCRGFTLTCGCHFTGKK

SEQ ID NO: 51 - protein, 55 aa, synthetic construct
  REGION 1..55: note = SYNTHETIC PEPTIDE SUBSTRATE SEQUENCE FOR HIS6-ElxA (L(-4)A)
MKKELFDLNLNKDIEAQKSDANPQSASIVKTTIKASKKLCRGFTLTCGCHFTGKK

SEQ ID NO: 52 - protein, 55 aa, synthetic construct
  REGION 1..55: note = SYNTHETIC PEPTIDE SUBSTRATE SEQUENCE FOR HIS6-ElxA (D(-5)A)
MKKELFDLNLNKDIEAQKSALNPQSASIVKTTIKASKKLCRGFTLTCGCHFTGKK

SEQ ID NO: 53 - protein, 55 aa, synthetic construct
  REGION 1..55: note = SYNTHETIC PEPTIDE SUBSTRATE SEQUENCE FOR HIS6-ElxA (F(-19)A)
MKKELADLNLNKDIEAQKSDLNPQSASIVKTTIKASKKLCRGFTLTCGCHFTGKK

SEQ ID NO: 54 - protein, 55 aa, synthetic construct
  REGION 1..55: note = SYNTHETIC PEPTIDE SUBSTRATE SEQUENCE FOR HIS6-ElxA (D(-18)A)
MKKELFALNLNKDIEAQKSDLNPQSASIVKTTIKASKKLCRGFTLTCGCHFTGKK

SEQ ID NO: 55 - protein, 55 aa, synthetic construct
  REGION 1..55: note = SYNTHETIC PEPTIDE SUBSTRATE SEQUENCE FOR HIS6-ElxA (L(-17)A)
MKKELFDANLNKDIEAQKSDLNPQSASIVKTTIKASKKLCRGFTLTCGCHFTGKK

SEQ ID NO: 56 - protein, 55 aa, synthetic construct
  REGION 1..55: note = SYNTHETIC PEPTIDE SUBSTRATE SEQUENCE FOR HIS6-ElxA (N(-16)A)
MKKELFDLALNKDIEAQKSDLNPQSASIVKTTIKASKKLCRGFTLTCGCHFTGKK

SEQ ID NO: 57 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
AAIVK

SEQ ID NO: 58 - protein, 4 aa, synthetic construct
  REGION 1..4: note = SYNTHETIC PEPTIDE SUBSTRATE SEQUENCE FOR His6-ElxO
  SITE 1: note = MISC_FEATURE - Pyr covalently attached to N-terminal residue.
AAIV

SEQ ID NO: 59 - protein, 13 aa, synthetic construct
  REGION 1..13: note = SYNTHETIC PEPTIDE SUBSTRATE SEQUENCE FOR His6-ElxO
  SITE 1: note = MISC_FEATURE - Pyr covalently attached to N-terminal residue.
AAIVKBBIKAAKK

SEQ ID NO: 60 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
AAIVA

SEQ ID NO: 61 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
AAIAK

SEQ ID NO: 62 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
AAAVK

SEQ ID NO: 63 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
RAIVK

SEQ ID NO: 64 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
KAIVK

SEQ ID NO: 65 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
DAIVK

SEQ ID NO: 66 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
NAIVK

SEQ ID NO: 67 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
PAIVK

SEQ ID NO: 68 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Pyr covalently attached to N-terminal residue.
MAIVK

SEQ ID NO: 69 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Obu covalently attached to N-terminal residue.
AAIVK

SEQ ID NO: 70 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Obu covalently attached to N-terminal residue.
RAIVK

SEQ ID NO: 71 - protein, 5 aa, synthetic construct
  REGION 1..5: note = SYNTHETIC PEPTIDE SUBSTRATE SEQUENCE FOR His6-ElxO
  SITE 1: note = MISC_FEATURE - Glx covalently attached to N-terminal residue.
AAIVK

SEQ ID NO: 72 - protein, 11 aa, synthetic construct
  REGION 1..11: note = SYNTHETIC CylL(L) PEPTIDE SUBSTRATE SEQUENCE FOR CylA.
GDVQAETTPVC

SEQ ID NO: 73 - protein, 11 aa, synthetic construct
  REGION 1..11: note = SYNTHETIC CylL(S) PEPTIDE SUBSTRATE SEQUENCE FOR CylA.
GDVQAETTPAC

SEQ ID NO: 74 - protein, 11 aa, synthetic construct
  REGION 1..11: note = SYNTHETIC HalA2 PEPTIDE SUBSTRATE SEQUENCE FOR CylA.
  SITE 7: note = MISC_FEATURE - HalM2-catalyzed addition of a MeLan modification at residue 7.
GDVQAETTWPC

SEQ ID NO: 75 - protein, 11 aa, synthetic construct
  REGION 1..11: note = SYNTHETIC HalA1 PEPTIDE SUBSTRATE SEQUENCE FOR CylA.
  SITE 7: note = MISC_FEATURE - HalM1-catalyzed addition of a Cys or disulfide linkage on residue C-7.
GDVQAECAWYN

SEQ ID NO: 76 - protein, 11 aa, synthetic construct
  REGION 1..11: note = SYNTHETIC NisA PEPTIDE SUBSTRATE SEQUENCE FOR CylA.
GDVQAEITSIS

SEQ ID NO: 77 - protein, 11 aa, synthetic construct
  REGION 1..11: note = SYNTHETIC NisA-l1C PEPTIDE SUBSTRATE SEQUENCE FOR CylA.
GDVQAECTSIS

SEQ ID NO: 78 - protein, 11 aa, synthetic construct
  REGION 1..11: note = SYNTHETIC ProcA1.7 PEPTIDE SUBSTRATE SEQUENCE FOR CylA.
GDVQAETIGGT

SEQ ID NO: 79 - protein, 11 aa, synthetic construct
  REGION 1..11: note = SYNTHETIC ProA1.7-T1C PEPTIDE SUBSTRATE SEQUENCE FOR CylA.
GDVQAECIGGT

SEQ ID NO: 80 - protein, 11 aa, synthetic construct
  REGION 1..11: note = SYNTHETIC ProA1.7-T1G PEPTIDE SUBSTRATE SEQUENCE FOR CylA.
GDVQAEGIGGT

SEQ ID NO: 81 - protein, 11 aa, synthetic construct
  REGION 1..11: note = SYNTHETIC ProA1.7-T1F PEPTIDE SUBSTRATE SEQUENCE FOR CylA.
GDVQAEFIGGT

SEQ ID NO: 82 - protein, 11 aa, synthetic construct
  REGION 1..11: note = SYNTHETIC ProA1.7-T1W PEPTIDE SUBSTRATE SEQUENCE FOR CylA.
GDVQAEWIGGT

SEQ ID NO: 83 - protein, 11 aa, synthetic construct
  REGION 1..11: note = SYNTHETIC His6-ProA1.7 PEPTIDE SUBSTRATE SEQUENCE FOR CylA.
GDVQAETMKHR

SEQ ID NO: 84 - DNA, 50 bp, synthetic construct
  misc_feature 1..50: note = ElxA Q-1A F SYNTHETIC OLIGONUCLEOTIDE PRIMER
  source 1..50: mol_type = other DNA
gcacaaaaaagtgacctaaatccggcatcagctagtattgttaaaacaac

SEQ ID NO: 85 - DNA, 50 bp, synthetic construct
  misc_feature 1..50: note = ElxA Q-1A R SYNTHETIC OLIGONUCLEOTIDE PRIMER
  source 1..50: mol_type = other DNA
gttgttttaacaatactagctgatgccggatttaggtcacttttttgtgc

SEQ ID NO: 86 - DNA, 50 bp, synthetic construct
  misc_feature 1..50: note = ElxA P-2A F SYNTHETIC OLIGONUCLEOTIDE PRIMER
  source 1..50: mol_type = other DNA
gcacaaaaaagtgacctaaatgcgcaatcagctagtattgttaaaacaac

SEQ ID NO: 87 - DNA, 50 bp, synthetic construct
  misc_feature 1..50: note = ElxA P-2A R SYNTHETIC OLIGONUCLEOTIDE PRIMER
  source 1..50: mol_type = other DNA
gttgttttaacaatactagctgattgcgcatttaggtcacttttttgtgc

SEQ ID NO: 88 - DNA, 39 bp, synthetic construct
  misc_feature 1..39: note = ElxA N-3A F SYNTHETIC OLIGONUCLEOTIDE PRIMER
  source 1..39: mol_type = other DNA
atcgaggcacaaaaaagtgacctagcaccgcaatcagct

SEQ ID NO: 89 - DNA, 55 bp, synthetic construct
  misc_feature 1..55: note = ElxA N-3A R SYNTHETIC OLIGONUCLEOTIDE PRIMER
  source 1..55: mol_type = other DNA
acttttttgtgcctcgatatctttattaagatttaaatcaaataattcttttttc

SEQ ID NO: 90 - DNA, 39 bp, synthetic construct
  misc_feature 1..39: note = ElxA L-4A F SYNTHETIC OLIGONUCLEOTIDE PRIMER
  source 1..39: mol_type = other DNA
atcgaggcacaaaaaagtgacgcaaatccgcaatcagct

SEQ ID NO: 91 - DNA, 55 bp, synthetic construct; identical to SEQ ID NO: 89
  misc_feature 1..55: note = ElxA L-4A R SYNTHETIC OLIGONUCLEOTIDE PRIMER
  source 1..55: mol_type = other DNA

SEQ ID NO: 92 - DNA, 39 bp, synthetic construct
  misc_feature 1..39: note = ElxA D-5A F SYNTHETIC OLIGONUCLEOTIDE PRIMER
  source 1..39: mol_type = other DNA
atcgaggcacaaaaaagtgcactaaatccgcaatcagct

SEQ ID NO: 93 - DNA, 55 bp, synthetic construct; identical to SEQ ID NO: 89
  misc_feature 1..55: note = ElxA D-5A R SYNTHETIC OLIGONUCLEOTIDE PRIMER
  source 1..55: mol_type = other DNA

SEQ ID NO: 94 - DNA, 52 bp, synthetic construct
  misc_feature 1..52: note = ElxA F-19A F SYNTHETIC OLIGONUCLEOTIDE PRIMER
  source 1..52: mol_type = other DNA
ggcagccatatgaaaaaagaattagctgatttaaatcttaataaagatatcg

SEQ ID NO: 95 - DNA, 52 bp, synthetic construct
  misc_feature 1..52: note = ElxA F-19A R SYNTHETIC OLIGONUCLEOTIDE PRIMER
  source 1..52: mol_type = other DNA
cgatatctttattaagatttaaatcagctaattcttttttcatatggctgcc

SEQ ID NO: 96 - DNA, 54 bp, synthetic construct
  misc_feature 1..54: note = ElxA D-18A F SYNTHETIC OLIGONUCLEOTIDE PRIMER
  source 1..54: mol_type = other DNA
cagccatatgaaaaaagaattatttgctttaaatcttaataaagatatcgaggc

SEQ ID NO: 97 - DNA, 54 bp, synthetic construct
  misc_feature 1..54: note = ElxA D-18A R SYNTHETIC OLIGONUCLEOTIDE PRIMER
  source 1..54: mol_type = other DNA
gcctcgatatctttattaagatttaaagcaaataattcttttttcatatggctg

SEQ ID NO: 98 - DNA, 52 bp, synthetic construct
  misc_feature 1..52: note = ElxA L-17A F SYNTHETIC OLIGONUCLEOTIDE PRIMER
  source 1..52: mol_type = other DNA
gccatatgaaaaaagaattatttgatgcaaatcttaataaagatatcgaggc

SEQ ID NO: 99 - DNA, 52 bp, synthetic construct
  misc_feature 1..52: note = ElxA L-17A R SYNTHETIC OLIGONUCLEOTIDE PRIMER
  source 1..52: mol_type = other DNA
gcctcgatatctttattaagatttgcatcaaataattcttttttcatatggc

SEQ ID NO: 100 - DNA, 54 bp, synthetic construct
  misc_feature 1..54: note = ElxA N-16A F SYNTHETIC OLIGONUCLEOTIDE PRIMER
  source 1..54: mol_type = other DNA
gccatatgaaaaaagaattatttgatttagctcttaataaagatatcgaggcac

SEQ ID NO: 101 - DNA, 54 bp, synthetic construct
  misc_feature 1..54: note = ElxA N-16A R SYNTHETIC OLIGONUCLEOTIDE PRIMER
  source 1..54: mol_type = other DNA
gtgcctcgatatctttattaagagctaaatcaaataattcttttttcatatggc

SEQ ID NO: 102 - DNA, 43 bp, synthetic construct
  misc_feature 1..43: note = NisA R-1Q F SYNTHETIC OLIGONUCLEOTIDE PRIMER
  source 1..43: mol_type = other DNA
catcaccacagattacaagtatttcgctatgtacaccccgttg

SEQ ID NO: 103 - DNA, 43 bp, synthetic construct
  misc_feature 1..43: note = NisA R-1Q R SYNTHETIC OLIGONUCLEOTIDE PRIMER
  source 1..43: mol_type = other DNA
cttgtaatctgtggtgatgcacctgaatctttcttcgaaacag

SEQ ID NO: 104 - DNA, 38 bp, synthetic construct
  misc_feature 1..38: note = NisA R-1Q Q-1_1IinsA F SYNTHETIC OLIGONUCLEOTIDE PRIMER
  source 1..38: mol_type = other DNA
gattcaggtgcatcaccacaggcaattacaagtatttc

SEQ ID NO: 105 - DNA, 39 bp, synthetic construct
  misc_feature 1..39: note = NisA R-1Q Q-1_1IinsA R SYNTHETIC OLIGONUCLEOTIDE PRIMER
  source 1..39: mol_type = other DNA
tgatgcacctgaatctttcttcgaaacagataccaaatc

SEQ ID NO: 106 - DNA, 48 bp, synthetic construct
  misc_feature 1..48: note = NisA G-5D A-4L S-3N R-1Q F SYNTHETIC OLIGONUCLEOTIDE PRIMER
  source 1..48: mol_type = other DNA
gtttcgaagaaagatagcgatctgaatccacagattacaagtatttcg

SEQ ID NO: 107 - DNA, 39 bp, synthetic construct
  misc_feature 1..39: note = NisA G-5D A-4L S-3N R-1Q R SYNTHETIC OLIGONUCLEOTIDE PRIMER
  source 1..39: mol_type = other DNA
atctttcttcgaaacagataccaaatccaagttaaaatc

SEQ ID NO: 108 - DNA, 50 bp, synthetic construct
  misc_feature 1..50: note = NisA G-5D A-4L S-3N R-1Q Q-1_1linsA F SYNTHETIC OLIGONUCLEOTIDE PRIMER
  source 1..50: mol_type = other DNA
gtttcgaagaaagattcagatctgaatccgcaggcaattacaagtatttc

SEQ ID NO: 109 - DNA, 52 bp, synthetic construct
  misc_feature 1..52: note = NisA G-5D A-4L S-3N R-1Q Q-1_1linsA R SYNTHETIC OLIGONUCLEOTIDE PRIMER
  source 1..52: mol_type = other DNA
tgaatctttcttcgaaacagataccaaatccaagttaaaatcttttgtactc

SEQ ID NO: 110 - DNA, 47 bp, synthetic construct
  misc_feature 1..47: note = ElxO.S139A.FP
  source 1..47: mol_type = other DNA
ggaaatccgcttaatcctgtaatagcagaaatatttactattgctcc

SEQ ID NO: 111 - DNA, 47 bp, synthetic construct
  misc_feature 1..47: note = ElxO.S139A.RP
  source 1..47: mol_type = other DNA
ggagcaatagtaaatatttctgctattacaggattaagcggatttcc

SEQ ID NO: 112 - DNA, 45 bp, synthetic construct
  misc_feature 1..45: note = ElxO.Y152F.FP
  source 1..45: mol_type = other DNA
gcggatttccttactctatattattcggtagcacaaaacatgctg

SEQ ID NO: 113 - DNA, 45 bp, synthetic construct
  misc_feature 1..45: note = ElxO.Y152F.RP
  source 1..45: mol_type = other DNA
cagcatgttttgtgctaccgaataatatagagtaaggaaatccgc

SEQ ID NO: 114 - DNA, 56 bp, synthetic construct
  misc_feature 1..56: note = ElxO.K156A.FP
  source 1..56: mol_type = other DNA
cctttagttaaaccaataacagcatgtgctgtgctaccgtataatatagagtaagg

SEQ ID NO: 115 - DNA, 56 bp, synthetic construct
  misc_feature 1..56: note = ElxO.K156A.RP
  source 1..56: mol_type = other DNA
ccttactctatattatacggtagcacagcacatgctgttattggtttaactaaagg

SEQ ID NO: 116 - DNA, 56 bp, synthetic construct
  misc_feature 1..56: note = ElxO.K156M.FP
  source 1..56: mol_type = other DNA
cctttagttaaaccaataacagcatgcattgtgctaccgtataatatagagtaagg

SEQ ID NO: 117 - DNA, 56 bp, synthetic construct
  misc_feature 1..56: note = ElxO.K156M.RP
  source 1..56: mol_type = other DNA
ccttactctatattatacggtagcacaatgcatgctgttattggtttaactaaagg

SEQ ID NO: 118 - DNA, 2985 bp, synthetic construct
  misc_feature 1..2985: note = SYNTHETIC DNA SEQUENCE FOR cylM OPEN READING FRAME (E. COLI CODON OPTIMIZED)
  source 1..2985: mol_type = other DNA
atggaagataatctgattaatgtgctgagcattaatgaacgttgctttctgctgaaacagagcggcaaagaaaaatatgatattaaaaatctgcaggcctggaaagaacgtaaaagcgttctgaaacaggatgatctggattatctgattaaatataaatatgaaagcctggataattttggcctgggtattaccccgattgaaaattttccggataaagaagtggccattcagtatattaaagatcagagttggtatattttttttgaaagcattctggatagctataatgatagcgaagaaaaactgctggaagttgatgcaagctatccgtttcgttattttctgcagtatgcacgtctgtttctgctggatctgaatagcgaactgaatatttgcaccaaagaatttattattaatctgctggaaaccctgacccaggaactgattcatctgaccagcaaaaccctggttctggatctgcatacctttaaaaaaaatgaaccgctgaaaggcaatgatagcagcaaacgctttatttattatctgaaaaaacgctttaatagcaaaaaagatattattgccttttatacctgctatccggaactgatgcgtattaccgttgttcgtatgcgctatttcctggataacaccaaacaaatgctgattcgtgttaccgaagatctgccgagcattcagaattgctttaatattcagagcagtgaactgaatagcattagcgaaagccagggtgatagccatagccgtggtaaaaccgttagcaccctgacctttagtgatggtaaaaaaattgtgtataaaccgaaaattaatagcgaaaataaactgcgcgatttttttgaatttctgaataaagaactggaagccgatatttatattgtgaaaaaagtgacccgcaatacctatttttatgaagaatatatcgataacattgaaatcaataacatcgaagaagtgaaaaaatattacgaacgctatggcaaactgattggcattgcctttctgtttaatgttaccgatctgcattatgaaaacatcattgcccatggcgaatatccggtgattattgataatgaaaccttttttcagcagaatattccgattgaatttggtaatagcgcaaccgttgatgccaaatacaaatatctggatagcattatggtgaccggtctggttccgtatctggcaatgaaagataaaagcgatagcaaagatgaaggcgttaatctgagcgcactgaattttaaagaacagagcgtgccgtttaaaattctgaaaattaaaaatacctttaccgatgaaatgcgctttgaatatcagacccatattatggataccgcaaaaaataccccgattatgaataatgaaaaaatcagctttatcagctatgaaaaatatattgtgaccggcatgaaaagcattctgatgaaagccaaagatagcaaaaaaaaaattctggcctatattaataataatctgcagaatctgattgtgcgcaatgttattcgtccgacccagcgttatgcagatatgctggaatttagctatcatccgaattgctttagcaatgccattgaacgtgaaaaagtgctgcataatatgtgggcctatccgtataaaaataaaaaagtggtgcattatgaattttcagatctgattgatggcgatattccgattttttataataatattagcaaaaccagcctgattgccagtgatggttgtctggttgaagatttttatcaggaaagtgccctgaatcgctgcctgaataaaattaatgatctgtgtgatgaagatattagcattcagaccgtgtggctggaaattgcactgaatatttataacccgtacaaatatatcaatgatctgaaaaaccagaatagcaacaaatatatttataccggtctggaactgaatggcaaaattattcaggcctgccagaaaattgaaaaaaaaatctttaaacgtgccatctttaacaaaaaaaccaataccgtgaattggattgatattaaactggatcaggattggaatgtgggcattctgaataataatatgtatgatggcctgcctggtatttttattttttatgtggccctgaaatatattaccaaaaaccataaatatgattatgtgatcgaatgcattaaaaatagcatttataccattccgagcgaagatattctgagcgcattttttggtaaaggcagcctgatttatccgctgctggttgattatcgcctgaataatgatattaatagcctgaatgtggccgtggaaattgccgatatgctgattgaaaaaaaaccgattaataatggcgaactgaaaaatgattggattcatggccataatagcattattaaagtgctgctgctgctgagcgaaattaccgaagatgaaaaatatcgcaaatttagcctggaaatttttgaaaaactgagcgaagaaccgtattttaattttcgtggttttggccatggcatttatagctatgttcatctgctgagcaaatttaatcgcattgataaagccaatagcctgctgcataaaattaaagaaagctattttgaagaagaaccgaaaaataattcctggtgtaaaggcaccgttggtgaactgctggcaaccattgaactgtatgatgataatattagtaacatcgatattaacaaaaccattgcctataaaaataaagattgcctgtgccatggcaatgcaggcaccctggaaggtctgattcagctggcaaaaaaagatccggaaacctatcagtataaaaaaaataaactgatcagctatatgctgaatgattttgaaaaaaataataccctgaaagtggcaggcagcgaatatctggaaagcctgggtttttttgttggtattagcggtgttggttatgaactgctgcgtaatctggatagcgaaattccgaatgcactgctgtttgaactgtaataa

SEQ ID NO: 119 - DNA, 210 bp, synthetic construct
  misc_feature 1..210: note = SYNTHETIC DNA SEQUENCE FOR cylL(L)
  source 1..210: mol_type = other DNA
atggaaaatctgagcgttgttccgagctttgaagaactgagcgttgaagaaatggaagcaattcagggtagcggtgatgttcaggcagaaaccacaccggtttgtgcagttgcagcaaccgcagcagcaagcagcgcagcatgtggttgggttggtggtggtatttttaccggtgttaccgttgttgttagcctgaaacattgctaataa

SEQ ID NO: 120 - DNA, 195 bp, synthetic construct
  misc_feature 1..195: note = SYNTHETIC DNA SEQUENCE FOR cylL(S)
  source 1..195: mol_type = other DNA
atgctgaataaagaaaatcaggaaaattattatagcaataaactggaactggtgggtccgagctttgaagaactgagcctggaagaaatggaagcaattcagggtagcggtgatgttcaggcagaaaccacaccggcatgttttaccattggtctgggtgttggtgcactgtttagcgcaaaattttgctaataa

SEQ ID NO: 121 - DNA, 30 bp, synthetic construct
  misc_feature 1..30: note = SYNTHETIC OLIGONUCLEOTIDE PRIMER CylLL_EcoRI_FP_Duet
  source 1..30: mol_type = other DNA
aaaaagaattcggaaaatctgagcgttgtt

SEQ ID NO: 122 - DNA, 31 bp, synthetic construct
  misc_feature 1..31: note = SYNTHETIC OLIGONUCLEOTIDE PRIMER CylLL_NotI_RP_Duet
  source 1..31: mol_type = other DNA
aaaaagcggccgcttagcaatgtttcaggct

SEQ ID NO: 123 - DNA, 30 bp, synthetic construct
  misc_feature 1..30: note = CylLS_EcoRI_FP SYNTHETIC OLIGONUCLEOTIDE PRIMER
  source 1..30: mol_type = other DNA
aaaaagaattcgctgaataaagaaaatcag

SEQ ID NO: 124 - DNA, 31 bp, synthetic construct
  misc_feature 1..31: note = CylLS_NotI_RP SYNTHETIC OLIGONUCLEOTIDE PRIMER
  source 1..31: mol_type = other DNA
aaaaagcggccgcttagcaaaattttgcgct

SEQ ID NO: 125 - DNA, 35 bp, synthetic construct
  misc_feature 1..35: note = HalA1_SacI_FP SYNTHETIC OLIGONUCLEOTIDE PRIMER
  source 1..35: mol_type = other DNA
cgccactcggagctcgatgacaaatcttttaaaag

SEQ ID NO: 126 - DNA, 35 bp, synthetic construct
  misc_feature 1..35: note = HalA1_SbfI_RP SYNTHETIC OLIGONUCLEOTIDE PRIMER
  source 1..35: mol_type = other DNA
atagtgatcctgcaggttagttgcaagaaggcatg

SEQ ID NO: 127 - DNA, 32 bp, synthetic construct
  misc_feature 1..32: note = HalA2_BamHI_FP SYNTHETIC OLIGONUCLEOTIDE PRIMER
  source 1..32: mol_type = other DNA
aaaaaggatccgatggtaaattcaaaagattt

SEQ ID NO: 128 - DNA, 31 bp, synthetic construct
  misc_feature 1..31: note = HalA2_HindIII_RP SYNTHETIC OLIGONUCLEOTIDE PRIMER
  source 1..31: mol_type = other DNA
aaaaaaagcttttagcactggcttgtacact

SEQ ID NO: 129 - DNA, 39 bp, synthetic construct
  misc_feature 1..39: note = ProcA1.7_EcoRI_FP SYNTHETIC OLIGONUCLEOTIDE PRIMER
  source 1..39: mol_type = other DNA
ggtgcgaggaattcgatgaagcatagacaactaaatctg

SEQ ID NO: 130 - DNA, 31 bp, synthetic construct
  misc_feature 1..31: note = ProcA1.7_NotI_RP SYNTHETIC OLIGONUCLEOTIDE PRIMER
  source 1..31: mol_type = other DNA
ataatatcgcggccgctcagcacattttccc

SEQ ID NO: 131 - DNA, 38 bp, synthetic construct
  misc_feature 1..38: note = NisA_BamHI_FP SYNTHETIC OLIGONUCLEOTIDE PRIMER
  source 1..38: mol_type = other DNA
ctagatggatccgatgagtacaaaagattttaacttgg

SEQ ID NO: 132 - DNA, 37 bp, synthetic construct
  misc_feature 1..37: note = NisA_HindIII_RP SYNTHETIC OLIGONUCLEOTIDE PRIMER
  source 1..37: mol_type = other DNA
ctagaagcttttatttgcttacgtgaatactacaatg

SEQ ID NO: 133 - DNA, 33 bp, synthetic construct
  misc_feature 1..33: note = CylA27-EcoRI_FP SYNTHETIC OLIGONUCLEOTIDE PRIMER
  source 1..33: mol_type = other DNA
aaaaagaattcgctgagcaacaatatcagcttc

SEQ ID NO: 134 - DNA, 37 bp, synthetic construct
  misc_feature 1..37: note = CylA-NotI_RP SYNTHETIC OLIGONUC;LEOTIDE PRIMER
  source 1..37: mol_type = other DNA
aaaaagcggccgcttatttaatgcgcacttctttgta

SEQ ID NO: 135 - DNA, 33 bp, synthetic construct
  misc_feature 1..33: note = CylA-E95A_QC_FP SYNTHETIC OLIGONUCLEOTIDE PRIMER
  source 1..33: mol_type = other DNA
ccggattttaaaccggcagaagtgaatagcagc

SEQ ID NO: 136 - DNA, 33 bp, synthetic construct
  misc_feature 1..33: note = CylA-E95A_QC_RP SYNTHETIC OLIGONUCLEOTIDE PRIMER
  source 1..33: mol_type = other DNA
gctgctattcacttctgccggtttaaaatccgg

SEQ ID NO: 137 - DNA, 36 bp, synthetic construct
  misc_feature 1..36: note = CylA-S359A_QC_FP SYNTHETIC OLIGONUCLEOTIDE PRIMER
  source 1..36: mol_type = other DNA
ggcaccgcactggcaacaccggaagttagcgcagca

SEQ ID NO: 138 - DNA, 45 bp, synthetic construct
  misc_feature 1..45: note = CylA- S359A_QC_RP SYNTHETIC OLIGONUCLEOTIDE PRIMER
  source 1..45: mol_type = other DNA
tgccagtgcggtgccaaagctcagggtataaccatccggaaaatc

SEQ ID NO: 139 - DNA, 18 bp, synthetic construct
  misc_feature 1..18: note = HalA2-GDVQAE_FP SYNTHETIC OLIGONUCLEOTIDE PRIMER
  source 1..18: mol_type = other DNA
acaacttggccttgcgct

SEQ ID NO: 140 - DNA, 48 bp, synthetic construct
  misc_feature 1..48: note = HalA2-GDVQAE_RP SYNTHETIC OLIGONUCLEOTIDE PRIMER
  source 1..48: mol_type = other DNA
gcaaggccaagttgtttctgcctgaacatcacctgaaccagctagaga

SEQ ID NO: 141 - DNA, 21 bp, synthetic construct
  misc_feature 1..21: note = HalA1-GDVQAE_FP SYNTHETIC OLIGONUCLEOTIDE PRIMER
  source 1..21: mol_type = other DNA
tgcgcatggtacaacatcagc

SEQ ID NO: 142 - DNA, 48 bp, synthetic construct
  misc_feature 1..48: note = HalA1-GDVQAE_RP SYNTHETIC OLIGONUCLEOTIDE PRIMER
  source 1..48: mol_type = other DNA
gttgtaccatgcgcattctgcctgaacatcacctagaatatcttggtc

SEQ ID NO: 143 - DNA, 18 bp, synthetic construct
  misc_feature 1..18: note = HalA2-GDVQAE-T2A_FP SYNTHETIC OLIGONUCLEOTIDE PRIMER
  source 1..18: mol_type = other DNA
acagcttggccttgcgct

SEQ ID NO: 144 - DNA, 48 bp, synthetic construct
  misc_feature 1..48: note = HalA2-GDVQAE-T2A_RP SYNTHETIC OLIGONUCLEOTIDE PRIMER
  source 1..48: mol_type = other DNA
gcaaggccaagctgtttctgcctgaacatcacctgaaccagctagaga

SEQ ID NO: 145 - DNA, 21 bp, synthetic construct
  misc_feature 1..21: note = ProcA1.7-GDVQAE_FP SYNTHETIC OLIGONUCLEOTIDE PRIMER
  source 1..21: mol_type = other DNA
accattgggggaaccattgtg

SEQ ID NO: 146 - DNA, 53 bp, synthetic construct
  misc_feature 1..53: note = ProcA1.7-GDVQAE_RP SYNTHETIC OLIGONUCLEOTIDE PRIMER
  source 1..53: mol_type = other DNA
ggttcccccaatggtttctgcctgaacatcacccagctcagcatcagacaggt

SEQ ID NO: 147 - DNA, 21 bp, synthetic construct
  misc_feature 1..21: note = NisA-GDVQAE_FP SYNTHETIC OLIGONUCLEOTIDE PRIMER
  source 1..21: mol_type = other DNA
attacaagtatttcgctatgt

SEQ ID NO: 148 - DNA, 53 bp, synthetic construct
  misc_feature 1..53: note = NisA-GDVQAE_RP SYNTHETIC OLIGONUCLEOTIDE PRIMER
  source 1..53: mol_type = other DNA
cgaaatacttgtaatttctgcctgaacatcaccatctttcttcgaaacagata

SEQ ID NO: 149 - DNA, 45 bp, synthetic construct
  misc_feature 1..45: note = ProcA1.7-GDVQAE-T1G_QC_FP SYNTHETIC OLIGONUCLEOTIDE PRIMER
  source 1..45: mol_type = other DNA
caggcagaaggtattgggggaaccattgtgtcgataacctgtgag

SEQ ID NO: 150 - DNA, 40 bp, synthetic construct
  misc_feature 1..40: note = ProcA1.7-GDVQAE-T1G_QC_RP SYNTHETIC OLIGONUCLEOTIDE PRIMER
  source 1..40: mol_type = other DNA
cccaataccttctgcctgaacatcacccagctcagcatca

SEQ ID NO: 151 - DNA, 45 bp, synthetic construct
  misc_feature 1..45: note = ProcA1.7-GDVQAE-T1F_QC_FP SYNTHETIC OLIGONUCLEOTIDE PRIMER
  source 1..45: mol_type = other DNA
caggcagaattcattgggggaaccattgtgtcgataacctgtgag

SEQ ID NO: 152 - DNA, 40 bp, synthetic construct
  misc_feature 1..40: note = A1.7-GDVQAE-T1F_QC_RP SYNTHETIC OLIGONUCLEOTIDE PRIMER
  source 1..40: mol_type = other DNA
cccaatgaattctgcctgaacatcacccagctcagcatca

SEQ ID NO: 153 - DNA, 44 bp, synthetic construct
  misc_feature 1..44: note = ProcA1.7-GDVQAE-T1W_QC_FP SYNTHETIC OLIGONUCLEOTIDE PRIMER
  source 1..44: mol_type = other DNA
aggcagaatggattgggggaaccattgtgtcgataacctgtgag

SEQ ID NO: 154 - DNA, 40 bp, synthetic construct
  misc_feature 1..40: note = ProcA1.7-GDVQAE-T1W_QC_RP SYNTHETIC OLIGONUCLEOTIDE PRIMER
  source 1..40: mol_type = other DNA
cccaatccattctgcctgaacatcacccagctcagcatca

SEQ ID NO: 155 - DNA, 33 bp, synthetic construct
  misc_feature 1..33: note = LicP25_BamHI_FP SYNTHETIC OLIGONUCLEOTIDE PRIMER
  source 1..33: mol_type = other DNA
aaaaaggatccgaaagaacaagcaggagaacag

SEQ ID NO: 156 - DNA, 34 bp, synthetic construct
  misc_feature 1..34: note = LicP_NotI_RP SYNTHETIC OLIGONUCLEOTIDE PRIMER
  source 1..34: mol_type = other DNA
aaaaagcggccgctcactccttgttcatcatttt

SEQ ID NO: 157 - DNA, 33 bp, synthetic construct
  misc_feature 1..33: note = LicA2_BamHI_FP SYNTHETIC OLIGONUCLEOTIDE PRIMER
  source 1..33: mol_type = other DNA
aaaaaggatccgatgaaaacaatgaaaaattca

SEQ ID NO: 158 - DNA, 34 bp, synthetic construct
  misc_feature 1..34: note = LicA2_NotI_RP SYNTHETIC OLIGONUCLEOTIDE PRIMER
  source 1..34: mol_type = other DNA
aaaaagcggccgcctagcatcggcttgtacactt

SEQ ID NO: 159 - DNA, 33 bp, synthetic construct; identical to SEQ ID NO: 155
  misc_feature 1..33: note = LicP_25_BamHI_FP SYNTHETIC OLIGONUCLEOTIDE PRIMER
  source 1..33: mol_type = other DNA

SEQ ID NO: 160 - DNA, 34 bp, synthetic construct; identical to SEQ ID NO: 156
  misc_feature 1..34: note = LicP_NotI_RP SYNTHETIC OLIGONUCLEOTIDE PRIMER
  source 1..34: mol_type = other DNA

SEQ ID NO: 161 - DNA, 33 bp, synthetic construct; identical to SEQ ID NO: 157
  misc_feature 1..33: note = LicA2_BamHI_FP SYNTHETIC OLIGONUCLEOTIDE PRIMER
  source 1..33: mol_type = other DNA

SEQ ID NO: 162 - DNA, 34 bp, synthetic construct; identical to SEQ ID NO: 158
  misc_feature 1..34: note = LicA2_NotI_RP SYNTHETIC OLIGONUCLEOTIDE PRIMER
  source 1..34: mol_type = other DNA

SEQ ID NO: 163 - DNA, 32 bp, synthetic construct
  misc_feature 1..32: note = LicM2_NdeI_FP SYNTHETIC OLIGONUCLEOTIDE PRIMER
  source 1..32: mol_type = other DNA
aaaaacatatggttttcttcgccaaagggatg

SEQ ID NO: 164 - DNA, 32 bp, synthetic construct
  misc_feature 1..32: note = LicM2_KpnI_RP SYNTHETIC OLIGONUCLEOTIDE PRIMER
  source 1..32: mol_type = other DNA
aaaaaggtacctcacctgcccgtcggaatatc

SEQ ID NO: 165 - DNA, 40 bp, synthetic construct
  misc_feature 1..40: note = G-LicA2_ _QC_FP SYNTHETIC OLIGONUCLEOTIDE PRIMER
  source 1..40: mol_type = other DNA
ccaggatggtatgaaaacaatgaaaaattcagctgcccgt

SEQ ID NO: 166 - DNA, 39 bp, synthetic construct
  misc_feature 1..39: note = G-LicA2_QC_RP SYNTHETIC OLIGONUCLEOTIDE PRIMER
  source 1..39: mol_type = other DNA
gttttcataccatcctggctgtggtgatgatggtgatgg

SEQ ID NO: 167 - DNA, 39 bp, synthetic construct; identical to SEQ ID NO: 129
  misc_feature 1..39: note = ProcA1.7_EcoRI_FP SYNTHETIC OLIGONUCLEOTIDE PRIMER
  source 1..39: mol_type = other DNA

SEQ ID NO: 168 - DNA, 31 bp, synthetic construct; identical to SEQ ID NO: 130
  misc_feature 1..31: note = ProcA1.7_NotI_RP SYNTHETIC OLIGONUCLEOTIDE PRIMER
  source 1..31: mol_type = other DNA

SEQ ID NO: 169 - DNA, 38 bp, synthetic construct; identical to SEQ ID NO: 131
  misc_feature 1..38: note = NisA_BamHI_FP SYNTHETIC OLIGONUCLEOTIDE PRIMER
  source 1..38: mol_type = other DNA

SEQ ID NO: 170 - DNA, 37 bp, synthetic construct; identical to SEQ ID NO: 132
  misc_feature 1..37: note = NisA_HindIII_RP SYNTHETIC OLIGONUCLEOTIDE PRIMER
  source 1..37: mol_type = other DNA

SEQ ID NO: 171 - DNA, 30 bp, synthetic construct
  misc_feature 1..30: note = LicP-S376A_QC_FP SYNTHETIC OLIGONUCLEOTIDE PRIMER
  source 1..30: mol_type = other DNA
ggaacagcattggccgccccgcaggtagct

SEQ ID NO: 172 - DNA, 42 bp, synthetic construct
  misc_feature 1..42: note = LicP-S376A_QC_RP SYNTHETIC OLIGONUCLEOTIDE PRIMER
  source 1..42: mol_type = other DNA
ggccaatgctgttccgtatgagagggaatatccctttgggat

SEQ ID NO: 173 - DNA, 39 bp, synthetic construct
  misc_feature 1..39: note = LicP-H186A_QC_FP SYNTHETIC OLIGONUCLEOTIDE PRIMER
  source 1..39: mol_type = other DNA
acaggagccggaacacaaacagccgggatgatcaatatc

SEQ ID NO: 174 - DNA, 33 bp, synthetic construct
  misc_feature 1..33: note = LicP-H186A_QC_RP SYNTHETIC OLIGONUCLEOTIDE PRIMER
  source 1..33: mol_type = other DNA
gttccggctcctgtcggatctccggatacaggc

SEQ ID NO: 175 - DNA, 37 bp, synthetic construct
  misc_feature 1..37: note = LicP-E100A_QC_FP SYNTHETIC OLIGONUCLEOTIDE PRIMER
  source 1..37: mol_type = other DNA
cagtaaacgcaacggaatcagtcatcagcggttcgcc

SEQ ID NO: 176 - DNA, 47 bp, synthetic construct
  misc_feature 1..47: note = LicP-E100A_QC_RP SYNTHETIC OLIGONUCLEOTIDE PRIMER
  source 1..47: mol_type = other DNA
gattccgttgcgtttactgctgtatttgcaatcggcttttcaatgac

SEQ ID NO: 177 - DNA, 31 bp, synthetic construct
  misc_feature 1..31: note = LicP-E100A-E102A_QC_FP SYNTHETIC OLIGONUCLEOTIDE PRIMER
  source 1..31: mol_type = other DNA
gcaacggcatcagtcatcagcggttcgcctg

SEQ ID NO: 178 - DNA, 40 bp, synthetic construct
  misc_feature 1..40: note = LicP-E100A-E102A _QC_RP SYNTHETIC OLIGONUCLEOTIDE PRIMER
  source 1..40: mol_type = other DNA
gactgatgccgttgcgtttactgctgtatttgcaatcggc

SEQ ID NO: 179 - DNA, 21 bp, synthetic construct
  misc_feature 1..21: note = ProcA1.7_core_FP SYNTHETIC OLIGONUCLEOTIDE PRIMER
  source 1..21: mol_type = other DNA
accattgggggaaccattgtg

SEQ ID NO: 180 - DNA, 53 bp, synthetic construct
  misc_feature 1..53: note = ProcA1.7- NDVNPE _RP SYNTHETIC OLIGONUCLEOTIDE PRIMER
  source 1..53: mol_type = other DNA
ggttcccccaatggtttcaggattgacgtcattcagctcagcatcagacaggt

SEQ ID NO: 181 - DNA, 21 bp, synthetic construct
  misc_feature 1..21: note = NisA_core _FP SYNTHETIC OLIGONUCLEOTIDE PRIMER
  source 1..21: mol_type = other DNA
attacaagtatttcgctatgt

SEQ ID NO: 182 - DNA, 53 bp, synthetic construct
  misc_feature 1..53: note = NisA- NDVNPE _RP SYNTHETIC OLIGONUCLEOTIDE PRIMER
  source 1..53: mol_type = other DNA
cgaaatacttgtaatttcaggattgacgtcattatctttcttcgaaacagata

SEQ ID NO: 183 - DNA, 48 bp, synthetic construct
  misc_feature 1..48: note = NisA-NDVNPE-I1G_QC_FP SYNTHETIC OLIGONUCLEOTIDE PRIMER
  source 1..48: mol_type = other DNA
caatcctgaaggtacaagtatttcgctatgtacacccggttgtaaaac

SEQ ID NO: 184 - DNA, 54 bp, synthetic construct
  misc_feature 1..54: note = NisA-NDVNPE-I1G_QC_RP SYNTHETIC OLIGONUCLEOTIDE PRIMER
  source 1..54: mol_type = other DNA
gaaatacttgtaccttcaggattgacgtcattatctttcttcgaaacagatacc

SEQ ID NO: 185 - DNA, 48 bp, synthetic construct
  misc_feature 1..48: note = NisA-NDVNPE-I1C_QC_FP SYNTHETIC OLIGONUCLEOTIDE PRIMER
  source 1..48: mol_type = other DNA
caatcctgaatgtacaagtatttcgctatgtacacccggttgtaaaac

SEQ ID NO: 186 - DNA, 54 bp, synthetic construct
  misc_feature 1..54: note = NisA-NDVNPE-I1C_QC_RP SYNTHETIC OLIGONUCLEOTIDE PRIMER
  source 1..54: mol_type = other DNA
gaaatacttgtacattcaggattgacgtcattatctttcttcgaaacagatacc

SEQ ID NO: 187 - DNA, 50 bp, synthetic construct
  misc_feature 1..50: note = NisA-NDVNPE-I1T_QC_FP SYNTHETIC OLIGONUCLEOTIDE PRIMER
  source 1..50: mol_type = other DNA
caatcctgaaaccacaagtatttcgctatgtacacccggttgtaaaacag

SEQ ID NO: 188 - DNA, 55 bp, synthetic construct
  misc_feature 1..55: note = NisA-NDVNPE-I1T_QC_RP SYNTHETIC OLIGONUCLEOTIDE PRIMER
  source 1..55: mol_type = other DNA
gaaatacttgtggtttcaggattgacgtcattatctttcttcgaaacagatacca

SEQ ID NO: 189 - DNA, 48 bp, synthetic construct
  misc_feature 1..48: note = NisA-NDVNPE-I1L_QC_FP SYNTHETIC OLIGONUCLEOTIDE PRIMER
  source 1..48: mol_type = other DNA
caatcctgaacttacaagtatttcgctatgtacacccggttgtaaaac

SEQ ID NO: 190 - DNA, 54 bp, synthetic construct
  misc_feature 1..54: note = NisA-NDVNPE-I1L_QC_RP SYNTHETIC OLIGONUCLEOTIDE PRIMER
  source 1..54: mol_type = other DNA
gaaatacttgtaagttcaggattgacgtcattatctttcttcgaaacagatacc

SEQ ID NO: 191 - DNA, 48 bp, synthetic construct
  misc_feature 1..48: note = NisA-NDVNPE-I1F_QC_FP SYNTHETIC OLIGONUCLEOTIDE PRIMER
  source 1..48: mol_type = other DNA
caatcctgaatttacaagtatttcgctatgtacacccggttgtaaaac

SEQ ID NO: 192 - DNA, 54 bp, synthetic construct
  misc_feature 1..54: note = NisA-NDVNPE-I1F_QC_RP SYNTHETIC OLIGONUCLEOTIDE PRIMER
  source 1..54: mol_type = other DNA
gaaatacttgtaaattcaggattgacgtcattatctttcttcgaaacagatacc

SEQ ID NO: 193 - DNA, 48 bp, synthetic construct
  misc_feature 1..48: note = NisA-NDVNPE-I1W_QC_FP SYNTHETIC OLIGONUCLEOTIDE PRIMER
  source 1..48: mol_type = other DNA
caatcctgaatggacaagtatttcgctatgtacacccggttgtaaaac

SEQ ID NO: 194 - DNA, 54 bp, synthetic construct
  misc_feature 1..54: note = NisA-NDVNPE-I1W_QC_RP SYNTHETIC OLIGONUCLEOTIDE PRIMER
  source 1..54: mol_type = other DNA
gaaatacttgtccattcaggattgacgtcattatctttcttcgaaacagatacc

SEQ ID NO: 195 - DNA, 48 bp, synthetic construct
  misc_feature 1..48: note = NisA-NDVNPE-I1K_QC_FP SYNTHETIC OLIGONUCLEOTIDE PRIMER
  source 1..48: mol_type = other DNA
caatcctgaaaaaacaagtatttcgctatgtacacccggttgtaaaac

SEQ ID NO: 196 - DNA, 54 bp, synthetic construct
  misc_feature 1..54: note = NisA-NDVNPE-I1K_QC_RP SYNTHETIC OLIGONUCLEOTIDE PRIMER
  source 1..54: mol_type = other DNA
gaaatacttgttttttcaggattgacgtcattatctttcttcgaaacagatacc

SEQ ID NO: 197 - DNA, 48 bp, synthetic construct
  misc_feature 1..48: note = NisA-NDVNPE-I1E_QC_FP SYNTHETIC OLIGONUCLEOTIDE PRIMER
  source 1..48: mol_type = other DNA
caatcctgaagaaacaagtatttcgctatgtacacccggttgtaaaac

SEQ ID NO: 198 - DNA, 54 bp, synthetic construct
  misc_feature 1..54: note = NisA-NDVNPE-I1E_QC_RP SYNTHETIC OLIGONUCLEOTIDE PRIMER
  source 1..54: mol_type = other DNA
gaaatacttgtttcttcaggattgacgtcattatctttcttcgaaacagatacc

SEQ ID NO: 199 - DNA, 48 bp, synthetic construct
  misc_feature 1..48: note = MBP-BamL-NDVNPE_QC_FP SYNTHETIC OLIGONUCLEOTIDE PRIMER
  source 1..48: mol_type = other DNA
aatgacgtcaatcctgaatctggttctgagaacctgtacttccaatcc

SEQ ID NO: 200 - DNA, 36 bp, synthetic construct
  misc_feature 1..36: note = MBP-BamL-NDVNPE_QC_RP SYNTHETIC OLIGONUCLEOTIDE PRIMER
  source 1..36: mol_type = other DNA
ttcaggattgacgtcattagatccacgcggaaccag

SEQ ID NO: 201 - DNA, 40 bp, synthetic construct
  misc_feature 1..40: note = LicA2-E-1A_QC_FP SYNTHETIC OLIGONUCLEOTIDE PRIMER
  source 1..40: mol_type = other DNA
tcaatcctgcaacaactcctgctacaacctcttcttggac

SEQ ID NO: 202 - DNA, 40 bp, synthetic construct
  misc_feature 1..40: note = LicA2-E-1A_QC_RP SYNTHETIC OLIGONUCLEOTIDE PRIMER
  source 1..40: mol_type = other DNA
gagttgttgcaggattgacgtcatttcctcctaccaaagc

SEQ ID NO: 203 - DNA, 40 bp, synthetic construct
  misc_feature 1..40: note = LicA2-E-1D_QC_FP SYNTHETIC OLIGONUCLEOTIDE PRIMER
  source 1..40: mol_type = other DNA
tcaatcctgatacaactcctgctacaacctcttcttggac

SEQ ID NO: 204 - DNA, 40 bp, synthetic construct
  misc_feature 1..40: note = LicA2-E-1D_QC_RP SYNTHETIC OLIGONUCLEOTIDE PRIMER
  source 1..40: mol_type = other DNA
gagttgtatcaggattgacgtcatttcctcctaccaaagc

SEQ ID NO: 205 - DNA, 40 bp, synthetic construct
  misc_feature 1..40: note = LicA2-E-1Q_QC_FP SYNTHETIC OLIGONUCLEOTIDE PRIMER
  source 1..40: mol_type = other DNA
tcaatcctcaaacaactcctgctacaacctcttcttggac

SEQ ID NO: 206 - DNA, 40 bp, synthetic construct
  misc_feature 1..40: note = LicA2-E-1Q_QC_RP SYNTHETIC OLIGONUCLEOTIDE PRIMER
  source 1..40: mol_type = other DNA
gagttgtttgaggattgacgtcatttcctcctaccaaagc

SEQ ID NO: 207 - DNA, 40 bp, synthetic construct
  misc_feature 1..40: note = LicA2-P-2A_QC_FP SYNTHETIC OLIGONUCLEOTIDE PRIMER
  source 1..40: mol_type = other DNA
acgtcaatgctgaaacaactcctgctacaacctcttcttg

SEQ ID NO: 208 - DNA, 45 bp, synthetic construct
  misc_feature 1..45: note = LicA2-P-2A_QC_RP SYNTHETIC OLIGONUCLEOTIDE PRIMER
  source 1..45: mol_type = other DNA
ttgtttcagcattgacgtcatttcctcctaccaaagctttcaatt

SEQ ID NO: 209 - DNA, 36 bp, synthetic construct
  misc_feature 1..36: note = LicA2-N-3A_QC_FP SYNTHETIC OLIGONUCLEOTIDE PRIMER
  source 1..36: mol_type = other DNA
gacgtcgctcctgaaacaactcctgctacaacctct

SEQ ID NO: 210 - DNA, 41 bp, synthetic construct
  misc_feature 1..41: note = LicA2-N-3A_QC_RP SYNTHETIC OLIGONUCLEOTIDE PRIMER
  source 1..41: mol_type = other DNA
ttcaggagcgacgtcatttcctcctaccaaagctttcaatt

SEQ ID NO: 211 - DNA, 41 bp, synthetic construct
  misc_feature 1..41: note = LicA2-V-4A_QC_FP SYNTHETIC OLIGONUCLEOTIDE PRIMER
  source 1..41: mol_type = other DNA
aaatgacgccaatcctgaaacaactcctgctacaacctctt

SEQ ID NO: 212 - DNA, 41 bp, synthetic construct
  misc_feature 1..41: note = LicA2-V-4A_QC_RP SYNTHETIC OLIGONUCLEOTIDE PRIMER
  source 1..41: mol_type = other DNA
caggattggcgtcatttcctcctaccaaagctttcaattcc

SEQ ID NO: 213 - DNA, 42 bp, synthetic construct
  misc_feature 1..42: note = LicA2-V-4L_QC_FP SYNTHETIC OLIGONUCLEOTIDE PRIMER
  source 1..42: mol_type = other DNA
gaaatgacctgaatcctgaaacaactcctgctacaacctctt

SEQ ID NO: 214 - DNA, 44 bp, synthetic construct
  misc_feature 1..44: note = LicA2-V-4L_QC_RP SYNTHETIC OLIGONUCLEOTIDE PRIMER
  source 1..44: mol_type = other DNA
tcaggattcaggtcatttcctcctaccaaagctttcaattcctc

SEQ ID NO: 215 - DNA, 42 bp, synthetic construct
  misc_feature 1..42: note = LicA2-V-4F_QC_FP SYNTHETIC OLIGONUCLEOTIDE PRIMER
  source 1..42: mol_type = other DNA
gaaatgacttcaatcctgaaacaactcctgctacaacctctt

SEQ ID NO: 216 - DNA, 44 bp, synthetic construct
  misc_feature 1..44: note = LicA2-V-4F_QC_RP SYNTHETIC OLIGONUCLEOTIDE PRIMER
  source 1..44: mol_type = other DNA
tcaggattgaagtcatttcctcctaccaaagctttcaattcctc

SEQ ID NO: 217 - DNA, 40 bp, synthetic construct
  misc_feature 1..40: note = LicA2-D-5A_QC_FP SYNTHETIC OLIGONUCLEOTIDE PRIMER
  source 1..40: mol_type = other DNA
aggaaatgccgtcaatcctgaaacaactcctgctacaacc

SEQ ID NO: 218 - DNA, 43 bp, synthetic construct
  misc_feature 1..43: note = LicA2-D-5A_QC_RP SYNTHETIC OLIGONUCLEOTIDE PRIMER
  source 1..43: mol_type = other DNA
gattgacggcatttcctcctaccaaagctttcaattcctcttc

SEQ ID NO: 219 - DNA, 45 bp, synthetic construct
  misc_feature 1..45: note = LicA2-D-5K_QC_FP SYNTHETIC OLIGONUCLEOTIDE PRIMER
  source 1..45: mol_type = other DNA
ggaggaaataaagtcaatcctgaaacaactcctgctacaacctct

SEQ ID NO: 220 - DNA, 46 bp, synthetic construct
  misc_feature 1..46: note = LicA2-D-5K_QC_RP SYNTHETIC OLIGONUCLEOTIDE PRIMER
  source 1..46: mol_type = other DNA
aggattgactttatttcctcctaccaaagctttcaattcctcttcg

SEQ ID NO: 221 - protein, 694 aa, synthetic construct
  REGION 1..694: note = Recombinant polypeptide sequence
MGSSHHHHHHSSGLVPRGSHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSHHHHHHANSVPLVPRNDVNPESGSENLYFQGAAAEIETIVRESEANRIQAQTWFSHPEKSKVSFRYDERETSSIRSISIETFLSFYSSKFNREPYSVLDIGCGQGQVIQYLNSRFQKIELTGIDSSAQAISSAKKLGINASFICSNAENIMQYVSKKQDIIFIHLCFGLFKNPIAIVNTLIHLLSDQSCIYIVDLDRNSLGEGLNTAQSREEEAYLKDQYRASLTMEEFKQLLHVVTKEQHGVSFHVGNSFIGGFDETSSQFFSLMRNRNLQDALRTSVGEQLKQSQMPALLHGWIIKNKRYT

SEQ ID NO: 222 - protein, 56 aa, synthetic construct
  REGION 1..56: note = SYNTHETIC SEQUENCE
  SITE 25: note = MISC_FEATURE - Xaa is dehydroalanine.
  SITE 27: note = MISC_FEATURE - Xaa is dehydroalanine.
  REGION 31..32: note = MISC_FEATURE - Xaa is dehydrobutyrine.
  SITE 44: note = MISC_FEATURE - Xaa is 2-aminobutyric acid.
  SITE 46: note = MISC_FEATURE - Xaa is 2-aminobutyric acid.
  SITE 52: note = MISC_FEATURE - Xaa is dehydrobutyrine.
MKKELFDLNLNKDIEAQKSDLNPQXAXIVKXXIKAAKKLARGFXLXAGAHFXGKKQ

SEQ ID NO: 223 - protein, 31 aa, synthetic construct
  REGION 1..31: note = SYNTHETIC SEQUENCE
  SITE 1: note = MISC_FEATURE - Xaa is dehydroalanine.
  SITE 3: note = MISC_FEATURE - Xaa is dehydroalanine.
  REGION 7..8: note = MISC_FEATURE - Xaa is dehydrobutyrine.
  SITE 20: note = MISC_FEATURE - Xaa is 2-aminobutyric acid.
  SITE 22: note = MISC_FEATURE - Xaa is 2-aminobutyric acid.
  SITE 28: note = MISC_FEATURE - Xaa is dehydrobutyrine.
XAXIVKXXIKAAKKLARGFXLXAGAHFXGKK

SEQ ID NO: 224 - protein, 68 aa, synthetic construct
  REGION 1..68: note = SYNTHETIC SEQUENCE
MENLSVVPSFEELSVEEMEAIQGSGDVQAETTPVCAVAATAAASSAACGWVGGGIFTGVTVVVSLKHC

SEQ ID NO: 225 - protein, 63 aa, synthetic construct
  REGION 1..63: note = SYNTHETIC SEQUENCE
MLNKENQENYYSNKLELVGPSFEELSLEEMEAIQGSGDVQAETTPACFTIGLGVGALFSAKFC

SEQ ID NO: 226 - protein, 32 aa, synthetic construct
  REGION 1..32: note = SYNTHETIC SEQUENCE
TTPATTSSWTCITAGVTVSASLCPTTKCSSRC

SEQ ID NO: 227 - protein, 32 aa, synthetic construct
  REGION 1..32: note = SYNTHETIC SEQUENCE
  REGION 1..2: note = MISC_FEATURE - Xaa is dehydrobutyrine.
  REGION 5..6: note = MISC_FEATURE - Xaa is dehydrobutyrine.
  REGION 7..8: note = MISC_FEATURE - Xaa is dehydroalanine.
  SITE 13: note = MISC_FEATURE - Xaa is dehydrobutyrine.
  SITE 17: note = MISC_FEATURE - Xaa is dehydrobutyrine.
  SITE 19: note = MISC_FEATURE - Xaa is dehydroalanine.
  REGION 25..26: note = MISC_FEATURE - Xaa is dehydrobutyrine.
  SITE 29: note = MISC_FEATURE - Xaa is dehydroalanine.
XXPAXXXXWTCIXAGVXVXASLCPXXKCXSRC

SEQ ID NO: 228 - protein, 32 aa, synthetic construct
  REGION 1..32: note = SYNTHETIC SEQUENCE
  REGION 1..2: note = MISC_FEATURE - Xaa is dehydrobutyrine.
  REGION 5..6: note = MISC_FEATURE - Xaa is dehydrobutyrine.
  REGION 7..8: note = MISC_FEATURE - Xaa is dehydroalanine.
  SITE 13: note = MISC_FEATURE - Xaa is dehydrobutyrine.
  SITE 17: note = MISC_FEATURE - Xaa is dehydrobutyrine.
  SITE 25: note = MISC_FEATURE - Xaa is 2-aminobutyric acid.
  SITE 26: note = MISC_FEATURE - Xaa is dehydrobutyrine.
XXPAXXAXWTCIXAGVXVAASLCPXXKCASRC

SEQ ID NO: 229 - protein, 32 aa, synthetic construct
  REGION 1..32: note = SYNTHETIC SEQUENCE
  SITE 1: note = MISC_FEATURE - Xaa is 2-oxobutyryl group.
  SITE 2: note = MISC_FEATURE - Xaa is dehydrobutyrine.
  REGION 5..6: note = MISC_FEATURE - Xaa is dehydrobutyrine.
  SITE 8: note = MISC_FEATURE - Xaa is dehydroalanine.
  SITE 13: note = MISC_FEATURE - Xaa is dehydrobutyrine.
  SITE 17: note = MISC_FEATURE - Xaa is dehydrobutyrine.
  SITE 25: note = MISC_FEATURE - Xaa is 2-aminobutyric acid.
  SITE 26: note = MISC_FEATURE - Xaa is dehydrobutyrine.
  SITE 29: note = MISC_FEATURE - Xaa is 2-aminobutyric acid.
XXPAXXAXWTAIXAGVXVAASLAPXXKAXSRA

SEQ ID NO: 230 - protein, 32 aa, synthetic construct
  REGION 1..32: note = SYNTHETIC SEQUENCE
  SITE 1: note = MISC_FEATURE - Xaa is 2-oxobutyryl group.
  SITE 3: note = MISC_FEATURE - Xaa is 2-aminobutyric acid.
  SITE 5: note = MISC_FEATURE - Xaa is dehydroalanine.
  SITE 6: note = MISC_FEATURE - Xaa is dehydrobutyrine.
  SITE 22: note = MISC_FEATURE - Xaa is 2-aminobutyric acid.
  SITE 24: note = MISC_FEATURE - Xaa is 2-aminobutyric acid.
XIXLXXAAILAKPLGNNGYLAXVXKEAMPSAN

SEQ ID NO: 231 - protein, 74 aa, synthetic construct
  REGION 1..74: note = SYNTHETIC SEQUENCE
MSKKEMILSWKNPMYRTESSYHPAGNILKELQEEEQHSIAGGTITLSTCAILSKPLGNNGYLCTVTKECMPSCN

SEQ ID NO: 232 - protein, 72 aa, synthetic construct
  REGION 1..72: note = SYNTHETIC SEQUENCE
MKTMKNSAAREAFKGANHPAGMVSEEELKALVGGNDVNPETTPATTSSWTCITAGVTVSASLCPTTKCTSRC

SEQ ID NO: 233 - protein, 23 aa, synthetic construct
  REGION 1..23: note = SYNTHETIC SEQUENCE
KDFNLDLVSVSKKDSGASPRITS

SEQ ID NO: 234 - protein, 23 aa, synthetic construct
  REGION 1..23: note = SYNTHETIC SEQUENCE
KDFNLDLVSVSKTDSGASTRITS

SEQ ID NO: 235 - protein, 23 aa, synthetic construct
  REGION 1..23: note = SYNTHETIC SEQUENCE
KDFNLDLVEVSKSNTGASTRITS

SEQ ID NO: 236 - protein, 25 aa, synthetic construct
  REGION 1..25: note = SYNTHETIC SEQUENCE
DLFDLDVKVNAKESNDSGAEPRIAS

SEQ ID NO: 237 - protein, 25 aa, synthetic construct
  REGION 1..25: note = SYNTHETIC SEQUENCE
ELFDLDVKVNAKESNDSGAEPRIAS

SEQ ID NO: 238 - protein, 26 aa, synthetic construct
  REGION 1..26: note = SYNTHETIC SEQUENCE
NVLDLDVQVKAKNDTSDSAGDERITS

SEQ ID NO: 239 - protein, 26 aa, synthetic construct
  REGION 1..26: note = SYNTHETIC SEQUENCE
KVLDLDVQVKGNNNTNDSAGDERITS

SEQ ID NO: 240 - protein, 24 aa, synthetic construct
  REGION 1..24: note = SYNTHETIC SEQUENCE
EDFNLDLIKISKENNSGASPRITS

SEQ ID NO: 241 - protein, 24 aa, synthetic construct
  REGION 1..24: note = SYNTHETIC SEQUENCE
ELFDLNLNKDIEAQKSDLNPQSAS

SEQ ID NO: 242 - protein, 24 aa, synthetic construct
  REGION 1..24: note = SYNTHETIC SEQUENCE
DLFDLEIKKDNMENNNELEAQSLG

SEQ ID NO: 243 - protein, 24 aa, synthetic construct
  REGION 1..24: note = SYNTHETIC SEQUENCE
NLFDLEIKKETSQNDTELEPQTAG

SEQ ID NO: 244 - protein, 23 aa, synthetic construct
  REGION 1..23: note = SYNTHETIC SEQUENCE
NIFDLDINKKMESTSEVSAQTWA

SEQ ID NO: 245 - protein, 23 aa, synthetic construct
  REGION 1..23: note = SYNTHETIC SEQUENCE
KDFNLDLVSVSKKDSGASPQITS

SEQ ID NO: 246 - protein, 24 aa, synthetic construct
  REGION 1..24: note = SYNTHETIC SEQUENCE
KDFNLDLVSVSKKDSGASPQAITS

SEQ ID NO: 247 - protein, 23 aa, synthetic construct
  REGION 1..23: note = SYNTHETIC SEQUENCE
KDFNLDLVSVSKKDSDLNPQITS

SEQ ID NO: 248 - protein, 24 aa, synthetic construct
  REGION 1..24: note = SYNTHETIC SEQUENCE
KDFNLDLVSVSKKDSDLNPQAITS

SEQ ID NO: 249 - protein, 36 aa, synthetic construct
  REGION 1..36: note = SYNTHETIC SEQUENCE
TPVLASVAVSMELLPTASVLYSDVAGCFKYSAKHHC

SEQ ID NO: 250 - protein, 65 aa, synthetic construct
  REGION 1..65: note = SYNTHETIC SEQUENCE
MVNSKDLRNPEFRKAQGLQFVDEVNEKELSSLAGSGDVHAQTTWPCATVGVSVALCPTTKCTSQC

SEQ ID NO: 251 - protein, 69 aa, synthetic construct
  REGION 1..69: note = SYNTHETIC SEQUENCE
MTNLLKEWKMPLERTHNNSNPAGDIFQELEDQDILAGVNGACAWYNISCRLGNKGAYCTLTVECMPSCN

SEQ ID NO: 252 - protein, 98 aa, synthetic construct
  REGION 1..98: note = SYNTHETIC SEQUENCE
MKHRQLNLMSEEQLKAFIAKVQADTSLQEQLKVEGADVVAIAKASGFAITTEDLKAHQANSQKNLSDAELEGVAGGTIGGTIVSITCETCDLLVGKMC

SEQ ID NO: 253 - protein, 57 aa, synthetic construct
  REGION 1..57: note = SYNTHETIC SEQUENCE
MSTKDFNLDLVSVSKKDSGASPRITSISLCTPGCKTGALMGCNMKTATCHCSIHVSK

SEQ ID NO: 254 - protein, 28 aa, synthetic construct
  REGION 1..28: note = SYNTHETIC SEQUENCE
NDVNPETIGGTIVSITCETCDLLVGKMC

SEQ ID NO: 255 - protein, 40 aa, synthetic construct
  REGION 1..40: note = SYNTHETIC SEQUENCE
NDVNPEITSISLCTPGCKTGALMGCNMKTATCHCSIHVSK

SEQ ID NO: 256 - protein, 40 aa, synthetic construct
  REGION 1..40: note = SYNTHETIC SEQUENCE
NDVNPEETSISLCTPGCKTGALMGCNMKTATCHCSIHVSK

SEQ ID NO: 257 - protein, 40 aa, synthetic construct
  REGION 1..40: note = SYNTHETIC SEQUENCE
NDVNPEFTSISLCTPGCKTGALMGCNMKTATCHCSIHVSK

SEQ ID NO: 258 - protein, 40 aa, synthetic construct
  REGION 1..40: note = SYNTHETIC SEQUENCE
NDVNPEKTSISLCTPGCKTGALMGCNMKTATCHCSIHVSK

SEQ ID NO: 259 - protein, 40 aa, synthetic construct
  REGION 1..40: note = SYNTHETIC SEQUENCE
NDVNPELTSISLCTPGCKTGALMGCNMKTATCHCSIHVSK

SEQ ID NO: 260 - protein, 40 aa, synthetic construct
  REGION 1..40: note = SYNTHETIC SEQUENCE
NDVNPEWTSISLCTPGCKTGALMGCNMKTATCHCSIHVSK

SEQ ID NO: 261 - protein, 40 aa, synthetic construct
  REGION 1..40: note = SYNTHETIC SEQUENCE
NDVNPEGTSISLCTPGCKTGALMGCNMKTATCHCSIHVSK

SEQ ID NO: 262 - protein, 40 aa, synthetic construct
  REGION 1..40: note = SYNTHETIC SEQUENCE
NDVNPECTSISLCTPGCKTGALMGCNMKTATCHCSIHVSK

SEQ ID NO: 263 - protein, 40 aa, synthetic construct
  REGION 1..40: note = SYNTHETIC SEQUENCE
NDVNPETTSISLCTPGCKTGALMGCNMKTATCHCSIHVSK

SEQ ID NO: 264 - protein, 68 aa, synthetic construct
  REGION 1..68: note = SYNTHETIC SEQUENCE
MSKIEAWKNPVARMNSQIVSPAGDLMDELSDSEMEMLAGGCEWYNISCQLGNKGQWCTLTKECQRSCK

SEQ ID NO: 265 - protein, 71 aa, synthetic construct
  REGION 1..71: note = SYNTHETIC SEQUENCE
MSHREMAAIYRDANKRANLEFSNPVGEVNEEELKNLAGAADVTPHTTPSSLPCGTLVTAVWCPSNACTSDC

SEQ ID NO: 266 - protein, 73 aa, synthetic construct
  REGION 1..73: note = SYNTHETIC SEQUENCE
MSELSMEKVVGETFEDLSIAEMTMVQGSGDINGEFTTSPACVYSVMVVSKASSAKCAAGASAVSGAILSAIRC

SEQ ID NO: 267 - protein, 59 aa, synthetic construct
  REGION 1..59: note = SYNTHETIC SEQUENCE
MSESNMKKVVGETFEDLSIAEMTKVQGSGDVMPESTPICAGFATLMSSIGLVKTIKGKC

SEQ ID NO: 268 - protein, 114 aa, synthetic construct
  REGION 1..114: note = SYNTHETIC SEQUENCE
MLKICNFAKLSKKFKSCKFITVENNKQINKKNERMNEMSKGYKFTKEELVEAWKDPQVREKLKDLPKHPSGKALNELSEEELAEIQGASDVQPETTPLCVGVIIGLTTSIKICK

SEQ ID NO: 269 - protein, 77 aa, synthetic construct
  REGION 1..77: note = SYNTHETIC SEQUENCE
MSKGYKFTKEELVEAWKDPQVREKLKDLPKHPSGKALNELSEEELAEIQGASDVQPETTPLCVGVIIGLTTSIKICK

SEQ ID NO: 270 - protein, 77 aa, synthetic construct
  REGION 1..77: note = SYNTHETIC SEQUENCE
MSKGYKFTKEELVEAWKDPQVREKLNDLPKHPSGKALNELSEEELAEIQGASDVQPETTPLCVGVIIGLTTSIKICK

SEQ ID NO: 271 - protein, 77 aa, synthetic construct
  REGION 1..77: note = SYNTHETIC SEQUENCE
MSKGYKFTKEELVEAWKDPQVREKLKDLPKHPSGKALNELSEEELAEIQGASDVQPETTPLCVGVIIGLTASIKICK

SEQ ID NO: 272 - protein, 73 aa, synthetic construct
  REGION 1..73: note = SYNTHETIC SEQUENCE
MSSKKVVESWKNPVLRSKNEDAPSHPAGEVDSKEIKELFGAGEGDVTPEGLSSWLGNKGGYCTLTKECMPSCN

SEQ ID NO: 273 - protein, 66 aa, synthetic construct
  REGION 1..66: note = SYNTHETIC SEQUENCE
MSKNEKLNKLRDQEFDTKELIGSVDENDLKQVAGAGDVNPETTPATPTIVAVSLGICPTTKCTSKC

SEQ ID NO: 274 - protein, 70 aa, synthetic construct
  REGION 1..70: note = SYNTHETIC SEQUENCE
MSKNEKLNKLRNQEFDTKELIGLVDENELKKVAGAGDVNPEIMSDPVTLTIVVPIFTNKLCPTVPCTLKG

SEQ ID NO: 275 - protein, 65 aa, synthetic construct
  REGION 1..65: note = SYNTHETIC SEQUENCE
MSKNEKLNKLRNQDLDTKELIGLIDENELKKIAGAGDVNPEIAPTPHTVTTIKFTTVCPITHSLV

SEQ ID NO: 276 - protein, 84 aa, synthetic construct
  REGION 1..84: note = SYNTHETIC SEQUENCE
MMNPDVVIQAWKDPGFRCGLAGTPAHPAGEAMERLDETELGAIYGGDVNADTTPATPWIVASSEACGFVIATGVIWALSQFKAC

SEQ ID NO: 277 - protein, 75 aa, synthetic construct
  REGION 1..75: note = SYNTHETIC SEQUENCE
MTREEIIRAWKSPEQRAAVALAAHPSGAAMSELDEIALQEVIGRGDVQAESTPLCAFGVGFLVGAGIGLVSVAKC

SEQ ID NO: 278 - protein, 90 aa, synthetic construct
  REGION 1..90: note = SYNTHETIC SEQUENCE
MVQNEAIILGWKNPALRNTLPAAVAHPAGDALAGLSEEELLAVYGGGDVQAETTPICYTAATVTASSAACAGVGAGIVAGVTIVLTIKRC

SEQ ID NO: 279 - protein, 72 aa, synthetic construct
  REGION 1..72: note = SYNTHETIC SEQUENCE
MREEIIRAWKNPERRTADAAAHPSGTSLMELTDGDLAFVQGAGDVNAESMTPGTITIPIWIYFGHRYSILFC

SEQ ID NO: 280 - protein, 51 aa, synthetic construct
  REGION 1..51: note = SYNTHETIC SEQUENCE
MEKIVGKAFEELTEEEMKKMQGSGDVQPETTPICGFTIGIGIGALASVKWC

SEQ ID NO: 281 - protein, 67 aa, synthetic construct
  REGION 1..67: note = SYNTHETIC SEQUENCE
MEKIIGQSFEELSKKEMEAMQGSGDVQAETTPVCAISATVAASSAACAGVGAGIGTGITIVLTFKRC

SEQ ID NO: 282 - protein, 67 aa, synthetic construct
  REGION 1..67: note = SYNTHETIC SEQUENCE
MEKIIGQSFEELSKKEMEAMQGSGDVQVETTPVCAISATVAASSAACAGVGAGIGTGITIVLTFKRC

SEQ ID NO: 283 - protein, 51 aa, synthetic construct
  REGION 1..51: note = SYNTHETIC SEQUENCE
MEEIVGKAFEELTEEEMKNMQGSGDVQPETTPLCGFTIGIGIGALVSVKWC

SEQ ID NO: 284 - protein, 66 aa, synthetic construct
  REGION 1..66: note = SYNTHETIC SEQUENCE
MNRNQIIEELAENHPAGAKLVEVSKDELSRTYGGGDVQPETSPACAVGGAVAGGLWVAHTVSYWNC

SEQ ID NO: 285 - protein, 66 aa, synthetic construct
  REGION 1..66: note = SYNTHETIC SEQUENCE
MNRNQIIEELAANHPAGAKLVEVSKDELARTYGGGDVQPETTPGCGIAAGITIAAWGAHQLSYYNC

SEQ ID NO: 286 - protein, 62 aa, synthetic construct
  REGION 1..62: note = SYNTHETIC SEQUENCE
MNRNQVIEELSVKHPAGAKLVEVSKEELTRISGGGDVQPETTPLCAFGGGVALGLSLSKVYC

SEQ ID NO: 287 - protein, 64 aa, synthetic construct
  REGION 1..64: note = SYNTHETIC SEQUENCE
MNRNQVIEELAVNHPAGAKLVEVSREELTRVYGGGDVQAETTPMTPTLYLNGITIGLALSKQSC

SEQ ID NO: 288 - protein, 77 aa, synthetic construct
  REGION 1..77: note = SYNTHETIC SEQUENCE
MVKKFKFTKEELVAAWKDPQVREKSKDLPNHPSGKVLNELSEEELAEVQGASDVQVATTPMCVRLTVLPATSIKLCK

SEQ ID NO: 289 - protein, 77 aa, synthetic construct
  REGION 1..77: note = SYNTHETIC SEQUENCE
MSKEYKFTKEELVEAWKDPQVREKLKDLPKHPSGKALNELSEEELAEIQGASDVQPETTPLCVGVIIGLTTSIKICK

SEQ ID NO: 290 - protein, 77 aa, synthetic construct; identical to SEQ ID NO: 269
  REGION 1..77: note = SYNTHETIC SEQUENCE

SEQ ID NO: 291 - protein, 77 aa, synthetic construct
  REGION 1..77: note = SYNTHETIC SEQUENCE
MSKGYKFTKEELVEAWKGPQVREKLKDLPKHPSGKALNELSEEELAEIQGASDVQPETTPLCVGVIIGLTTSIKICK

SEQ ID NO: 292 - protein, 77 aa, synthetic construct
  REGION 1..77: note = SYNTHETIC SEQUENCE
MSREYKFTKEELVEAWKDPQVREKLKDLPKHPSGKALNELSEEELAEIQGASDVQPETTPLCVGVIIGITASIKICK

SEQ ID NO: 293 - protein, 69 aa, synthetic construct
  REGION 1..69: note = SYNTHETIC SEQUENCE
MSREEKINALKNPESRNHEMENPVGKTMSELSSEELAGIQGASDVNAETTPLCIAASIGATIIFSMRNC

SEQ ID NO: 294 - protein, 71 aa, synthetic construct
  REGION 1..71: note = SYNTHETIC SEQUENCE
MTKQQIIDAWKNPEIRDGIEKVTPHPSGKAFNELTMDELAEVQGASDVAPETTPACVAAFATGMLFSIKYC

SEQ ID NO: 295 - protein, 66 aa, synthetic construct
  REGION 1..66: note = SYNTHETIC SEQUENCE
MRNLKEDVVGLSMKKLNTLEMEKIYGASGVDTRTHSMAIVATTTTRTVGQTGPVISRVLSLNTICL

SEQ ID NO: 296 - protein, 51 aa, synthetic construct
  REGION 1..51: note = SYNTHETIC SEQUENCE
MKNISEKSVGLSMKRLDTTEMEKIYGASGVDTRTSSKACLSGVSAISGLLS

SEQ ID NO: 297 - protein, 65 aa, synthetic construct
  REGION 1..65: note = SYNTHETIC SEQUENCE
MKNISEKSVGLSMKKLDATEMEKIYGASGVDTRTHPTVVVVSRASSKFCVTVAASAVLSYNMNKC

SEQ ID NO: 298 - protein, 68 aa, synthetic construct
  REGION 1..68: note = SYNTHETIC SEQUENCE
MKNISEKSVGLSMKKLDTTEMEKIYGASGVDTRTHPTVIVVSRASSKACLSGVSAISGLLSYNKDCIG

SEQ ID NO: 299 - protein, 62 aa, synthetic construct
  REGION 1..62: note = SYNTHETIC SEQUENCE
MKNISEKSVGLSMKKLDTTEMEKIYGASGVDPRTTPSPLLASFVASYIASAQYRCGKDNKGK

SEQ ID NO: 300 - protein, 67 aa, synthetic construct
  REGION 1..67: note = SYNTHETIC SEQUENCE
MRNLKEDVVGLSMKKLNTLEMEKIYGASGVDTRTHSMAIVATTTTRTVGQTGPVTISKVLSLNTICL

SEQ ID NO: 301 - protein, 68 aa, synthetic construct
  REGION 1..68: note = SYNTHETIC SEQUENCE
MKNISEKSVGLSMKRLDTTEMEKIYGASGVDTRTHPTVIVVSRTSSKACLSGVSAISGLLSYNKDCIG

SEQ ID NO: 302 - protein, 65 aa, synthetic construct; identical to SEQ ID NO: 27
  REGION 1..65: note = SYNTHETIC SEQUENCE

SEQ ID NO: 303 - protein, 62 aa, synthetic construct
  REGION 1..62: note = SYNTHETIC SEQUENCE
MKNISEKSVGLSMKKLDTTEMEKIYGASGVDPRTTPSPLLASFVASYIASAEFRCGKDNKGK

SEQ ID NO: 304 - protein, 62 aa, synthetic construct
  REGION 1..62: note = SYNTHETIC SEQUENCE
MSTLKENVVGLSMKKLDTTEMEKIYGASGVDPRTTPSPLLASFVASYIASAQYRCGKDNKGK

(19) United States
(12) Patent Application Publication
(10) Pub. No.: US 2024/0018501 A1
(43) Pub. Date: Jan. 18, 2024

(54) HIGHER PERFORMANCE PROTEASES FOR SCARLESS PEPTIDE TAG REMOVAL

(71) Applicant: The Board of Trustees of the University of Illinois, Urbana, IL (US)

(72) Inventors: Wilfred A. van der Donk, Urbana, IL (US); Weixin Tang, Somerville, MA (US)

(21) Appl. No.: 18/070,554

(22) Filed: Nov. 29, 2022

Related U.S. Application Data
(63) Continuation of application No. 15/311,188, filed on Mar. 3, 2017, now abandoned.

(51) Int. Cl. C12N 9/52; C07K 14/195; C07K 14/315; C07K 14/32; C12P 21/06; C12P 21/02; C07K 14/31; C12N 15/62

(52) U.S. Cl. CPC C12N 9/52; C07K 14/195; C07K 14/315; C07K 14/32; C12P 21/06; C12P 21/02; C07K 14/31; C12N 15/62; C12Y 304/21; C07K 2319/50; C07K 2319/20; C07K 2319/01; C07K 2319/40; C07K 2319/70; C07K 2319/21; C07K 2319/24

(57) ABSTRACT

An isolated nucleic acid that includes an open reading frame encoding a lanthipeptide protease polypeptide for scarless tag removal from a polypeptide is presented. Reagents, expression constructs and methods are also provided for preparing a scarless tag polypeptide product from a tagged polypeptide precursor containing a lanthipeptide protease cleavage site. The reagents are directed to novel lanthipeptide proteases and expression constructs and polypeptide precursors that include highly specific lanthipeptide protease substrate recognition sequence. Methods are provided that enable scarless tag removal from a cognate lanthipeptide, a non-cognate lanthipeptide or a heterologous polypeptide that includes extraneous amino acid sequences, such as leader peptides and tags.

### CROSS-REFERENCE TO RELATED APPLICATIONS

The present application is a continuation of U.S. patent application Ser. No. 15/311,188, filed Mar. 3, 2017, which is the U.S. National Stage Application of PCT/US15/30437, filed May 12, 2015, which claims benefit of priority under 35 U.S.C. 119 to U.S. provisional patent application Ser. No. 61/992,193, filed May 12, 2014, and entitled “HIGHER PERFORMANCE PROTEASES FOR SCARLESS TAG REMOVAL,” the contents of which are herein incorporated by reference in its entirety.

### STATEMENT REGARDING FEDERALLY SPONSORED RESEARCH OR DEVELOPMENT

This invention was made with government support under R01GM58822 and 5T32GM070421 awarded by the National Institutes of Health. The government has certain rights in the invention.

### SEQUENCE LISTING

The instant application contains a Sequence Listing that has been submitted in XML format via Patent Centrer and is hereby incorporated by reference in its entirety. Said XML copy, created on Sep. 25, 2023, is named UIU01-015-PCT.ST25.txt, and is 156,356 bytes in size.

### FIELD OF THE INVENTION

This invention pertains to post-translational processing enzymes and methods for preparing a polypeptide product from a polypeptide precursor containing a lanthipeptide protease cleavage site.

### BACKGROUND OF THE INVENTION

The use of proteases has led to advances in analytical chemistry, proteomics, medicine, and the food, detergent and leather industries. Sequence-specific proteases are of particular interest as precise cleavage of proteins or peptides has great utility in many settings. Although much effort has been spent on engineering of proteases with desired sequence specificity using both rational design and high throughput screening, few such efforts have reached the stage of commercial applications. Major challenges are the sacrifice of efficiency and stability when engineering new substrate specificity, or the loss of sequence specificity when focusing on improving protein robustness. Thus, nature is still the major source of proteases with novel recognition sequences. The biosynthetic machinery responsible for the production of natural products known as ribosomally synthesized and post-translationally modified peptides (RiPPs) is a promising area for discovering new sequence-specific proteases, as dedicated proteolytic enzymes are employed to remove leader peptides with highly diverse P and P′ positions.

Lanthipeptides are (methyl)lanthionine-containing peptides that belong to the growing class of RiPPs. Similar to most RiPPs, lanthipeptides are synthesized as a precursor peptide (LanA) composed of an N-terminal leader peptide and a C-terminal core region harboring the different post-translational modification sites. The (methyl)lanthionine residues in lanthipeptides are installed in a two-step biosynthetic process. First, a lanthipeptide dehydratase catalyzes the elimination of water from Ser and Thr residues in the core region to yield dehydroalanine (Dha) and dehydrobutyrine (Dhb), respectively. A lanthipeptide cyclase then catalyzes the Michael type addition of cysteinyl thiols onto the dehydroamino acids. Following the modifications of the C-terminal core peptide, the modified precursor peptide is usually processed by a lanthipeptide peptidase that removes the N-terminal leader peptide (FIG. 1A). Failure to remove the leader peptide usually results in a final product devoid of biological activity. In the case of epilancin 15X, a lantibiotic produced by Staphylococcus epidermidis 15X154 that is remarkably potent against antibiotic-resistant strains of S. aureus and Enterococcus faecalis, leader peptide removal exposes an N-terminal Dha on the post-translationally modified core peptide. This Dha hydrolyzes to the corresponding pyruvyl group (Pyr), and the short chain dehydrogenase ElxO then reduces the ketone of the Pyr group to generate an N-terminal lactyl moiety (Lac) in the final step of maturation (FIG. 1B).

Lanthipeptides are classified into four classes (class I-IV) on the basis of differences in the biosynthetic machinery responsible for installing the (methyl)lanthionines (FIG. 1C). For class I lanthipeptides, the dehydration and cyclization reactions are catalyzed by separate enzymes named LanB and LanC, whereas for class II lanthipeptides the two reactions are carried out by a single bi-functional enzyme LanM. Class III and IV lanthionine synthetases are trifunctional enzymes with Ser/Thr kinase, phosphoThr/phosphoSer lyase, and cyclase domains.

Compared to the well-characterized lanthionine synthetases, the proteases responsible for leader peptide removal to generate mature lanthipeptides are much less studied. Three different types of proteases have been reported, including the subtilisin-like LanP proteases found in both class I and class II lanthipeptide biosynthesis (e.g. NisP for nisin, EpiP for epidermin, ElxP for epilancin 15X, CytA for cytolysin, LicP for lichenicidin, and CerP for cerecidins), the cysteine protease domain in bi-functional LanT transporter proteins encountered in class II lanthipeptide biosynthesis (e.g. LctT for lacticin 481 and NukT for nukacin), and a prolyloligopeptidase-type protease identified for the biosynthesis of the class III lanthipeptide flavipeptin (FIG. 1D). The papain-like cysteine protease domain of LanT proteins typically cleaves the amide bonds after a double Gly-type motif at the C terminus of the leader peptide. In contrast to the LanT protease domain, much less is known about the substrate specificity of LanP proteases. Thus far, the only LanP protease characterized in vitro with respect to substrate specificity is ElxP involved in the biosynthesis of the class I lantibiotic epilancin 15X. In contrast to LanT protease domains, the sequence specificity of subtilisin-like LanP proteins remains mostly elusive. Thus far, only two class I LanP proteases have been heterologously expressed and characterized in vitro, whereas no such studies have been performed for class II LanPs, which fall into a different phylogenetic clade.

Although most class II lanthipeptides employ LanT proteins for leader peptide removal, a few use LanP proteases. Most of these LanPs appear to remove short N-terminal oligopeptides after LanT proteins remove the majority of leader peptides at a double Gly-type cleavage site. For example, CylA is an extracellular serine protease required for biosynthesis of the enterococcal cytolysin. After installation of the thioether rings in the precursor peptides CylL_{L} and CylL_{S}, Cyl_{B} (a LanT protein) removes the majority of their leader peptides to generate CylL_{L}′ and CylL_{S}′ (FIG. 1D). CylA then trims these peptides further by removal of six amino acids at the N-terminus, resulting in CylL_{L}″ and CylL_{S}″ (cytolysin L and S) as the two units that make up mature cytolysin. In another example, LicP is an extracellularly located serine protease expressed by some strains of Bacillus licheniformis and is required for the production of the two-component lantibiotic lichenicidin (FIG. 1E). After installation of the thioether rings in the precursor peptides LicA1 and LicA2, LicT removes the leader peptide of modified LicA1 to generate Licα as well as the majority of the leader peptide from modified LicA2 to generate NDVNPE-Licβ (hereafter LicA2′)(FIG. 1F). The maturation of Licβ requires one more cleavage step outside the cell, where LicP trims off the six remaining amino acids at the N-terminus of LicA2′ (FIG. 1F).

CylA contains an N-terminal secretion signal peptide and was reported to lack the first 95 amino acids when purified from the producing strain. Similar observations were also reported for two class I LanPs, NisP and EpiP, which lacked the first 195 and 99 amino acids in their mature forms, respectively. The removal of such a pro-sequence may activate the proteases for their LanA substrates. However, no activity comparison has been performed between the mature, processed form of LanP and its full-length version to confirm such activation. For CylA, the mature form was reported to exhibit the desired activity against CylL_{L}′ and CylL_{S}′ when purified from the producing strain supernatant, but no studies have been performed to define its substrate specificity.

A homology model of NisP, the peptidase involved in nisin biosynthesis, suggested that the substrate specificity of NisP relies on electrostatic and hydrophobic interactions between the S1/S4 NisP pockets and the residues in the −1 (Arg) and −4 positions (Ala) of nisin's precursor peptide NisA (FIG. 2A; negative numbers are used for the residues in the leader peptide counting back from the protease cleavage site). Mutating these two positions in the leader peptide of nisin precluded the removal of the leader peptide as observed by the in vivo accumulation of modified precursor nisin with the leader peptide still attached. In addition, NisP only removes the N-terminal leader peptide from the modified precursor peptide NisA.

In general, the understanding of the substrate specificity of LanP enzymes is still limited in part because of the lack of detailed in vitro mechanistic studies as a result of the intrinsic low expression and poor solubility associated with these enzymes. Having a greater insight to these activities will improve the efficiency of using these lanthipeptide enzymes and their associated substrates in biotechnological, medical, and diagnostic applications.

### BRIEF SUMMARY OF THE INVENTION

In a first aspect, an isolated nucleic acid that includes an open reading frame encoding a lanthipeptide protease polypeptide for scarless tag removal from a polypeptide is disclosed.

In a second aspect, an expression cassette that includes an open reading frame for a polypeptide, wherein the open reading frame encodes a substrate recognition sequence for a lanthipeptide protease polypeptide, is disclosed.

In a third aspect, a method of scarless tag removal from a polypeptide is disclosed. The method includes two steps. The first step includes providing the polypeptide, wherein the polypeptide includes the structure: T-R-P, wherein T comprises a tag motif, R comprises a lanthipeptide protease substrate recognition sequence and P comprises an open reading frame encoding a polypeptide without the tag motif and lanthipeptide protease substrate recognition sequence. The second step includes subjecting the polypeptide to a lanthipeptide protease having specificity for catalyzing proteolytic cleavage at the lanthipeptide protease substrate recognition sequence, thereby providing the polypeptide without a tag scar.

In a fourth aspect, a kit for expressing a polypeptide without a tag scar is disclosed. The kit includes an expression vector that includes an expression cassette, wherein the expression cassette encoding a polypeptide including the structure: T-R-P, wherein T includes a tag motif, R includes a lanthipeptide protease substrate recognition sequence and P includes an open reading frame encoding a polypeptide without the tag motif and lanthipeptide protease substrate recognition sequence. The kit also includes a lanthipeptide protease having specificity for catalyzing proteolytic cleavage at the lanthipeptide protease substrate recognition sequence, thereby providing the polypeptide without the tag scar.

In a fifth aspect, isolated polypeptide including the structure T-R-P is disclosed. The T includes a tag motif, the R includes a lanthipeptide protease substrate recognition sequence and the P includes an open reading frame encoding a polypeptide without the tag motif and lanthipeptide protease substrate recognition sequence.

### BRIEF DESCRIPTION OFT OF FIGURES

FIG. A depicts biosynthesis of epilancin 15X, involving dehydration of Ser and Thr residues by ElxB to yield dehydroalanine (Dha, green), and dehydrobutyrine (Dhb, purple), formation of lanthionine (red) or methyllanthionine (blue) rings catalyzed by ExC, removal of the leader peptide (bold) by the peptidase ElxP.

FIG. 1B depicts reduction of the N-terminal pyruvyl moiety catalyzed by ExO. Abu (2-aminobutyric acid), Pyr (pyruvyl), Lac (lactyl).

FIG. 1C depicts lanthionine synthetases and proteases involved in the biosynthesis of four classes of lanthipeptides.

FIG. 1D depicts biosynthetic gene cluster of the enterococcal cytolysin and the sequential cleavage event employed during cytolysin maturation. Abu, α-aminobutyric acid.

FIG. 1E depicts an exemplary biosynthetic pathway of class II lanthipeptides with lichenicidin β shown as an example. Also shown are the structures of both components that make up lichenicidin. Obu, 2-oxobutyryl group resulting from hydrolysis of an N-terminal Dhb; Abu, α-aminobutyric acid.

FIG. 1F depicts Lanthionine synthetases and proteases involved in the biosynthesis of class I and class II lanthipeptides (panel (i)) and the biosynthetic gene cluster of lichenicidin and the cleavage events employed during lichenicidin maturation (panel(ii)).

FIG. 2A depicts a sequence alignment of selected LanA leader peptides for which the final products (class I lanthipeptides) have been structurally characterized. The conserved “FDLN” motif is highlighted in green. The putative LanP recognition motifs are shown in blue and red boxes for the NisA-group and the ExA-group, respectively. LanP cleavage sites are shown with an arrow.

FIG. 2B depicts MCMC phylogenetic tree of LanP enzymes corresponding to the LanA substrates shown in FIG. 2A. Bayesian inferences of posterior probabilities are shown above or below the branches. Two LanPs involved in class II lanthipeptide biosynthesis (LicP for lichenicidin and CylA for cytolysin) served as the out group of the tree.

FIG. 3A depicts an exemplary kinetic characterization of ElxP peptidase activity for His₆-ElxA, wherein different concentrations of the purified peptide were digested with ElxP and leader peptide formation was monitored at different time points by HPLC. The rate of MBP-ElxP catalysis was plotted as a function of different substrate concentrations. The data was fit to the Michealis-Menten equation to give the kinetic parameters shown. Data is presented as the average±standard error of two independent experiments.

FIG. 3B depicts an exemplary kinetic characterization of ElxP peptidase activity for mutant peptide ExA Q-1A, wherein different concentrations of the purified peptide were digested with ElxP and leader peptide formation was monitored at different time points by HPLC. The kinetic analysis was conducted as described in FIG. 3A.

FIG. 3C depicts an exemplary kinetic characterization of ElxP peptidase activity for mutant peptide ExA L-4A, wherein different concentrations of the purified peptide were digested with ElxP and leader peptide formation was monitored at different time points by HPLC. The kinetic analysis was conducted as described in FIG. 3A.

FIG. 3D depicts an exemplary kinetic characterization of ElxP peptidase activity for mutant peptide ExA P-2A, wherein different concentrations of the purified peptide were digested with ElxP and leader peptide formation was monitored at different time points by HPLC. The kinetic analysis was conducted as described in FIG. 3A.

FIG. 4A depicts leader peptide sequences of NisA variants. The cleavage recognition sequence of NisA is shown in blue. Amino acid mutations in the NisA variants are shown in red. ExA leader peptide sequence is shown for reference. ElxP cleavage site is shown with an arrow.

FIG. 4B depicts exemplary MALDI-MS data on cleavage of NisA and NisA mutants by ElxP, wherein NisA was treated with ElxP. Key: PP-precursor peptide, CP-core peptide, and LP-leader peptide. MS Spectrum key: His₆-NisA (7992 m/z).

FIG. 4C depicts exemplary MALDI-MS data on cleavage of NisA and NisA mutants by ElxP, wherein NisA G-5D/A-4L/S-3N/R-1Q/Q-1_IlinsA was treated with ElxP. Key is as presented in FIG. 4B. MS Spectrum key: His₆ NisA-G-5D/A-4L/S-3N/R-1Q/Q-1_IlinsA unmodified core peptide (3568 m/z), and leader peptide (4064 m/z).

FIG. 4D depicts exemplary MALDI-MS data on cleavage of NisA and NisA mutants by ElxP, wherein NisA R-1Q was treated with ElxP. Key is as presented in FIG. 4B. MS Spectrum key: His₆-NisA R-1Q (7412 m/z); *Ion corresponding to peptide with gluconoylation of the His₆-tag of NisA. His₆-NisA R-1Q (7412 m/z)

FIG. 4E depicts exemplary MALDI-MS data on cleavage of NisA and NisA mutants by ElxP, wherein NisA R-1Q/Q-1_IlinsA was treated with ElxP. Key is as presented in FIG. 4B. MS Spectrum key: His₆-NisA R-1Q/Q-1_IlinsA (7485 m/z).

FIG. 4F depicts exemplary MALDI-MS data on cleavage of NisA and NisA mutants by ElxP, wherein NisA G-5D/A-4L/S-3N/R-1Q was treated with ElxP. Key is as presented in FIG. 4B. MS Spectrum key: His₆-NisA-G-5D/A-4L/S-3N/R-1Q (7542 m/z); *Ion corresponding to peptide with gluconoylation of the His₆-tag of NisA. His₆-NisA R-1Q (7412 m/z).

FIG. 5A depicts a schematic structure of the lantibiotic lactocin S that contains an N-terminal Pyr group, which was converted to dihydrolactocin S as evidenced by LC-MS analysis.

FIG. 5B depicts an exemplary MS analysis of lactocin S (calculated m/z=3762.8851) incubated with NADPH in the absence of His₆-ElxO. The peak at m/z=3794.9348 corresponds to oxidized lactocin S (M+O).

FIG. 5C depicts an exemplary MS analysis of dihydrolactocin S (calculated m/z=3764.8851) generated after incubation of lactocin S with both NADPH and His₆-ElxO. The peaks at m/z=3786.8765 and 3808.8447 correspond to the sodium and disodium adducts of dihydrolactocin S.

FIG. 6A depicts an exemplary single concentration agar diffusion bioactivity assay. The samples spotted were enzymatically synthesized dihydrolactocin S (sample 1) and control samples lacking enzyme (sample 2), cofactor (sample 3), or both (sample 4) and incubated under the same reaction conditions. Sample 5 was a control assay lacking lactocin S.

FIG. 6B depicts exemplary serial dilution agar diffusion bioactivity assays. The samples are as described in FIG. 6A.

FIG. 7A depicts an exemplary SDS-PAGE gel of His₆-CylA-27-412.

FIG. 7B depicts an exemplary MALDI-TOF mass spectrum of His₆-CylA-27-412 showing the mass range 5000-22,000 Da for the N-terminal fragment His₆-CylA-27-95.

FIG. 7C depicts an MALDI-TOF mass spectrum of His₆-CylA-27-412 showing a mass range of 22,000-50,000 Da for the C-terminal fragment CylA-96-412 and full length protein His₆-CylA-27-412 (c).

FIG. 7D depicts an exemplary MALDI-TOF mass spectrum of His₆-CylA-27-412-E95A.

FIG. 7E depicts an exemplary MALDI-TOF mass spectrum of His₆-CylA-27-412-S359A.

FIG. 8A depicts exemplary MALDI-TOF mass spectrum of CylL_{L}″ with a 5-amino acid peptide remaining from the leader (subpanel (i)) and CylL_{S}″ with a 5-amino acid N-terminal peptide (subpanel (ii)) incubated with (magenta) or without (blue) CylA.

FIG. 8B depicts exemplary MALDI-TOF mass spectrum of modified CylL_{L}(subpanel i) and CylL_{S} (subpanel ii) incubated with CylA.

FIG. 8C depicts an exemplary assay of antimicrobial activities of protease digested peptides against Lactococcus lactis HP. Spot 1, CylM-modified CylL_{L}-E-1K peptide processed by trypsin; spot 2, CylM-modified CylL_{S}-E-1K peptide processed by trypsin; spot 3, samples 1+2; spot 4, CylM-modified CylL_{L} peptide processed by CylA; spot 5, CylM-modified CylL_{S} peptide processed by CylA; spot 6, samples 4+5. For all samples, 500 pmol were spotted.

FIG. 8D depicts an exemplary assay of hemolytic activity of mature cytolysin obtained by CylA digestion against rabbit red blood cells. The amounts of compounds applied are indicated. Error bars indicate the standard deviation of three separate experiments. The data points were fit with a Growth-Sigmoidal-Dose Response function, which does not necessarily indicate the kinetics of the lytic process.

FIG. 8E depicts an exemplary MALDI-TOF mass spectrum of linear CylL_{L} incubated with CylA. Unmodified CylL_{L} core peptide was not observable presumably due to its high hydrophobicity.

FIG. 8F depicts an exemplary MALDI-TOF mass spectrum of linear CylL_{S} incubated with CylA.

FIG. 9A depicts an exemplary MALDI/TOF mass spectrum for HalM2-modified HalA2-GDVQAE peptide.

FIG. 9B depicts and exemplary MALDI-TOF mass spectrum of modified HalA2-GDVQAE peptide incubated with CylA.

FIG. 9C depicts an exemplary assay of antimicrobial activity of mature Halβ obtained by CylA in combination with Halα against Lactococcus lactis HP. Key: 1, 500 pmol Halα+500 pmol Halβ; 2, 500 pmol Halα; 3, 500 pmol Halβ; 4, 500 pmol Halα+500 pmol HalA2-GDVQAE; 5, 500 pmol HalA2-GDVQAE; 6, 500 pmol Halα+500 pmol HalA2-GDVQAE treated with CylA; 7, 500 pmol HalA2-GDVQAE treated with CylA; 8, 100 pmol nisin.

FIG. 10 illustrates the precursor peptide sequences of some of the lanthipeptides disclosed herein, wherein the leader sequences (yellow) and core sequences (blue) are shown.

FIG. 11A depicts an exemplary MALDI/TOF mass spectra for HaIM1-modified HalA1-GDVQAE peptide without the treatment of CylA.

FIG. 11B depicts an exemplary MALDI/TOF mass spectrum for HaIM1-modified HalA1-GDVQAE peptide with the treatment of CylA.

FIG. 12A depicts an exemplary MALDI/TOF mass spectrum for ProcA1.7-GDVQAE peptide treated with CylA.

FIG. 12B depicts an exemplary MALDI/TOF mass spectrum for NisA-GDVQAE peptide treated with CylA.

FIG. 13A depicts an exemplary MALDI/TOF mass spectrum for ProcA1.7-GDVQAE-T1G treated with CylA. The observation of core peptides in the inset was achieved by using a different instrument setting optimized for samples with lower molecular weights.

FIG. 13B depicts an exemplary MALDI/TOF mass spectrum for ProcA1.7-GDVQAE-T1F treated with CylA. The observation of core peptides in the inset was achieved by using a different instrument setting optimized for samples with lower molecular weights.

FIG. 13C depicts an exemplary MALDI/TOF mass spectrum for ProcA1.7-GDVQAE-T1W treated with CylA. The observation of core peptides in the inset was achieved by using a different instrument setting optimized for samples with lower molecular weights.

FIG. 14A depicts an exemplary kinetic analysis of CylA's proteolytic activities against modified CylL_{S} (36 μM final concentration) with full length CylA (CylA-96-412, 22 nM final concentration). Proteolytic reactions were stopped at different time points by 1% TFA and analyzed by LC/MS. Extracted ion chromatographs for mature CylL_{S}″ that was produced were overlayed. Proteolytic reactions that were allowed to proceed for 24 hours were treated with the same procedure and serve as positive controls with 100% product formation.

FIG. 14B depicts an exemplary kinetic analysis of CylA's proteolytic activities against modified CylL_{S} (36 μM final concentration) with CylA after autoproteolytic processing (His₆-CylA-27-412-E95A, 110 nM final concentration). Proteolytic reactions were conducted and analyzed as in FIG. 14A.

FIG. 15A depicts an exemplary SDS-PAGE gel purified His₆-LicP-25-433.

FIG. 15B depicts an exemplary MALDI-TOF mass spectrum of purified His₆-LicP-25-433.

FIG. 16 depicts an exemplary SDS-PAGE image of soluble His₆-LicP-25-433-S376A.

FIG. 17 depicts an exemplary SDS-PAGE image of His₆-LicP-25-433 (consisting of a complex of His₆-LicP-25-100 and LicP-101-433) incubated with His₆-LicP-25-433-S376A in a 1:1 ratio. The reaction was monitored by SDS-PAGE to determine if wild type LicP catalyzes the proteolytic cleavage of His₆-LicP-25-433-S376A. When incubated separately, His₆-LicP-25-433 and His₆-LicP-25-433-S376A did not show any changes throughout the 19-hour incubation period, whereas the full length protein His₆-LicP-25-433-S376A was consumed gradually when incubated with His₆-LicP-25-433, suggesting that cleavage of LicP can take place intermolecularly. Proteins were supplied at a final concentration of 0.1 mg/mL each.

FIG. 18A depicts an exemplary SDS-PAGE analysis of His₆-LicP-25-433-E100A.

FIG. 18B depicts an exemplary MALDI-TOF MS analysis of His₆-LicP-25-433-E100A. His₆-LicP-25-102-E100A, calculated M: 10,096, average; observed M+H⁺: 10,099, average. LicP-103-433, calculated M: 37,219, average; observed M+H⁺: 37,207, average.

FIG. 19A depicts an exemplary MALDI-TOF mass spectrum of linear LicA2. LicA2, calculated M: 8,930, average; observed M+H⁺: 8,929, average.

FIG. 19B depicts an exemplary MALDI-TOF mass spectrum of LicM2-modified LicA2. LicM2-modified LicA2, calculated M-12H₂O: 8,714, average; observed M-12H₂O+H⁺: 8,713, average. Gluconoylation at the N terminus of LicA2 was introduced when expressing the peptide in E. coli BL21(DE3), resulting in a +178 Da peak in addition to the peak with the desired mass.

FIG. 20A depicts exemplary MALDI-TOF mass spectra of DVNPE-Licβ peptide with (magenta) or without (blue) incubation with LicP. (For DVNPE-Licβ, calculated M: 3572.6, monoisotopic; observed M+H⁺: 3573.6, monoisotopic. For Licβ, calculated M: 3019.4, monoisotopic; observed M+H⁺: 3020.5, monoisotopic.)

FIG. 20B depicts time-dependent MALDI-TOF MS analysis of modified LicA2 and linear G-LicA2 treated with LicP, monitoring the production of leader peptides. For all traces, both peptides were each supplied with a final concentration of 17 μM. For the purple trace, 2.1 μM of His₆-LicP-25-433 was added and the reaction was incubated at room temperature for 12 h (asterisk); for the other traces, 21 nM His₆-LicP-25-433 was employed.

FIG. 21A depicts an exemplary MALDI-TOF mass spectrum for LicM2 modified LicA2 incubated with LicP. Licβ, calculated M: 3,019.4, monoisotopic; observed M+H⁺: 3,020.6, monoisotopic. LicA2 leader peptide, calculated M: 5,711, average; observed M+H⁺: 5,711, average.

FIG. 21B depicts an exemplary MALDI-TOF mass spectrum for linear LicA2 incubated with LicP. LicP, calculated M: 3,019.4, monoisotopic; observed M+H⁺: 3,020.6, monoisotopic. LicA2 leader peptide, calculated M: 5,711, average; observed M+H⁺: 5,713, average. Unmodified LicA2 core peptide was not observed presumably due to poor ionization efficiency.

FIG. 22A depicts an exemplary time-dependent MALDI-TOF MS analysis of modified LicA2 and linear G-LicA2 peptides treated with LicP, monitoring the consumption of precursor peptides. Modified LicA2 and linear G-LicA2 were each supplied with a final concentration of 17 μM. For the purple trace, 2.1 μM of His₆-LicP-25-433 was added and the reaction was incubated at room temperature for 12 h (asterisk); for the other traces, 21 nM His₆-LicP-25-433 was employed. The intensity of the highest peak observed in the region of 8,600-9,000 Da was set to 100%. No signal was observed in this region for the purple trace.

FIG. 22B depicts an expanded-view MALDI-TOF mass spectra of modified LicA2 and linear G-LicA2 peptides treated with or without LicP. Conditions are as described in FIG. 23A.

FIG. 23A depicts an exemplary MALDI-TOF mass spectrum for ProcA1.7-NDVNPE. ProcA1.7-NDVNPE, calculated M: 12,244, average; observed M+H⁺: 12,246, average.

FIG. 23B depicts an exemplary MALDI-TOF mass spectrum for NisA-NDVNPE. NisA-NDVNPE, calculated M: 7,557, average; observed M+H⁺: 7,558, average. Gluconoylation at the N terminus of NisA-NDVNPE was introduced when expressing the peptide in E. coli BL21(DE3), resulting in a +178 Da peak in addition to the peak with the desired mass.

FIG. 23C depicts the primary sequences of NisA and ProcA1.7.

FIG. 24A depicts an exemplary MALDI-TOF mass spectrum for ProcA1.7-NDVNPE incubated with LicP. ProcA1.7 core peptide, calculated M: 2,256.1, monoisotopic; observed M+H⁺: 2,257.6, monoisotopic. ProcA1.7-NDVNPE leader peptide, calculated M: 10,004, average; observed M+H⁺: 10,003, average.

FIG. 24B depicts an exemplary MALDI-TOF mass spectrum for NisA-NDVNPE incubated with LicP. NisA core peptide, calculated M: 3,495.6, monoisotopic; observed M+H⁺: 3,496.4, monoisotopic. NisA-NDVNPE leader peptide, calculated M: 4,074.9, monoisotopic; observed M+H⁺: 4,075.5, monoisotopic.

FIG. 25 depicts an exemplary SDS-PAGE analysis of MBP-BamL protein (SEQ ID NO.: 221) incubated with TEV or LicP. MBP-BamL (50 μM) was incubated with LicP or TEV (final concentration 0.54 μM) at 4° C. and the reactions were quenched at different time points before analysis by SDS-PAGE. O/N: overnight.

FIG. 26 depicts exemplary MALDI-TOF mass spectra for NisA-NDVNPE peptides with various P1′ substitutions. Gluconoylation at the N terminus of NisA-NDVNPE peptides was introduced when expressing these peptides in E. coli BL21(DE3), resulting in a +178 Da peak in addition to the peak of the desired mass. An unidentified modification of +58 Da was installed on NisA-NDVNPE-I1K, but MALDI-TOF MS analysis suggests that the desired species is the major product.

FIG. 27 depicts exemplary MALDI-TOF mass spectra for NisA-NDVNPE peptides with various P1′ substitutions incubated with LicP. For all reactions, 0.5 mg/mL (65 μM) of NisA variants were included. For NisA-NDVNPE-I1T and NisA-NDVNPE-I1C, LicP was supplied at a final concentration of 0.01 mg/mL (210 nM) (enzyme:substrate=310:1) and the reactions were incubated at room temperature for 20 hours. For other NisA variants, LicP was supplied at a final concentration of 0.1 mg/mL (2.1 μM) (enzyme:substrate=31:1) and the reactions were incubated at room temperature for 30 hours.

FIG. 28 depicts an exemplary LicP assay with wild type LicA2, LicA2-E-1A, and LicA2-E-1Q. LicA2 analogs (100 μM) were incubated with 0.4 μM LicP for 30 min, 2 h, or 7.5 h. The reaction was quenched with formic acid, and the assay was analyzed by SDS-PAGE with consecutive coomassie and silver staining. Lanes: 1: #161-0326 ladder; 2: wild type LicA2; 3: wild type LicA2, 30 min with LicP; 4: wild type LicA2, 2 h with LicP; 5: wild type LicA2, 7.5 h with LicP; 6: LicP; 7: LicA2-E-1A; 8: LicA2-E-1A, 30 min with LicP; 9: LicA2-E-1A, 2 h with LicP; 10: LicA2-E-1A, 7.5 h with LicP; 11: LicA2-E-1Q; 12: LicA2-E-1Q, 30 min with LicP; 13: LicA2-E-1Q, 2 h with LicP; 14: LicA2-E-1Q, 7.5 h with LicP; 15: #161-0326 ladder.

FIG. 29 depicts an exemplary LicP assay with wild type LicA2, LicA2-E-1D, and LicA2-D-5K. LicA2 analogs (100 μM) were incubated with 0.4 μM LicP for 30 min, 2 h, or 7.5 h. Then the reaction was quenched with formic acid, and the assay was analyzed by SDS-PAGE with consecutive coomassie and silver staining. Lanes: 1: #161-0326 ladder; 2: wild type LicA2; 3: wild type LicA2, 30 min with LicP; 4: wild type LicA2, 2 h with LicP; 5: wild type LicA2, 7.5 h with LicP; 6: LicP; 7: LicA2-E-1D; 8: LicA2-E-1D, 30 min with LicP; 9: LicA2-E-1D, 2 h with LicP; 10: LicA2-E-1D, 7.5 h with LicP; 11: LicA2-D-5K; 12: LicA2-D-5K, 30 min with LicP; 13: LicA2-D-5K, 2 h with LicP; 14: LicA2-D-5K, 7.5 h with LicP; 15: #161-0326 ladder.

FIG. 30 depicts an exemplary LicP assay with wild type LicA2, LicA2-V-4F, and LicA2-D-5A. LicA2 analogs (100 μM) were incubated with 0.4 μM LicP for 30 min, 2 h, or 7.5 h. The reaction was quenched with formic acid, and the assay was analyzed by SDS-PAGE with consecutive coomassie and silver staining. Lanes: 1: #161-0326 ladder; 2: wild type LicA2; 3: wild type LicA2, 30 min with LicP; 4: wild type LicA2, 2 h with LicP; 5: wild type LicA2, 7.5 h with LicP; 6: LicA2-V-4F; 7: LicA2-V-4F, 30 min with LicP; 8: LicA2-V-4F, 2 h with LicP; 9: LicA2-V-4F, 7.5 h with LicP; 10: LicA2-D-5A, 11: LicA2-D-5A, 30 min with LicP; 12: LicA2-D-5A, 2 h with LicP; 13: LicA2-D-5A, 7.5 h with LicP; 14: #161-0326 ladder.

FIG. 31 depicts an exemplary LicP assay with wild type LicA2, LicA2-V-4A, and LicA2-V-4L. LicA2 analogs (100 μM) were incubated with 0.4 μM LicP for 15 min, 1 h, or 2 h. The reaction was quenched with formic acid, and the assay was analyzed by SDS-PAGE with consecutive coomassie and silver staining. Lanes: 1: #161-0326 ladder; 2: wild type LicA2; 3: wild type LicA2, 15 min with LicP; 4: wild type LicA2, 1 h with LicP; 5: wild type LicA2, 2 h with LicP; 6: LicA2-V-4A; 7: LicA2-V-4A, 15 min with LicP; 8: LicA2-V-4A, 1 h with LicP; 9: LicA2-V-4A, 2 h with LicP; 10: LicA2-V-4L; 11: LicA2-V-4L, 15 min with LicP; 12: LicA2-V-4L, 1 h with LicP; 13: LicA2-V-4L, 2 h with LicP; 14: #161-0326 ladder.

FIG. 32 depicts an exemplary LicP assay with wild type LicA2, LicA2-P-2A, and LicA2-N-3A. LicA2 analogs (100 μM) were incubated with 0.4 μM LicP for 15 min, 1 h, or 2 h. The reaction was quenched with formic acid, and the assay was analyzed by SDS-PAGE with consecutive coomassie and silver staining. Lanes: 1: #161-0326 ladder; 2: wild type LicA2; 3: wild type LicA2, 15 min with LicP; 4: wild type LicA2, 1 h with LicP; 5: wild type LicA2, 2 h with LicP; 6: LicP; 7: LicA2-P-2A; 8: LicA2-P-2A, 15 min with LicP; 9: LicA2-P-2A, 1 h with LicP; 10: LicA2-P-2A, 2 h with LicP; 11: LicA2-N-3A; 12: LicA2-N-3A, 15 min with LicP; 13: LicA2-N-3A, 1 h with LicP; 14: LicA2-N-3A, 2 h with LicP; 15: #161-0326 ladder.

FIG. 33 depicts twelve class II lanthipeptide biosynthetic gene clusters containing LanP genes. Genes with unknown functions are indicated with X. Clusters that are annotated using the standard lanthipeptide biosynthesis nomenclature contain: LanM proteins catalyze the dehydration and cyclization reactions, LanA peptides are the lanthipeptide precursors, LanT proteins are transporters with an N-terminal Cys protease domain, LanEFGHI proteins are immunity conferring proteins, LanHR are regulatory proteins. The cytolysin cluster is annotated differently: The substrates are CylL_{L} and CylL_{S}, Cyl_{B} is a transporter with a protease domain, and CylA is the class II LanP. The LanP polypeptides have the following corresponding SEQ ID NOs: CylA (SEQ ID NO: 9); LicP (SEQ ID NO: 12); B. licheniformis 9945A (SEQ ID NO: 14). CerP of B. cereus QI (SEQ ID NO: 15); B. cereus FRI-35 (SEQ ID NO: 16); Kyrpidia tusciae DSM 2912 (SEQ ID NO: 17); E. caccae ATCC BAA-1240 (SEQ ID NO: 18); B. cereus VPC1401 (SEQ ID NO: 19); Bacillus bombysepticus LanP (SEQ ID NO: 20); Bacillus bombysepticus LanP (SEQ ID NO: 21); Bacillus thuringiensis DB27 LanP (SEQ ID NO: 22); Planomicrobium glaciei CHR43 LanP (SEQ ID NO: 23); Bacillus cereus VD045 LanP (SEQ ID NO: 24)); and Bacillus cereus VD156 LanP (SEQ ID NO: 25).

FIG. 34 depicts twelve other class II lanthipeptide biosynthetic gene clusters containing LanP genes as in FIG. 33. Substrate LanA sequences are listed under the genetic pathways and the putative LanP recognition sequences are highlighted. The predicted sites were verified for the proteins from Bacillus licheniformis 9945A and Bacillus cereus VD045 (see FIG. 35). Clusters were annotated using the standard lanthipeptide biosynthesis nomenclature: LanM proteins catalyze the dehydration and cyclization reactions, LanA peptides are the lanthipeptide precursors, LanT proteins are transporters with an N-terminal Cys protease domain, LanEFGHI proteins are immunity-conferring proteins, and LanR are regulatory proteins. The cytolysin cluster has historically been annotated differently: The substrates are CylL_{L} and CylL_{S}, Cyl_{B} is a transporter with a protease domain, and CylA is the class II LanP. Genes with unknown functions are indicated with X.

FIG. 35A depicts an exemplary MALDI-TOF mass spectrum of a LanA precursor peptide treated with its corresponding LanP protease identified in the genome of B. licheniformis 9945A. For the dehydrated and cyclized LanA2 peptide from B. licheniformis 9945A, fully modified core peptide is observed after protease treatment (inset): calculated [M+Na]⁺: 2,493.0, monoisotopic mass; observed [M+Na]⁺: 2,493.4, monoisotopic mass; the leader peptide is also observed: calculated M: 6,478, average mass; observed [M+H]⁺: 6,482, average mass.

FIG. 35B depicts an exemplary MALDI-TOF mass spectrum of a LanA precursor peptide treated with its corresponding LanP protease identified in the genome of B. cereus VD156. For the LanA3 peptide encoded by the genome of B. cereus VD156, the core peptide is detected: calculated M: 3,382.7, monoisotopic mass; observed [M+H]+: 3,381.8, monoisotopic mass; and the leader peptide is also observed: calculated M: 5,126.5, monoisotopic mass; observed [M+H]+: 5,127.9, monoisotopic mass.

### DETAILED DESCRIPTION OF THE INVENTION

Reagents, expression constructs and methods are disclosed herein for preparing a scarless tag polypeptide product from a tagged polypeptide precursor containing a lanthipeptide protease cleavage site. The reagents are directed to the use of novel lanthipeptide proteases for processing polypeptide precursors that include highly specific lanthipeptide protease substrate recognition sequence. Methods are provided that enable scarless tag removal from a cognate lanthipeptide, a non-cognate lanthipeptide or a heterologous polypeptide that includes extraneous amino acid sequences, such as leader peptides and tags.

### Definitions

To aid in understanding the invention, several terms are defined below.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter), termination of RNA transcription (for example, a ribozyme), or translation of protein. The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those having ordinary skill in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The term “natural polymer” refers to any polymer comprising natural monomers found in biology. For example, polypeptides are natural polymers made from natural amino acids, where the term “amino acid” includes organic compounds containing both a basic amino group and an acidic carboxyl group. Natural protein occurring amino acids, which make up natural polymers, include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine.

The term “non-natural polymer” refers to any polymer comprising natural and non-natural monomers found in biology. For example, a ribosome can be designed to produce a non-naturally occurring biopolymer based on amino acids where naturally occurring and/or synthetic versions of naturally occurring components are used. For example, non-natural polymers could be made that comprise both natural and unnatural amino acids. These unnatural amino acids could comprise modified and unusual amino acids (e.g., D-amino acids and β-amino acids), as well as amino acids which are known to occur biologically in free or combined form but usually do not occur in proteins. Natural non-protein amino acids include arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5,-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, α-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as substrate for transcribing at least one RNA that can be translated into a polypeptide or protein. Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product of transcription from an expression template that can be used by ribosomes to synthesize polypeptide or protein in vitro or in vivo.

As used herein, “cognate” as it modifies polypeptide with respect to protease substrates disclosed herein, refers to the natural polypeptide as expressed from an endogenous gene in the native cellular context. A protease that acts upon a cognate polypeptide is an endogenous protease that would usually act upon the polypeptide in the same native cellular context. By contrast, “non-cognate” as it modifies polypeptide, refers to a polypeptide substrate for a protease from a different native cellular context. Furthermore, a “heterologous” as it modifies polypeptide includes non-cognate polypeptides, fusion polypeptides and recombinant polypeptides that derive from a different native cellular context with respect to a given protease.

As used herein, “expression cassette” refers to a nucleic acid sequence that enables expression of an RNA having a defined nucleic acid sequence. The nucleic acid sequence can be either DNA or RNA, and the defined nucleic acid sequence can encode a polypeptide. The nucleic acid sequence can include sequences for initiating transcription (e.g., promoter and enhance elements) and terminating transcription; sequences for enhancing translation of the RNA to form polypeptides; and sequences that encode in-frame polypeptide leader and post-translational processing signals. For expression cassettes that produce polypeptides, the nucleic acid sequence can include sequences that encode for affinity tag motifs in-frame with the coding sequence for the polypeptide to enable affinity purification of the resultant polypeptide. For nucleic acid sequences composed of DNA, the expression cassette can include multiple cloning sites or polylinkers to enable cloning of polypeptide-coding genes in-frame with flanking sequences encoding for affinity tag motif(s), leader sequences and/or post-translational processing signals.

The term “tag” (or “tag motif”) refers to a sequence motif that does not normally form part of the native polypeptide to which the sequence motif is covalently linked. In this regard, a tag is a heterogeneous, non-cognate sequence motif with respect to the remainder of the polypeptide sequence. Where a polypeptide is initially synthesized as a precursor polypeptide that includes a leader peptide sequence, a tag also includes the leader peptide sequence with respect to the mature polypeptide. A tag may be covalently linked to the N-terminus, C-terminus or at an internal site (for example, a amino acid side chain) of a polypeptide. A tag can be used to detect, identify, select, enrich or purify the polypeptide to which the tag is covalently linked. A tag (or tag motif) can include a leader peptide sequence and/or an affinity tag.

The term “affinity tag” refers to a sequence that permits detection and/or selection of a polypeptide sequence. For the purposes of this disclosure, a recombinant gene that encodes a recombinant polypeptide may include an affinity tag. In particular, an affinity tag is positioned typically at either the N-terminus or C-terminus of the coding sequence for a polypeptide through the use of recombination technology. Exemplary affinity tags include polyhistine (for example, (His₆)), maltose binding protein, glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, among others.

“Recombinant,” as used herein, refers to an amino acid sequence or a nucleotide sequence that has been intentionally modified by recombinant methods. By the term “recombinant nucleic acid” herein is meant a nucleic acid, originally formed in vitro, in general, by the manipulation of a nucleic acid by endonucleases, in a form not normally found in nature. Thus an isolated nucleic acid in a linear form, or an expression vector formed in vitro by ligating DNA molecules that are not normally joined, are both considered recombinant for the purposes of this invention. It is understood that once a recombinant nucleic acid is made and reintroduced into a host cell, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. A “recombinant protein” is a protein made using recombinant techniques, i.e., through the expression of a recombinant nucleic acid as depicted above.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

The term “vector” refers to a piece of DNA, typically double-stranded, which may have inserted into it a piece of foreign DNA. The vector may be, for example, of plasmid origin. Vectors contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. The vector is used to transport the foreign or heterologous DNA into a suitable host cell. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

As used herein, “scar” refers to a remnant of polypeptide sequence attached to a mature polypeptide that does not form part of the natural amino acid sequence of the mature polypeptide. An example of a scar includes a portion of a leader sequence that is not proteolytically processed accurately from a natural precursor polypeptide to yield a natural mature polypeptide so that the portion of the leader sequence remains attached to the mature polypeptide. Another example of a scar includes a portion of a tag peptide of a precursor recombinant polypeptide that is not completely removed by a protease to generate the recombinant polypeptide without the tag.

As used herein “scarless tag removal” refers to the processing of a precursor polypeptide that contains a tag motif with a protease to yield a polypeptide product having no scar of the tag motif.

As used herein, “codon optimized” refers to a nucleic acid encoding an open reading frame of a polypeptide in which the codons have been selected to permit efficient expression of the polypeptide in a particular host organism or host cell. Exemplary host organisms and host cells (“expression hosts”) for expressing polypeptides (for example, recombinant proteins) include E. coli, S. cerevisiae, S. pombe, P. pastoris, insect cells (for Baculovirus expression), and various mammalian cell lines (for example, HeLa, Jurkat, 293, CHO and COS, among others). Model expression hosts for expressing heterologous polypeptides are known in the art and codon optimized heterologous gene sequences can be deduced from codon usage frequencies of highly expressed polypeptides in such organisms.

As used herein, “substantially identical” as the term modifies a biological composition, such as a nucleotide sequence or a polypeptide sequence, refers to a first primary sequence, including fragments thereof, having at least 75% identity of the intact primary sequence of the reference nucleotide sequence or a polypeptide sequence and/or a second primary sequence having at least 80% sequence homology of the primary sequence of the reference nucleotide sequence or a polypeptide sequence, wherein the first and second primary sequences have at least 70% of the functional activity of the reference nucleotide sequence or a polypeptide sequence.

As used herein, “biological composition” refers to a composition that includes a biological molecule, including for example, a nucleic acid or a polypeptide.

As used herein, “an equivalent thereof” refers to a biological composition, such as a nucleotide sequence or a polypeptide sequence, that encodes the identical or substantially identical structurally- and functionally-defined biological composition as the biological composition being referenced. Sequence homology among nucleotide sequences include nucleotide sequences having degenerate codons and codon-optimized sequences for expression in particular host organisms such that the nucleic acid sequences encode the same polypeptide. Sequence homology among polypeptide sequences include amino sequences having conservative structural changes in terms of hydrophobic, hydrophilic, and ionic side-chain properties such that the resultant polypeptides encode the same functional activity (e.g., identical substrate specificity).

As used herein, “a derivative thereof” refers to a biological composition having at least 10% of the activity of a reference biological composition. More preferably, “a derivative thereof” refers to a biological composition having greater than about 50% of the activity of a reference biological composition, such as about 60%, about 70%, about 90% and about 100% of the activity of a reference biological composition. An example of a LanP protease derivative includes a LanP polypeptide that lacks the signal sequence of the pro-form, nascent LanP polypeptide, such as a LanP polypeptide encoded within an organism genome. Additional examples of a LanP protease derivative includes a LanP polypeptide modified to include tag, such as an affinity tag. An example of a derivative of a recognition substrate for a LanP protease (“lanthipeptide protease substrate recognition sequence”) includes a polypeptide sequence having a P-X motif, wherein the P includes a polypeptide sequence recognized by the LanP protease and X includes an amino acid, wherein the LanP protease catalyzes cleavage of a recognition substrate at the bond between the P and X moieties of the P-X motif. With regard to a derivative of a recognition substrate for a LanP protease, activity refers to one of the rate of catalyzed reaction or the specificity of the cleavage. Thus, sequence variations in either P or X are included in a derivative of a recognition substrate for a LanP protease.

Class I and class II LanP proteases having sequence-specific protease activity are presented. Each of the proteases can serve as an efficient sequence-specific traceless protease for general traceless tag removal applications. Compositions and methods for preparing and using the novel proteases are disclosed herein.

### ElxP as a LanP Having Exquisite Substrate Sequence Specificity

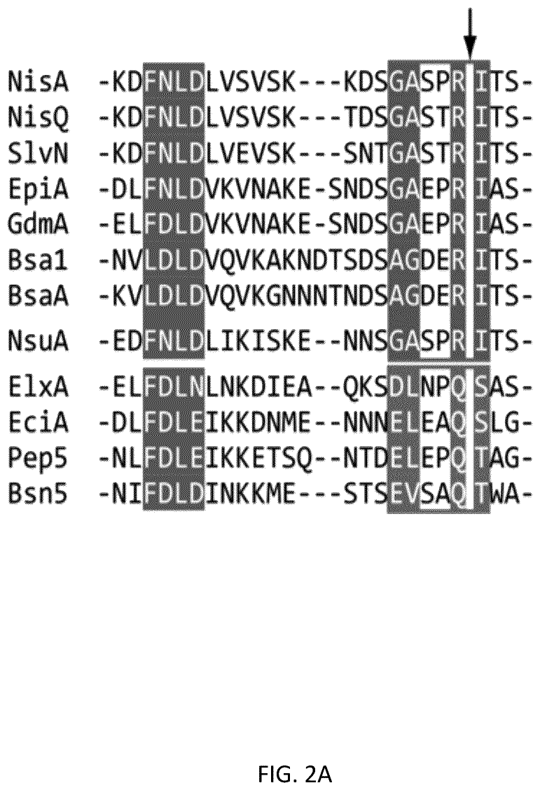

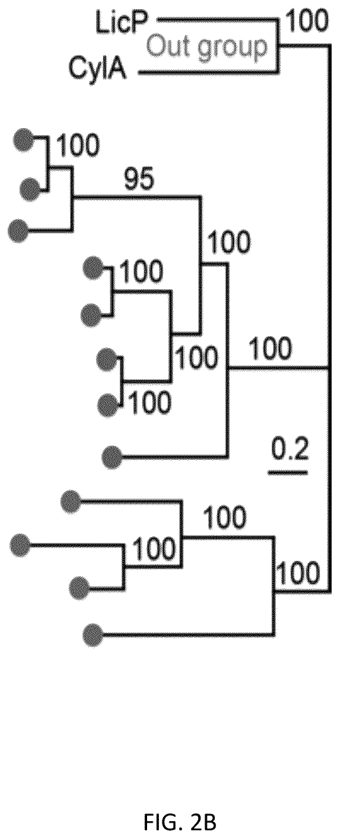

To analyze the substrate specificity of ElxP, the sequences of several LanAs from class I lanthipeptides were aligned. The alignment clearly shows that the sequences near the proteolytic cleavage site group into two different types (FIG. 2A). The first group, from here on named NisA-group, contains a conserved G/A-A/G-x-x-R motif (SEQ ID NO: 1) at the C-terminus of the leader peptide and a Ile residue at the first position of the core peptide. The second group, from here on named ElxA-group, contains a conserved E/D-L/V-x-x-Q motif (SEQ ID NO: 2) at the C-terminus of the leader peptide and a dehydratable residue (Ser/Thr) as the first amino acid of the core peptide. To interrogate whether LanP enzymes are correlated with this grouping of LanAs, a Markov chain Monte Carlo (MCMC) phylogenetic tree of LanPs was constructed with very high fidelity. Our analysis shows that LanPs involved in class I lanthipeptide biosynthesis fall into two major clades that correspond well with the grouping of LanAs (FIG. 2B). This analysis suggests that the conserved motifs of LanAs near the proteolytic cleavage site likely are the recognition elements for LanP enzymes.

To test the hypothesis that the conserved motif in ElxA is important for ElxP activity, ElxA was expressed in Escherichia coli as an N-terminally hexahistidine tagged peptide and purified by metal affinity chromatography. ElxP [(SEQ ID NO: 4) (DNA); (SEQ ID NO: 5) polypeptide] was expressed in E. coli fused to the C-terminus of maltose binding protein (MBP-ElxP; SEQ ID NO: 6 (DNA); SEQ ID NO: 7 (polypeptide)). We then performed alanine scanning mutagenesis on the E/D-L/V-x-x-Q motif present in the ElxA leader peptide. Indeed, single alanine mutations at the Q-1, L-4, and D-5 positions in the leader peptide of ElxA significantly reduced the cleavage efficiency of MBP-ElxP as observed by matrix-assisted laser desorption ionization time-of-flight mass spectrometry (MALDI-TOF MS) (Table 1).

TABLE 1
MBP-ElxP cleavage of wild type and leader
peptide His₆-ElxA mutant peptides¹

| | full length | leader | unmodified | |
|---|---|---|---|---|
| peptide | (calcd) | (calcd) | core (calcd) | result |
| His₆-ElxA wt | 8161 (8162) | 4865 (4864) | 3315 (3316) | +++ |
| Q-1A | 8107 (8105) | 4809 (4807) | 3315 (3316) | + |
| P-2A | 8134 (8136) | 4838 (4838) | 3314 (3316) | ++ |
| N-3A | 8118 (8119) | 4822 (4821) | 3313 (3316) | +++ |
| L-4A | 8119 (8120) | 4822 (4822) | 3314 (3316) | + |
| D-5A | 8115 (8118) | 4819 (4820) | 3313 (3316) | + |
| F-19A | 8086 (8086) | 4789 (4788) | 3315 (3316) | ++ |
| D-18A | 8118 (8118) | 4818 (4820) | 3314 (3316) | +++ |
| L-17A | 8118 (8120) | 4823 (4822) | 3315 (3316) | +++ |
| N-16A | 8119 (8119) | 4819 (4821) | 3314 (3316) | +++ |

¹Observed masses are shown with the calculated mass in parenthesis. MBP-ElxP (5 μM) was incubated with mutant or wild type His₆-ElxA (50 μM in reaction buffer (50 mM Tris HCl pH 8.0) at room temperature for 2 h. Reactions were analyzed by MALDI-TOF MS. Results are based on the amount of substrate left after the assay based on normalized ion intensities.
Complete cleavage (+++), partial cleavage (++), marginal cleavage (+).

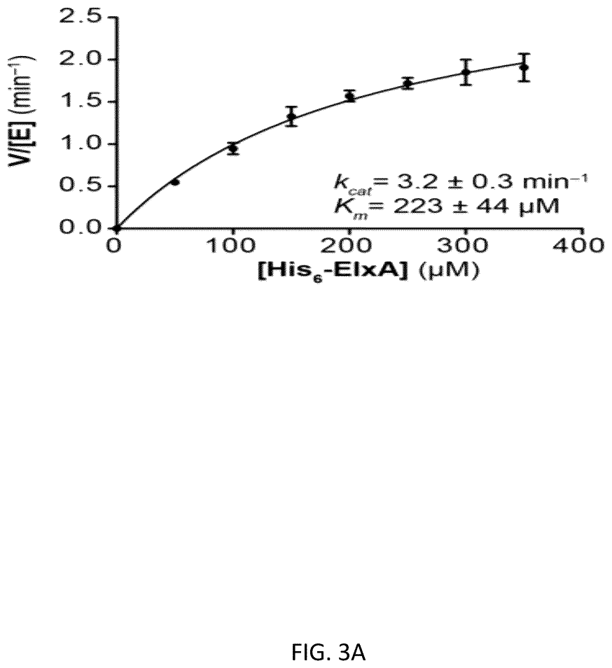

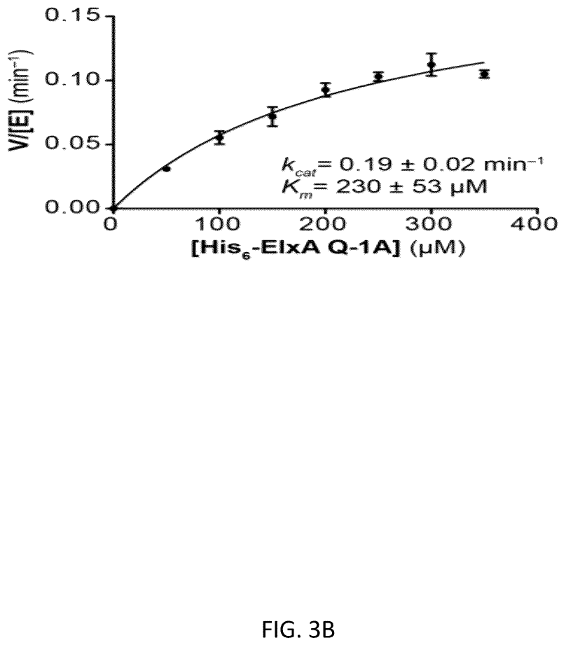

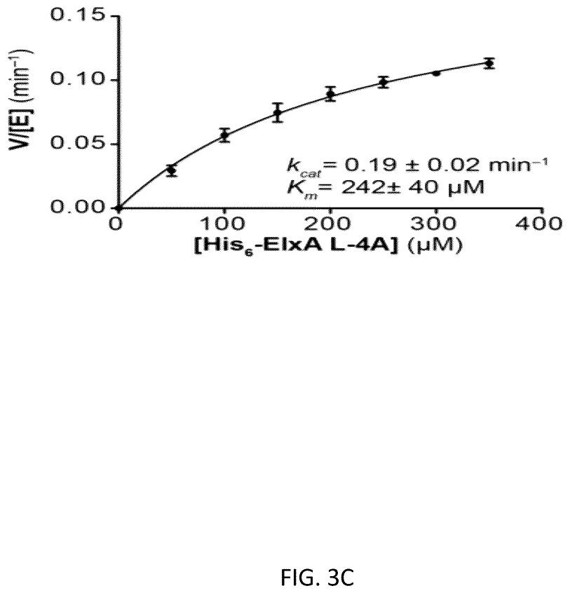

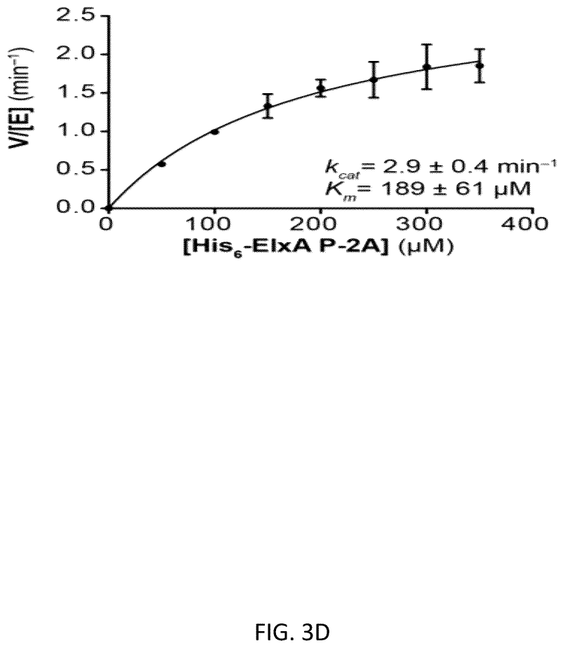

To quantify and distinguish the contribution of each amino acid to the substrate specificity of ElxP, we next determined the kinetic parameters of MBP-ElxP by using the wild type peptide and three ElxA mutants Q-1A, P-2A and L-4A as substrates. Reversed phase high performance liquid chromatography (RP-HPLC) was used to monitor N-terminal leader peptide formation at different substrate concentrations in the assay. The analysis showed that MBP-ElxP cleaved the wild type ElxA with an efficiency of ˜240 M-'s 1 (FIG. 3A), whereas mutating the Q-1 position or L-4 position to Ala resulted in a reduction of ˜14-fold in the ElxP catalytic efficiency (FIGS. 3B and 3C). In contrast, single alanine substitutions at the P-2 and N-3 positions in ElxA did not obviously decrease the efficiency of MBP-ElxP to remove the N-terminal leader peptide from ElxA (Table 1 and FIG. 3D), supporting that only the conserved amino acids in the E/D-L/V-x-x-Q motif of ElxA are important for ElxP activity.

Although it is possible that proteolysis by wild type ElxP of the ElxA peptide possessing the thioether rings would be more efficient, the catalytic efficiency of MBP-ElxP observed in this study with linear peptides is sufficient for application of the enzyme as a sequence specific protease.

Many leader peptides of class I lanthipeptides share an F-D/N-L-N/D sequence motif (FIG. 2A). Previous studies have shown that this conserved motif is important for substrate recognition by the enzymes involved in (methyl)lanthionine incorporation. To probe whether this region is also important for recognition and efficient cleavage by ElxP, we performed alanine scanning mutagenesis on the F-D-L-N motif present in ElxA and analyzed the cleavage efficiency by MALDI-TOF MS. Our results show that the conserved F-D-L-N motif is not essential for ElxP recognition (Table 1), suggesting that LanP enzymes do not recognize the same amino acid motif needed by the enzymes responsible for installing the lanthionine rings (e.g., LanBs and LanCs for the biosynthesis of class I lanthipeptides). This finding is in line with other studies that have found that different parts of the leader peptide are recognized by different post-translational modification enzymes during RiPP biosynthesis.

Insertion of the ElxP Recognition Motif into the NisA Peptide.

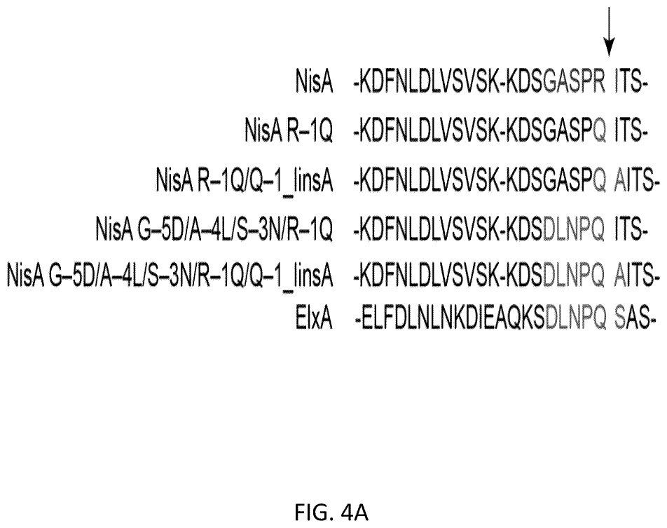

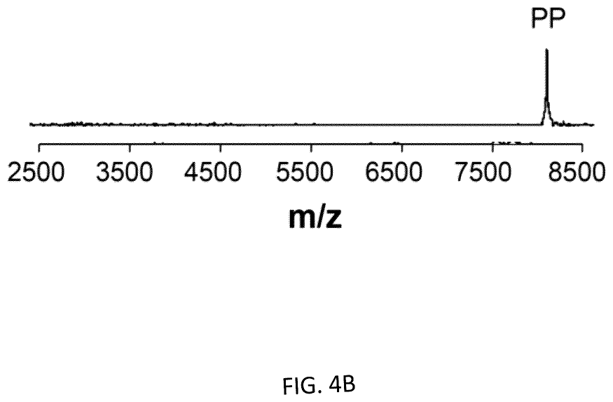

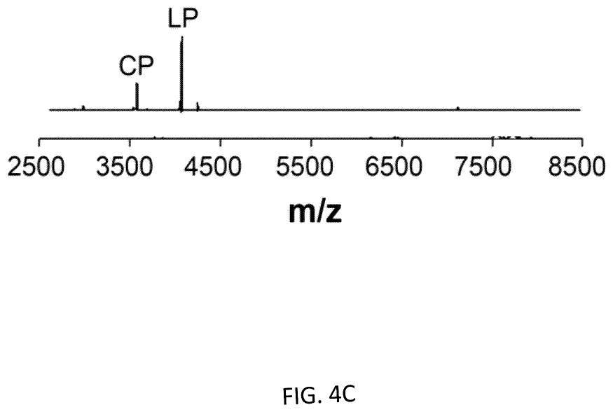

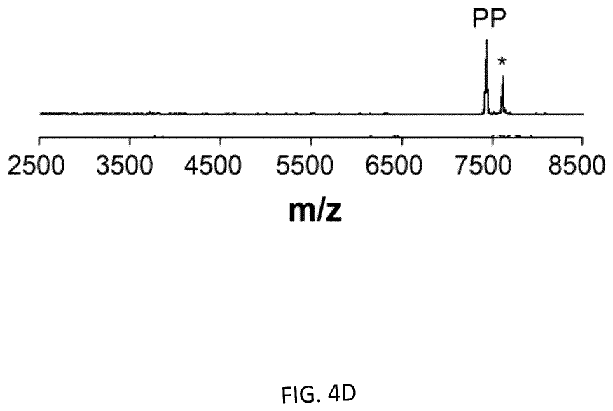

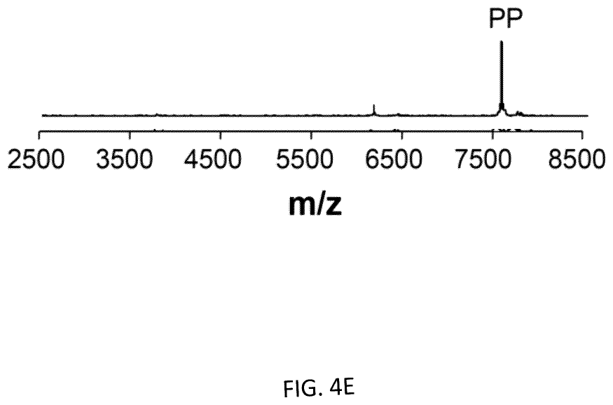

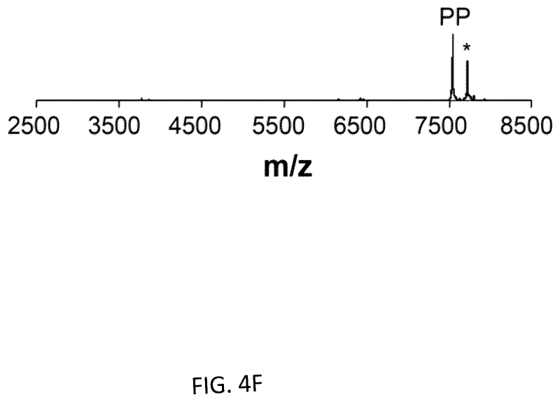

Based on our data, the conserved E/D-L/V-x-x-Q-T1/S1 motif (SEQ ID NO: 3) present in the ElxA-group of LanAs likely serves as the main recognition element for their LanPs. This sequence could possibly be used as a tool to selectively remove tags from fusion proteins or leader peptides from other RiPPs. To determine this potential, we analyzed the ElxP activity on the non-cognate lanthipeptide precursor peptide NisA and NisA mutants (FIG. 4A). Upon incubation of wild type NisA with ElxP, no formation of new peaks corresponding to the N-terminal leader or core peptide masses were observed by MALDI-TOF MS analysis, suggesting that ElxP does not cleave wild type NisA (FIG. 4B). However, when we replaced the G-A-S-P-R-I sequence of NisA to a similar sequence present in ElxA (D-L-N-P-Q-A, in which an Ala residue is used to mimic the dehydrated Ser1 residue of ElxA), the resultant mutant peptide (NisA-G-5D/A-4L/S-3N/R-1Q/Q-1_IlinsA) was completely cleaved by ElxP (FIG. 4C). Other NisA mutants with partial permutations in the G-A-S-P-R-I sequence, including NisA-R-1Q, NisA-G-5D/A-4L/S-3N/R-1Q, and NisA-R-1Q/Q-1_IlinsA, were not cleaved by ElxP (FIG. 4D-F). These results support the model that the ElxP specificity relies on the complete E/D-L/V-x-x-Q-T1/S1 sequence motif.

### Synthesis of Substrate Analogues and Substrate Specificity Analysis of ElxO.

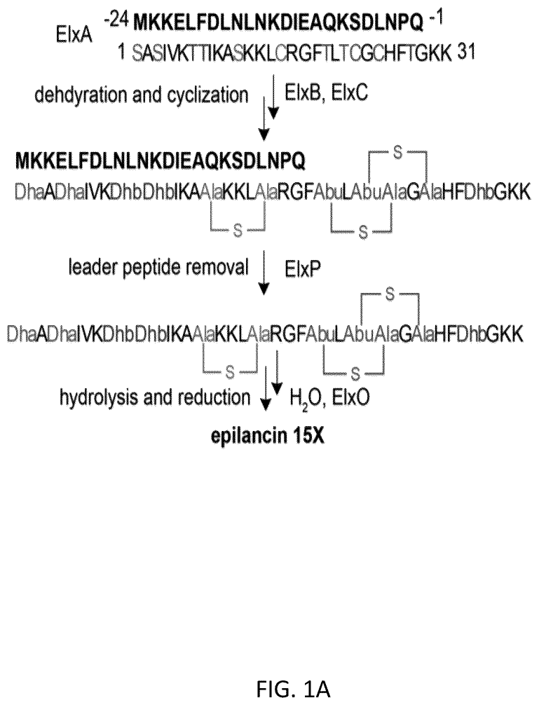

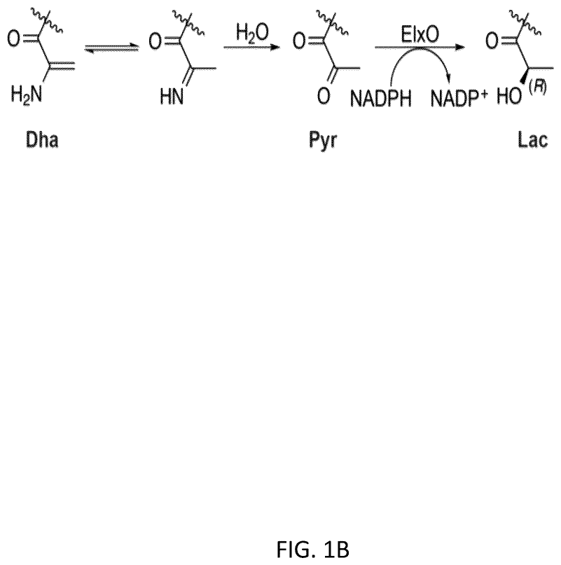

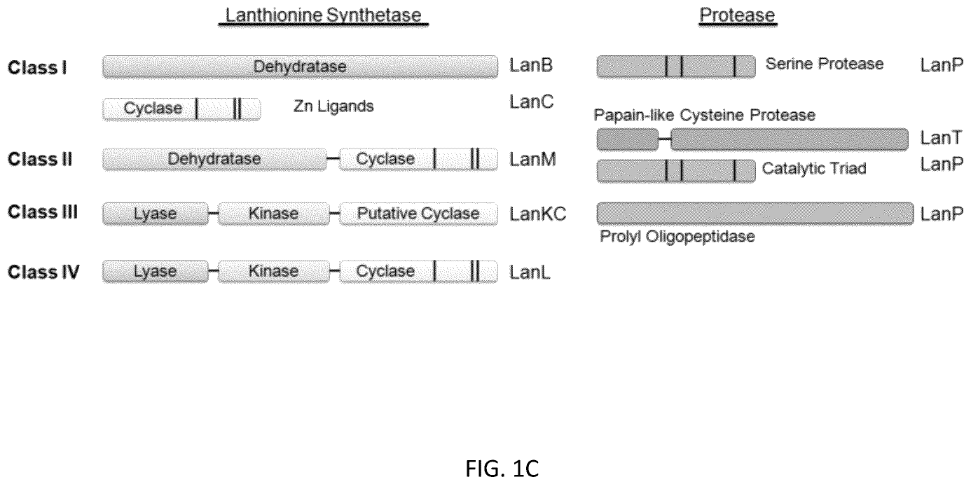

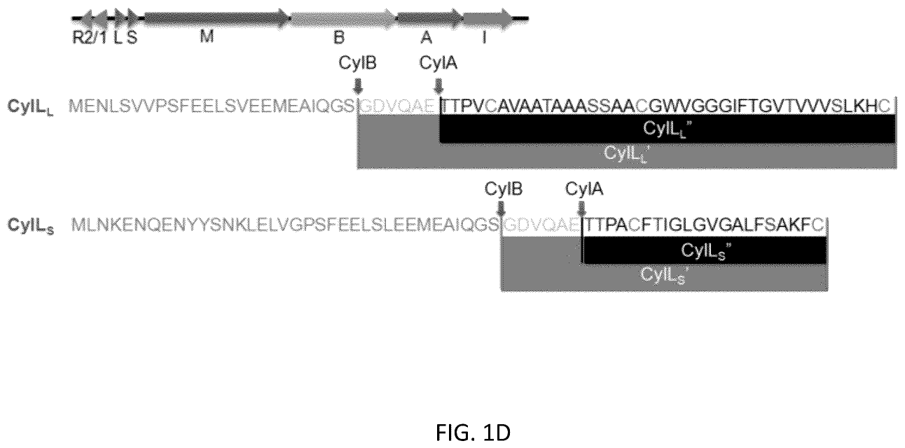

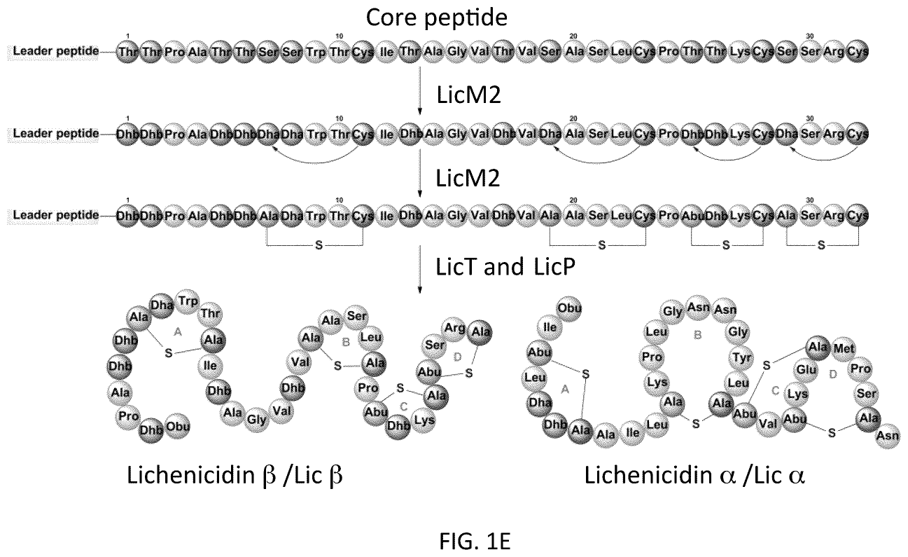

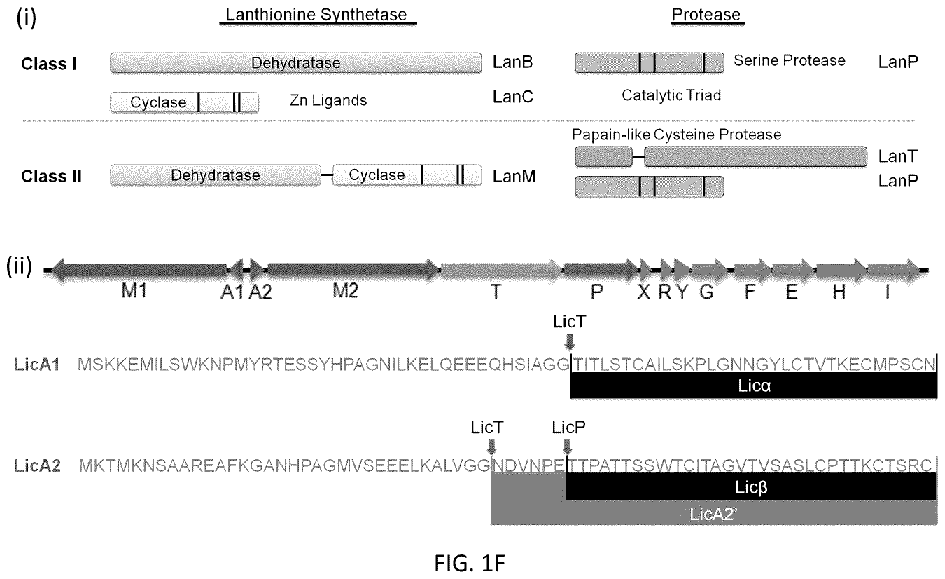

Previous attempts to use the dehydratase ElxB, the cyclase ElxC, and the peptidase ElxP to generate dehydroepilancin 15X, the substrate of the dehydrogenase ElxO, were unsuccessful. However, we showed that His₆-ElxO catalyzes the conversion of the synthetic peptide Pyr-AAIVK, resembling the N-terminal region of dehydroepilancin 15X (FIG. 1A), to D-Lac-AAIVK, demonstrating that the (methyl)lanthionine residues and full length ElxA peptide are not strictly required for substrate recognition by ElxO. Thus, ElxO could be potentially used to introduce N-terminal alcohols to other peptides or proteins that contain N-terminal Pyr or 2-oxobutyryl (OBu) groups, thus enhancing their chemical stability and resistance against degradation by aminopeptidases. To explore the substrate specificity of the enzyme, a series of small potential substrates were synthesized by Fmoc-based solid phase peptide synthesis (SPPS) followed by coupling of pyruvic acid using hydroxybenzotriazole (HOBt) and diisopropylcarbodiimide (DIC) as activating reagents to produce the Pyr-containing substrates. Single residues of the originally tested substrate, Pyr-AAIVK, were replaced systematically with Ala, and Ala2 was changed to a wide variety of amino acids, including polar, nonpolar, acidic, and basic residues to obtain a set of alternative substrates (see Table 2). In addition, the Pyr group was replaced with an N-terminal OBu group, which is generated upon hydrolysis of an N-terminal Dhb residue in the lanthipeptides Pep5, lacticin 3147 A2, lichenicidin, and prochlorosin 1.7. To release the peptides from the solid support, the resin-linked peptides were treated with TFA cleavage cocktails that did not contain triisopropylsilane since the presence of this reagent resulted in the chemical reduction of the ketone-containing peptides, as also observed previously in other work. The peptides were purified by reversed-phase high performance liquid chromatography and the identities of the compounds were confirmed by electrospray ionization mass spectrometry (ESI-MS). The purified peptides were then incubated with His₆-ElxO in the presence of NADPH and the change in the absorbance at 340 nm over time was monitored by UV spectrophotometry (see Scheme (I)).

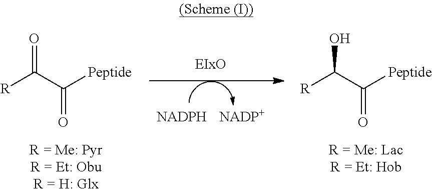

Attempts to determine the steady state kinetic parameters k_{cat} and Kₘ using a subset of peptides were not successful, since it was not possible to saturate the enzyme with the substrates before reaching the peptide solubility limits. Therefore, the kinetic constants k_{cat}/Kₘ were determined by measuring the reaction rates at various peptide concentrations (Table 2).

TABLE 2
Substrates tested for reduction by His₆-ElxO.

| | | k_{cat}/Kₘ | Relative |
|---|---|---|---|
| Entry | Substrate | (M⁻¹s⁻¹) | (k_{cat}/Kₘ) |
| 1 | Pyr-AAIVK | 2.43 ± 0.06 | 1.00 |
| 2 | Pyr-AAIV | 1.13 ± 0.00 | 0.47 |
| 3 | Pyr-AAI | 0.06 ± 0.00 | 0.02 |
| 4 | Pyr-AA | <0.03 | <0.01 |
| 5 | Pyr-A | <0.03 | <0.01 |
| 6 | Pyr-AAIVKBBIKAAKK | 14.2 ± 0.4 | 5.83 |
| 7 | Pyr-AAIVA | 1.33 ± 0.01 | 0.55 |
| 8 | Pyr-AAIAK | 1.59 ± 0.02 | 0.65 |
| 9 | Pyr-AAAVK | 0.29 ± 0.01 | 0.12 |
| 10 | Pyr-RAIVK | 5.50 ± 0.05 | 2.26 |
| 11 | Pyr-KAIVK | 4.22 ± 0.04 | 1.74 |
| 12 | Pyr-DAIVK | 0.29 ± 0.02 | 0.12 |
| 13 | Pyr-NAIVK | 7.60 ± 0.04 | 3.13 |
| 14 | Pyr-PAIVK | 0.13 ± 0.01 | 0.05 |
| 15 | Pyr-MAIVK | 15.5 ± 0.1 | 6.40 |
| 16 | Obu-AAIVK | 0.92 ± 0.03 | 0.38 |
| 17 | Obu-RAIVK | 1.51 ± 0.02 | 0.62 |
| 18 | Glx-AAIVK | <0.03 | <0.01 |

For all tested substrates, the values of k_{cat}/Kₘ were relatively small, presumably because the peptides are lacking structural features compared to the expected physiological substrate, such as the thioether rings or additional amino acids. The smaller peptides Pyr-AAIV and Pyr-AAI (Table 2, entries 2 and 3), but not Pyr-AA and Pyr-A (Table 2, entries 4 and 5), were reduced by His₆-ElxO in the presence of NADPH based on LC-MS analysis, although with considerably lower reaction rates compared with Pyr-AAIVK. In contrast, the peptide Pyr-AAIVKBBIKAAKK, where B stands for L-2-aminobutyric acid, was converted at a higher rate (Table 2, entry 6), suggesting that the length of the peptide is important for substrate recognition. The Ala scanning analysis performed along the sequence of Pyr-AAIVK (Table 2, entries 7-9) indicated that the enzyme was able to reduce all the peptides tested albeit with a lower k_{cat}/K_{M} for Pyr-AAAVK (Table 2, entry 9).

Next, we evaluated peptides containing amino acids with nonpolar (Pro, Met, Gly, Ile, Val, Phe; entries 14, 15, and 19), polar (Asn, Thr, Tyr; entries 13 and 20), acidic (Asp; entry 12), or basic (Arg, Lys, His; Table 2, entries 10, 11, and 21) residues at position 2, and found that they were all transformed to the reduced products. These results suggest that no substrate residue is absolutely required for enzymatic activity and that the minimal length of the peptide to be accepted as substrate is four residues. Interestingly, Pyr-RAIVK, Pyr-KAIVK, Pyr-NAIVK, and Pyr-MAIVK were better substrates for ElxO than Pyr-AAIVK (Table 2, entries 10, 11, 13, 15), whereas Pyr-DAIVK and Pyr-PAIVK were converted considerably less efficiently (Table 2, entries 12 and 14), suggesting that negatively charged residues and Pro in position 2 are not well tolerated.

OBu-AAIVK and OBu-RAIVK were also accepted as substrates leading to the formation of an N-terminal 2-hydroxybutyryl group (Hob), although at lower rates (Table 2, entries 16 and 17). Similarly, the peptides OBu-AAAVK and OBu-AAIAK were substrates for the enzyme. However, when Pyr was substituted by a glyoxyl (Glx) group, such as in the peptide Glx-AAIVK (Table 2, entry 18), no significant formation of the reduced peptide was observed.

### Evaluation of the Potential to Use ElxO to Reduce Other Lanthipeptides.

In addition to epilancin 15X, two other lantibiotics, epilancin K7 and epicidin 280, contain an N-terminal Lac moiety. To explore the potential of using His₆-ElxO for the synthesis of other lantibiotics, peptides mimicking the N-terminal portion of dehydroepilancin K7 (Pyr-AAVLK) and dehydroepicidin 280 (Pyr-LGPAIK) were synthesized and tested as substrates. His₆-ElxO reduced both peptides, even though their sequences are quite different from the N-terminus of epilancin 15X. Similarly, peptides resembling the N-terminus of lactocin S (Pyr-APVLA and Pyr-BPVLAAVAVAKKK) and Pep5 (OBu-AGPAIR) were incubated with His₆-ElxO in the presence of NADPH. All the peptides were reduced as confirmed by LC-MS analysis.

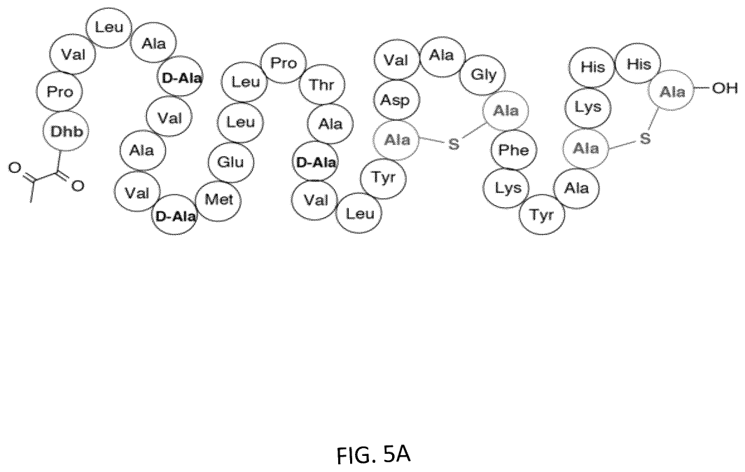

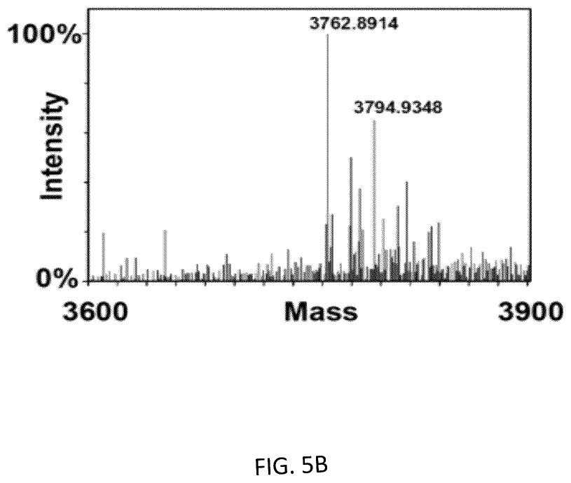

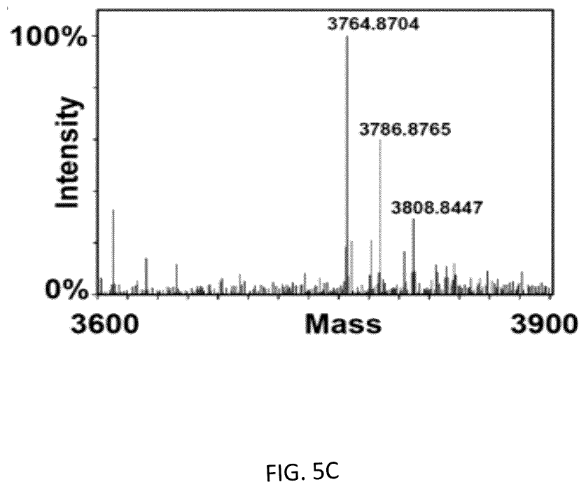

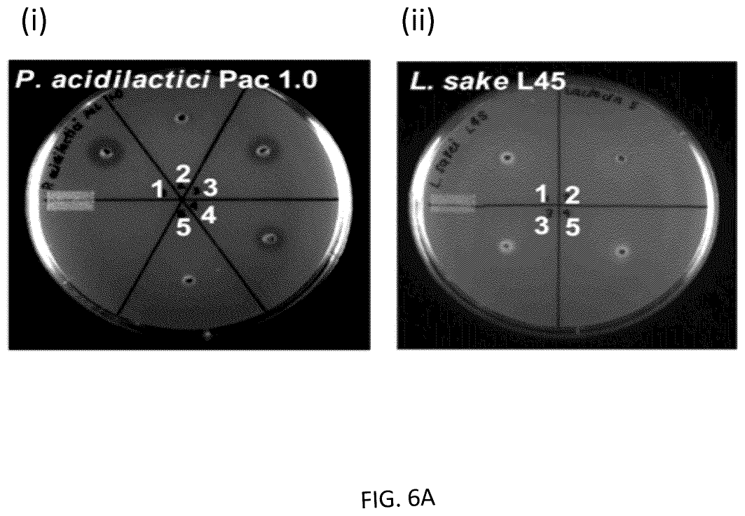

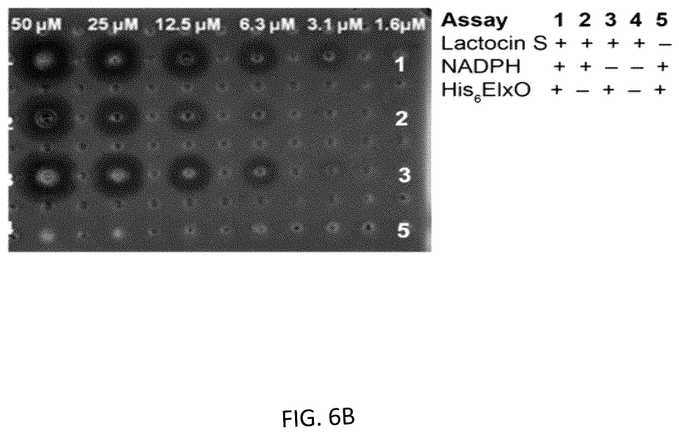

Encouraged by these results with short peptides we next turned to lactocin S, a 37-residue lantibiotic (FIG. 5A) produced by Lactobacillus sake L45 that contains an N-terminal Pyr. To evaluate if lactocin S would be a substrate for ElxO, a synthetic sample of the lantibiotic was incubated with NADPH in the presence of His₆-ElxO and the reduction of the peptide was monitored by high-resolution LC-MS (FIG. 5B, C), confirming the formation of dihydrolactocin S. Samples containing the reduced peptide and control samples containing lactocin S were tested by agar diffusion bioactivity assays using either Pediococcus acidilactici Pac1.0 as indicator strain (FIG. 6A (panel (i)) and FIG. 6B) or the lactocin S producer strain (FIG. 6A (panel (ii)). All the peptides were active against P. acidilactici Pac1.0 but not against L. sake L45 suggesting that the N-terminal Pyr is not involved in self-immunity. From the serial dilution assay (FIG. 6B), the sizes of the inhibition zones were determined and the concentrations at which no inhibition zones were observed were calculated. Interestingly, the sample containing dihydrolactocin S produced slightly larger inhibition zones and smaller critical concentrations than control samples containing the native peptide, illustrating that the N-terminal Pyr of lactocin S is not essential for bioactivity. Similar results were obtained upon determination of apparent minimal inhibitory concentrations (MIC) by a serial dilution bioactivity assay in liquid media.

Although all LanPs characterized to date belong to the subtilisin-like serine endopeptidases superfamily, their cleavage sequences are more diverse than those of class II lanthipeptide peptidases. Our current work provides evidence that the LanA and LanP proteins likely co-evolved and that LanP sequence specificity is mainly determined by the amino acids near the proteolytic site. Specifically, the conserved E/D-L/V-x-x-Q-T1/S1 sequence motif (SEQ ID NO: 3) present in the ElxA-group of LanAs provides the full recognition elements for ElxP. This recognition sequence may find use in applications of ElxP for cleaving off fusion tags or removing leader peptides from RiPPs.

### CylA as a LanP Having Enhanced Substrate Sequence Context Tolerance

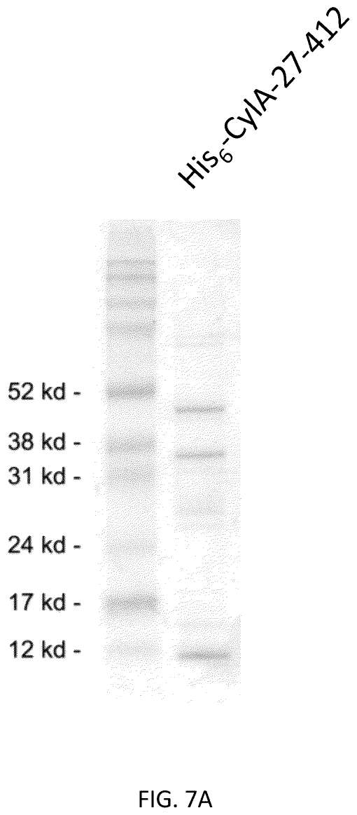

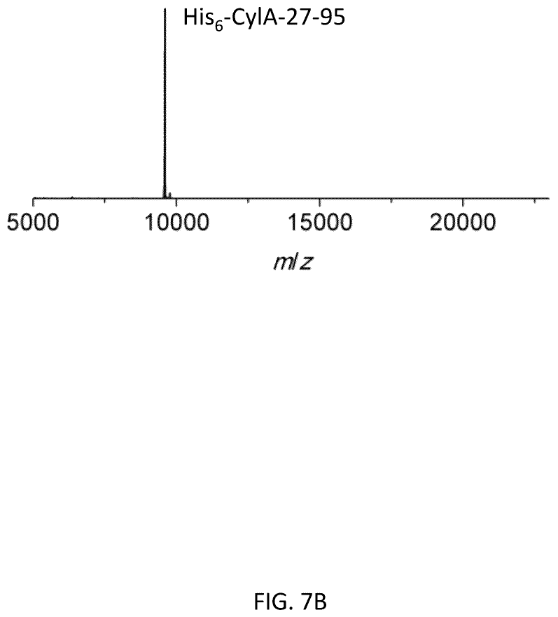

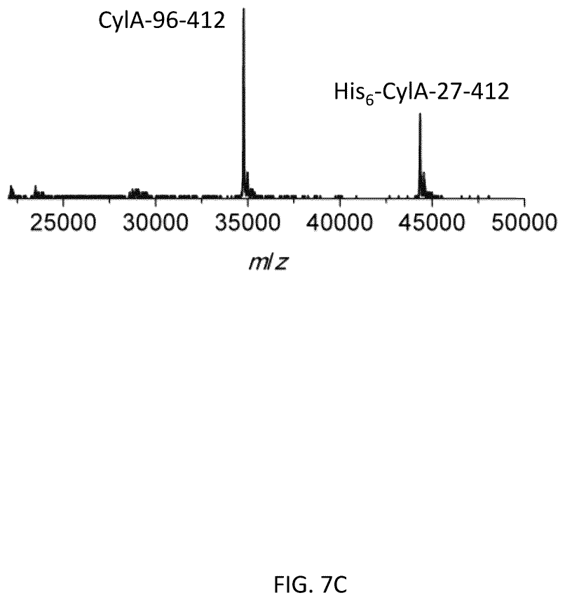

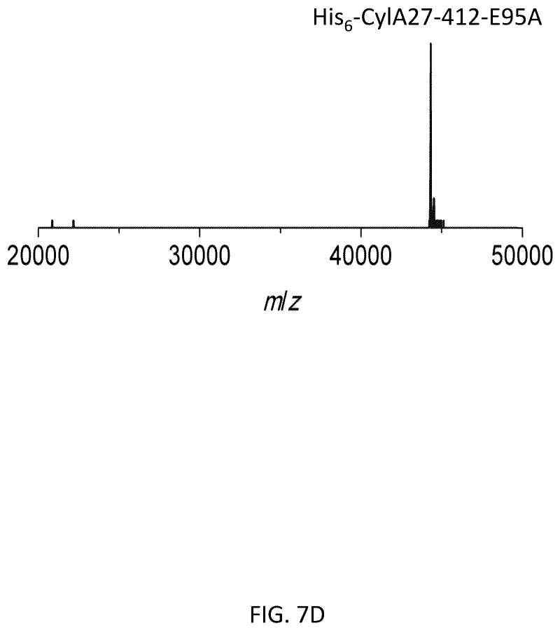

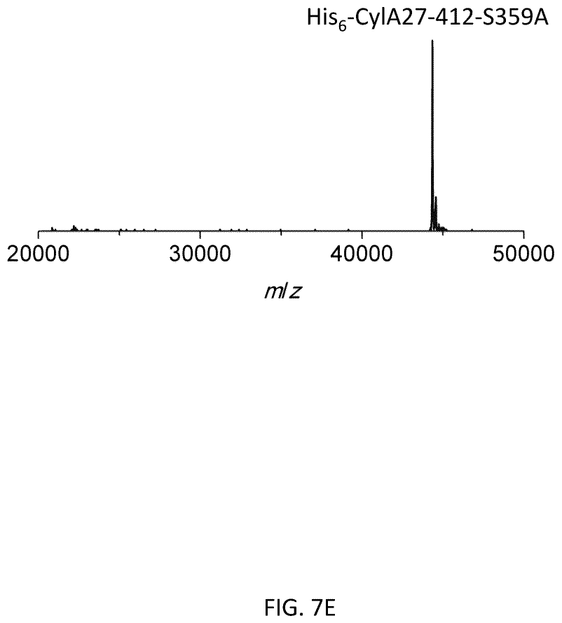

CylA shows considerable homology to class I LanPs but is located in a separate clade in a Markov chain Monte Carlo phylogenetic tree (FIG. 2B), suggesting it may have properties that are different from class I LanPs. The gene encoding CylA was synthesized with codons optimized for E. coli expression and cloned into the multiple cloning site 1 (MCS1) of a pRSFDuet vector. Residues 1-26 that are predicted to constitute a secretion signal peptide were removed to improve overall solubility. The protein was expressed in E. coli with a hexa-histidine tag fused to the N terminus (SEQ ID NO: 10 (polypeptide)) and purified by immobilized metal affinity chromatography. Surprisingly, purified CylA protein showed 3 bands by SDS-PAGE, with one band corresponding to the full length His₆-CylA-27-412 (SEQ ID NO: 10) and two other bands appearing at molecular weights of about 35 kDa and 10 kDa (FIG. 7A). Matrix-assisted laser desorption ionization time-of-flight mass spectrometry (MALDI-TOF MS) analysis indicated masses of 9,591 Da and 34,815 Da (FIG. 7B, C), consistent with two fragments of His₆-CylA-27-412: an N-terminal fragment His₆-CylA-27-95 with a calculated mass of 9,591 Da and a C-terminal fragment CylA-96-412 with a calculated mass of 34,812 Da. This observation suggested a cleavage event after residue Glu95 during CylA expression or purification, in accordance with a previous report. In order to confirm the cleavage site, we constructed a CylA mutant in which the putative cleavage site was changed from Glu to Ala. His₆-CylA-27-412-E95A (SEQ ID NO: 11) was expressed in E. coli and purified using the same procedure as that for His₆-CylA-27-412. Indeed, only one band corresponding to the full length protein was observed, with a molecular weight of 44,335 Da determined by MALDI-TOF MS (FIG. 7D).

Based on its sequence, CylA is a subtilisin-like serine protease with a conserved catalytic triad consisting of aspartate, histidine and serine. Given that CylA cleaves its substrates CylL_{L} and CylL_{S} after a Glu, we wondered whether the observed cleavage of CylA could be autocatalytic. If so, the proteolysis should be abolished by disrupting the catalytic triad. We therefore constructed another CylA mutant with the catalytic Ser359 changed to Ala. The mutant protein was expressed and purified using the same procedure as that for wild type CylA. Indeed, only one band was observed by SDS-PAGE for purified His₆-CylA-27-412-S359A, corresponding to the full length protein. The expression of full length His₆-CylA-27-412-S359A was further confirmed by MALDI-TOF MS (FIG. 7E). Therefore, we conclude that CylA catalyzes the cleavage at position 95. Our findings mirror those of a very recent report on the class I lanthipeptide protease EpiP, which was shown to use an autocatalytic mechanism of cleavage between Lys87 and Thr88.

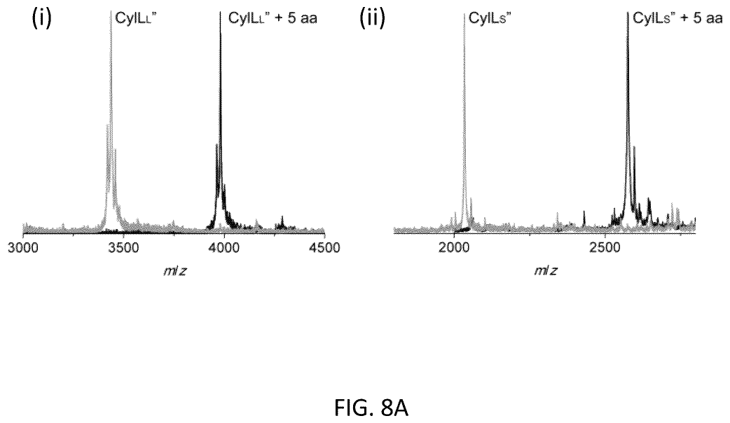

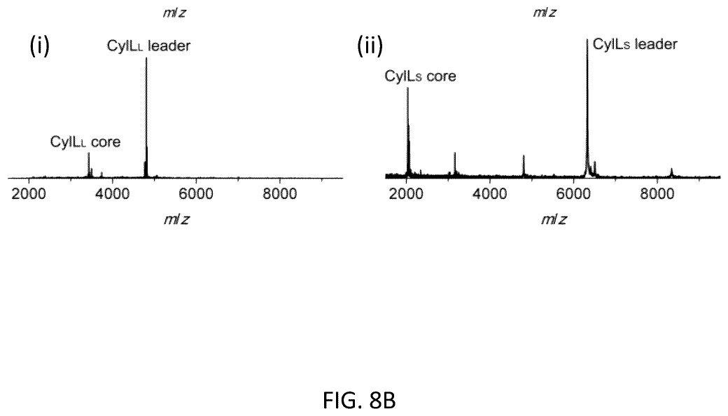

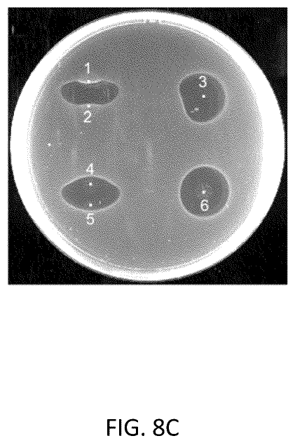

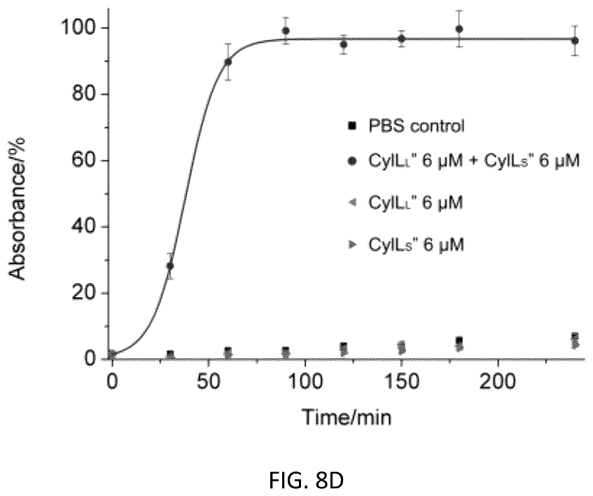

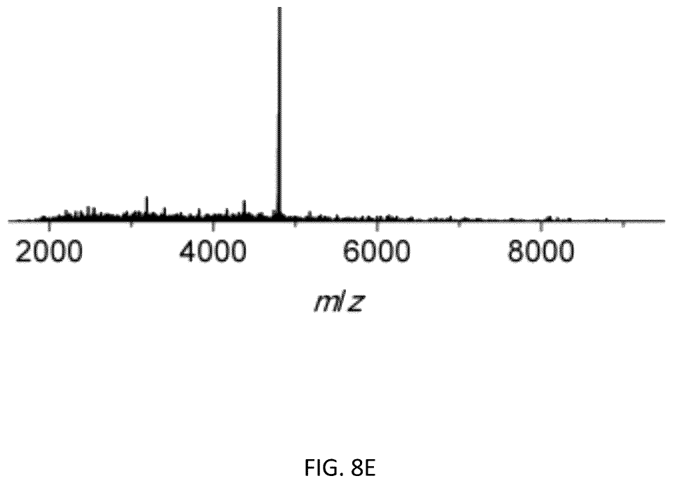

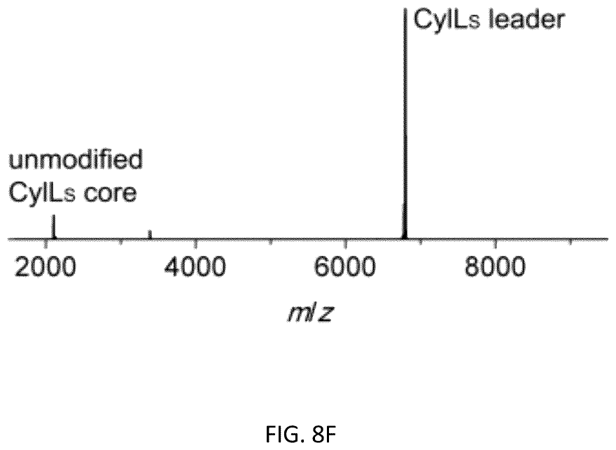

With purified CylA in hand, we set out to test its proteolytic activity with the peptides CylL_{L} and CylL_{S}. A previous study reported that CylA catalyzed the removal of the 6-residue sequence GDVQAE from the N-terminus of both modified core peptides subsequent to leader peptide removal at the GS motif by Cyl_{B} in the producing strain Enterococcus faecalis. In this work, dehydrated and cyclized CylL_{L} and CylL_{S} peptides were obtained by co-expression of CylL_{L} and CylL_{S} peptides with their lanthionine synthetase CylM in E. coli. Instead of using the membrane bound protein Cyl_{B}, we employed the commercial protease AspN, which specifically cleaved N-terminal to Asp-5, leaving 5 amino acids (DVQAE) on the core peptides. These peptides were then incubated with CylA and the 5 amino acids were successfully removed (FIG. 8A), demonstrating CylA purified from E. coli exhibited the desired activity. We also incubated CylA with full-length modified CylL_{L} and CylL_{S}, and MS analysis demonstrated that surprisingly the enzyme also removed the entire leader peptides (FIG. 8B). Cytolysin obtained in this way exhibited the anticipated antimicrobial activity against Lactococcus lactis HP (FIG. 8C) and hemolytic activity against rabbit red blood cells (FIG. 8D). Importantly, CylA did not require post-translational modifications of the precursor peptides as it also removed the leader peptides from linear CylL_{L} and CylL_{S} (FIG. 8E, F).

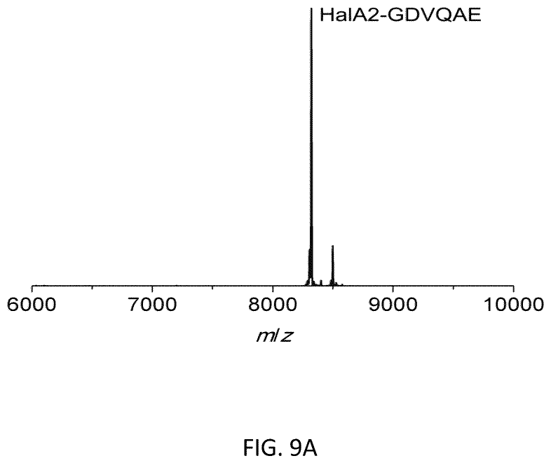

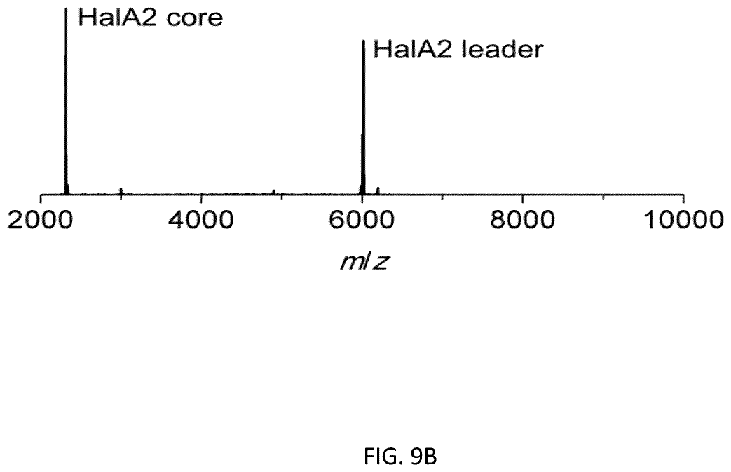

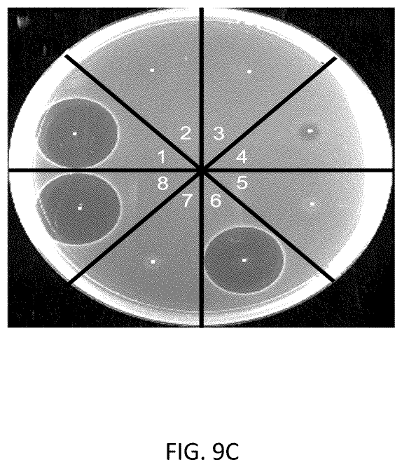

Based on these results, we hypothesized that CylA specifically recognizes the GDVQAE sequence (SEQ ID NO: 46). To test this hypothesis, the GDVQAE sequence was engineered into HalA2, the precursor peptide for the lantibiotic haloduracin β (Halβ). Halβ and haloduracin α (Halα) constitute a two-component lantibiotic. The putative recognition sequence was installed between the HalA2 leader and core peptides by substituting the residues at positions −6 to −1. This HalA2-GDVQAE peptide was co-expressed with the cognate lanthionine synthetase HalM2 in E. coli, resulting in the desired 7 dehydrations (FIG. 9A). The modified peptide was then incubated with CylA and the leader peptide was successfully removed as monitored by MALDI-TOF MS (FIG. 9B). To confirm the efficiency of the proteolytic reaction, we compared the antimicrobial activities of the product Halβ against Lactococcus lactis HP with that obtained from a previously reported method utilizing the commercial protease factor Xa. The zones of growth inhibition were identical for the Halβ obtained by either method when applied at the same precursor peptide concentration in combination with Halα (FIG. 9C).

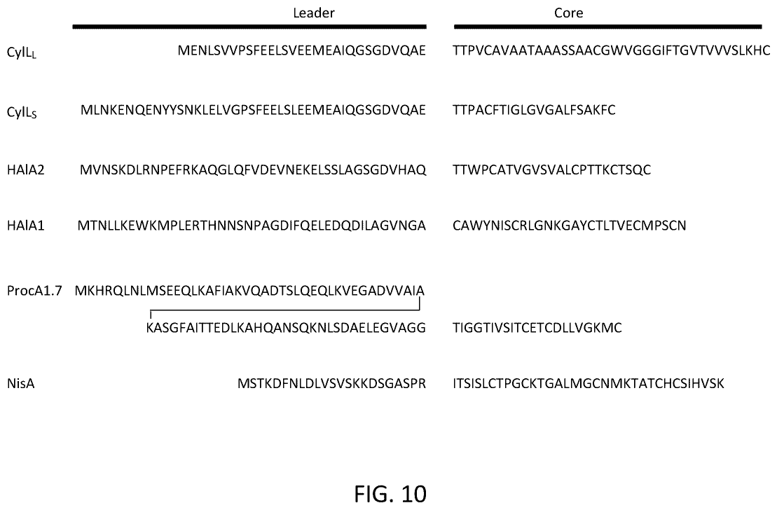

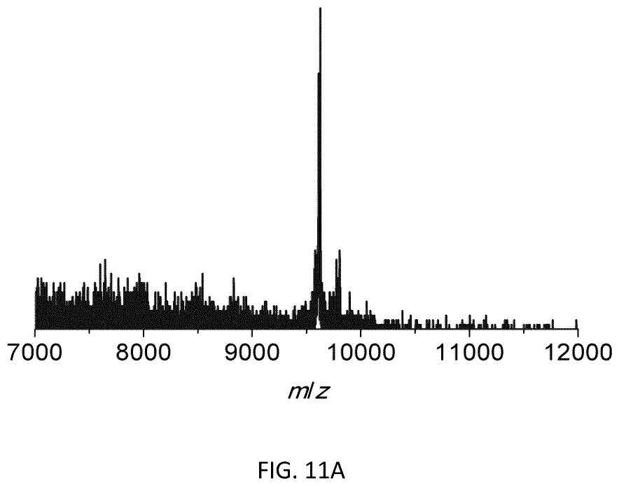

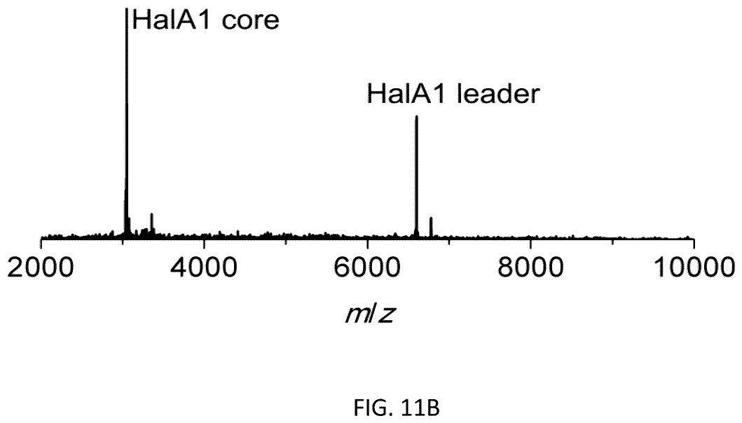

To further evaluate the substrate scope of CylA, we explored its activity against a variant of the precursor peptide for Halα. Again, the residues at positions −6 to −1 were replaced with GDVQAE by site-directed mutagenesis to produce HalA1-GDVQAE. Halα does not show any sequence homology with the two cytolysin peptides or Halβ (FIG. 10), and thus incubation of HalA1-GDVQAE would further test the substrate tolerance of CylA with respect to the P′ positions. HalA1-GDVQAE was co-expressed with its cognate lanthionine synthetase HalM1 in E. coli and was fully modified (FIG. 11A). The modified peptide was successfully digested upon incubation with CylA, affording 2 fragments corresponding to the leader peptide and the modified core peptide corresponding to Halα (FIG. 11B). Proteolysis was successful regardless of the presence of the reducing agent triscarboxyethyl phosphine (TCEP), indicating that CylA tolerated both cysteine and cysteine in the P1′ position.

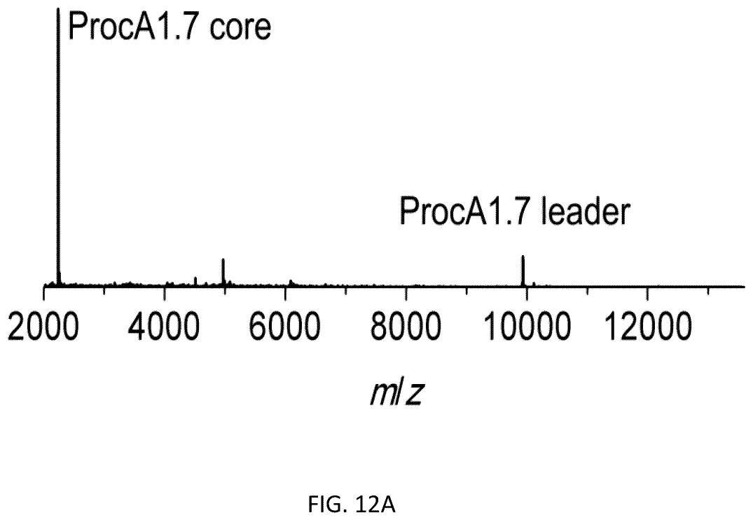

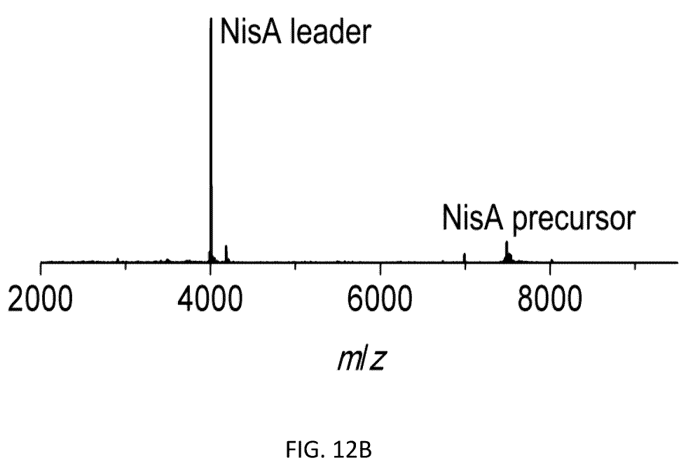

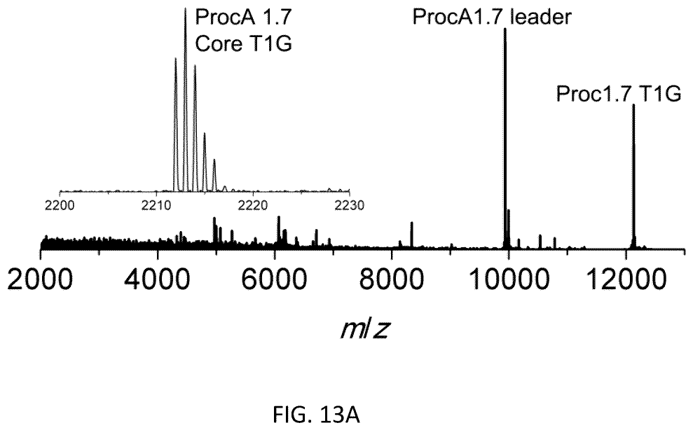

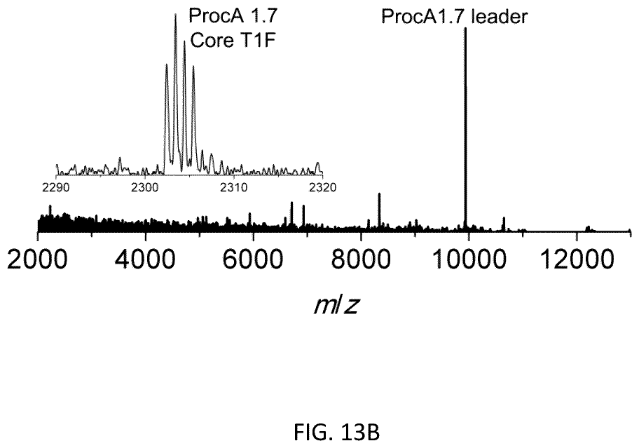

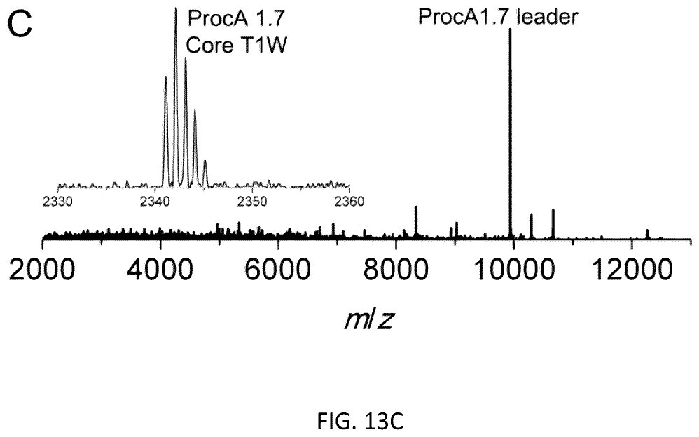

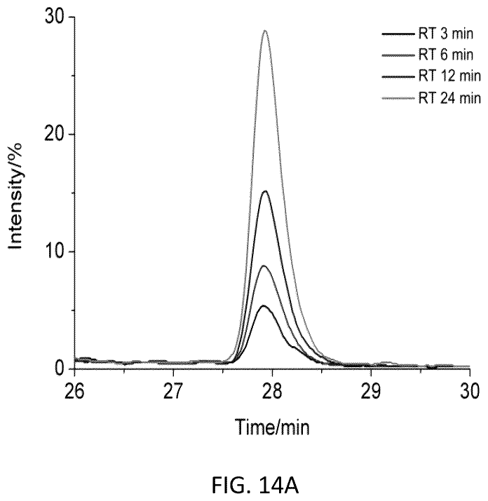

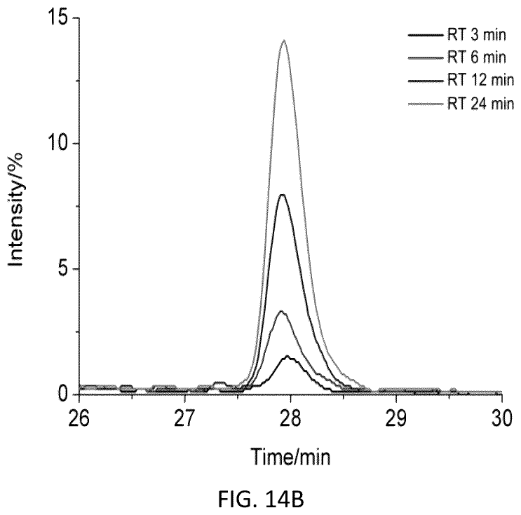

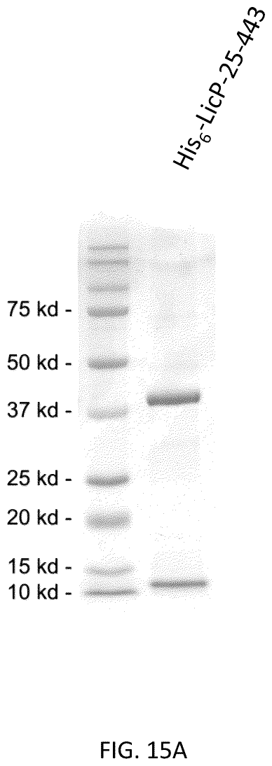

To test whether CylA accepts linear peptides containing the GDVQAE sequence other than CylL_{L} and CylL_{S}, we engineered the recognition sequence into two more peptides—ProcA1.7 and NisA. Upon incubation with CylA, the leader peptides of both His₆-ProcA1.7-GDVQAE and His₆-NisA-GDVQAE were successfully removed, indicating the broad substrate scope of CylA (FIG. 12). We note that although the leader peptide of ProcA1.7 contains 7 glutamates (FIG. 10), CylA only cleaved after the engineered glutamate at position −1, suggesting that CylA is highly specific for the GDVQAE sequence. To further probe the utility of CylA as a traceless protease, we switched the P1′ position of ProcA1.7-GDVQAE from threonine to glycine, phenylalanine or tryptophan. CylA specifically cleaved after Glu-1 for all three mutant peptides, albeit with a lower efficiency for the T-1G mutant as demonstrated by MALDI-TOF MS (FIG. 13). Encouraged by these results, we also engineered the cleavage site between the His₆-tag and the ProcA1.7 peptide. Incubation with CylA indeed resulted in cleavage and removal of the His₆-tag sequence. Finally, we engineered a Cys in the P1′ position of ProcA1.7-GDVQAE and NisA-GDVQAE. CylA cleanly removed the leader peptide from both substrates. This finding suggests that CylA may be a very useful protease to prepare peptides and proteins with N-terminal Cys residues, which have great utility in native and expressed protein ligation. Collectively, these results show that the activity of CylA is highly portable (Table 3).

TABLE 3
Peptides with different P′ sequences that are accepted
by CylA. All peptides had the GDVQAE sequence inserted
before the P1′ position.

| Peptide | P′ position |
|---|---|
| CyIL_{L}^{[a]} | TTPVC |
| CylL_{S}^{[a]} | TTPAC |
| HalA2^{[b]} | TTWPC (LL-MeLan) |
| HalA1^{[c]} | CAWYN |
| NisA | ITSIS |
| NisA-I1C | CTSIS |
| ProcA1.7 | TIGGT |
| ProcA1.7-T1C | CIGGT |
| ProcA1.7-T1G | GIGGT |
| ProcA1.7-T1F | FIGGT |
| ProcA1.7-T1W | WIGGT |
| His₆-ProcA1.7^{[d]} | TMKHR |

^{[a]}These peptides were substrates with the linear sequences shown and also after the Cys in the 5^{th} position formed a methyllanthionine with the Thr at position 1.
^{[b]}Peptide was modified by HalM2 with a MeLan residue at the P1′ position.
^{[c]}Peptide was modified by HalM1 with either a Cys or a disulfide linkage at the P1′ position.
^{[d]}GDVQAE sequence inserted between the plasmid encoded His₆-tag sequence and ProcA1.7.

The enzyme tolerates hydrophobic, hydrophilic, branched and aromatic amino acids in the P1′ position. Its substrate scope is not limitless, however, as it did not accept Glu or Lys in the P1′ position.

We next returned to the importance of the autocatalytic processing step for activity. Incubation of modified CylL_{S} with His₆-CylA-27-412-E95A resulted in cleavage, suggesting that the processing event is not absolutely required for proteolytic activity. To assess the effect of self-cleavage on the rate of cleavage, CylA-96-412 and His₆-CylA-27-412-E95A were incubated with modified CylL_{S}, and the formation of CylL_{S}″ was monitored by liquid chromatography MS (LC/MS). CylA-96-412 catalyzed the proteolysis of 20 substrate peptides per minute under the condition we tested, whereas His₆-CylA-27-412-E95A exhibited an approximate 10-fold lower rate of producing CylL_{S}″ (FIG. 14). Thus, the self-cleaving event leads to the activation of CylA, although the autoprotolysis is not absolutely required for the protease activity. The recent X-ray structure of the class I protease EpiP illustrates that upon cleavage, the prodomain interacts non-covalently with the catalytic domain through complementary electrostatic surfaces. The active site of the processed enzyme is more exposed, which may explain the increased activity observed in this work for processed CylA.

### LicP as an Efficient Sequence-Specific Traceless Protease

In vitro characterization of LicP, a class II LanP protease, involved in the biosynthesis of the lantibiotic lichenicidin, revealed a self-cleavage step that removes 100 amino acids from the N-terminus. Investigation of its substrate specificity demonstrated that LicP can serve as an efficient sequence-specific traceless protease. Encouraged by these findings for LicP, we identified 12 other class II LanPs, nine of which were previously unknown, and suggest that these proteins may serve as a pool of proteases with diverse recognition sequences for general traceless tag removal applications, expanding the current toolbox of proteases.

### Expression of LicP Reveals a Self-Cleavage Maturation Process

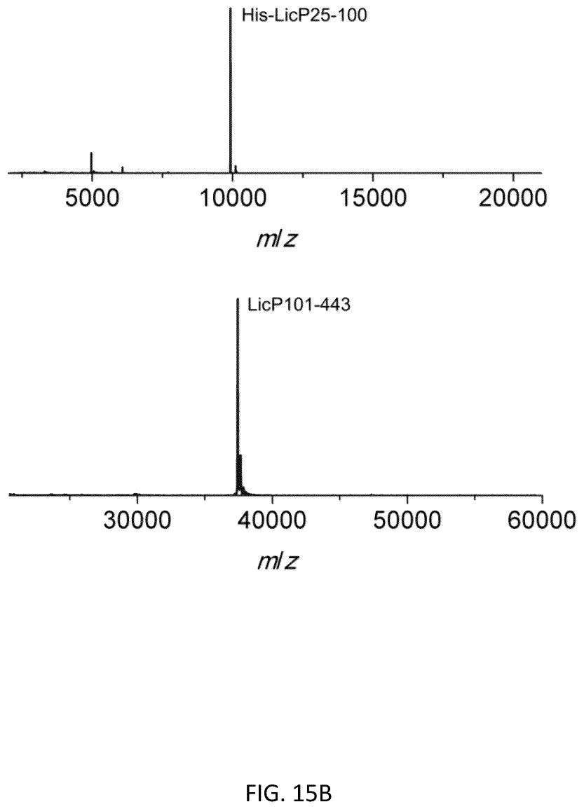

The licP gene was amplified from genomic DNA of B. licheniformis ATCC 14580 and cloned into an expression vector. A hexa-histidine tag was installed at its N terminus, and the first 24 amino acids of LicP, which correspond to a secretion signal peptide, were omitted. Upon expression in Escherichia coli BL21 (DE3) and purification using immobilized metal affinity chromatography, two bands were observed by gel electrophoresis (FIG. 15A). Analysis by matrix-assisted laser desorption ionization time-of-flight mass spectrometry (MALDI-TOF MS) demonstrated masses of 9,923 Da and 37,431 Da (FIG. 15B). These molecular weights are in agreement with two fragments of LicP, an N-terminal portion His₆-LicP-25-100 with a calculated mass of 9,924 Da and a C-terminal portion LicP-101-433 with a calculated mass of 37,449 Da, suggesting that a cleavage event occurred during the expression of His₆-LicP-25-433 (SEQ ID NO: 13) (hereafter referred to as wild type LicP).

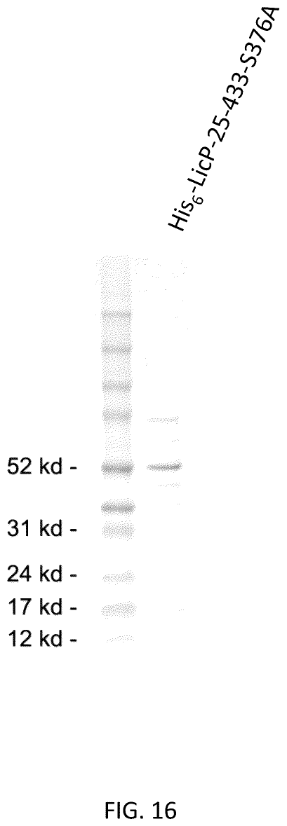

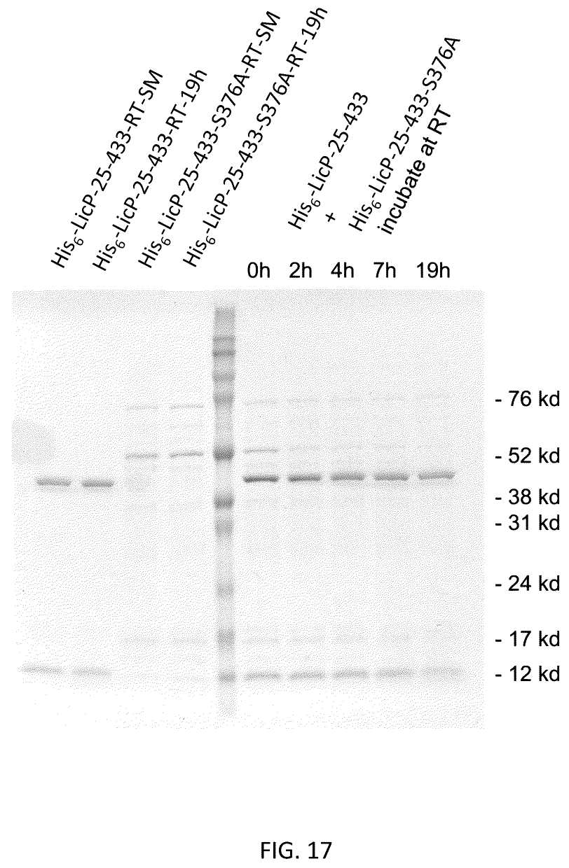

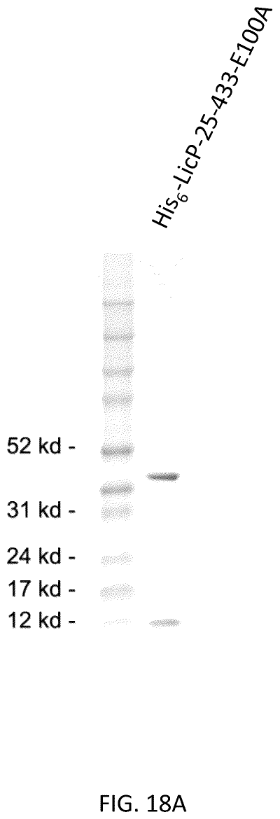

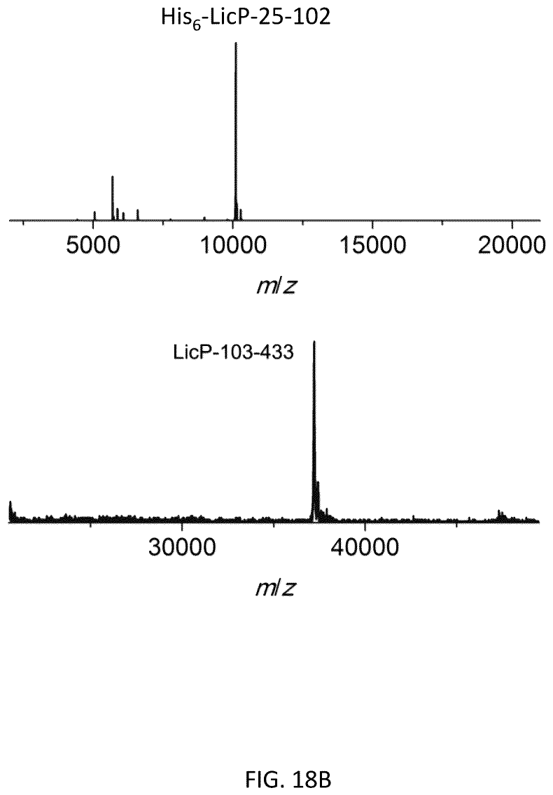

Such proteolytic processing has been reported for several extracellular class I LanP proteases and was suggested to be autocatalytic like other subtilisins. To test whether this mechanism applied to the class II enzyme LicP, we substituted the predicted catalytic Ser376 with Ala. Unfortunately, His₆-LicP-25-433-S376A was expressed almost exclusively in the insoluble fraction. We also mutated His186 predicted to be involved in the catalytic triad, but His₆-LicP-25-433-H186A was also expressed insolubly. We eventually were able to obtain a very small amount of soluble His₆-LicP-25-433-S376A, demonstrating that indeed the proteolytic cleavage after Glu100 was abolished (FIG. 16). The observation that an inactivated LicP was expressed as the full length protein indicates that the cleavage event is catalyzed by LicP rather than E. coli proteases. Our findings mirror those of a very recent report on a protease from a non-lanthipeptide producing organism that has sequence homology with the lanthipeptide protease EpiP, which employs an autocatalytic mechanism of cleavage between Lys87 and Thr88. Using His₆-LicP-25-433 and its S376A mutant, we showed that autoprotolysis can take place intermolecularly, albeit slowly (FIG. 17). To obtain an active form of LicP with the pro-sequence covalently attached, we substituted Glu100 with Ala. However, His₆-LicP-25-433-E100A was again expressed and purified as two fragments (FIG. 18). Surprisingly, the resulting fragments corresponded to a shifted cleavage site from residue Ala100 to Glu102 (FIG. 18). Further mutation of Glu102 to Ala abolished the production of soluble protein (not shown).

### In Vitro Characterization of LicP

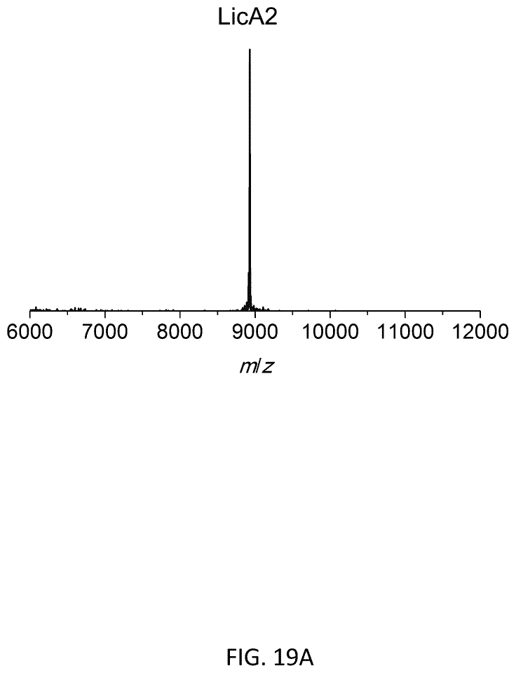

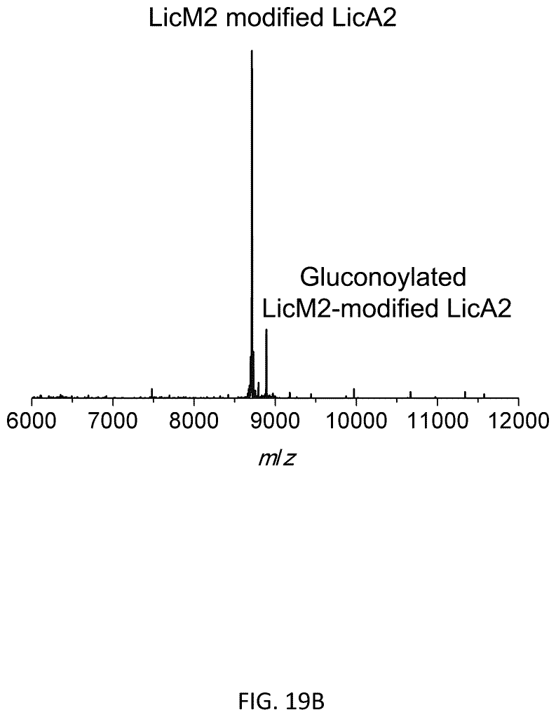

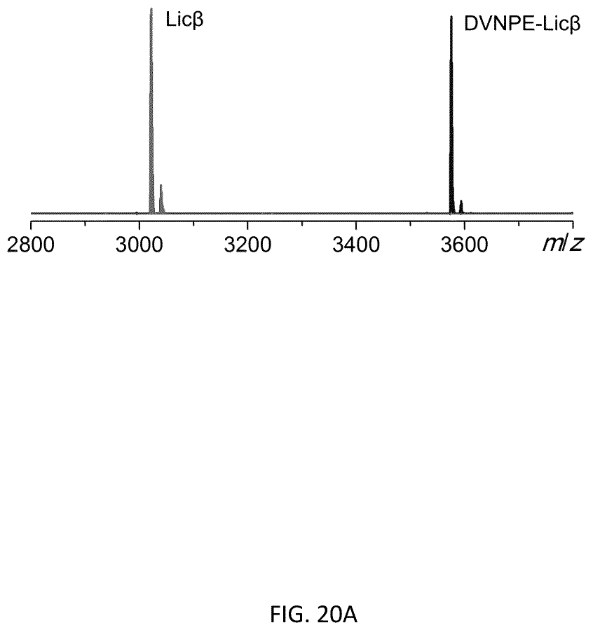

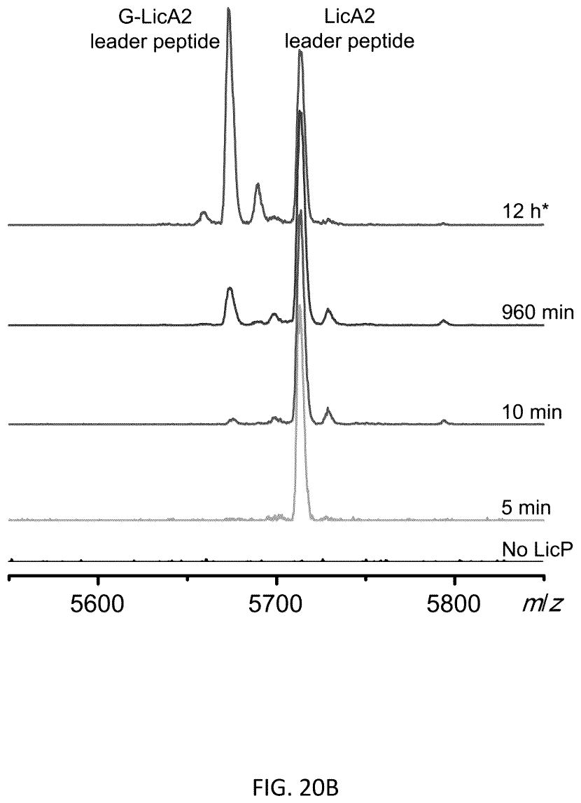

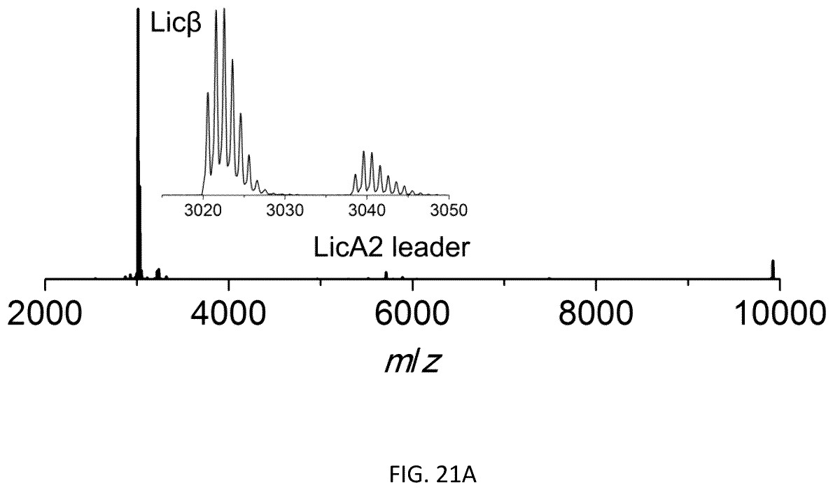

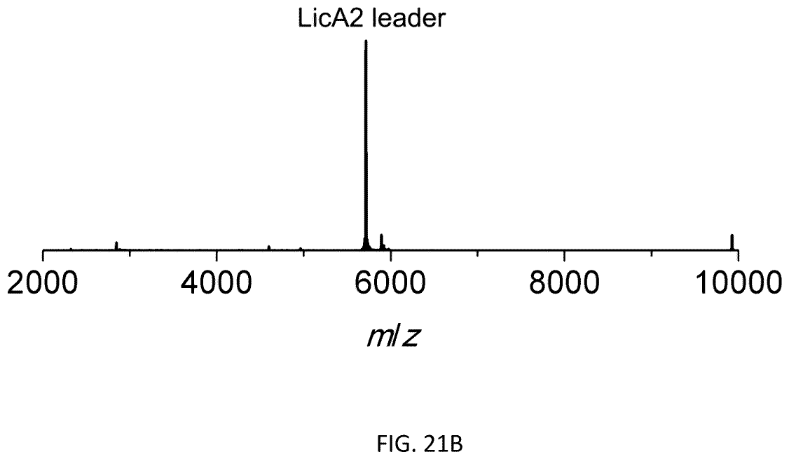

We next tested LicP activity against the substrate peptide. It has been suggested that LicP trims off the 6-residue oligopeptide NDVNPE from LicA2′ to generate mature LicP. In this work, dehydrated and cyclized LicA2 was obtained by co-expressing LicA2 with its cognate lanthionine synthetase LicM2 in E. coli (FIG. 19). Instead of using the membrane-bound protein LicT to produce LicA2′, we employed the commercial protease AspN to generate DVNPE-Lic. Upon incubation with wild type LicP, the 5-residue oligopeptide was successfully removed (FIG. 20A), confirming LicP's anticipated proteolytic activity. When the full length modified LicA2 was incubated with LicP, the peptide was also consumed, resulting in two fragments corresponding to the leader peptide and LicP (FIG. 21A). This observation suggests that LicP does not require prior proteolysis by LicT to produce LicP. We next incubated linear LicA2 with LicP to test whether unmodified LicA2 was also a substrate. Cleavage of the unmodified LicA2 peptide was observed (FIG. 21B), indicating that post-translational modifications are not required for substrate recognition by LicP.

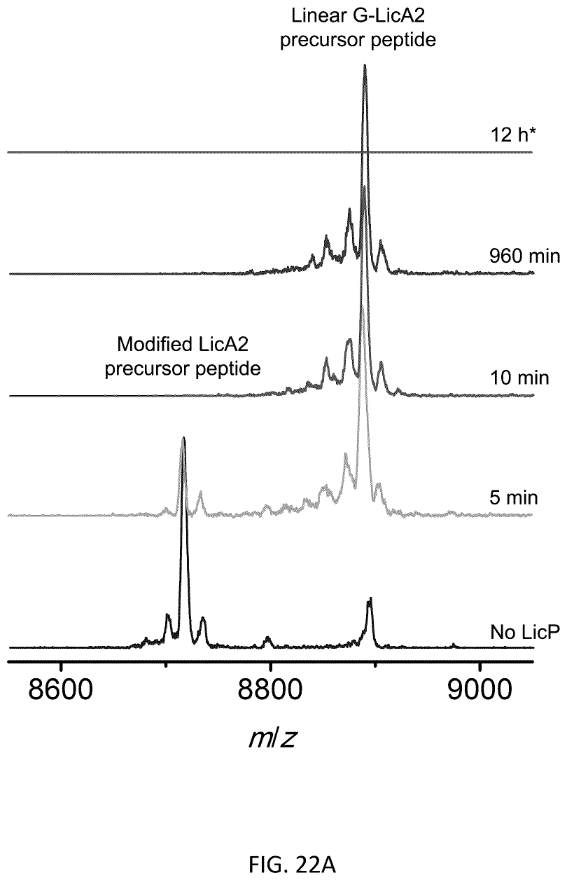

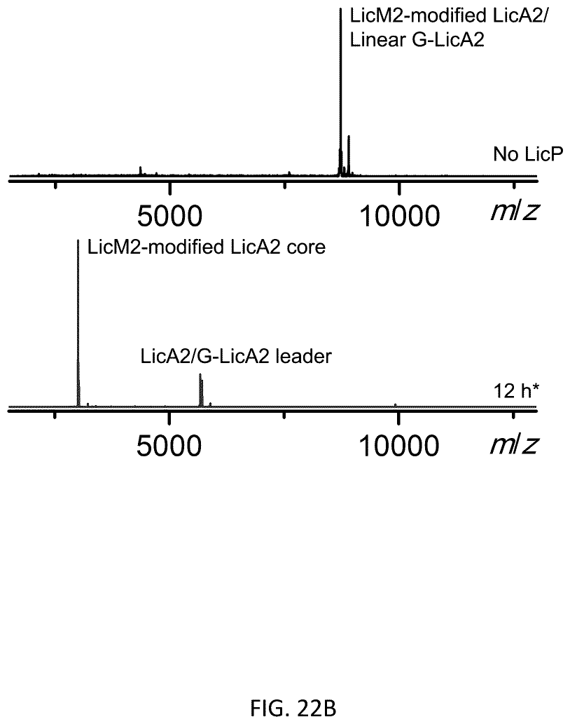

We further investigated whether the enzyme displays a preference for modified or linear LicA2. Liquid chromatography-based kinetic analysis of the time and concentration dependence of the cleavage reactions was hampered by the poor solubility of the LicA2 and Licβ peptides. Instead, we employed a competitive MALDI-TOF MS assay for a semi-quantitative time-dependent analysis at one substrate concentration, in which LicP was supplied to an equimolar mixture of modified and linear LicA2 and the production of leader peptides was monitored over time. In order to differentiate the otherwise identical leader peptides after proteolysis, we introduced a Pro to Gly mutation between the hexa-histidine tag and the precursor peptide in linear LicA2 (G-LicA2). The leader peptides obtained by complete proteolysis of equimolar amounts of modified and linear LicA2 exhibited comparable signal intensities when monitored by MALDI-TOF MS, confirming that the Pro to Gly mutation does not alter the ionization efficiency significantly (FIG. 20B). LicP was incubated with an 800-fold excess of modified and linear LicA2 (i.e. enzyme:combined substrates=1:1600), and MALDI-TOF MS analysis illustrated complete consumption of modified LicA2 peptide within 10 min, corresponding to a rate of at least 80 min⁻¹, whereas the cleavage of linear LicA2 only started after the modified LicA2 had been consumed and required more enzyme to be completed (FIG. 20B and FIG. 22). Collectively, our observations indicate that although both are substrates for the enzyme, LicP strongly prefers modified LicA2.

### LicP can Serve as a Sequence-Specific Traceless Protease

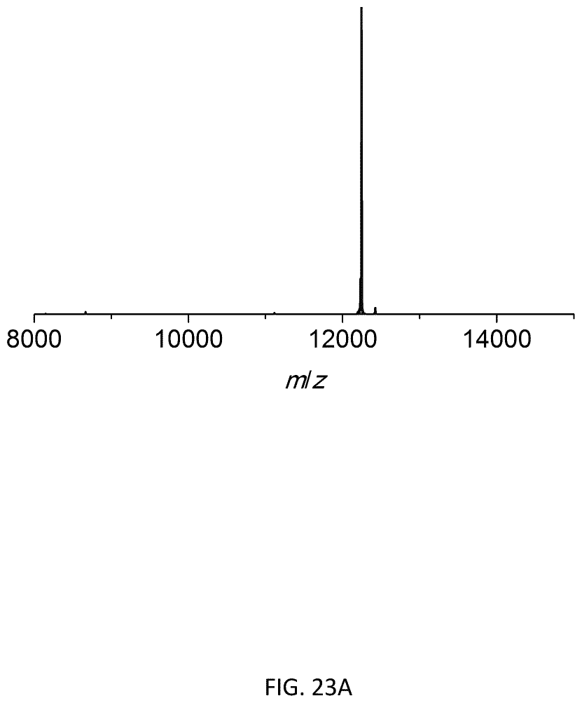

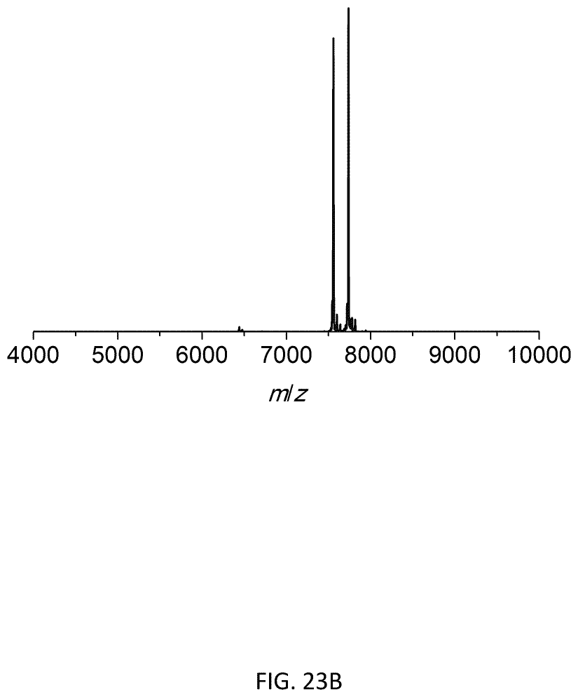

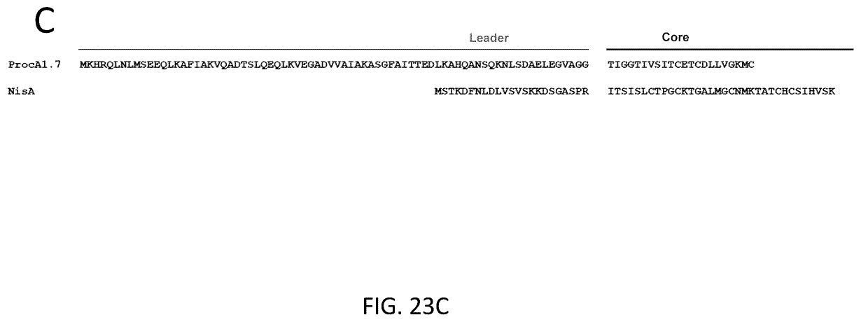

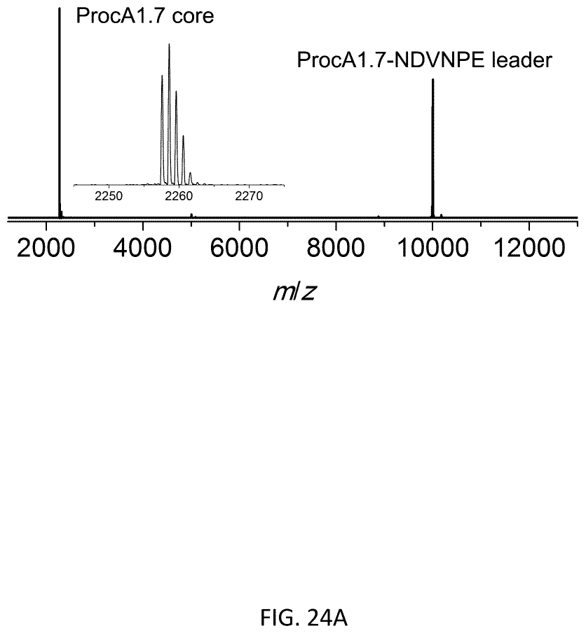

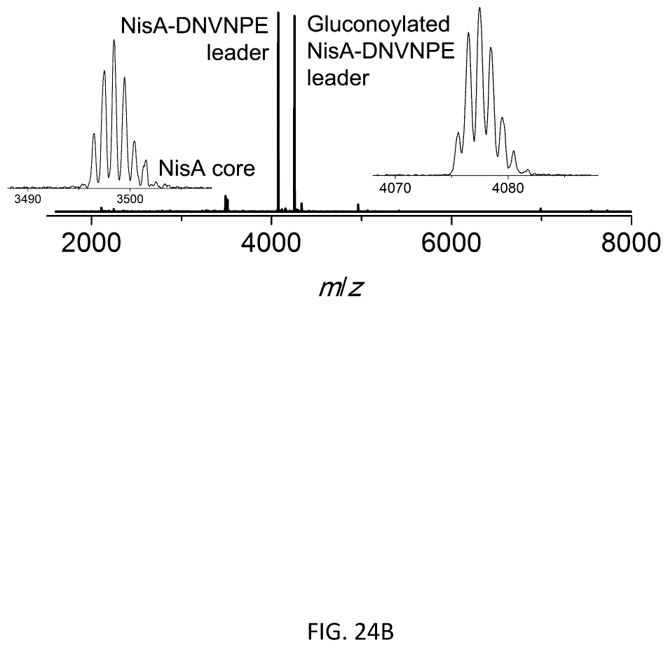

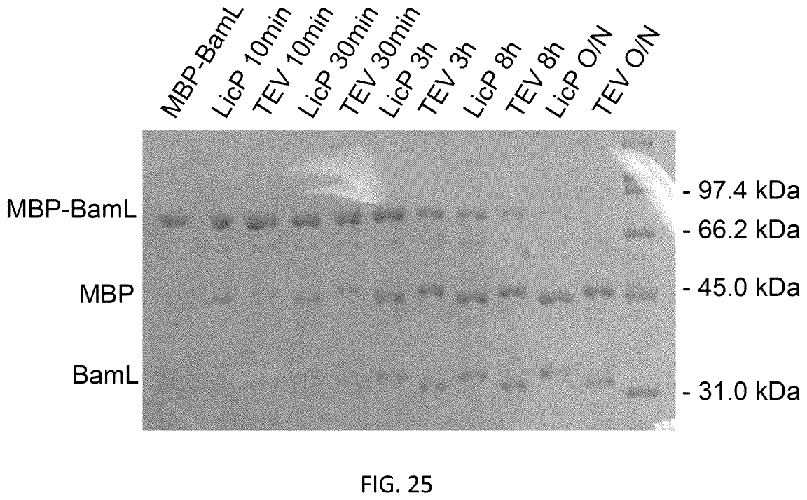

The observation that LicP removes the oligopeptide NDVNPE and the entire leader peptide from modified or linear LicA2 suggests that it specifically recognizes the NDVNPE sequence but is rather tolerant of other regions of the peptides. We decided to test this hypothesis in the lanthipeptide family, as site-specific removal of leader peptides is critical for producing lanthipeptides in vitro and this step is often challenging as only a limited choice of proteases is available. ProcA1.7 and NisA, the precursor peptides of the lanthipeptides prochlorosin 1.7 and nisin, were mutated to substitute the last six residues of their leader peptides with the NDVNPE sequence (FIG. 23). ProcA1.7-NDVNPE and NisA-NDVNPE were incubated with LicP, resulting in successful removal of their leader peptides (FIG. 24). Encouraged by these observations, we further explored LicP's potential of removing an expression tag and compared its activity with that of the widely used sequence-specific Tobacco Etch Virus (TEV) protease. The methyltransferase BamL was expressed with a maltose-binding protein (MBP) fused at the N-terminus and the protease recognition sequences were installed between MBP and BamL (SEQ ID NO.: 221). LicP was capable of removing the MBP-tag in front of the BamL protein with similar efficiency as TEV (FIG. 25), confirming that the substrate scope of LicP is not limited to peptides.

The experiments with linear and modified LicA2 as well as ProcA1.7-NDVNPE, NisA-NDVNPE and MBP-BamL demonstrated that LicP tolerates Dhb, Thr, Ser and Ile in the P1′ position. To further evaluate its tolerance, we altered the P1′ position in NisA-NDVNPE from Ile to eight other amino acids (Gly, Cys, Thr, Leu, Phe, Trp, Glu and Lys;

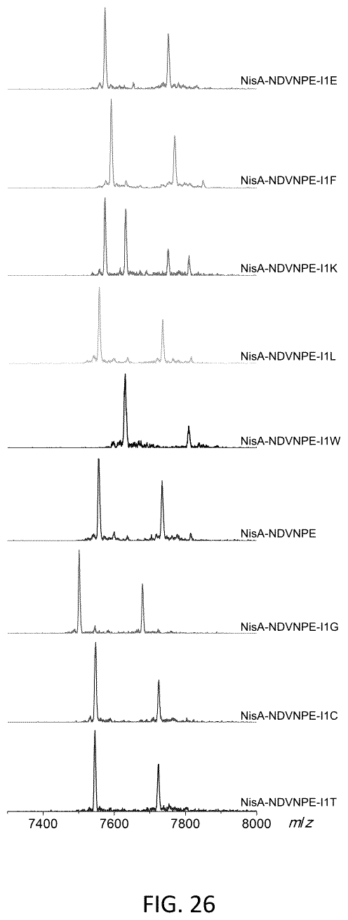

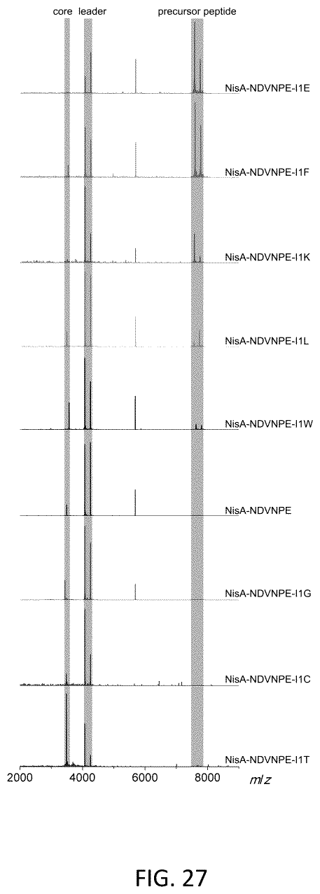

FIG. 26). All these mutants were accepted by LicP as in all cases removal of the NisA leader peptide was observed when substrate peptides were supplied in 30 to 300 fold excess over the enzyme (FIG. 27). The highest proteolytic efficiency was obtained when the P1′ position was occupied by Thr or Cys; traceless removal of tags in front of Cys is highly valuable for cysteine-based ligation chemistry. The removal of the NisA leader peptide in front of a Gly or Ile residue was slightly less efficient, but complete consumption of precursor peptides was still observed. NisA-NDVNPE analogs with Trp, Leu, and Lys at the P1′ position were also accepted by LicP, although some substrate still remained after 30 hours of incubation. Peptides with Glu or Phe in the P1′ position turned out to be poor substrates. Collectively, these results show that LicP serves as a sequence-specific protease for non-native substrates and that its activity is highly portable with respect to the P1′ position (Table 4).

TABLE 4
Peptides containing cleavage sites with different P and P′ sequences that
are accepted by LicP.

| | | | | Incubation | |
|---|---|---|---|---|---|
| | P and P′ | | | time at | Complete |
| Substrate sequences | positions^{a} | [LicP] | [Substrate] | RT^{b} | Reaction? |
| LicP-95-105 | NTAVNE\| | — | — | — | Y |
| (SEQ ID NO: 31) | TESVI | | | | |
| LicP-E100A-97-107 | AVNATE\| | — | — | — | Y |
| (SEQ ID NO: 32) | SVISG | | | | |
| Modified LicA2 | NDVNPE\| | 21 nM | 17 μM | 10 min | Y |
| (SEQ ID NO: 33) | DhbDhbPADhb | | | | |
| Linear LicA2 | NDVNPE\| | 1 μM | 290 μM | 6 h | Y |
| (SEQ ID NO: 34) | TTPAT | | | | |
| ProcA1.7-NDVNPE | NDVNPE\| | 0.2 μM | 180 μM | 4 h | Y |
| (SEQ ID NO: 35) | TIGGT | | | | |
| NisA-NDVNPE | NDVNPE\| | 2 μM | 65 μM | 30 h | Y |
| (SEQ ID NO: 36) | ITSIS | | | | |
| MBP-BamL | NDVNPE\| | 0.5 μM | 50 μM | 12 h^{c} | Y |
| (SEQ ID NO: 37) | SGSEN | | | | |
| NisA-NDVNPE-I1T | NDVNPE\| | 0.2 μM | 65 μM | 20 h | Y |
| (SEQ ID NO: 38) | TTSIS | | | | |
| NisA-NDVNPE-I1C | NDVNPE\| | 0.2 μM | 65 μM | 20 h | Y |
| (SEQ ID NO: 39) | CTSIS | | | | |
| NisA-NDVNPE-I1G | NDVNPE\| | 2 μM | 65 μM | 30 h | Y |
| (SEQ ID NO: 40) | GTSIS | | | | |
| NisA-NDVNPE-I1W | NDVNPE\| | 2 μM | 65 μM | 30 h | N |
| (SEQ ID NO: 41) | WTSIS | | | | |
| NisA-NDVNPE-I1L | NDVNPE\| | 2 μM | 65 μM | 30 h | N |
| (SEQ ID NO: 42) | LTSIS | | | | |
| NisA-NDVNPE-I1K | NDVNPE\| | 2 μM | 65 μM | 30 h | N |
| (SEQ ID NO: 43) | KTSIS | | | | |
| NisA-NDVNPE-I1F | NDVNPE\| | 2 μM | 65 μM | 30 h | N |
| (SEQ ID NO: 44) | FTSIS | | | | |
| NisA-NDVNPE-I1E | NDVNPE\| | 2 μM | 65 μM | 30 h | N |
| (SEQ ID NO: 45) | ETSIS | | | | |

^{a}he vertical bar (|) separating the P and P′ positions denotes the cleavage site for the specified substrate.
^{b}RT: room temperature.
^{c}Performed at 4º C.

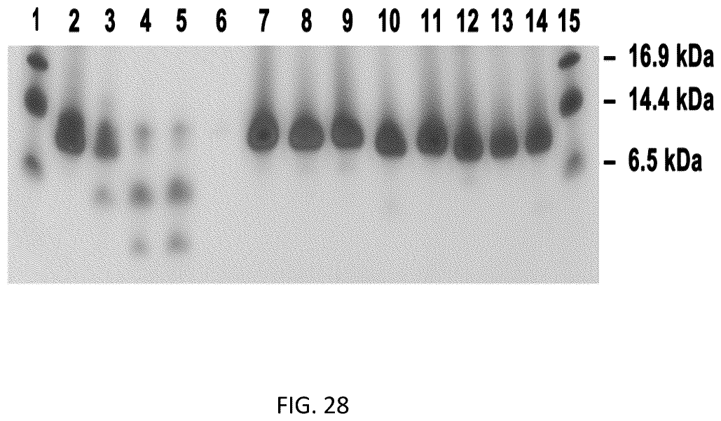

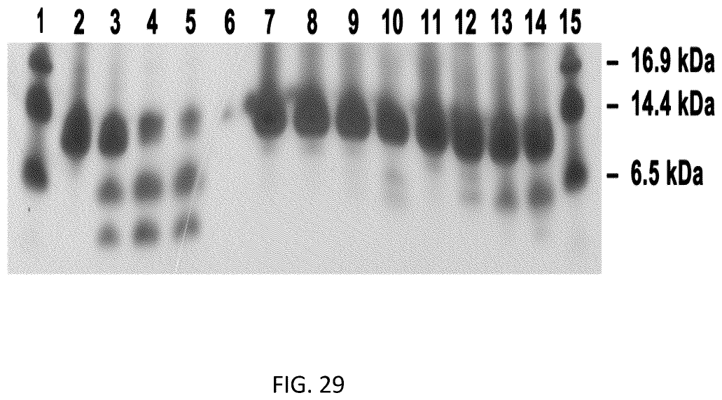

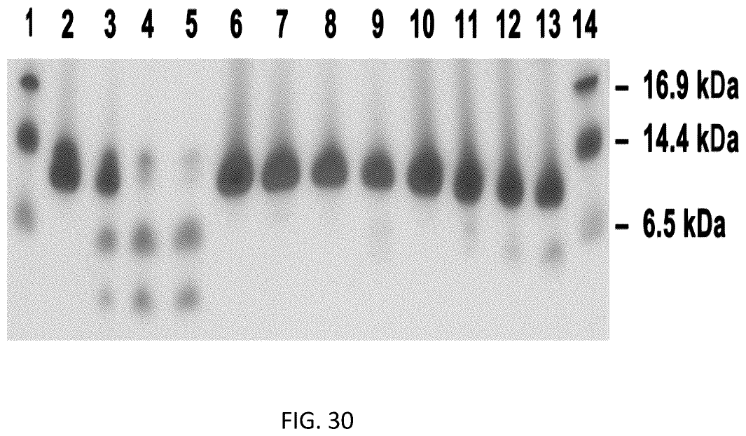

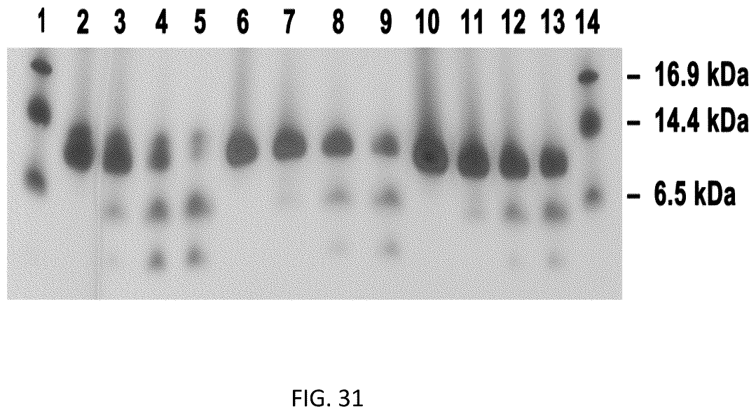

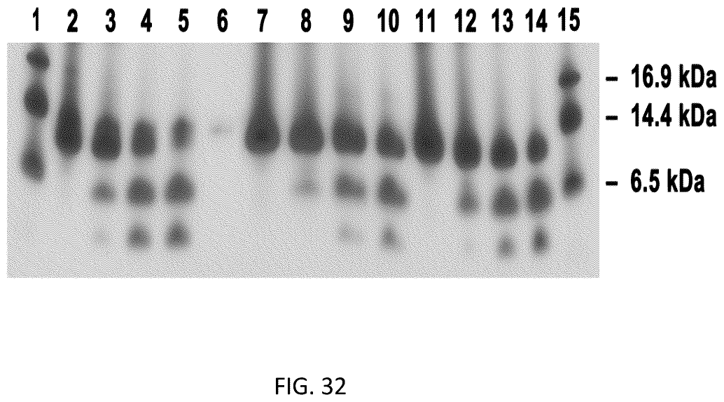

The substrate specificity of LicP was further evaluated by a gel-based assay monitoring the time-dependent cleavage of mutants of linear LicA2. The presence of a Glu at the P1 position was critical for LicP activity as LicA2-E-1A was not a substrate under the assay conditions and even substitution with structurally related amino acids Asp and Gln was not tolerated (FIG. 28). The importance of the P5 position was tested by mutating Asp to Lys or Ala. We found that LicA2-D-5A and LicA2-D-5K are processed much more slowly, suggesting that the P5 position of the substrate is also important for LicP's activity (FIG. 29 and FIG. 30). The P4 position of LicA2 was substituted with three hydrophobic amino acids of varying size, Ala, Leu and Phe. LicA2-V-4A and LicA2-V-4L were still accepted by LicP with a slightly reduced cleavage efficiency (FIG. 31). However, LicA2-V-4F was no longer processed (FIG. 30), indicating that only relatively small amino acids are tolerated at the P4 position of the substrate. The P2 and P3 positions (Pro and Asn, respectively) are not critical for LicP recognition as alanine substitution at both sites did not alter the processing efficiency of LicP significantly (FIG. 32).

### Class II LanP Proteins: A Pool of Sequence-Specific Proteases

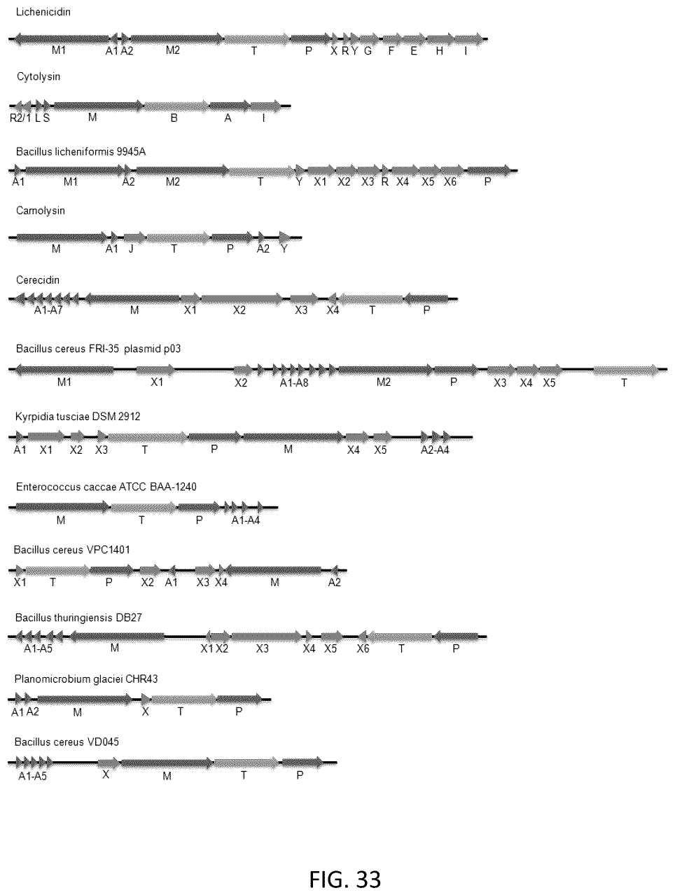

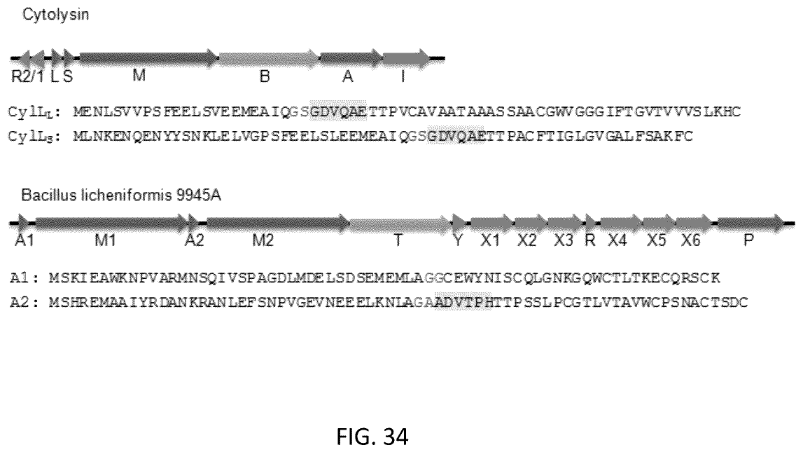

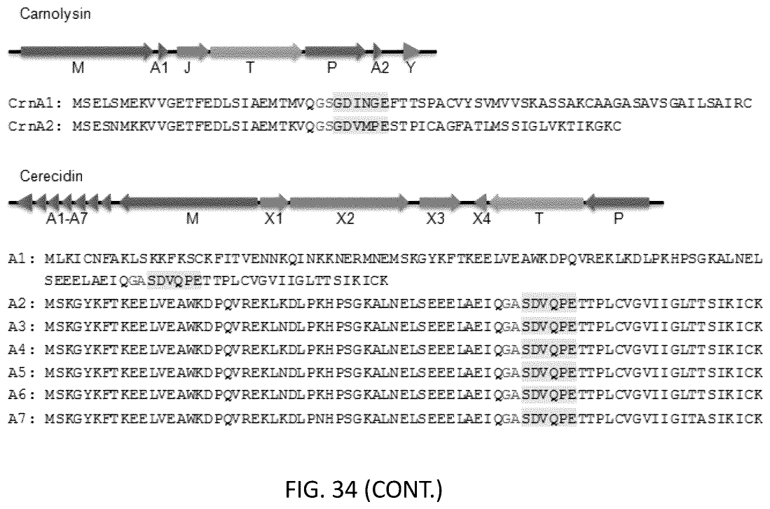

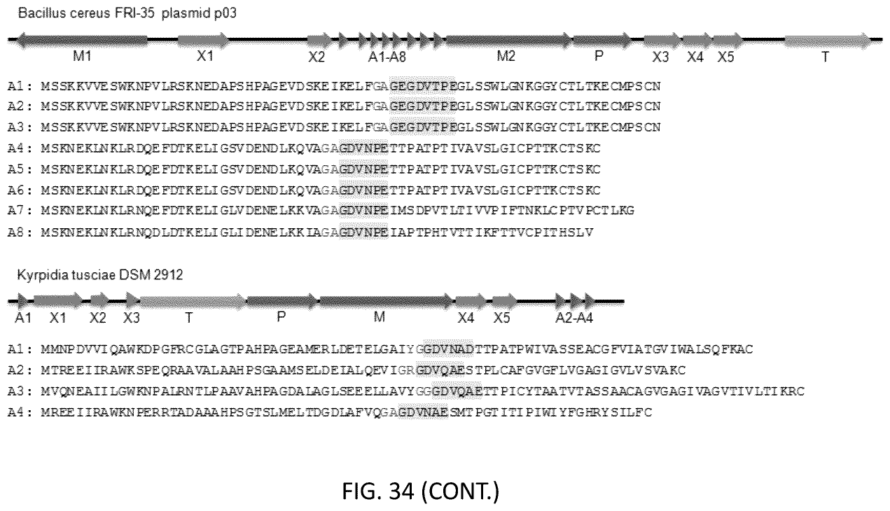

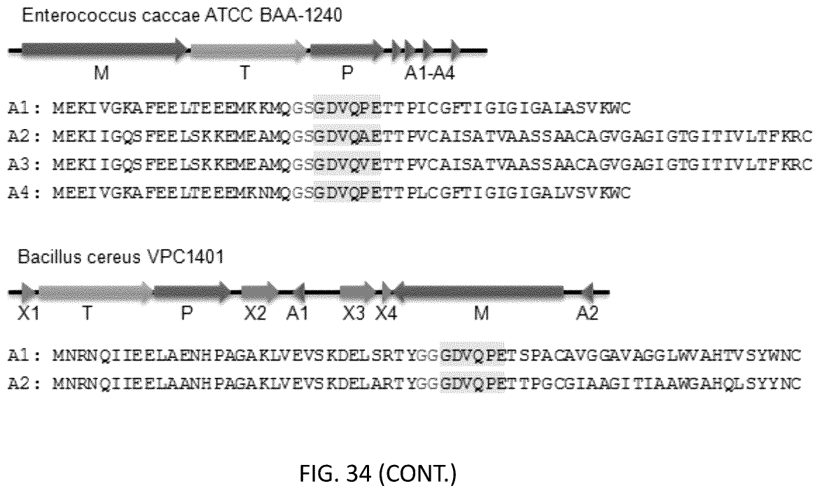

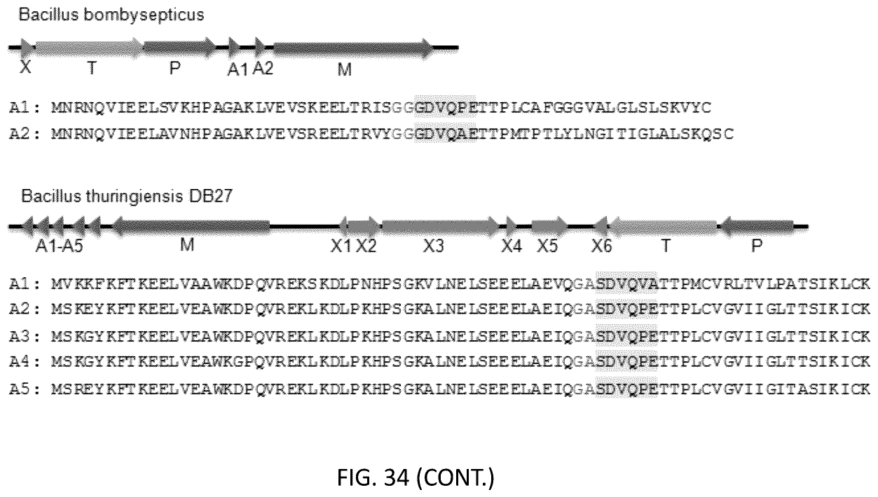

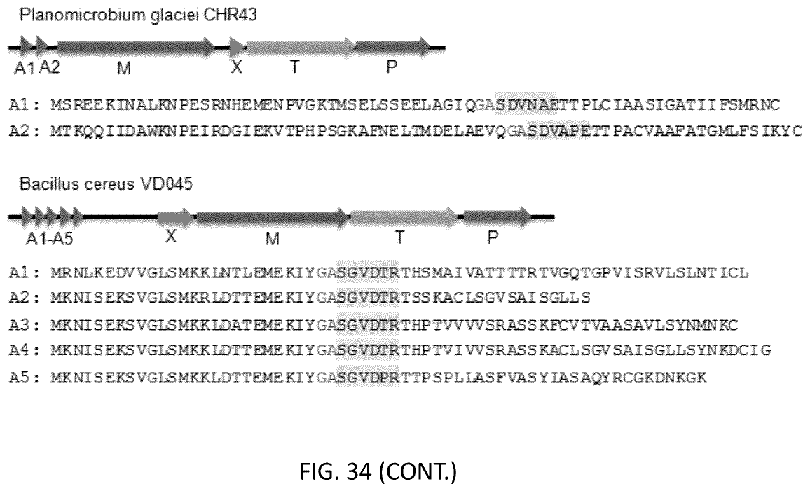

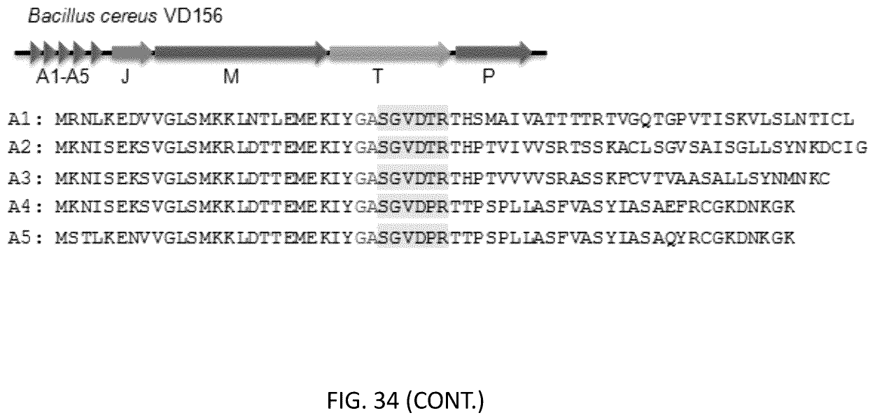

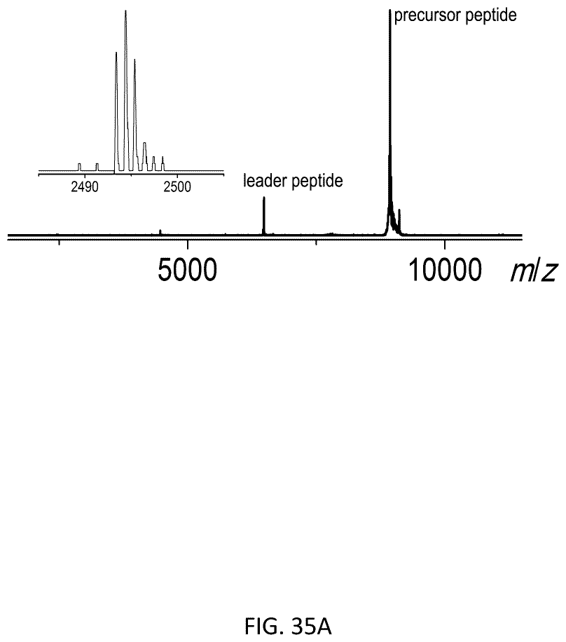

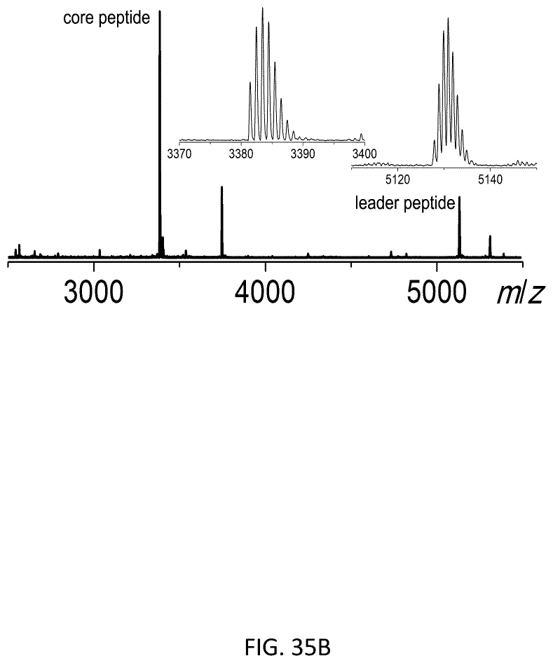

LanP genes are not often found in class II lanthipeptide gene clusters. Only four class II LanPs have been reported to date—LicP, CylA, CerP and CmP, which have been suggested to remove six-residue sequences at the N terminus of Licβ, cytolysin, cerecidins and carnolysin, respectively. Among them, the proteolytic activity, and hence the identity of the cleavage sites, has been confirmed for CylA, CerP and LicP. To identify additional class II LanP proteins and potentially identify additional recognition sequences that might be useful, we performed a search of the UniProtKB database with the LicP protease domain (LicP-101-433) as a query and the non-redundant protein sequence database with LicA2 as a query using the default Blast parameters for proteins provided by the National Center for Biotechnology Information (NCBI) website. The first 250 hits were subjected to further analysis and several were correlated to class II lanthipeptide biosynthesis by the observation of nearby genes encoding LanM proteins. Nine representative class II lanthipeptide gene clusters with LanP genes are shown in FIG. 33 (see also FIG. 34 and Table 5), all of which contain multiple genes for LanA substrates. These LanP proteins share a minimal sequence identity of 30% with the LicP protease domain with E values lower than e⁻²⁶. The putative cleavage sites for these LanP enzymes are proposed to locate immediately C-terminal to the double Gly-type motif used by LanT enzymes and upstream of the Thr/Ser/Cys rich core peptides (FIG. 34). The predicted cleavage sites were confirmed for two representative examples (Bacillus licheniformis 9945A and Bacillus cereus VD045) by incubating the purified LanP and an associated LanA substrate (FIG. 35). Interestingly, all putative LanP recognition sequences consist of six residues with the exception of the A1-A3 peptides from Bacillus cereus FIR-35, which contain eight residues with two additional amino acids at the N terminus. An Asp at the P5 position and a Val at P4 position are conserved among most clusters with the precursor peptides from Bacillus cereus VD045 being exceptions. Pro and Ala are frequently found at the P2 position, whereas the P1 position is almost exclusively occupied by polar/charged residues, such as Asp, Glu, His or Arg, but the putative P1 position of the A1 peptide from Bacillus thuringiensis DB27 is occupied by Ala.

TABLE 5
Class II LanP proteins and their predicted secretion signal peptide
sequences.ª

| | | | Signal |
|---|---|---|---|
| | | Accession | peptide |
| Name | Organism | number | (residue) |
| LicP | B. licheniformis ATCC 14580 | AAU42937.1 | 1-24 |
| (SEQ ID NO: 12) | | | |
| CylA | Enterococcus faecalis | AFJ74725.1 | 1-24 |
| (SEQ ID NO: 9) | | | |
| LanP | B. licheniformis 9945A | AGN34600.1 | 1-37 |
| (SEQ ID NO: 14) | | | |
| CerP | Bacillus cereus Q1 | ACM15351.1 | 1-36 |
| (SEQ ID NO: 15) | | | |
| LanP | Bacillus cereus FRI-35 | AFQ13336.1 | 1-25 |
| (SEQ ID NO: 16) | | | |
| LanP | Kyrpidia tusciae DSM 2912 | ADG07479.1 | 1-31 |
| (SEQ ID NO: 17) | | | |
| LanP | Enterococcus caccae ATCC | EOL44526.1 | 1-28 |
| (SEQ ID NO: 18) | BAA-1240 | | |
| LanP | Bacillus cereus VPC1401 | YP_004050051.1 | 1-31 |
| (SEQ ID NO: 19) | | | |
| CrnP | Carnobacterium maltaromaticum | AHF21241.1 | 1-30 |
| (SEQ ID NO: 20) | | | |
| LanP | Bacillus bombysepticus | AHX21587.1 | 1-31 |
| (SEQ ID NO: 21) | | | |
| LanP | Bacillus thuringiensis DB27 | CDN38711.1 | 1-36 |
| (SEQ ID NO: 22) | | | |
| LanP | Planomicrobium glaciei CHR43 | ETP67278.1 | 1-30 |
| (SEQ ID NO: 23) | | | |
| LanP | Bacillus cereus VD045 | EJR29324.1 | 1-27 |
| (SEQ ID NO: 24) | | | |
| LanP | Bacillus cereus VD156 | EJR72593.1 | 1-27 |
| (SEQ ID NO: 25) | | | |

ªSecretion signal peptide sequences are predicted using an online tool PrediSi.

Thus, the first heterologous expression of LanP proteins responsible for class II lanthipeptide biosynthesis is provided herein. We successfully reconstituted CylA's activity in vitro. In addition to its physiological role of removing the six residues at the N terminus of CylL_{L}′ and CylL_{S}′, CylA was capable of removing the entire leader peptides of modified CylL_{L} and CylL_{S}. A turnover rate of 20 min⁻¹ was observed, indicating CylA is an efficient protease. In contrast to NisP and FlaP that have been reported to exhibit a preference for modified NisA and FlaA over the unmodified peptides, CylA also removed the leader peptides of linear CylL_{L} and CylL_{S}. Although multiple groups have reported that mature LanPs purified from their producing strains lacked 95-195 residues from the N terminus, our results serve as the first evidence with that a LanP protease employs an autocatalytic activation mechanism to cleave its lanthipeptide substrate. Our observations for CylA and LicP combined with the reported results for NisP and EpiP strongly suggest that all secreted LanPs may undergo self-cleavage and employ the self-cleaving-activation mechanism.

CylA was also active against unrelated peptide substrates as long as the recognition sequence was installed—it accepted a range of residues in the P1′ site such as glycine, aromatic residues (phenylalanine, tryptophan), branched residues (isoleucine), or modified residues (MeLan, disulfide linked cysteine), strongly suggesting its potential as a general traceless tag removal protease. CylA protein was stable at −20° C. and no obvious decrease of activity was observed after multiple rounds of freeze-thaw. The identification of new LanP-containing gene clusters for class II lanthipeptide biosynthesis indicates that class II LanPs occur more widely than previously believed. Although the recognition sequences of these currently identified class II LanPs show a certain level of homology, they also exhibit considerable diversity. As a result, these LanPs may serve as a basis to construct a protease pool for general traceless tag removal purposes.

Although LicP favors modified LicA2 over linear LicA2, which indicates that post-translational modifications in the core peptide contribute to LicP's substrate recognition in addition to the NDVNPE sequence, our observations with substrate analogs demonstrate its application as a sequence-specific protease for traceless removal of leader peptides and an expression tag. The substrate specificity of LicP was identified using both structural information and biochemical characterizations. The P5, P4 and P1 residues of LicA2 were found to be important for LicP recognition. These three sites were also suggested as the origin of specificity for a class I LanP, ElxP, as determined by kinetic analysis based on LC quantification. The similarity in the important positions suggests a general substrate recognition mechanism by the entire subtilisin-like LanP family.

The thermostability of subtilisin BPN′ and related proteases is enhanced significantly in the presence of calcium ions, which are necessary for maturation, and subsequent stabilization of a large loop in the catalytic domain. The calcium dependence constitutes a drawback for industrial utility of subtilisin BPN′. Much effort has been spent on engineering thermostable mutants of subtilisin that function in a calcium-independent manner. The structural and biochemical analysis of LicP (data not shown) reveals an elegant solution to this limitation, as maturation and subsequent stabilization of the enzyme is facilitated not by metal ions, but rather by the insertion of Trp111, liberated by cleavage of the linker between the prodomain and the catalytic domain, into a hydrophobic pocket located in the same vicinity as the calcium-binding site in subtilisin BPN′. A recent structure of the class I lanthipeptide protease NisP also demonstrated loss of a calcium binding site, although unlike the structure of LicP, the prodomain was not present in the NisP structure and its substrate specificity was not investigated.

Over the past several decades, the toolbox of useful proteases has been significantly enlarged. Several proteases with strict recognition sequences have been commercialized for biochemical or industrial applications, including factor Xa, enterokinase, and TEV protease. Factor Xa and enterokinase exhibit trypsin-like activity and cleave after an Arg or Lys. TEV protease recognizes a larger motif and exhibits better reliability in terms of specificity, but TEV protease requires either a Gly or Ser at the P1′ position for efficient cleavage. LicP is complementary in that it specifically cleaves after a Glu in the NDVNPE sequence, and is quite tolerant of various residues in the P1′ position. LicP accepted a range of residues at the P1′ site (Table 4) such as glycine, small polar residues (Ser, Thr, Cys) and large aliphatic residues (Ile). LicP also processed peptides with Leu, aromatic (Phe, Trp) and charged (Lys, Glu) residues at the P1′ position albeit with reduced efficiency. Additional favorable properties include its stability demonstrated by the persistent activity of LicP after 12 weeks at 4° C., and no obvious decrease in activity after multiple rounds of freeze-thaw procedures.

This disclosure identifies ten new class II lanthipeptide gene clusters containing lanP genes, suggesting they are more widely distributed than previously expected. Although the putative recognition sequences of these newly identified LanPs show a certain level of homology, they also exhibit considerable diversity. Similar to other proteases, most of these LanPs are predicted to cleave after charged residues such as arginine, glutamate or aspartate, but a few appear to cleave after unusual P1 residues such as histidine or alanine that are rarely the site of cleavage for other proteases. We confirmed the predicted sites for two examples that have a His and an Arg in the P1 position. Hence, this naturally occurring protease family may serve as a basis to construct a general protease pool for traceless tag removal purposes.

### Utility and Biotechnology Applications

The discovery of novel lanthipeptide protease polypeptides and their substrate recognition rules, including the robust portability of their substrate recognition sites into heterologous polypeptide contexts, provides fundamentally new and non-obvious approaches to generating mature polypeptides or recombinant polypeptides completely devoid of extraneous N-terminal sequences, such as leader sequences or tag sequences (that is, scarless or traceless of leader or tag sequences). The present disclosure provides several aspects having broad utility in biotechnology, medical and diagnostic applications of lanthipeptide proteases for processing cognate lanthipeptides, noncognate lanthipeptide and heterologous peptides that can be suitably processed by lanthipeptide proteases to yield scarless tag polypeptide products.

### Nucleic Acid Reagents.

In one aspect, an isolated nucleic acid comprises an open reading frame encoding a lanthipeptide protease polypeptide for scarless tag removal from a polypeptide is provided. In this regard, the lanthipeptide protease is codon optimized for expression in an expression host. The expression host can be selected from E. coli, S. cerevisiae, S. pombe, P. pastoris, an insect cell, a HeLa cell, a Jurkat cell, a 293 cell, a CHO cell and a COS cell. In these aspects, the lanthipeptide protease polypeptide can be selected from SEQ ID NOS: 5, 7, 9-25, 29 and 30, including equivalents thereof and derivatives thereof. Furthermore, in this respect, the lanthipeptide protease polypeptide can recognize a substrate recognition sequence selected from SEQ ID NOS: 1-3, 27, 31-46 and sequences of Table 3, including equivalents thereof and derivatives thereof. Additionally, the polypeptide can be a cognate lanthipeptide, a non-cognate lanthipeptide or a heterologous polypeptide.

The isolated nucleic acid can further include a vector having a transcription controlling signal, wherein the isolated nucleic acid can be operably linked to the transcriptional controlling signal to enable expression of the lanthipeptide protease polypeptide. In this regard, the transcriptional controlling signal of the nucleic acid can include a transcriptional initiation element. In this regard, the transcriptional controlling signals can further include a transcriptional termination element. The isolated nucleic acid can further include a translational controlling signal. Exemplary translational controlling signals include at least one selected from a translational enhancer and a post-translational processing element.

Recombinant DNA and molecular biology and biochemical methods for carrying out the preparations of these reagents are well understood in the art.

### Expression Cassette Reagents.

In another aspect, an expression cassette including an open reading frame for a polypeptide, wherein the open reading frame encodes a substrate recognition sequence for a lanthipeptide protease polypeptide, is provided. In this aspect, the substrate recognition sequence is selected from SEQ ID NOS: 1-3, 27, 31-46 and sequences of Table 3, including equivalents thereof and derivatives thereof. In this aspect, the lanthipeptide protease polypeptide is selected from SEQ ID NOS: 5, 7, 9-25, 29 and 30, including equivalents thereof and derivatives thereof. In this aspect, the polypeptide is selected from a cognate lanthipeptide, a non-cognate lanthipeptide or a heterologous polypeptide.

In another respect, this aspect further includes a transcription controlling signal, wherein the isolated nucleic acid is operably linked to the transcriptional controlling signal to enable expression of the polypeptide. In this regard, the transcriptional controlling signal includes a transcriptional initiation element. In one respect, the transcriptional controlling signals can further include a transcriptional termination element. In these latter respects, a translational controlling signal can be included. For example, the translational controlling signal can include at least one selected from a translational enhancer and a post-translational processing element.

Recombinant DNA and molecular biology and biochemical methods for carrying out the preparations of these reagents are well understood in the art.

### Novel Polypeptide Precursor Substrates for Lanthipeptide Protease Processing.

In another aspect, an isolated polypeptide comprising the structure: T-R-P is provided, wherein T comprises a tag motif, R comprises a lanthipeptide protease substrate recognition sequence and P comprises an open reading frame encoding a polypeptide without the tag motif and lanthipeptide protease substrate recognition sequence.

In this respect, the isolated polypeptide can be codon optimized for expression in an expression host. Preferred expression hosts in this aspect include those selected from E. coli, S. cerevisiae, S. pombe, P. pastoris, an insect cell, a HeLa cell, a Jurkat cell, a 293 cell, a CHO cell and a COS cell. Protein expression in these systems is well known in the art. Moreover, the lanthipeptide protease substrate recognition sequence in this aspect can be selected from SEQ ID NOS: 1-3, 27, 31-46 and sequences of Table 3, including equivalents thereof and derivatives thereof.

With respect to the T comprising a tag motif, the tag motif can include an affinity tag. In this regard, the affinity tag can be preferably selected from polyhistine, maltose binding protein, glutathione-S-transferase, HaloTag®, AviTag, Calmodulin-tag, polyglutamate tag, FLAG-tag, HA-tag, Myc-tag, S-tag, SBP-tag, Softag 3, V5 tag and Xpress tag. In the foregoing aspects, the polypeptide is a cognate lanthipeptide, a non-cognate lanthipeptide or a heterologous polypeptide. These tag systems are well known in the art.

### Methods for Precursor Polypeptide Processing Using Lanthipeptide Proteases.

In another aspect, a method of scarless tag removal from a polypeptide is provided. The method includes two steps. The first step includes providing the polypeptide, wherein the polypeptide includes the structure: T-R-P. T comprises a tag motif, R comprises a lanthipeptide protease substrate recognition sequence and P comprises an open reading frame encoding a polypeptide without the tag motif and lanthipeptide protease substrate recognition sequence. The second step includes subjecting the polypeptide to a lanthipeptide protease having specificity for catalyzing proteolytic cleavage at the lanthipeptide protease substrate recognition sequence, thereby providing the polypeptide without a tag scar. In another aspect, the method further includes a step of purifying the polypeptide without a tag scar.

In these aspects, the lanthipeptide protease can be codon optimized for expression in an expression host. Suitable expression hosts in this regard include any of those selected from E. coli, S. cerevisiae, S. pombe, P. pastoris, an insect cell, a HeLa cell, a Jurkat cell, a 293 cell, a CHO cell and a COS cell. Protein expression in these systems is well known in the art. With respect to all of these aspects, the lanthipeptide protease polypeptide can be selected from SEQ ID NOS: 5, 7, 9-25, 29 and 30, including equivalents thereof and derivatives thereof. Likewise with respect to these aspects, lanthipeptide protease substrate recognition sequence is selected from SEQ ID NOS: 1-3, 27, 31-46 and sequences of Table 3, including equivalents thereof and derivatives thereof.

Similarly, the tag motif can include an affinity tag. In some aspects, the affinity tag can be selected from polyhistine, maltose binding protein, glutathione-S-transferase, HaloTag®, AviTag, Calmodulin-tag, polyglutamate tag, FLAG-tag, HA-tag, Myc-tag, S-tag, SBP-tag, Softag 3, V5 tag and Xpress tag. In some aspects, the polypeptide can be selected from a cognate lanthipeptide, a non-cognate lanthipeptide or a heterologous polypeptide. These tag systems are well known in the art.

In all of these aspects, the polypeptide is expressed in vivo or in vitro. In one respect, the polypeptide is expressed in vivo from an expression cassette in an expression host. In this regard, a suitable expression host can be selected from E. coli, S. cerevisiae, S. pombe, P. pastoris, an insect cell, a HeLa cell, a Jurkat cell, a 293 cell, a CHO cell and a COS cell. In another aspect, polypeptide is expressed in vitro from an expression cassette in a coupled transcription-translation system or from a translation template in a translation system. These systems and methods of expression are well known in the art.

### Kits.

In another aspect, a kit for expressing a polypeptide without a tag scar is provided. The kit includes two components. The first component includes an expression vector that includes an expression cassette, wherein the expression cassette can encodes a polypeptide that includes the structure: T-R-P. T comprises a tag motif, R comprises a lanthipeptide protease substrate recognition sequence and P comprises an open reading frame encoding a polypeptide without the tag motif and lanthipeptide protease substrate recognition sequence. The second component includes a lanthipeptide protease having specificity for catalyzing proteolytic cleavage at the lanthipeptide protease substrate recognition sequence, thereby providing the polypeptide without the tag scar. In a further refinement of this aspect, the kit includes a reagent to purify the polypeptide without the tag scar.

With respect to both of these aspects, an additional refinement includes an expression host. In this respect, the expression host can be selected from E. coli, S. cerevisiae, S. pombe, P. pastoris, an insect cell, a HeLa cell, a Jurkat cell, a 293 cell, a CHO cell and a COS cell.

The basic kit can include an lanthipeptide protease that is codon optimized for expression in the expression host. In this regard, the expression host can be selected from E. coli, S. cerevisiae, S. pombe, P. pastoris, an insect cell, a HeLa cell, a Jurkat cell, a 293 cell, a CHO cell and a COS cell. With respect to any of the foregoing aspects, the lanthipeptide protease polypeptide can be selected from SEQ ID NOS: 5, 7, 9-25, 29 and 30, including equivalents thereof and derivatives thereof. With respect to any of the foregoing aspects, the lanthipeptide protease substrate recognition sequence can be selected from SEQ ID NOS: 1-3, 27, 31-46 and sequences of Table 3, including equivalents thereof and derivatives thereof.

With respect to any of the foregoing aspects, the tag motif comprises an affinity tag. The affinity tag can be selected from polyhistine, maltose binding protein, glutathione-S-transferase, HaloTag®, AviTag, Calmodulin-tag, polyglutamate tag, FLAG-tag, HA-tag, Myc-tag, S-tag, SBP-tag, Softag 3, V5 tag and Xpress tag. With respect to any of the foregoing aspects, the polypeptide is a cognate lanthipeptide, a non-cognate lanthipeptide or a heterologous polypeptide.

### EXAMPLES

Examples 1-9 are directed to ElxP protease activity, substrate recognition rules and demonstration of ElxP-mediated cleavage of heterologous fusion polypeptides. Examples 10-24 are directed to CylA and LicP protease activities and substrate recognition rules.

### Example 1. Organisms, Media, and Growth Conditions

All oligonucleotides used in this study were purchased from Integrated DNA Technologies and are presented in Table 6 For microorganisms used, see Table 7

TABLE 6
Oligonucleotide Sequences

| Name | 5′ Sequence 3′ |
|---|---|
| ElxA Q-1A F | GCA CAA AAA AGT G?C CTA AAT CCG GCA TCA GCT AGT ATT GTT AAAACAAC |
| ElxA Q-1A R | GTT GTT TTA ACA ATA CTA GCT GAT GCC GGA TTT AGG TCA CTT TTT TGT GC |
| ElxA P-2A F | GCA CAA AAA AGT G?C CTA AAT GCG CAA TCA GCT AGT ATT GTT AAA ACA AC |
| ElxA P-2A R | GTT GTT TTA ACA ATA CTA GCT GAT TGC GCA TTT AGG TCA CTT TTT TGT GC |
| ElxA N-3A F | ATC GAG GCA CAA AAA AGT G?C CTA GCA CCG CAA TCA GCT |
| ElxA N-3A R | ACT TTT TTG TGC CTC GAT ATC TTT ATT AAG ATT TAA ATC AAA TAA TTC TTT TTT C |
| ElxA L-4A F | ATC GAG GCA CAA AAA AGT G?C GCA AAT CCG CAA TCA GCT |
| ElxA L-4A R | ACT TTT TTG TGC CTC GAT ATC TTT ATT AAG ATT TAA ATC AAA TAA TTC TTT TTT C |
| ElxA D-5A F | ATC GAG GCA CAA AAA AGT GCA CTA AAT CCG CAA TCA GCT |
| ElxA D-5A R | ACT TTT TTG TGC CTC GAT ATC TTT ATT AAG ATT TAA ATC AAA TAA TTC TTT TTT C |
| ElxA F-19A F | GGC AGC CAT ATG AAA AAA GAA TTA GCT GAT TTA AAT CTT AAT AAA GAT ATC G |
| ElxA F-19A R | CGA TAT CTT TAT TAA GAT TTA AAT CAG CTA ATT CTT TTT TCA TAT GGC TGC C |
| ElxA D-18A F | CAG CCA TAT GAA AAA AGA ATT ATT TGC TTT AAA TCT TAA TAA AGA TAT CGA GGC |
| ElxA D-18A R | GCC TCG ATA TCT TTA TTA AGA TTT AAA GCA AAT AAT TCT TTT TTC ATA TGG CTG |
| ElxA L-17A F | GCC ATA TGA AAA AAG AAT TAT TTG ATG CAA ATC TTA ATA AAG ATA TCG AGG C |
| ElxA L-17A R | GCC TCG ATA TCT TTA TTA AGA TTT GCA TCA AAT AAT TCT TTT TTC ATA TGG C |
| ElxA N-16A F | GCC ATA TGA AAA AAG AAT TAT TTG ATT TAG CTC TTA ATA AAG ATA TCG AGG CAC |
| ElxA N-16A R | GTG CCT CGA TAT CTT TAT TAA GAG CTA AAT CAA ATA ATT CTT TTT TCA TAT GGC |
| NisA R-1Q F | CAT CAC CAC AGA TTA CAA GTA TTT CGC TAT GTA CAC CCG GTT G |
| NisA R-1Q R | CTT GTA ATC TGT GGT GAT GCA CCT GAA TCT TTC TTC GAA ACA G |
| NisA R-1Q Q-1_ | GAT TCA GGT GCA TCA CCA CAG GCA ATT ACA AGT ATT TC |
| I1insA F | |
| NisA R-1Q Q-1_ | TGA TGC ACC TGA ATC TTT CTT CGA AAC AGA TAC CAA ATC |
| I1insA R | |
| NisA G-5D A-4L | GTT TCG AAG AAA GAT AGC GAT CTG AAT CCA CAG ATT ACA AGT ATT TCG |
| S-3N R-1Q F | |
| NisA G-5D A-4L | ATC TTT CTT CGA AAC AGA TAC CAA ATC CAA GTT AAA ATC |
| S-3N R-1Q R | |
| NisA G-5D A-4L | GTT TCG AAG AAA GAT TCA GAT CTG AAT CCG CAG GCA ATT ACA AGT ATT TC |
| S-3N R-1Q Q-1_ | |
| I1insAF | |
| NisA G-5D A-4L | TGA ATC TTT CTT CGA AAC AGA TAC CAA ATC CAA GTT AAA ATC TTT TGT ACT C |
| S-3N R-1Q Q-1_ | |
| I1insA R | |
| ElxO.S139A.FP | GGA AAT CCG CTT AAT CCT GTA ATA GCA GAA ATA TTT ACT ATT GCT CC |
| ElxO.S139A.RP | GGA GCA ATA GTA AAT ATT TCT GCT ATT ACA GGA TTA AGC GGA TTT CC |
| ElxO.Y152F.FP | GCG GAT TTC CTT ACT CTA TAT TAT TCG GTA GCA CAA AAC ATG CTG |
| ElxO.Y152F.RP | CAG CAT GTT TTG TGC TAC CGA ATA ATA TAG AGT AAG GAA ATC CGC |
| ElxO.K156A.FP | CCT TTA GTT AAA CCA ATA ACA GCA TGT GCT GTG CTA CCG TAT AAT ATA GAG TAA GG |
| ElxO.K156A.RP | CCT TAC TCT ATA TTA TAC GGT AGC ACA GCA CAT GCT GTT ATT GGT TTA ACT AAA GG |
| ElxO.K156M.FP | CCT TTA GTT AAA CCA ATA ACA GCA TGC ATT GTG CTA CCG TAT AAT ATA GAG TAA GG |
| ElxO.K156M.RP | CCT TAC TCT ATA TTA TAC GGT AGC ACA ATG CAT GCT GTT ATT GGT TTA ACT AAA GG |

TABLE 7
Organisms¹

| Strain or plasmid | Relevant characteristics |
|---|---|
| Escherichia coli | |
| DH5α | λpir/ϕ80dlacZΔM15 Δ(lacZYA-argF)U169 |
| | recA1 hsdR17 deoR thi-1 supE44 gyrA96 relA1 |
| BL21 DE3 | F⁻ ompT gal dcm lon hsdS_{B}(r_{B}⁻ m_{B}⁻) λ(DE3 [lacI |
| | lacUV5-T7 gene 1 ind1 sam7 nin5]) |
| Rosetta2 | F⁻ ompT gal dcm lon hsdS_{B}(r_{B}⁻ m_{B}⁻) λ(DE3 [lacI |
| | lacUV5-T7 gene 1 ind1 sam7 nin5]) pRARE2 |
| | pLysS |
| Lactobacillus sake L45 | Lactocin S producer strain |
| Pediococcus acidilactici Pac1.0 | Lactocin S sensitive strain |

¹Sources/References: DH5α (Grant, S. G. et al. Proc. Natl. Acad. Sci. U.S.A. 87, 4645-4649 (1990); BL21 DE3 and Rosetta2 (Novagen [EMD Millipore]); Lactobacillus sake L45 and Pediococcus acidilactici Pac 1.0 (Mortvedt, C. I. et al. Appl. Environ. Microbiol. 57, 1829-1834 (1991)).

Reagents used for molecular biology were purchased from New England BioLabs, Thermo Fisher Scientific, or Gold Biotechnology. Plasmid sequencing was performed by ACGT Inc. unless otherwise noticed. Escherichia coli DH5C and BL21 (DE3) were used for plasmid maintenance and protein or peptide overexpression, respectively. The strains L sake L45 and P. acidilactici Pac 1.0 were grown in de Man-Rogosa-Sharpe (MRS) solid agar or broth. MALDI-TOF measurements were performed using a Bruker UltrafleXtreme MALDI-TOF-TOF instrument using a positive reflective mode and sinapinic acid as a matrix unless otherwise noted.

### Example 2. Markov Chain Monte Carlo Phylogenetic Tree Analysis

The LanP sequences were aligned in ClustalX using default parameters with iteration at each alignment step, and the alignments were manually fine-tuned afterwards. Bayesian inference was used to calculate posterior probability of clades utilizing the program MrBayes (version 3.2). Final analyses consisted of two sets of eight chains each (one cold and seven heated), run to reach a convergence with standard deviation of split frequencies <0.005. Posterior probabilities were averaged over the final 75% of trees (25% burn in). The analysis utilized a mixed amino acid model with a proportion of sites designated invariant, and rate variation among sites modeled after a gamma distribution divided into eight categories, with all variable parameters estimated by the program based on BioNJ starting trees. Accession numbers are listed in Table 8.

TABLE 8
Accession numbers of some of the proteases described herein.

| | Accession | |
|---|---|---|
| Protease | Number | Organism |
| CylA | AFJ74725.1 | Enterococcus faecalis |
| LicP | AAU42937.1 | Bacillus licheniformis DSM 13 |
| NisP | ADJ56357.1 | Lactococcus lactis subsp. lactis |
| NiqP | BAG71484.1 | Lactococcus lactis |
| SlvP | AEX55163.1 | Streptococcus salivarius |
| EpiP | CAA44257.1 | Staphylococcus epidermidis |
| GdmP | ABC94907.1 | Staphylococcus gallinarum |
| BsaA | BAB95626.1 | Staphylococcus aureus subsp. |
| associated | | aureus MW2 |
| Bsa1 | YP_005737288.1 | Staphylococcus aureus subsp ED133 |
| associated | | |
| NsuP | ABA00872.1 | Streptococcus uberis |
| ElxP | AFN69433.1 | Staphylococcus epidermidis |
| EciP | CAA74349.1 | Staphylococcus epidermidis |
| PepP | CAA90024.1 | Staphylococcus epidermidis |
| Bsn5 | YP_004206152.1 | Bacillus subtilis BSn5 |
| associated | | |

### Example 3. Cloning, Expression, and Purification of MBP-ElxP, His₆-ElxA, His₆-NisA, and Mutant Peptides

Cloning, Expression, and Purification of MBP-EIxP

Primers used for the construction of mutant substrates are listed in Table 6. The cloning of the gene encoding ElxP is described in Velasquez, J. E. et al. Chem. Biol. 18, 857-867 (2011). An aliquot of 50 ng of pelB-mbp-elxP-pET28b was used to transform 50 L of electrocompetent E. coli BL21 (DE3) Rosetta 2 cells following standard procedures. After the incubation period, cells were plated on LB agar (LBA) plates supplemented with kanamycin (kn, 25 μg mL⁻¹) and chloramphenicol (cm, 12.5 μg mL⁻¹) and grown at 37° C. overnight (O/N). A single colony was used to inoculate 1 mL of fresh LB supplemented with kn and cm and 1 μL was plated for a second time on LBA plates supplemented with kn and cm. Plates were kept at 37° C. O/N. For overexpression, 1 mL of LB supplemented with kn and cm was used to scrape the cells out of the O/N LBA plates to be used as initial inoculum. Overexpression was performed in 6 L of LB supplemented with kn and cm with a starter OD₆₀₀ of 0.025. Cultures were incubated at 37° C., 250 rpm until the OD₆₀₀ reached ˜1.0. At this OD, cultures were chilled on ice for 15 min and protein expression was induced with 0.1 mM isopropyl-β-D-1-thiogalactopyranoside (IPTG). Protein overexpression was carried out at 18° C., 250 rpm for 16 h. The cells were harvested (6976×g, 20 min, 4° C.), resuspended in 50 mL of lysis buffer (20 mM Tris-HCl pH 8.0, 500 mM NaCl, 10% (v/v) glycerol, 1 mM EDTA) and lysed using an EmulsiFlex-C3 homogenizer with a pressure of less than 500 bar. To remove cell debris, the lysed fraction was centrifuged (22,789×g, 4° C., 30 min) and the supernatant was cleared through a 0.45 μm syringe-tip filter (Millipore). The protein was purified by affinity chromatography using an ÄKTA purifier (GE healthcare) equipped with an MBPTrap HP 5 mL column pre-packed with Dextrin Sepharose™ (GE healthcare) according to the manufacturer's protocol. After loading the supernatant into the column pre-equilibrated with lysis buffer, the column was washed with lysis buffer until a stable baseline based on the absorbance at 280 nm was reached. Protein was eluted with a linear gradient from 0-100% (v/v) of elution buffer (20 mM Tris-HCl pH 8.0, 500 mM NaCl, 1 mM EDTA, 10 mM maltose) in lysis buffer over 30 min. Collected fractions were analyzed by SDS-PAGE using a 4-20% TGX mini-protean gel (BioRad) and visualized by Coomassie staining. Fractions containing the desired MBP-ElxP were collected and desalted using gel filtration chromatography on a HiLoad 16/60 column packed with Superdex 200 PG eluting with 1 mL min⁻¹ of gel filtration buffer (300 mM NaCl, 20 mM Tris-HCl pH 8.0, 10% (v/v) glycerol). Protein was concentrated using an Amicon Ultra centrifuge tube of 30 kDa molecular weight cut off (MWCO) (3,488×g, 30 min, 4° C.) (Millipore). The concentration of the purified protein was determined spectrophotometrically using a calculated molar extinction coefficient of 113,485 M⁻¹ cm⁻¹ and a molecular weight of 80.504 kDa. Purity was assessed by SDS-PAGE analysis. Aliquots were frozen in liquid nitrogen for future use and stored at −80° C.

Cloning, Expression, and Purification of His₆-ElxA and Mutant Variants

The cloning of the gene encoding ElxA into pET28b is described in Velasquez, J. E. et al. Chem. Biol. 18, 857-867 (2011). The ElxA leader peptide variants were constructed using QuikChange mutagenesis following the method of Liu, H., and Naismith, J. H. BMC biotechnology 8, 91 (2008). The his₆-elxA-pET28b was used as a template to introduce the different mutations by PCR using the corresponding primer pair listed in the Table 6. A typical PCR reaction consisted of 1× HF Buffer, 0.2 mM DNTP, 1 μM forward and reverse primers, 10 ng template DNA, 3% (v/v) DMSO and 0.02 U μL⁻¹ Phusion polymerase (NEB). After PCR, reactions were incubated at 37° C. for 2 h with DpnI (5 U). After treatment with DpnI, samples were purified using the QIAquick PCR purification kit (Qiagen). An aliquot of 10 μL was used to transform E. coli DH5ca cells using the heat shock method and cells were plated on LBA plates supplemented with kn (50 μg mL⁻¹). Plates were incubated at 37° C. O/N. A single colony was inoculated in LB supplemented with kan, grown at 37° C. O/N, and plasmid was extracted using the QIAprep spin mini prep kit (Qiagen). The desired mutations were verified by DNA sequencing.

Overexpression and purification of wild type ElxA and mutant variants was carried out using a previously described method with minor modifications (Velasquez, J. E. et al. Chem. Biol. 18, 857-867 (2011)). An aliquot of 50 ng of recombinant DNA from wild type ElxA and each of the mutants described above was used to transform 50 μL of electrocompetent E. coli BL21 (DE3) cells and plated on LBA supplemented with kan (50 μg mL⁻¹) at 37° C. O/N. A single colony was used to inoculate a starter inoculum of LB supplemented with kn and incubated at 37° C. for 12 h. After the incubation period, 6 L of Terrific Broth (TB) supplemented with kn, and glycerol (4 mL L⁻¹), were inoculated with the starter culture to obtain an initial OD₆₀₀ of 0.025. Flasks were incubated at 37° C., 250 rpm, and peptide expression was induced at 18° C., 250 rpm for 18 h with 1.0 mM IPTG when the OD₆₀₀ reached 1.0. Cells were harvested by centrifugation (6,976×g, 20 min, 4° C.), resuspended in 30 mL of LanA Buffer 1 (6 M guanidine hydrochloride, 20 mM NaH₂PO₄ pH 7.5, 500 mM NaCl and 0.5 mM imidazole) and lysed by sonication (35% amplitude, 4.0 s pulse, 9.9 s pause, 15 min). Cell debris was removed by centrifugation (22789×g, 30 min, 4° C.) and supernatant was filtered through a 0.45 μm syringe filter unit. Purification was carried out using IMAC with a 5 mL HisTrap column pre-packed with Ni Sepharose™. After loading the supernatant into the column, the column was washed with 10 column volumes (CV) of LanA Buffer 1 followed by 10 CV of LanA Buffer 2 (4 M guanidine hydrochloride, 20 mM NaH₂PO₄ pH 7.5, 300 mM NaCl and 30 mM imidazole) to remove any non-specifically bound proteins, followed by peptide elution in 3 CV of LanA Elution Buffer (4 M guanidine hydrochloride, 20 mM Tris HCl pH 7.5, 100 mM NaCl, 1 M imidazole). To remove excess salts, the peptide was purified by reversed phase high performance liquid chromatography (RP-HPLC) using a C₄ Waters Delta Pak cartridge column with a linear gradient of 2% (v/v) of solvent A [80% (v/v) MeCN, 20% (v/v) H₂O, 0.086% (v/v) trifluoroacetic acid (TFA)] in solvent B [0.1% (v/v) TFA in H₂O] to 75% (v/v) solvent A over 30 min. Fractions were analyzed for the desired peptide by MALDI-TOF MS. Fractions containing peptide were freeze dried using a lyophilizer (Labconco) and stored at −20° C. The purity of each peptide was assessed by analytical HPLC.

Cloning, Expression, and Purification of His₆-NisA and Mutant Variants

The cloning of the gene encoding NisA into pRSF Duet-I is described in Garg, N. et al. Proc. Natl. Acad. Sci. U.S.A. 110, 7258-7263 (2013). The NisA leader peptide variants were constructed using QuikChange mutagenesis. The his₆-nisA-pRSF Duet-I plasmid was used as a template to introduce the different mutations by PCR using the corresponding primer pair listed in Table 6. PCR conditions, overexpression, and purification of peptides were performed as described above.

### Example 4. MBP-ElxP Activity Assay by MS

A typical activity assay consisted of 50 mM Tris HCl pH 8.0, 50 μM peptide, 5 μM MBP-ElxP in a final volume of 100 μL. The sample was incubated for 2 h at room temperature. To monitor cleavage activity, samples were desalted using a zip tip concentrator (Millipore), mixed in a 1:1 ratio with sinapinic acid, and spotted on a MALDI-TOF Bruker plate. Ion intensities for the resulting precursor peptide, leader peptide and core peptide were normalized and the proteolytic efficiency was measure as the amount of substrate left after cleavage reaction (Table 1). Reactions were performed with tagged enzyme and substrates unless otherwise noticed.

### Example 5. Determination of ElxP Kinetic Parameters

The kinetic parameters of MBP-ElxP were determined using an HPLC based assay following the method of Ishii, S. et al. J. Biol. Chem. 281, 4726-4731 (2006). The peptidase activity of tagged ElxP was assayed in a 100 μL reaction mixture containing 50 mM Tris pH 8.0, and various concentrations of wild type His₆-ElxA or mutant variants. The reaction was started by adding MBP-ElxP to a final concentration such as to consume less than 10% of the initial substrate concentration in the time frame of the assay. The enzyme concentration ranged from 0.25 μM to 1 μM. The reaction was incubated at room temperature and quenched in 0.1% (v/v) TFA and 5 mM TCEP at different time points. The reactions were loaded on a Hypersil Gold C₄(250×4.6 mm, 5μ analytical column (Thermo Fisher Scientific)) connected to an Agilent 1260 Liquid Chromatography (HPLC) system (Agilent Technologies). Product formation was detected by monitoring the increase in the peak area of the leader peptide at 220 nm. The leader peptide was separated from the unmodified core and precursor peptide using a linear gradient from 2% to 75% (v/v) of solvent A [80% (v/v) MeCN, 20% (v/v) H₂O, 0.086% (v/v) TFA] in solvent B [0.1% (v/v) TFA in H₂O] over 30 min at a flow rate of 1 mL min⁻¹ at room temperature. The concentration of the leader peptide was calculated by converting the area under the leader peptide peak to leader peptide concentration using a calibration curve made from purified leader peptide. Rates of leader peptide formation were then plotted against substrate concentration and the resulting graph was fit to the Michaelis-Menten equation. Values were plotted as the average and standard error of two independent experiments. Reactions were performed with tagged enzymes and substrates unless otherwise noticed.

### Example 6. Synthesis of ElxO Substrate Analogs

Peptides were synthesized by standard Fmoc-based solid phase peptide synthesis.

### Example 7. Cloning, Expression and Purification of Wild Type His₆-ElxO and Mutant Variants

To generate pHis₆-ElxO(S139A), pHis₆-ElxO(Y152F), pHis₆-ElxO(K156A), and pHis₆-ElxO(K156M), the entire pHis₆-ElxO reported previously was amplified by PCR using Pfu Turbo hot-start DNA polymerase (Stratagene) or iProof high-fidelity polymerase (BioRad) with the appropriate mutagenesis primers ElxO.S139A.FP and ElxO.S139.RP, ElxO.Y152F.FP and ElxO.Y152F.RP, ElxO.K156A.FP and ElxO.K156A.RP, or ElxO.K156M.FP and ElxO.K156M.RP, followed by treatment with DpnI (New England Biolabs) and transformation of Escherichia coli DH5a cells. The correct sequence of the insert was confirmed by sequencing at the W. M. Keck Center for Comparative and Functional Genomics at the University of Illinois at Urbana-Champaign. The proteins were expressed and purified using a HisTrap HP column (GE Healthcare) as described elsewhere for His₆-ElxO, followed by further purification by size exclusion chromatography using an ÄKTA purifier equipped with a HiLoad 16/60 Superdex 200 column (GE Healthcare) and a flow of 1.5 mL min⁻¹ of running buffer (50 mM HEPES, 300 mM NaCl, 10% (v/v) glycerol, pH 7.4).

### Example 8. Wild Type and Mutant His₆-ElxO Activity Assays

Wild type or mutant His₆-ElxO (2 or 10 μM) and purified peptide (0.1 to 5 mM) were incubated with NADPH (2.5 mM) in assay buffer (100 mM HEPES, 500 mM NaCl, pH 7.5) at 25° C. Reaction progress was monitored by UV spectrophotometry to measure initial rates, measuring the disappearance of NADPH absorbance at 340 nm. Formation of reduced peptides was confirmed by LC-MS using an Agilent 1200 instrument equipped with a single quadruple multimode ESI/APCI ion source mass spectrometry detector and a Synergi Fusion-RP column (4.6 mm×150 mm, Phenomenex). The mobile phase was 0.1% (v/v) formic acid in water (A) and methanol (B). A gradient of 0-70% (v/v) B in A over 30 minutes and a flow rate of 0.5 mL min⁻¹ were used.

### Example 9. Production of Dihydrolactocin S and Bioactivity Assays

Synthetic lactocin S (50 μM), obtained from Prof. J. Vederas (University of Alberta), was incubated with His₆-ElxO (50 μM) and NADPH (10 mM) in assay buffer (100 mM HEPES, 500 mM NaCl, pH 7.5) at room temperature for 12 h. The formation of reduced peptide was confirmed by LC-MS using a Waters SYNAPT™ mass spectrometry system equipped with a ACQUITY UPLC®, an ESI ion source, a quadrupole time-of-flight detector, and a ACQUITY Bridged Ethyl Hybrid (BEH) C8 column (2.1 mm×50 mm, 1.7 μm, Waters). A gradient of 3-97% (v/v) B (0.10% (v/v) formic acid in methanol) in A (0.1% (v/v) formic acid in water) over 12 min was used.

Agar diffusion bioactivity assays were performed using de Man-Rogosa-Sharpe (MRS) agar media. For each assay, aliquots of agar medium inoculated with overnight cultures of indicator strain (1/100 dilution) were poured into sterile plates. Aliquots of 20 μL of sample were placed into wells made on the solidified agar and the plates were incubated at 37° C. overnight. For determination of critical concentration, the diameter of the inhibition zones were determined and fitted to the equation D=a+b×(C), where D is the diameter of the inhibition zone, C is the concentration of bacteriocin, and a and b are constant parameters. For MIC determinations, serial dilutions of peptides were prepared in MRS broth and aliquots of 50 μL were dissolved in 150 μL of a 1 to 50 dilution of an overnight culture of indicator strain in fresh MRS broth on 96-well plates. The cultures were incubated at 37° C. overnight and the wells with no bacterial growth (OD₆₀₀<0.3) were determined.

### Example 10. General Materials and Methods

The gene encoding CylA was synthesized by GeneArt (Invitrogen) with codon usage optimized for E. coli expression. The DNA sequences for cylM, cylA, cylL_{L} and cylL_{S} are listed in Table 9. All other oligonucleotides were synthesized by Integrated DNA Technologies and used as received. Restriction endonucleases, DNA polymerases, and T4 DNA ligase were obtained from New England Biolabs. Media components were purchased from Difco Laboratories. Trypsin was purchased from Worthington Biochemical Corporation; Factor Xa was obtained from New England Biolabs and other endoproteinases were ordered from Roche Biosciences. Defibrinated rabbit blood was purchased from Hemostat Laboratories and used within 10 days of receipt. Chemicals were ordered from Sigma Aldrich or Fisher Scientific unless specified otherwise. Miniprep, gel extraction and PCR purification kits were purchased from Qiagen.

All polymerase chain reactions (PCRs) were carried out on a C1000™ thermal cycler (Bio-Rad). DNA sequencing was performed by ACGT, Inc. Preparative HPLC was performed using a Waters Delta 600 instrument equipped with appropriate columns. Solid phase extraction was performed with a Strata X-L polymeric reverse phase column (Phenomenex). MALDI-TOF MS was carried out on a Bruker Daltonics UltrafleXtreme MALDI TOF/TOF instrument (Bruker) or a Voyager-DE-STR instrument (Applied Biosystems). LC-ESI-Q/TOF MS analyses were conducted using a Micromass Q-Tof Ultima instrument (Waters) equipped with a Vydac C18 column (5 μm; 100 Å; 250×1.0 mm). Absorbance of rabbit hemoglobin solution was measured in 96-well plates with a Synergy™ H4 Microplate Reader (BioTek). Negative numbers are used for amino acids in the leader peptide counting backwards from the leader peptide cleavage site.

### Example 11. Strains and Plasmids

The indicator strain, Lactococcus lactis HP and the lichenicidin producing strain, Bacillus licheniformis DSM 13=ATCC 14580 were both obtained from American Type Culture Collection. E. coli DH5a and E. coli BL21 (DE3) cells were used as host for cloning and plasmid propagation, and host for protein expression, respectively. The co-expression vector pRSFDuet-1 was obtained from Novagen.

### Example 12. Construction of pRSFDuet-1 Derivatives for Expression of CylA, CylL_{L} and CylL_{S}

The cylA, cylL_{L} and cylL_{S} genes were synthesized with codon usage optimized for E. coli expression, amplified using appropriate primers and cloned into the MCS1 of a pRSFDuet-1 vector using restriction sites EcoRI and NotI to generate the plasmids pRSFDuet-1/CylA-27-412, pRSFDuet-1/CylL_{L} and pRSFDuet-1/CylL_{S}. Primer sequences are listed in Table 10.

### Example 13. Construction of pRSFDuet-1 Derivatives for Co-Expression of HalM2 and HalM1 with HalA2-GDVQAE, HalA2-GDVQAE-T2A, and HalA1-GDVQAE

Genes encoding the mutant peptides were amplified by multi-step overlap extension PCR. First, the amplification of the 5′ leader part was carried out by 30 cycles of denaturing (95° C. for 10 s), annealing (55° C. for 30 s), and extending (72° C. for 15 s) using forward primers for halA2 and halA1 and appropriate reverse primers (Table 10) to generate a forward megaprimer (FMP). In parallel, PCR reactions using appropriate forward primers and reverse primers for halA2 and halA1 (Table 10) were performed to produce 3′ fragments (termed reverse megaprimer, RMP). The 5′ FMP fragment and the 3′ RMP fragment were purified by 2% agarose gel followed by use of a Qiagen gel extraction kit. The 2 fragments were combined in equimolar amounts (approximately 20 ng each for a 50 μL PCR) and amplified using the same PCR conditions as above with halA2 and halA1 primers. The resulting PCR products were purified, digested and then cloned into the MCS1 of pRSFDuet-1/HalM2-2 and pRSFDuet-1/HalM1-2, respectively, to generate pRSFDuet-1/HalA2-GDVQAE/HalM2-2, pRSFDuet-1/HalA2-GDVQAE-T2A/HalM2-2 and pRSFDuet-1/HalA1-GDVQAE/HalM1-2 vectors.

### Example 14. Construction of pRSFDuet-1 Derivatives for Expression of ProcA1.7-GDVQAE and NisA-GDVQAE Peptides

Mutant peptide genes were generated by a similar multi-step overlap extension PCR procedure as described above and cloned in the MCS1 of a pRSFDuet vector to generate pRSFDuet-1/ProcA1.7-GDVQAE and pRSFDuet-1/NisA-GDVQAE vectors. Primer sequences are listed in Table 10.

### Example 15. Construction of pRSFDuet-1 Derivatives for Expression of ProcA1.7-GDVQAE-T1G, ProcA1.7-GDVQAE-T1F, ProcA1.7-GDVQAE-T1W Peptides and CylA-27-412-E95A, CylA-27-412-S359A Proteins

The expression vectors pRSFDuet-1/ProcA1.7-GDVQAE-T1G, pRSFDuet-1/ProcA1.7-GDVQAE-T1F, pRSFDuet-1/ProcA1.7-GDVQAE-T1W, pRSFDuet-1/CylA-27-412-E95A and pRSFDuet-1/CylA-27-412-S359A were generated using quick change methodology based on the pRSFDuet-1/ProcA1.7-GDVQAE and pRSFDuet-1/CylA-27-412 vectors, respectively. Primer sequences are listed in Table 10.

### Example 16. Construction of pRSFDuet-1 Derivatives for Expression of LicA2 Peptide and LicP Protein

Bacillus licheniformis DSM 13=ATCC 14580 was grown in LB at 37° C. for 12 h with vigorously shaking and plasmid was extracted using a Qiagen miniprep kit. LicA2 and LicP genes were amplified from the plasmid using appropriate primers and cloned into the MCS1 of a pRSFDuet-1 vector using restriction sites BamHI and NotI to generate pRSFDuet-1/LicA2 and pRSFDuet-1/LicP-25-433, respectively. Primer sequences are listed in Table 10.

### Example 17. Expression and Purification of CylA, LicP and CylA Mutant Proteins

E. coli BL21 (DE3) cells were transformed with pRSFDuet-1/CylA-27-412, pRSFDuet-1/CylA-27-412-E95A, pRSFDuet-1/CylA-27-412-S359A or pRSFDuet-1/LicP-25-433 vectors and plated on an LB plate containing 50 mg/L kanamycin. A single colony was picked and grown in 20 mL of LB with kanamycin at 37° C. for 12 h and the resulting culture was inoculated into 2 L of LB. Cells were cultured at 37° C. until the OD at 600 nm reached 0.5, cooled and IPTG was added to a final concentration of 0.1 mM. The cells were cultured at 18° C. for another 10 h before harvesting. The cell pellet was resuspended on ice in LanP start buffer (20 mM HEPES, 1 M NaCl, pH 7.5 at 25° C.) and lysed by homogenization. The lysed sample was centrifuged at 23,700×g for 30 min and the pellet was discarded. The supernatant was passed through 0.45-μm syringe filters and the protein was purified by immobilized metal affinity chromatography (IMAC) loaded with nickel as described in B. Li et al. Methods Enzymol. 2009, 458, 533. The proteins were generally eluted from the column at an imidazole concentration between 150 mM and 300 mM and the buffer was exchanged using a GE PD-10 desalting column pre-equilibrated with LanP start buffer. Protein concentration was quantified by its absorbance at 280 nm. The extinction coefficient for His₆-CylA-27-412, His₆-CylA-27-412-E95A and His₆-CylA-27-412-S359A was calculated as 30,830 M⁻¹ cm⁻¹. The extinction coefficient for His₆-LicP-25-433 was calculated as 46,300 M⁻¹ cm⁻¹. Aliquoted protein solutions were flash-frozen and kept at −80° C. until further usage.

### Example 18. Expression and Purification of Modified His₆-CylL_{L}, His₆-CylL_{S}, His₆-HalA2-GDVQAE, His₆-HalA2-GDVQAE-T2A and His₆-HalA1-GDVQAE

Modified peptides were obtained using a similar procedure described previously using the corresponding co-expression vectors (Y. Shi, et al. J. Am. Chem. Soc. 2011, 133, 2338; W. Tang and W. A. van der Donk, Nat. Chem. Biol. 2013, 9, 157).

### Example 19. Expression and Purification of Unmodified His₆-CylL_{L}, His₆-CylL_{S}, His₆-ProcA1.7-GDVQAE, His₆-NisA-GDVQAE, His₆-ProcA1.7-GDVQAE-T1G, His₆-ProcA1.7-GDVQAE-T1F, His₆-ProcA1.7-GDVQAE-T1W and His₆-LicA2

E. coli BL21 (DE3) cells were transformed with pRSFDuet-1/CylL_{L}, pRSFDuet-1/CylL_{S}, pRSFDuet-1/ProcA1.7-GDVQAE, pRSFDuet-1/NisA-GDVQAE, pRSFDuet-1/ProcA1.7-GDVQAE-T1G, pRSFDuet-1/ProcA1.7-GDVQAE-T1F, pRSFDuet-1/ProcA1.7-GDVQAE-T1W or pRSFDuet-1/LicA2 plasmids and plated on an LB plate containing 50 mg/L kanamycin. A single colony was picked and grown in 10 mL of LB with kanamycin at 37° C. for 12 h and the resulting culture was inoculated into 1 L of LB. Cells were cultured at 37° C. until the OD at 600 nm reached 0.5 and IPTG was added to a final concentration of 0.2 mM. The cells continued to be cultured at 37° C. for another 3 h before harvesting. The cell pellet was resuspended at room temperature in LanA start buffer (20 mM NaH₂PO₄, pH 7.5 at 25° C., 500 mM NaCl, 0.5 mM imidazole, 20% glycerol) and lysed by sonication. The sample was centrifuged at 23,700×g for 30 min and the supernatant was discarded. The pellet was then resuspended in LanA buffer 1 (6 M guanidine hydrochloride, 20 mM NaH₂PO₄, pH 7.5 at 25° C., 500 mM NaCl, 0.5 mM imidazole) and sonicated again. The insoluble portion was removed by centrifugation at 23,700×g for 30 min and the soluble portion was passed through 0.45-μm syringe filters. His₆-tagged peptides were purified by immobilized metal affinity chromatography (IMAC) loaded with nickel as described in B. Li et al. Methods Enzymol. 2009, 458, 533.

The eluted fractions were desalted using reverse phase HPLC equipped with a Waters Delta-pak C4 column (15 μm; 300 Å; 25×100 mm) or a Strata XL polymeric reverse phase SPE column. The desalted peptides were lyophilized and stored at −20° C. for future use.

### Example 20. Proteolytic Cleavage of the Leader Peptides

Targeted peptides were dissolved in H₂O to a final concentration of 3 mg/mL. To a 85 μL solution of peptides, 10 μL of 500 mM HEPES buffer (pH 7.5) was added followed by 5 μL of 0.5 mg/mL AspN protease (for modified CylL_{L} and CylL_{S} peptides), 0.1 mg/mL CylA protease (for modified and unmodified CylL_{L}, CylL_{S} peptides, and modified HalA2-GDVQAE peptide) or 0.1 mg/mL LicP protease (for LicA2). For cleavage tests of the engineered GDVQAE peptides, CylA was added to a final concentration of 0.01 mg/mL, whereas the peptide was added to a concentration of 0.3 mg/mL with 50 mM HEPES buffer (pH 7.5). The protease cleavage reaction mixtures were kept at 25° C. for 1 to 48 h. Osmotic pressure was adjusted with NaCl solution with a final concentration of 150 mM. The digested peptide mixture was directly used for antimicrobial and hemolytic assay. For the kinetic analysis of CylA and its mutant proteins, His₆-CylA-27-412 and His₆-CylA-27-412-E95A proteins were added to a final concentration of 500 ng/mL and 2.5 μg/mL (22 nM and 110 nM), respectively, with modified CylL_{S} supplied at a concentration of 0.3 mg/mL (36 μM). The reactions were allowed to proceed at room temperature and were stopped by 1% TFA at different time points for LC/MS analysis. Halα and Halβ were obtained by factor Xa cleavage of modified HalA1Xa and HalA2 Xa peptides using the procedure of Y. Shi, et al. J. Am. Chem. Soc. 2011, 133, 2338. CylL_{L}″ and CylL_{S}″ were prepared in the same way using modified CylL_{L}-E-1K and CylL_{S}-E-1K peptides as described in W. Tang and W. A. van der Donk, Nat. Chem. Biol. 2013, 9, 157.

### Example 21. Kinetic Analysis of Full Length CylA and CylA-96-412 Against Modified CylL_{S}

As full length CylA is not available due to its self-cleavage, His₆-CylA-27-412-E95A was chosen to serve as a substituent of full length CylA as the self-cleavage was abolished whereas the conserved catalytic C-terminal region of CylA remained unchanged. To obtain the mature protease CylA-96-412, His₆-CylA-27-412 was aged at 4° C. for 12 hours to allow the self-cleavage to proceed until CylA-96-412 was the dominant peak monitored by MALDI-TOF MS. The aged protein mixture was directly used as a substituent of CylA-96-412. To test the proteases' activities, CylA-96-412 and His₆-CylA-27-412-E95A were supplied with a final concentration of 22 nM and 110 nM, respectively, with modified CylL_{S} served at a concentration of 36 μM. The reactions were stopped at 3 minute, 6 minute, 12 minute and 24 minute by 1% TFA and the formation of mature CylL_{S}″ was monitored by liquid chromatography MS (LC/MS). A 5 μL volume of sample obtained from the cleavage reaction was applied to the column that was pre-equilibrated in aqueous solvent A. The solvents used for LC were: solvent A=0.1% formic acid in 95% water/5% acetonitrile and solvent B=0.1% formic in 95% acetonitrile/5% water. A solvent gradient of 0%-80% B over 30 min was employed and the fractionated sample was directly subjected to ESI-Q/TOF MS analysis. The production of core peptide was analyzed by extracted ion chromatography monitoring the desired product mass 1017 (M+2H⁺).

### Example 22. Antimicrobial Assay

L. lactis HP cells were grown in GM17 media under anaerobic conditions at 25° C. for 16 h. Agar plates were prepared by combining 15 mL of molten GM17 agar (cooled to 42° C.) with 150 μL of dense cell culture. The seeded agar was poured into a sterile 100 mm round dish (VWR) to solidify. Peptide samples were directly spotted on the solidified agar. Plates were incubated at 30° C. for 16 h and the antimicrobial activity was determined by the size of the zone of growth inhibition.

### Example 23. Hemolytic Assay for Cytolysin

A sample of 1 mL of defibrinated rabbit blood was added into 20 mL of PBS in a 50 mL conical tube and mixed gently. The PBS-diluted blood sample was centrifuged at 800×g for 5 min at 4° C. and the supernatant containing lysed blood cells and released hemoglobin was discarded. The process was repeated 2 to 4 times until the supernatant was clear. The blood cells were then diluted with PBS to make a 5% solution, which was immediately used to test the hemolytic activity of the peptides. To an Eppendorf tube, 50 μL of 5% washed red blood cell sample was added followed by the addition of the desired peptide samples or controls. PBS was used to adjust the final volume to 85 μL. All tubes were kept in a 37° C. incubator to allow the lytic reaction to proceed. At each time point, 8 or 10 μL of reaction mixture was taken out, diluted with 190 μL of fresh PBS and centrifuged at 800×g for 5 min. The supernatant (170 μL) was transferred to a new well and the absorbance was measured at 415 nm. The absorbance of prepared blood sample at each time point was analyzed in triplicate and the maximum absorbance was determined by adding 35 μL of 0.1% Triton in PBS to 50 μL of 5% blood sample and using the same analysis procedure.

TABLE 9
The DNA sequences for cylM, cylA, cylL_{L} and cylL_{S} with codon usage
optimized for E. coli expression.

cylM
ATGGAAGATAATCTGATTAATGTGCTGAGCATTAATGAACGTTGCTTTCTGCTG
AAACAGAGCGGCAAAGAAAAATATGATATTAAAAATCTGCAGGCCTGGAAAG
AACGTAAAAGCGTTCTGAAACAGGATGATCTGGATTATCTGATTAAATATAAA
TATGAAAGCCTGGATAATTTTGGCCTGGGTATTACCCCGATTGAAAATTTTCCG
GATAAAGAAGTGGCCATTCAGTATATTAAAGATCAGAGTTGGTATATTTTTTTT
GAAAGCATTCTGGATAGCTATAATGATAGCGAAGAAAAACTGCTGGAAGTTGA
TGCAAGCTATCCGTTTCGTTATTTTCTGCAGTATGCACGTCTGTTTCTGCTGGAT
CTGAATAGCGAACTGAATATTTGCACCAAAGAATTTATTATTAATCTGCTGGAA
ACCCTGACCCAGGAACTGATTCATCTGACCAGCAAAACCCTGGTTCTGGATCT
GCATACCTTTAAAAAAAATGAACCGCTGAAAGGCAATGATAGCAGCAAACGCT
TTATTTATTATCTGAAAAAACGCTTTAATAGCAAAAAAGATATTATTGCCTTTT
ATACCTGCTATCCGGAACTGATGCGTATTACCGTTGTTCGTATGCGCTATTTCC
TGGATAACACCAAACAAATGCTGATTCGTGTTACCGAAGATCTGCCGAGCATT
CAGAATTGCTTTAATATTCAGAGCAGTGAACTGAATAGCATTAGCGAAAGCCA
GGGTGATAGCCATAGCCGTGGTAAAACCGTTAGCACCCTGACCTTTAGTGATG
GTAAAAAAATTGTGTATAAACCGAAAATTAATAGCGAAAATAAACTGCGCGAT
TTTTTTGAATTTCTGAATAAAGAACTGGAAGCCGATATTTATATTGTGAAAAAA
GTGACCCGCAATACCTATTTTTATGAAGAATATATCGATAACATTGAAATCAAT
AACATCGAAGAAGTGAAAAAATATTACGAACGCTATGGCAAACTGATTGGCAT
TGCCTTTCTGTTTAATGTTACCGATCTGCATTATGAAAACATCATTGCCCATGG
CGAATATCCGGTGATTATTGATAATGAAACCTTTTTTCAGCAGAATATTCCGAT
TGAATTTGGTAATAGCGCAACCGTTGATGCCAAATACAAATATCTGGATAGCA
TTATGGTGACCGGTCTGGTTCCGTATCTGGCAATGAAAGATAAAAGCGATAGC
AAAGATGAAGGCGTTAATCTGAGCGCACTGAATTTTAAAGAACAGAGCGTGCC
GTTTAAAATTCTGAAAATTAAAAATACCTTTACCGATGAAATGCGCTTTGAATA
TCAGACCCATATTATGGATACCGCAAAAAATACCCCGATTATGAATAATGAAA
AAATCAGCTTTATCAGCTATGAAAAATATATTGTGACCGGCATGAAAAGCATT
CTGATGAAAGCCAAAGATAGCAAAAAAAAAATTCTGGCCTATATTAATAATAA
TCTGCAGAATCTGATTGTGCGCAATGTTATTCGTCCGACCCAGCGTTATGCAGA
TATGCTGGAATTTAGCTATCATCCGAATTGCTTTAGCAATGCCATTGAACGTGA
AAAAGTGCTGCATAATATGTGGGCCTATCCGTATAAAAATAAAAAAGTGGTGC
ATTATGAATTTTCAGATCTGATTGATGGCGATATTCCGATTTTTTATAATAATAT
TAGCAAAACCAGCCTGATTGCCAGTGATGGTTGTCTGGTTGAAGATTTTTATCA
GGAAAGTGCCCTGAATCGCTGCCTGAATAAAATTAATGATCTGTGTGATGAAG
ATATTAGCATTCAGACCGTGTGGCTGGAAATTGCACTGAATATTTATAACCCGT
ACAAATATATCAATGATCTGAAAAACCAGAATAGCAACAAATATATTTATACC
GGTCTGGAACTGAATGGCAAAATTATTCAGGCCTGCCAGAAAATTGAAAAAAA
AATCTTTAAACGTGCCATCTTTAACAAAAAAACCAATACCGTGAATTGGATTG
ATATTAAACTGGATCAGGATTGGAATGTGGGCATTCTGAATAATAATATGTAT
GATGGCCTGCCTGGTATTTTTATTTTTTATGTGGCCCTGAAATATATTACCAAA
AACCATAAATATGATTATGTGATCGAATGCATTAAAAATAGCATTTATACCATT
CCGAGCGAAGATATTCTGAGCGCATTTTTTGGTAAAGGCAGCCTGATTTATCCG
CTGCTGGTTGATTATCGCCTGAATAATGATATTAATAGCCTGAATGTGGCCGTG
GAAATTGCCGATATGCTGATTGAAAAAAAACCGATTAATAATGGCGAACTGAA
AAATGATTGGATTCATGGCCATAATAGCATTATTAAAGTGCTGCTGCTGCTGAG
CGAAATTACCGAAGATGAAAAATATCGCAAATTTAGCCTGGAAATTTTTGAAA
AACTGAGCGAAGAACCGTATTTTAATTTTCGTGGTTTTGGCCATGGCATTTATA
GCTATGTTCATCTGCTGAGCAAATTTAATCGCATTGATAAAGCCAATAGCCTGC
TGCATAAAATTAAAGAAAGCTATTTTGAAGAAGAACCGAAAAATAATTCCTGG
TGTAAAGGCACCGTTGGTGAACTGCTGGCAACCATTGAACTGTATGATGATAA
TATTAGTAACATCGATATTAACAAAACCATTGCCTATAAAAATAAAGATTGCC
TGTGCCATGGCAATGCAGGCACCCTGGAAGGTCTGATTCAGCTGGCAAAAAAA
GATCCGGAAACCTATCAGTATAAAAAAAATAAACTGATCAGCTATATGCTGAA
TGATTTTGAAAAAAATAATACCCTGAAAGTGGCAGGCAGCGAATATCTGGAAA
GCCTGGGTTTTTTTGTTGGTATTAGCGGTGTTGGTTATGAACTGCTGCGTAATCT
GGATAGCGAAATTCCGAATGCACTGCTGTTTGAACTGTAATAA
cylA (SEQ ID NO: 8)
ATGAAAAAACGCGGTCTGACCTATATTCTGATCAGCTATATCTTTCTGATTCTG
GGCACCACCGGTTATGCAAGCGATCTGAGCAACAATATCAGCTTCTTTATTGAT
AATAGCCAGACCACCGCCATCGAAGAAATTGAAAGCGAACTGAGCAGCGAGA
AAGTGGATTACATTCAAGAAATTGGTCTGGTGAGCTTCAAAAACCTGGATGAT
AGCGATCGCAAATTCATCGGCAAATATTTCAATGTGAGCGAAGGTAAAAAACT
GCCGGATTTTAAACCGGAAGAAGTGAATAGCAGCATCCTGAACATTAACATCC
TGAATAAAGATTTCAAAAGCTTTAATTGGCCGTACAAAAAAATCCTGAGCCAT
ATTGATCCGGTGAAAGAACAGCTGGGTAAAGATATTACCATTGCCCTGATTGA
TAGCGGTATTGATCGTCTGCATCCGAATCTGCAGGACAATAATCTGCGTCTGAA
AAACTATGTGAACGACATCGAACTGGATGAATATGGTCATGGCACCCAGGTTG
CCGGTGTTATTGATACCATTGCACCGCGTGTTAATCTGAACAGCTATAAAGTTA
TGGATGGCACCGATGGCAATAGCATTAATATGCTGAAAGCAATTGTGGATGCC
ACCAATGATCAGGTGGATATTATCAATGTTAGCCTGGGCAGCTACAAAAACAT
GGAAATTGATGATGAACGCTTTACCGTTGAAGCCTTTCGTAAAGTTGTTAATTA
CGCACGCAAAAATAACATCCTGATTGTTGCAAGCGCAGGTAATGAAAGCCGTG
ATATTAGCACCGGTAACGAAAAACACATTCCGGGTGGTCTGGAAAGCGTTATT
ACCGTTGGTGCAACCAAAAAAAGCGGTGATATTGCCGATTACAGCAATTATGG
TAGCAACGTGAGCATTTATGGTCCGGCAGGCGGTTATGGTGATAATTACAAAA
TCACCGGTCAGATTGATGCCCGTGAAATGATGATGACCTATTATCCGACCAGC
CTGGTTAGTCCGCTGGGTAAAGCAGCAGATTTTCCGGATGGTTATACCCTGAGC
TTTGGCACCAGCCTGGCAACACCGGAAGTTAGCGCAGCACTGGCAGCAATTAT
GAGCAAAAATGTGGATAACAGCAAAGACAGCAATGAAGTTCTGAACACCCTG
TTTGAAAATGCCGATAGCTTCATCGATAAAAACAGCATGCTGAAATACAAAGA
AGTGCGCATTAAATAA
cylL_{L}
ATGGAAAATCTGAGCGTTGTTCCGAGCTTTGAAGAACTGAGCGTTGAAGAAAT
GGAAGCAATTCAGGGTAGCGGTGATGTTCAGGCAGAAACCACACCGGTTTGTG
CAGTTGCAGCAACCGCAGCAGCAAGCAGCGCAGCATGTGGTTGGGTTGGTGGT
GGTATTTTTACCGGTGTTACCGTTGTTGTTAGCCTGAAACATTGCTAATAA
cylL_{S}
ATGCTGAATAAAGAAAATCAGGAAAATTATTATAGCAATAAACTGGAACTGGT
GGGTCCGAGCTTTGAAGAACTGAGCCTGGAAGAAATGGAAGCAATTCAGGGT
AGCGGTGATGTTCAGGCAGAAACCACACCGGCATGTTTTACCATTGGTCTGGG
TGTTGGTGCACTGTTTAGCGCAAAATTTTGCTAATAA

TABLE 10
Primer sequences for cloning of cylA-27-412, cylA-27-412-E95A, cylA-
27-412-S359A, cylL_{L}, cylL_{S}, halA2-GDVQAE, halA2-GDVQAE-T2A, halA1-GDVQAE,
procA1.7-GDVQAE, nisA-GDVQAE, procA1.7-GDVQAE-T1G, procA1.7-GDVQAE-
T1F, procA1.7-GDVQAE-T1W, licA2 and licP-25-433.

| Primer Name | Primer Sequence (5′-3′) |
|---|---|
| CylL_{L}_EcoRI_FP_Duet | AAAAAGAATTCGGAAAATCTGAGCGTTGTT |
| CylL_{L}_NotI_RP_Duet | AAAAAGCGGCCGCTTAGCAATGTTTCAGGCT |
| CylL_{S}_EcoRI_FP | AAAAAGAATTCGCTGAATAAAGAAAATCAG |
| CylL_{S}_NotI_RP | AAAAAGCGGCCGCTTAGCAAAATTTTGCGCT |
| HalA1_SacI_FP | CGCCACTCGGAGCTCGATGACAAATCTTTTAAAAG |
| HalA1_SbfI_RP | ATAGTGATCCTGCAGGTTAGTTGCAAGAAGGCATG |
| HalA2_BamHI_FP | AAAAAGGATCCGATGGTAAATTCAAAAGATTT |
| HalA2_HindIII_RP | AAAAAAAGCTTTTAGCACTGGCTTGTACACT |
| ProcA1.7_EcoRI_FP | GGTGCGAGGAATTCGATGAAGCATAGACAACTAAAT CTG |
| ProcA1.7_NotI_RP | ATAATATCGCGGCCGCTCAGCACATTTTCCC |
| NisA_BamHI_FP | CTAGATGGATCCGATGAGTACAAAAGATTTTAACTTGG |
| NisA_HindIII_RP | CTAGAAGCTTTTATTTGCTTACGTGAATACTACAATG |
| CylA27-EcoRI_FP | AAAAAGAATTCGCTGAGCAACAATATCAGCTTC |
| CylA-NotI_RP | AAAAAGCGGCCGCTTATTTAATGCGCACTTCTTTGTA |
| CylA-E95A_QC_FP | CCGGATTTTAAACCGGCAGAAGTGAATAGCAGC |
| CylA-E95A_QC_RP | GCTGCTATTCACTT CTGCCGGTTTAAAATCCGG |
| CylA-S359A_QC_FP | GGCACCGCACTGGCAACACCGGAAGTTAGCGCAGCA |
| CylA-S359A_QC_RP | TGC CAGTGCGGTGCCAAAGCTCAGGGTATAACCATCCGG |
| | AAAATC |
| HalA2-GDVQAE_FP | ACAACTTGGCCTTGCGCT |
| HalA2-GDVQAE_RP | GCAAGGCCAAGTIGT TTCTGCCTGAACATCACCTGAACCA |
| | GCTAGAGA |
| HalA1-GDVQAE_FP | TGCGCATGGTACAACATCAGC |
| HalA1-GDVQAE_RP | GTTGTACCATGCGCATTCTGCCTGAACATCACCTAGAA |
| | TATCTTGGTC |
| HalA2-GDVQAE-T2A_FP | ACAGCTTGGCCTTGCGCT |
| HalA2-GDVQAE-T2A_RP | GCAAGGCCAAGCTGTTTCTGCCTGAACATCACCTGAACCA |
| | GCTAGAGA |
| ProcA1.7-GDVQAE_FP | ACCATTGGGGGAACCATTGTG |
| ProcA1.7-GDVQAE_RP | GGTTCCCCCAATGGTTTCTGCCTGAACATCACCCAGCTCAGC |
| | ATCAGACAGGT |
| NisA-GDVQAE_FP | ATTACAAGTATTTCGCTATGT |
| NisA-GDVQAE_RP | CGAAATACTTGTAATTTCTGCCTGAACATCACCATCTTTCTTC |
| | GAAACAGATA |
| ProcA1.7-GDVQAE-T1G_QC_FP | CAGGCAGAAGGTATTGGGGGAACCATTGTGTCGATAACCTGT |
| | GAG |
| ProcA1.7-GDVQAE-T1G_QC_RP | CCCAATACCTTCTGCCTGAACATCACCCAGCTCAGCATCA |
| ProcA1.7-GDVQAE-T1F_QC_FP | CAGGCAGAATTCATTGGGGGAACCATTGTGTCGATAACCTGT |
| | GAG |
| ProcA1.7-GDVQAE-T1F_QC_RP | CCCAATGAATTCTGCCTGAACATCACCCAGCTCAGCATCA |
| ProcA1.7-GDVQAE-T1W_QC_FP | AGGCAGAATGGATTGGGGGAACCATTGTGTCGATAACCTGTG |
| | AG |
| ProcA1.7-GDVQAE-T1W_QC_RP | CCCAATCCATTCTGCCTGAACATCACCCAGCTCAGCATCA |
| LicP25_BamHI_FP | AAAAAGGATCCGAAAGAACAAGCAGGAGAACAG |
| LicP_NotI_RP | AAAAAGCGGCCGCTCACTCCTTGTTCATCATTTT |
| LicA2_BamHI_FP | AAAAAGGATCCGATGAAAACAATGAAAAATTCA |
| LicA2_NotI_RP | AAAAAGCGGCCGCCTAGCATCGGCTTGTACACTT |

### Example 24. LicP and LicA Substrates Preparation and Evaluation

General methods. All polymerase chain reactions (PCRs) were carried out on a C1000 thermal cycler (Bio-Rad). DNA sequencing was performed by ACGT, Inc. Preparative HPLC was performed using a Waters Delta 600 instrument equipped with a Waters Delta-pak C4 column (15 μm 300 Å25×100 mm). Solid phase extraction was performed with a Strata-X polymeric reversed phase column (Phenomenex) or Vydac BioSelect C4 reversed phase column. FPLC was carried out using an AKTA FPLC system (Amersham Pharmacia Biosystems). MALDI-TOF MS was carried out on a Bruker Daltonics UltrafleXtreme MALDI TOF/TOF instrument (Bruker). The detection of peptides with low molecular weights (700-3,500 Da), peptides with medium molecular weights (3,500-20,000 Da) and proteins with high molecular weights (20,000-50,000 Da) was achieved by using different instrument settings optimized for these mass ranges.

Materials. All oligonucleotides were synthesized by Integrated DNA Technologies and used as received. Restriction endonucleases, DNA polymerases, and T4 DNA ligase were obtained from New England Biolabs. Media components were purchased from Difco Laboratories and Fisher Scientific. Chemicals were ordered from Sigma Aldrich or Fisher Scientific unless otherwise specified. Miniprep, gel extraction and PCR purification kits were purchased from Qiagen and 5 PRIME. An UltraClean microbial DNA isolation kit was obtained from Mo Bio Laboratories, Inc.

Strains and plasmids. The lichenicidin producing strain, Bacillus licheniformis ATCC 14580, was obtained from the American Type Culture Collection. E. coli DH5U and E. coli BL21 (DE3) cells were used as hosts for cloning and plasmid propagation, and hosts for protein expression, respectively. The expression vector pRSFDuet-1 was obtained from Novagen.

Extraction of genomic DNA from Bacillus licheniformis ATCC 14580. Bacillus licheniformis ATCC 14580 was cultured in LB medium at 37° C. aerobically for 12 h and the genomic DNA was extracted using an UltraClean microbial DNA isolation kit following the manufacturer's protocol.

Construction of pRSFDuet-1 derivatives for expression of LicP-25-433 and LicA2. licP and licA2 genes were amplified from the genomic DNA of Bacillus licheniformis ATCC 14580 using appropriate primers and cloned into the multiple cloning site 1 (MCS1) of a pRSFDuet-1 vector to generate pRSFDuet-1/LicP-25-433 and pRSFDuet-1/LicA2 plasmids, respectively. Primer sequences are listed in Table 11.

Construction of pRSFDuet-1 derivatives for expression of ProcA1.7-NDVNPE and NisA-NDVNPE. Engineered peptide genes were generated by multi-step overlap extension PCR. First, the amplification of the 5′ leader part was carried out by 30 cycles of denaturing (95° C. for 10 s), annealing (55° C. for 30 s), and extending (72° C. for 15 s) using forward primers for procA1.7 and nisA and appropriate leader peptide reverse primers containing the mutations (Table 11) to generate a forward megaprimer (FMP). In parallel, PCR reactions using forward primers and reverse primers for procA1.7 and nisA core peptides (Table 11) were performed to produce the 3′ core fragments (termed reverse megaprimer, RMP). The 5′ FMP fragment and 3′ RMP fragment were purified by 2% agarose gel, combined in equimolar amounts and amplified using the same PCR conditions as above with procA1.7 and nisA primers. The resulting PCR products were purified, digested and then cloned into the MCS1 of a pRSFDuet-1 vector to generate pRSFDuet-1/ProcA1.7-NDVNPE and pRSFDuet-1/NisA-NDVNPE plasmids.

Construction of pRSFDuet-1 derivatives for expression of LicP-25-433-S376A, LicP-25-433-H186A, LicP-25-433-E100A, LicP-25-433-E100A-E102A, G-LicA2, NisA-NDVNPE-I1G, NisA-NDVNPE-I1T, NisA-NDVNPE-I1C, NisA-NDVNPE-I1L, NisA-NDVNPE-I1F, NisA-NDVNPE-I1W, NisA-NDVNPE-I1K, NisA-NDVNPE-I1E, LicA2-E-1A, LicA2-E-1D, LicA2-E-1Q, LicA2-P-2A, LicA2-N-3A, LicA2-V-4A, LicA2-V-4L, LicA2-V-4F, LicA2-D-5A, and LicA2-D-5K. The expression plasmids pRSFDuet-1/LicP-25-433-S376A, pRSFDuet-1/LicP-25-433-H186A, pRSFDuet-1/LicP-25-433-E100A, pRSFDuet-1/LicP-25-433-E100A-E102A, pRSFDuet-1/G-LicA2, pRSFDuet-1/NisA-NDVNPE-I1G, pRSFDuet-1/NisA-NDVNPE-I1T, pRSFDuet-1/NisA-NDVNPE-I1C, pRSFDuet-1/NisA-NDVNPE-I1L, pRSFDuet-1/NisA-NDVNPE-I1F, pRSFDuet-1/NisA-NDVNPE-I1W, pRSFDuet-1/NisA-NDVNPE-I1K, pRSFDuet-1/NisA-NDVNPE-I1E, pRSFDuet-1/LicA2-E-1A, pRSFDuet-1/LicA2-E-1D, pRSFDuet-1/LicA2-E-1Q, pRSFDuet-1/LicA2-P-2A, pRSFDuet-1/LicA2-N-3A, pRSFDuet-1/LicA2-V-4A, pRSFDuet-1/LicA2-V-4L, pRSFDuet-1/LicA2-V-4F, pRSFDuet-1/LicA2-D-5A, and pRSFDuet-1/LicA2-D-5K were generated using QuikChange methodology based on pRSFDuet-1/LicP-25-433, pRSFDuet-1/LicA2 and pRSFDuet-1/NisA-NDVNPE as templates. Primer sequences are listed in Table 11.

Construction of pRSFDuet-1 derivatives for co-expression of LicM2 with LicA2. LicM2 was amplified from the genomic DNA of Bacillus licheniformis ATCC 14580 using appropriate primers and cloned into the MCS2 of a pRSFDuet-1 vector to generate pRSFDuet-1/LicM2-2. The expression plasmid pRSFDuet-1/LicA2/LicM2-2 was constructed by inserting the licA2 gene into the MCS1 of the pRSFDuet-1/LicM2-2 plasmid. Primer sequences are listed in Table 11.

Construction of pET28b-MBP-BamL plasmid with LicP recognition sequence. Oligonucleotides corresponding to the LicP recognition sequence NDVNPE/SGS (SEQ ID NO.: 37) were inserted into the pET28b-MBP-BamL plasmid (1) in front of the DNA sequences corresponding to the TEV cleavage site using QuikChange methodology. Primer sequences are listed in Table 11. The resultant plasmid expressed the recombinant fusion protein MBP-BamL with the LicP recognition sequence installed between the two polypeptide portions for MBP and BamL (SEQ ID NO.: 221).

Expression and purification of LicP and LicP mutant proteins. E. coli BL21 (DE3) cells were transformed with one of the following plasmids: pRSFDuet-1/LicP-25-433, pRSFDuet-1/LicP-25-433-S376A, pRSFDuet-1/LicP-25-433-H186A, pRSFDuet-1/LicP-25-433-E100A or pRSFDuet-1/LicP-25-433-E100A-E102A, and plated on an LB plate containing 50 mg/L kanamycin. A single colony was picked and grown in 20 mL of LB containing 50 mg/L kanamycin at 37° C. for 12 h and the resulting culture was inoculated into 2 L of LB containing 50 mg/L kanamycin. Cells were cultured at 37° C. until the OD at 600 nm reached 0.5, cooled and IPTG was added to a final concentration of 0.1 mM. The cells were cultured at 18° C. for another 10 h before harvesting. The cell pellet was resuspended on ice in LanP buffer (20 mM HEPES, 1 M NaCl, pH 7.5 at 25° C.) and lysed by homogenization. The lysed sample was centrifuged at 23,700×g for 30 min and the pellet was discarded. The supernatant was passed through 0.45-μm syringe filters and the protein was purified by immobilized metal affinity chromatography (IMAC) loaded with nickel. The proteins were generally eluted from the column at an imidazole concentration between 150 mM and 300 mM and the buffer was exchanged using a GE PD-10 desalting column or a gel-filtration column pre-equilibrated with LanP buffer. Protein concentration was quantified by the absorbance at 280 nm. The extinction coefficient for His₆-LicP-25-433 was calculated as 46,300 M⁻¹ cm⁻¹. His₆-LicP-25-433-S376A was predominantly expressed in inclusion bodies.

Soluble protein was obtained by combining fractions eluted from the nickel column containing the desired protein and concentrating to a small volume. No gel filtration was performed for the mutant protein. The yield was determined to be about 50 μg for 1 L of culture. Aliquoted protein solutions were flash-frozen and kept at −80° C. until further usage.

Expression and purification of modified His₆-LicA2. Modified LicA2 was obtained using a procedure similar to that reported previously using the corresponding co-expression plasmid pRSFDuet-1/LicA2/LicM2-2.

Expression and purification of unmodified His₆-LicA2, His₆-G-LicA2, His₆-ProcA1.7-NDVNPE, His₆-NisA-NDVNPE, His₆-NisA-NDVNPE-I1G, His₆-NisA-NDVNPE-I1T, His₆-NisA-NDVNPE-I1C, His₆-NisA-NDVNPE-I1L, His₆-NisA-NDVNPE-I1F, His₆-NisA-NDVNPE-I1W, His₆-NisA-NDVNPE-I1K and His₆-NisA-NDVNPE-I1E. E. coli BL21 (DE3) cells were transformed with one of the following plasmids: pRSFDuet-1/LicA2, pRSFDuet-1/G-LicA2, pRSFDuet-1/ProcA1.7-NDVNPE, pRSFDuet-1/NisA-NDVNPE, pRSFDuet-1/NisA-NDVNPE-I1G, pRSFDuet-1/NisA-NDVNPE-I1T, pRSFDuet-1/NisA-NDVNPE-I1C, pRSFDuet-1/NisA-NDVNPE-I1L, pRSFDuet-1/NisA-NDVNPE-I1F, pRSFDuet-1/NisA-NDVNPE-I1W, pRSFDuet-1/NisA-NDVNPE-I1K or pRSFDuet-1/NisA-NDVNPE-I1E. Then the cells were plated on an LB plate containing 50 mg/L kanamycin. A single colony was picked and grown in 10 mL of LB containing 50 mg/L kanamycin at 37° C. for 12 h and the resulting culture was inoculated into 1 L of LB containing 50 mg/L kanamycin. Cells were cultured at 37° C. until the OD at 600 nm reached 0.5 and IPTG was added to a final concentration of 0.2 mM. The cells continued to be cultured at 37° C. for another 3 h before harvesting. The cell pellet was resuspended at room temperature in LanA start buffer (20 mM NaH₂PO₄, pH 7.5 at 25° C., 500 mM NaCl, 0.5 mM imidazole, 20% glycerol) and lysed by sonication. The sample was centrifuged at 23,700×g for 30 min and the supernatant was discarded. The pellet was then resuspended in LanA buffer 1 (6 M guanidine hydrochloride, 20 mM NaH₂PO₄, pH 7.5 at 25° C., 500 mM NaCl, 0.5 mM imidazole) and sonicated again. The insoluble portion was removed by centrifugation at 23,700×g for 30 min and the soluble portion was passed through 0.45-μm syringe filters. His₆-tagged peptides were purified by IMAC as previously described. The eluted fractions were desalted using reversed phase HPLC or a Strata X polymeric reversed phase SPE column. The desalted peptides were lyophilized and stored at −20° C. for future use.

Expression and purification of unmodified His₆-LicA2-E-1A, His₆-LicA2-E-1D, His₆-LicA2-E-1Q, His₆-LicA2-P-2A, His₆-LicA2-N-3A, His₆-LicA2-V-4A, His₆-LicA2-V-4L, His₆-LicA2-V-4F, His₆-LicA2-D-5A, and His₆-LicA2-D-5K. E. coli BL21 (DE3) cells were transformed with one of the following plasmids: pRSFDuet-1/LicA2-E-1A, pRSFDuet-1/LicA2-E-1D, pRSFDuet-1/LicA2-E-1Q, pRSFDuet-1/LicA2-P-2A, pRSFDuet-1/LicA2-N-3A, pRSFDuet-1/LicA2-V-4A, pRSFDuet-1/LicA2-V-4L, pRSFDuet-1/LicA2-V-4F, pRSFDuet-1/LicA2-D-5A, or pRSFDuet-1/LicA2-D-5K. Then the cells were plated on an LB plate containing 50 mg/L kanamycin. A single colony was picked and grown in 7 or 20 mL of LB containing 50 mg/L kanamycin at 37° C. for 14.5-16.5 h and the resulting culture was used to inoculate 750 mL of LB containing 50 mg/L kanamycin. Cells were cultured at 37° C. until the OD at 600 nm reached 0.5-0.6 and IPTG was added to a final concentration of 0.2 mM. The cells continued to be cultured at 37° C. for another 3 h before harvesting. The cell pellet was resuspended in LanA start buffer and lysed by sonication. The sample was centrifuged at 15,377×g for 30 min and the supernatant was discarded. The pellet was then resuspended in LanA buffer 1 and sonicated again. The insoluble portion was removed by centrifugation at 15,377×g for 30 min and the soluble portion was passed through 0.45-μm syringe filters. His₆-tagged peptides were purified by IMAC as previously described (2). Eluted fractions were desalted using a Vydac Bioselect C4 reversed phase SPE column. The desalted peptides were lyophilized, dissolved in water to a final concentration of 3 mg/mL and stored at −20° C. for future use.

Intermolecular cleavage of His₆-LicP-25-433-S376A by His₆-LicP-25-433. His₆-LicP-25-433-S376A and His₆-LicP-25-433 proteins were both diluted with LanP buffer to a final concentration of 0.2 mg/mL. Parallel reactions were set up for His₆-LicP-25-433 with a final protein concentration of 0.1 mg/mL in LanP buffer, His₆-LicP-25-433-S376A with a final protein concentration of 0.1 mg/mL in LanP buffer, and His₆-LicP-25-433-S376A and His₆-LicP-25-433 combined with a final protein concentration of 0.1 mg/mL each. The three reactions were allowed to proceed at room temperature for 0, 2, 4, 7 and 19 h before being stopped by addition of SDS loading buffer and boiling at 95° C. for 10 min and analyzed by SDS-PAGE.

Sequential proteolytic cleavage of modified LicA2. HPLC-purified LicM2-modified LicA2 was dissolved in H₂O to a final concentration of 3 mg/mL (340 μM). To a 17 μL solution of peptide (final peptide concentration 290 μM), 2 μL of 500 mM HEPES buffer (pH 7.5) was added followed by 1 μL of 0.5 mg/mL AspN. The reaction mixture was kept at room temperature for 12 h, and then 0.5 μL of 0.1 mg/mL LicP (final protein concentration 50 nM) was added. The reaction was then incubated at room temperature for one more hour. MALDI-TOF MS analysis was performed after each step.

Competition assay of LicP activity with modified and linear LicA2. To a reaction vessel containing 70 μL deionized H₂O, 5 μL each of 3 mg/mL modified LicA2 and linear G-LicA2 peptides were added (final peptide concentration 17 μM each) followed by 10 μL of 500 mM HEPES buffer (pH 7.5). Then, 10 μL of 0.01 mg/mL LicP was supplied (final protein concentration 21 nM) and the reaction was incubated at room temperature before being quenched by addition of formic acid to a final concentration of 1% at different time points. To observe the complete consumption of both peptides, substrates were incubated as above except that 10 μL of 1 mg/mL LicP was added (final protein concentration 2.1 μM). The reaction mixture was kept at room temperature for 12 h before being quenched with 1% formic acid for MS analysis.

Comparison of the proteolytic activity of LicP and TEV on MBP-BamL. A sample of 1 mL of MBP-BamL ((SEQ ID NO.: 221); 50 μM) was incubated with the same molar amount of LicP or TEV (final concentration 0.54 μM) at 4° C. At different time points, the reaction was quenched by adding an equal volume of loading dye and heating for 10 min at 90° C. The results were analyzed by Coomassie-stained SDS-PAGE.

The size difference between MBP and BamL bands is due to different recognition site locations of LicP and TEV in the construct.

LicP assay and Gel Analysis for wild type His₆-LicA2 and His₆-LicA2 mutants. A sample containing 100 μM peptide was incubated with 0.4 μM His₆-LicP(25-433) and 2 mM DTT in 50 mM HEPES (pH 7.5) buffer with a total reaction volume of 300 μL. After 15 min, 30 min, 1 h, 2 h, 4 h, and 7.5 h, the reactions were centrifuged for 30 s at 2000×g (because of observed precipitation) then 40 μL aliquots were removed and quenched by addition of 10.4 μL 5% aqueous formic acid to a final concentration of 1%.

Formic-acid quenched samples were diluted 25% with 95:5 NuPAGE LDS sample buffer (4×):β-mercaptoethanol to a final concentration of 60 μM peptide and 0.24 μM LicP. Solutions of 60 μM LicA2 substrates, 0.24 μM LicP, and 40-fold diluted Polypeptide Standards (#161-0326) were prepared, each containing 25% 95:5 NuPAGE LDS sample buffer (4×):β-mercaptoethanol. All SDS-PAGE samples were heated for 10 min at 70° C. then a 10-20% Mini-Protean Tris-Tricine gel (#456-3116) loaded with 5 μL per lane was run at 100 V for 125 min in 100 mM Tris, 100 mM Tricine, 0.1% SDS buffer while cooling the entire apparatus in ice.

The gels were subjected to consecutive coomassie and silver staining as described herein. The gels were rocked for 1 h in 50% MeOH/7% AcOH followed by rocking for 45 min in coomassie stain (0.25% coomassie/50% MeOH/10% AcOH), rinsing with H₂O, rocking overnight in 20% MeOH/10% AcOH, and then rinsing with H₂O again. All of the following steps were conducted with agitation on a Barnstead LabLine multipurpose rotator. The gels were washed with 50% MeOH/7% AcOH for 1.5 h, followed by washing with H₂O (3×10 min). The gels were then sensitized 1 min in 0.02% Na₂S₂O₃, then washed with H₂O (2×1 min), followed by staining for 30 min in a solution containing 44 mL of H₂O, 5.9 mL of 0.1 M AgNO₃, and 37.5 μL of 37% formaldehyde. The gels were then washed with H₂O (<1 min) then developed in a solution containing 100 mL of 3% Na₂CO₃, 2 mL of 0.02% Na₂S₂O₃, and 50 μL of 37% formaldehyde for <5 min. Developing was quenched by washing with 12% AcOH for 30 min, followed by washing with H₂O (2×30 min). Images of gels were acquired using an HP scanjet 8250.

Identification of the cleavage sites of the LanP proteases encoded in the genome of B. licheniformis 9945A and Bacillus cereus VD045. LanP proteases were purified using a similar procedure as described for LicP. Dehydrated and cyclized LanA2 encoded in the genome of B. licheniformis 9945A was obtained by coexpressing the precursor peptide with its corresponding LanM synthetase in E. coli, whereas linear LanA3 encoded in the genome of Bacillus cereus VD045 was obtained by expression in E. coli. The precursor peptides were incubated with their corresponding proteases and the results were analyzed using MALDI-TOF MS.

Removal of leader peptides of modified or linear LicA2. Modified or linear LicA2 peptides were dissolved in H₂O to make a 3 mg/mL solution (340 μM). To a 17 μL solution of peptide (final peptide concentration 290 μM), 2 μL of 500 mM HEPES buffer (pH 7.5) was added followed by 1 μL of 1 mg/mL LicP (final protein concentration 1.1 μM). The reaction was incubated at room temperature for 6 h followed by MS analysis.

Proteolytic cleavage of the leader peptides of engineered peptides. ProcA1.7-NDVNPE was dissolved in H₂O to a final concentration of 3 mg/mL (250 μM), whereas for NisA-NDVNPE and its mutant peptides, a 10 mg/mL peptide solution was made (1.3 mM). For ProcA1.7-NDVNPE, 15 μL of peptide solution (final peptide concentration 190 μM) was pre-mixed with 1 μL of 50 mM DTT and 2 μL of 500 mM HEPES buffer (pH 7.5), to which 2 μL of 0.1 mg/mL LicP (final protein concentration 210 nM) was added. The reaction was incubated at room temperature for 4 h before analysis. For NisA-NDVNPE-I1T and NisA-NDVNPE-I1C, 1 μL of peptide (final peptide concentration 65 μM) was pre-mixed with 1 μL of 50 mM DTT and 2 μL of 500 mM HEPES buffer (pH 7.5) in 14 μL H₂O, then 2 μL of 0.1 mg/mL LicP (final protein concentration 210 nM) was added. The reaction was incubated at room temperature for 20 h before analysis. For NisA-NDVNPE and other NisA mutant peptides, 1 mg/mL LicP (final protein concentration 2.1 μM) was employed instead of 0.1 mg/mL LicP and the reaction was kept at room temperature for 30 h before analysis.

### Example 25. Demonstration of Cleavage Activities of Other LanP Proteases

Materials. All oligonucleotides were synthesized by Integrated DNA Technologies and used as received. Restriction endonucleases, DNA polymerases, and T4 DNA ligase were obtained from New England Biolabs. Media components were purchased from Difco Laboratories and Fisher Scientific. Chemicals were ordered from Sigma Aldrich or Fisher Scientific unless otherwise specified. Miniprep, gel extraction and PCR purification kits were purchased from Qiagen and 5 PRIME. Synthetic genes were obtained from IDT, Inc. For LanP from Bacillus cereus VD156, the DNA was ordered in two gBlocks, whereas for the substrate it was ordered as one oligonucleotide. An UltraClean microbial DNA isolation kit was obtained from Mo Bio Laboratories, Inc.

Strains and plasmids. Bacillus licheniformis ATCC 14580 and Bacillus licheniformis ATCC 9945A were obtained from American Type Culture Collection. E. coli DH5a and E. coli BL21 (DE3) cells were used as hosts for cloning and plasmid propagation, and hosts for protein expression, respectively. The expression vector pRSFDuet-1 was obtained from Novagen.

Extraction of genomic DNA from B. licheniformis ATCC 14580 and B. licheniformis ATCC 9945A. Bacteria were cultured in LB medium at 37° C. aerobically for 12 h and the genomic DNA was extracted using an UltraClean microbial DNA isolation kit following the manufacturer's protocol.

Construction of pRSFDuet-1 derivatives for co-expression of LanM2-9945A with LanA2-9945A, and for expression of LanP-42-476-9945A. The genes for the LanM2 and LanA2 encoded in the genome of B. licheniformis ATCC 9945A (hereafter LanM2-9945A and LanA2-9945A, respectively) were amplified from the genomic DNA using appropriate primers and cloned into a pRSFDuet-1 vector to generate pRSFDuet-1/LanA2-9945A/LanM2-9945A-2 using Gibson assembly (LanA2 in MCS1 and LanM2 in MCS2). The gene encoding residues 42-476 of the class II LanP (designated LanP-42-476-9945A) was amplified from the genomic DNA of B. licheniformis ATCC 9945A using appropriate primers and cloned into the MCS1 of a pRSFDuet-1 vector to generate pRSFDuet-1/LanP-42-476-9945A using Gibson assembly. Primer sequences are listed in Table 11.

TABLE 11
Primer sequences for cloning of licP-25-433, licM2, licA2, licP-25-433-S376A, licP-
25-433-H186A, licP-25-433-E100A, licP-25-433-E100A-E102A, G-licA2, procA1.7-NDVNPE,
nisA-NDVNPE, nisA-NDVNPE-I1G, nisA-NDVNPE-I1T, nisA-NDVNPE-I1C, nisA-NDVNPE-I1L,
nisA-NDVNPE-I1F, nisA-NDVNPE-I1W, nisA-NDVNPE-I1K, nisA-NDVNPE-I1E,MBP-BamL-NDVNPE,
licA2-E-1A, licA2-E-1D, licA2-E-1Q, licA2-P-2A, licA2-N-3A, licA2-V-4A,
licA2-V-4L, licA2-V-4F, licA2-D-5A, and licA2-D-5K.

| Primer Name | Primer Sequence (5′-3′) |
|---|---|
| LicP_25_BamHI_FP | AAAAA GGATCCG AAAGAACAAGCAGGAGAACAG |
| LicP_NotI_RP | AAAAA GCGGCCGC TCACTCCTTG TTCATCATTT T |
| LicA2_BamHI_FP | AAAAA GGATCCG ATGAAAACAA TGAAAAATTC A |
| LicA2_NotI_RP | AAAAA GCGGCCGC CTAGCATCGG CTTGTACACT T |
| LicM2_NdeI_FP | AAAAA CATATG GTTTTCT TCGCCAAAGG GATG |
| LicM2_KpnI_RP | AAAAA GGTACC TCACCTGCCC GTCGGAATAT C |
| G-LicA2_QC_FP | CCAGGAT GGT ATGAAAAC AATGAAA AATTCAGCTGCCCGT |
| G-LicA2_QC_RP | GTTTTCAT ACC ATCCTGG CT GTGGTGATGA TGGTGATGG |
| ProcA1.7_EcoRI_FP | GGT GCG AGG AAT TCG ATG AAG CAT AGA CAA CTA AAT CTG |
| ProcA1.7_NotI_RP | ATA ATA TCG CGG CCG CTC AGC ACA TTT TCC C |
| NisA_BamHI_FP | CTA GAT GGA TCC GAT GAG TAC AAA AGA TTT TAA CTT GG |
| NisA_HindIII_RP | CTA GAA GCT TTT ATT TGC TTA CGT GAA TAC TAC AAT G |
| LicP-S376A_QC_FP | GGAACA GCA TTGGCC GCCCCG CAGGTAGCT |
| LicP-S376A_QC_RP | GG CCAA TGC TGT TCC GTATGAG AGGGAATATC CCTTTGGGAT |
| LicP-H186A_QC_FP | ACA GGA GCC GGAAC ACAA ACAGCCGGGATGATCAATATC |
| LicP-H186A_QC_RP | G TTCC GGC TCC TGT CGGATCT CCGGATACAG GC |
| LicP-E100A_QC_FP | CAGTAAAC GCA ACGGAATC A GTCATCAGCGGTTCGCC |
| LicP-E100A_QC_RP | GATTCCGTTGCGTTTACTG CTGTATT TGCAATCGGC |
| | TTTTCAATGAC |
| LicP-E100A-E102A_QC_FP | GCAACG GCA TCAGTC ATCAGCGGTTCGCCTG |
| LicP-E100A-E102A_QC_RP | GACTGA TGC CGTTGC GTTTA CTGCTGTATT TGCAATCGGC |
| ProcA1.7_core_FP | ACCATTGGGGGA ACCATTGTG |
| ProcA1.7-NDVNPE_RP | GGTTCC CCCAATGGT TTCAGGATTGACGTCATT CAG |
| | CTCAGCATCA GACAGGT |
| NisA_core_FP | ATTACAAGTATTTCGCTATGT |
| NisA-NDVNPE_RP | CGAAATACTT GTAAT TTCAGGATTGACGTCATT ATCTTTC |
| | TTCGAAACAG ATA |
| NisA-NDVNPE-I1G_QC_FP | CAATCCTGAA GGT ACAAGTATTTC GCTATGTACACC |
| | CGGTTGTAAAAC |
| NisA-NDVNPE-I1G_QC_RP | GAAATACTTGT ACC TTCAGGATTG |
| | ACGTCATTATCTTTCTTCGAAACAGATACC |
| NisA-NDVNPE-I1C_QC_FP | CAATCCTGAA TGT ACAAGTATTTC GCTATGTACACC |
| | CGGTTGTAAAAC |
| NisA-NDVNPE-I1C_QC_RP | GAAAT ACTTGT ACA TTCAGGATTG |
| | ACGTCATTATCTTTCTTCGAAACAGATACC |
| NisA-NDVNPE-I1T_QC_FP | CAATCCTGAA ACC ACAAGTATTTC GCTATGTACACC |
| | CGGTTGTAAAAC AG |
| NisA-NDVNPE-I1T_QC_RP | GAAATACTTGT GGT TTCAGGATTG |
| | ACGTCATTATCTTTCTTCGAAACAGATACCA |
| NisA-NDVNPE-I1L_QC_FP | CAATCCTGAA CTT ACAAGTATTTC GCTATGTACACC |
| | CGGTTGTAAAAC |
| NisA-NDVNPE-I1L_QC_RP | GAAATACTTGT AAG TTCAGGATTG |
| | ACGTCATTATCTTTCTTCGAAACAGATACC |
| NisA-NDVNPE-I1F_QC_FP | CAATCCTGAA TTT ACAAGTATTTC GCTATGTACACC |
| | CGGTTGTAAAAC |
| NisA-NDVNPE-I1F_QC_RP | GAAATACTTGT AAA TTCAGGATTG |
| | ACGTCATTATCTTTCTTCGAAACAGATACC |
| NisA-NDVNPE-I1W_QC_FP | CAATCCTGAA TGG ACAAGTATTTC GCTATGTACACC |
| | CGGTTGTAAAAC |
| NisA-NDVNPE-I1W_QC_RP | GAAATACTTGT CCA TTCAGGATTG |
| | ACGTCATTATCTTTCTTCGAAACAGATACC |
| NisA-NDVNPE-I1K_QC_FP | CAATCCTGAA AAA ACAAGTATTTC GCTATGTACACC |
| | CGGTTGTAAAAC |
| NisA-NDVNPE-I1K_QC_RP | GAAATACTTGT TTT TTCAGGATTG |
| | ACGTCATTATCTTTCTTCGAAACAGATACC |
| NisA-NDVNPE-I1E_QC_FP | CAATCCTGAA GAA ACAAGTATTTC GCTATGTACACC |
| | CGGTTGTAAAAC |
| NisA-NDVNPE-I1E_QC_RP | GAAATACTTGT TTC TTCAGGATTG |
| | ACGTCATTATCTTTCTTCGAAACAGATACC |
| MBP-BamL-NDVNPE_QC_FP | AATGACGTCAATCCTGAA TCTGGTTCT |
| | GAGAACCTGTACTTCCAATCC |
| MBP-BamL-NDVNPE_QC_RP | TTCAGGATTGACGTCATTAGATCCACGCG GAACCAG |
| LicA2-E-1A_QC_FP | TCAATCCT GCA ACAACTC CTGCTACAACCTCTTCTTGG AC |
| LicA2-E-1A_QC_RP | GAGTTGT TGC AGGATTGA CGTCATTTCC TCCTACCAAA GC |
| LicA2-E-1D_QC_FP | TCAATCCT GAT ACAACTC CTGCTACAACCTCTTCTTGG AC |
| LicA2-E-1D_QC_RP | GAGTTGT ATC AGGATTGA CGTCATTTCC TCCTACCAAA GC |
| LicA2-E-1Q_QC_FP | TCAATCCT CAA ACAACTC CTGCTACAACCTCTTCTTGG AC |
| LicA2-E-1Q_QC_RP | GAGTTGT TTG AGGATTGA CGTCATTTCC TCCTACCAAA GC |
| LicA2-P-2A_QC_FP | ACGTCAAT GCT GAA ACAA CTCCTGCTACAACCTCTTCTTG |
| LicA2-P-2A_QC_RP | TTGTT TC AGC ATTGA CGT CATTTCC TCCTACCAAA |
| | GCTTTCAATT |
| LicA2-N-3A_QC_FP | GACGTC GCT CCTGAA ACAACTCCTGCTACAACCTCT |
| LicA2-N-3A_QC_RP | TTCAGG AGC GACGTC ATTTCC TCCTACCAAA GCTTTCAATT |
| LicA2-V-4A_QC_FP | AAATGAC GCC AATCCTG AA ACAACTCCTGCTACAACC TCTT |
| LicA2-V-4A_QC_RP | CAGGATT GGC GTCATTT CC TCCTACCAAA GCTTTCAATT CC |
| LicA2-V-4L_QC_FP | GAAATGAC CTG AATCCTGA A ACAACTCCTGCTACAACC TCTT |
| LicA2-V-4L_QC_RP | TCAGGATT CAG GTCATTTC C TCCTACCAAA GCTTTCAATT CCTC |
| LicA2-V-4F_QC_FP | GAAATGAC TTC AATCCTGA A ACAACTCCTGCTACAACC TCTT |
| LicA2-V-4F_QC_RP | TCAGGATT GAA GTCATTTC C TCCTACCAAA GCTTTCAATT CCTC |
| LicA2-D-5A_QC_FP | AGGAAAT GCC GTCAATC CT GAA ACAACTCCTGCTACAACC |
| LicA2-D-5A_QC_RP | GATTGAC GGC ATTTCC T CC TACCAAA GCTTTCAATT CCTCTTC |
| LicA2-D-5K_QC_FP | GGAGGAAAT AAA GTCAATCCT GAA |
| | ACAACTCCTGCTACAACCTCT |
| LicA2-D-5K_QC_RP | AGGATTGAC TTT ATTTCC TCC TACCAAA GCTTTCAATT |
| | CCTCTTCG |

Expression plasmids for LanP and one LanA substrate from B. cereus VD156. The lanP gene and one of the genes for its putative substrates were synthesized as codon-optimized dsDNA oligos and cloned into pRSFDuet vector between BamHI and NotI restriction sites via Gibson Assembly. For expression in E. coli, the N-terminal secretion signal of the protease (the first 27 amino acid as predicted by signalP, see sequence shown in red in Table S2) was removed, and E. coli B121(DE3) cells were transformed with the resulting plasmids containing the truncated protease gene (VD156P(del)) or the four substrate genes. The cells were incubated at 37° C. until the OD600 reached 0.5-0.8, then induced with 0.20 mM IPTG, and expressed at 18° C. for 20 h. The synthetic gene sequences are listed in Table 12.

Identification of the cleavage sites of the LanP proteases encoded in the genome of B. licheniformis 9945A and B. cereus VD156. The LanP proteases from B. licheniformis 9945A and B. cereus VD156 were purified using a similar procedure as described for LicP. A His₆-tagged analog of dehydrated and cyclized LanA2 encoded in the genome of B. licheniformis 9945A was obtained by coexpression of the precursor peptide with its corresponding LanM synthetase in E. coli using the same procedure as described herein for LicA/LicM. Linear LanA3 encoded in the genome of B. cereus VD156 was also obtained by expression in E. coli as an N-terminal His₆-tagged peptide. The peptides were incubated with their corresponding purified proteases and the results were analyzed using MALDI-TOF MS.

TABLE 12
Sequences of the synthetic genes for the protease from B. cereus VD156
optimized for expression in E. coli.

| | | |
|---|---|---|
| Name | Accession number | Sequenceª |

Strain: B. cereus VD156

| | | |
|---|---|---|
| LanA3 | EJR72967.1 | MKNISEKSVGLSMKKLDTTEMEKIYGASGVDTRTH |
| | (SEQ ID NO: 27) | PTVVVVSRASSKFCVTVAASALLSYNMNKC |
| | optimized nucleotide | ATGAAAAATA TTTCAGAGAA ATCAGTAGGG |
| | sequence | CTTAGTATGA AGAAGCTGGA TACCACCGAA |
| | (SEQ ID NO: 28) | ATGGAAAAAA TCTATGGCGC GTCTGGTGTA |
| | | GACACGCGTA CGCATCCCAC AGTCGTCGTA |
| | | GTGAGCCGTG CCAGCTCAAA ATTTTGCGTC |
| | | ACCGTAGCTG CATCTGCACT GCTCTCGTAT |
| | | AACATGAATA AGTGTTAA |
| LanP | EJR72593.1 | MYRFKKYCLSIISFILIISFFPNNTNATQIAYYSILIRD |
| | (SEQ ID NO: 25) | NTDFNTVLDKLSKDNQEVVYSIPEVNLIQVKGEKGK |
| | | IISGIGLESIEEINPSTGEFKTYTPNINDQKVLDNKAIW |
| | | DVQWDIKRITNNGESYKLHSPSGKVSVALIDSGYPE |
| | | NHPDIKSISIQKSKNLVPKGGYKGNEENETGNIHQLT |
| | | DRTGHGTSVLSQVNADGLMKGVAPGMPVNMYRVF |
| | | GESSAEGSWIIKGIIEAAKDKNDVINISAGSYLLKNG |
| | | TYSDGSGNNRAEIKAYEKAIHYANKKGSIVVSALGN |
| | | DSININIYSELLSILNSKIKDEGKSATGIVQDIPAQLAQ |
| | | VVSVASTGMDSKVSSFSNYGKNIIDFTAPGGDIKLL |
| | | NKFGADVWMAEEMFKKEMILVAHPQGGYYFNYG |
| | | NSLATPKVSGALALVIDKYGYKNKPNKAINHLKRN |
| | | TNAENEIDLYKALQE |
| | optimized nucleotide | ATGTATCGCT TCAAGAAATA TTGCCTCAGC |
| | sequence | ATTATCTCGT TCATTCTGAT CATCAGTTTC |
| | (SEQ ID NO: 26) | TTCCCGAACA ATACCAACGC GACTCAAATC |
| | | GCCTATTACT CAATTCTGAT CCGGGATAAC |
| | | ACTGACTTCA ACACAGTACT GGACAAATTG |
| | | AGTAAGGATA ACCAGGAGGT GGTCTATAGC |
| | | ATCCCGGAAG TCAATCTGAT TCAAGTCAAA |
| | | GGCGAAAAAG GTAAGATTAT TAGTGGTATC |
| | | GGTTTAGAGA GTATTGAAGA GATTAACCCG |
| | | TCAACAGGCG AATTCAAAAC CTACACCCCG |
| | | AATATTAATG ACCAAAAAGT GCTGGATAAC |
| | | AAAGCCATCT GGGACGTCCA GTGGGATATC |
| | | AAACGCATTA CGAACAACGG CGAAAGTTAT |
| | | AAATTACACT CTCCGAGCGG GAAAGTGAGT |
| | | GTTGCACTGA TTGATAGCGG TTATCCGGAA |
| | | AACCACCCAG ATATCAAATC AATCTCCATC |
| | | CAGAAAAGCA AGAACCTTGT TCCGAAGGGG |
| | | GGCTATAAGG GAAATGAAGA AAACGAAACC |
| | | GGTAACATTC ACCAGCTGAC GGATCGCACT |
| | | GGTCACGGGA CCAGCGTGCT GTCTCAAGTG |
| | | AACGCCGATG GTTTAATGAA AGGAGTTGCT |
| | | CCTGGCATGC CTGTGAACAT GTATCGTGTC |
| | | TTCGGCGAAT CATCGGCAGA GGGGAGTTGG |
| | | ATCATCAAAG GAATTATCGA GGCCGCCAAG |
| | | GATAAGAATG ATGTTATCAA CATTTCAGCG |
| | | GGCAGCTATT TACTGAAGAA CGGCACCTAT |
| | | TCTGACGGAA GCGGCAACAA TCGGGCAGAA |
| | | ATCAAAGCAT ACGAAAAGGC GATCCATTAT |
| | | GCGAACAAAA AAGGAAGCAT TGTTGTGAGC |
| | | GCTCTGGGCA ACGACAGCAT TAACATTAAT |
| | | ATCTACTCCG AACTGCTGAG CATCCTGAAT |
| | | AGCAAAATTA AAGATGAAGG CAAAAGTGCC |
| | | ACGGGCATCG TGCAAGACAT CCCGGCTCAG |
| | | CTGGCACAAG TTGTGTCTGT CGCGTCGACG |
| | | GGCATGGATA GCAAAGTGTC AAGCTTTTCG |
| | | AACTACGGAA AAAACATTAT TGATTTTACC |
| | | GCCCCGGGAG GGGATATTAA ACTTCTCAAT |
| | | AAATTCGGCG CTGATGTTTG GATGGCTGAA |
| | | GAAATGTTCA AGAAGGAGAT GATTTTGGTC |
| | | GCTCACCCGC AGGGTGGCTA CTACTTCAAT |
| | | TACGGTAACT CCTTAGCTAC GCCCAAGGTC |
| | | AGCGGTGCCC TCGCTCTCGT GATCGATAAA |
| | | TATGGTTACA AAAATAAACC CAACAAAGCT |
| | | ATTAATCATC TGAAACGTAA TACTAACGCG |
| | | GAGAACGAAA TTGATTTGTA CAAAGCACTC |
| | | CAAGAGTAA |

^{a}The sequence shown in red is predicted to be a secretion signal and was removed in the expression constructs. Also shown is the sequence of the LanA3 precursor peptide. The observed cleavage occurred after the Arg shown in blue.

Additional nucleic acid and amino acid sequences described herein are listed in Table 13.

TABLE 13
Additional isolated nucleic acid and amino acid sequences disclosed
herein.

| Name | |
|---|---|
| [SEQ ID NO:] | Nucleic Acid Sequence or Amino Acid Sequence |
| ElxP | atggataattttcttagttggcctaataaaaataaatattttgatgaaataaaagatgaagttaaaa |
| (SEQ ID NO: 4) | tattatatatagatagtgggtgtgatataaatcatatcgaagttaaagaaaatatattgataaatga |
| | atctaaatcttttgtggacaatgatagtgaattatatgactatacgggacatggaacacaaattatt |
| | agtgcaataacaggtaagcataatatgattggactatatcctagaagtaaaattgtaatatataa |
| | aataactaattataaaggtgaaactaaatttgaatggttatataaagcattatataaagctataaa |
| | aatggactataaaattattaacataagttattcaggatacacccaaaataattacataatatctaa |
| | attcaaaagattaatagaacaagcagttaaaaaaaatatacatattttatgtagtgctagtaatg |
| | atgaagtggaaaaaggtttttcaataccttctgattttaaaggagtctataaaattgcgagtataa |
| | atattgaagataaatattctagttatatttctaaatctaatgctgaatactttgctcctggaggagata |
| | attatttaaagacacagaatccacaatcatttattttgttagctaatagttctatttctaactttaatatt |
| | ggttctgattttggtatagataaaaggtatactttaaattttggtaatagtattgcatgctcctatgtttct |
| | tgttgtattgggctagtagtaacacgaagaaaaattaaatttaacaaagatacttctaaaaggta |
| | tatagattgtttatataataaatacaagcatataagtttgaatgtaatcaaaaacacaaaggaga |
| | ttattactaatgaacatatttaa |
| ElxP | MDNFLSWPNKNKYFDEIKDEVKILYIDSGCDINHIEVKENILINESKS |
| (SEQ ID NO: 5) | FVDNDSELYDYTGHGTQIISAITGKHNMIGLYPRSKIVIYKITNYKGE |
| | TKFEWLYKALYKAIKMDYKIINISYSGYTQNNYIISKFKRLIEQAVKKN |
| | IHILCSASNDEVEKGFSIPSDFKGVYKIASINIEDKYSSYISKSNAEYF |
| | APGGDNYLKTQNPQSFILLANSSISNFNIGSDFGIDKRYTLNFGNSI |
| | ACSYVSCCIGLVVTRRKIKFNKDTSKRYIDCLYNKYKHISLNVIKNTK |
| | EIITNEHI |
| His6-MBP-ElxP | atggatatcggaattaatactagtcatcatcatcatcatcacagcagcggcctggtgccgcgcg |
| (SEQ ID NO: 6) | gcagccatatgaaaatcgaagaaggtaaactggtaatctggattaacggcgataaaggctat |
| | aacggtctcgctgaagtcggtaagaaattcgagaaagataccggaattaaagtcaccgttga |
| | gcatccggataaactggaagagaaattcccacaggttgcggcaactggcgatggccctgac |
| | attatcttctgggcacacgaccgctttggtggctacgctcaatctggcctgttggctgaaatcacc |
| | ccggacaaagcgttccaggacaagctgtatccgtttacctgggatgccgtacgttacaacggc |
| | aagctgattgcttacccgatcgctgttgaagcgttatcgctgatttataacaaagatctgctgccg |
| | aacccgccaaaaacctgggaagagatcccggcgctggataaagaactgaaagcgaaagg |
| | taagagcgcgctgatgttcaacctgcaagaaccgtacttcacctggccgctgattgctgctgac |
| | gggggttatgcgttcaagtatgaaaacggcaagtacgacattaaagacgtgggcgtggataa |
| | cgctggcgcgaaagcgggtctgaccttcctggttgacctgattaaaaacaaacacatgaatgc |
| | agacaccgattactccatcgcagaagctgcctttaataaaggcgaaacagcgatgaccatca |
| | acggcccgtgggcatggtccaacatcgacaccagcaaagtgaattatggtgtaacggtactg |
| | ccgaccttcaagggtcaaccatccaaaccgttcgttggcgtgctgagcgcaggtattaacgcc |
| | gccagtccgaacaaagagctggcgaaagagttcctcgaaaactatctgctgactgatgaagg |
| | tctggaagcggttaataaagacaaaccgctgggtgccgtagcgctgaagtcttacgaggaag |
| | agttggcgaaagatccacgtattgccgccaccatggaaaacgcccagaaaggtgaaatcat |
| | gccgaacatcccgcagatgtccgctttctggtatgccgtgcgtactgcggtgatcaacgccgcc |
| | agcggtcgtcagactgtcgatgaagccctgaaagacgcgcagactaattcgagctcccacca |
| | tcaccatcaccacgcgaattcggtaccgctggttccgcgtggatctgagaacctgtacttccaat |
| | ccggatccatggataattttcttagttggcctaataaaaataaatattttgatgaaataaaagatga |
| | agttaaaatattatatatagatagtgggtgtgatataaatcatatcgaagttaaagaaaatatattg |
| | ataaatgaatctaaatcttttgtggacaatgatagtgaattatatgactatacgggacatggaaca |
| | caaattattagtgcaataacaggtaagcataatatgattggactatatcctagaagtaaaattgta |
| | atatataaaataactaattataaaggtgaaactaaatttgaatggttatataaagcattatataaa |
| | gctataaaaatggactataaaattattaacataagttattcaggatacacccaaaataattacat |
| | aatatctaaattcaaaagattaatagaacaagcagttaaaaaaaatatacatattttatgtagtg |
| | ctagtaatgatgaagtggaaaaaggtttttcaataccttctgattttaaaggagtctataaaattgc |
| | gagtataaatattgaagataaatattctagttatatttctaaatctaatgctgaatactttgctcctgg |
| | aggagataattatttaaagacacagaatccacaatcatttattttgttagctaatagttctatttctaa |
| | ctttaatattggttctgattttggtatagataaaaggtatactttaaattttggtaatagtattgcatgctc |
| | ctatgtttcttgttgtattgggctagtagtaacacgaagaaaaattaaatttaacaaagatacttcta |
| | aaaggtatatagattgtttatataataaatacaagcatataagtttgaatgtaatcaaaaacaca |
| | aaggagattattactaatgaacatatttaa |
| His6-MBP-ElxP | MDIGINTSHHHHHHSSGLVPRGSHMKIEEGKLVIWINGDKGYNGLA |
| (SEQ ID NO: 7) | EVGKKFEKDTGIKVTVEHPDKLEEKFPQVAATGDGPDIIFWAHDRF |
| | GGYAQSGLLAEITPDKAFQDKLYPFTWDAVRYNGKLIAYPIAVEAL |
| | SLIYNKDLLPNPPKTWEEIPALDKELKAKGKSALMFNLQEPYFTWP |
| | LIAADGGYAFKYENGKYDIKDVGVDNAGAKAGLTFLVDLIKNKHMN |
| | ADTDYSIAEAAFNKGETAMTINGPWAWSNIDTSKVNYGVTVLPTFK |
| | GQPSKPFVGVLSAGINAASPNKELAKEFLENYLLTDEGLEAVNKDK |
| | PLGAVALKSYEEELAKDPRIAATMENAQKGEIMPNIPQMSAFWYA |
| | VRTAVINAASGRQTVDEALKDAQTNSSSHHHHHHANSVPLVPRGS |
| | ENLYFQSGSMDNFLSWPNKNKYFDEIKDEVKILYIDSGCDINHIEVK |
| | ENILINESKSFVDNDSELYDYTGHGTQIISAITGKHNMIGLYPRSKIVI |
| | YKITNYKGETKFEWLYKALYKAIKMDYKIINISYSGYTQNNYIISKFK |
| | RLIEQAVKKNIHILCSASNDEVEKGFSIPSDFKGVYKIASINIEDKYSS |
| | YISKSNAEYFAPGGDNYLKTQNPQSFILLANSSISNFNIGSDFGIDK |
| | RYTLNFGNSIACSYVSCCIGLVVTRRKIKFNKDTSKRYIDCLYNKYK |
| | HISLNVIKNTKEIITNEHI |
| Full length CylA | MKKRGLTYILISYIFLILGTTGYASDLSNNISFFIDNSQTTAIEEIESE |
| (SEQ ID NO: 9) | LSSEKVDYIQEIGLVSFKNLDDSDRKFIGKYFNVSEGKKLPDFKPE |
| | EVNSSILNINILNKDFKSFNWPYKKILSHIDPVKEQLGKDITIALIDS |
| | GIDRLHPNLQDNNLRLKNYVNDIELDEYGHGTQVAGVIDTIAPRV |
| | NLNSYKVMDGTDGNSINMLKAIVDATNDQVDIINVSLGSYKNMEI |
| | DDERFTVEAFRKVVNYARKNNILIVASAGNESRDISTGNEKHIPG |
| | GLESVITVGATKKSGDIADYSNYGSNVSIYGPAGGYGDNYKITGQI |
| | DAREMMMTYYPTSLVSPLGKAADFPDGYTLSFGTSLATPEVSAA |
| | LAAIMSKNVDNSKDSNEVLNTLFENADSFIDKNSMLKYKEVRIK |
| CylA with His₆-tag, | MGSSHHHHHHSQDPNSLSNNISFFIDNSQTTAIEEIESELSSEKVD |
| without the | YIQEIGLVSFKNLDDSDRKFIGKYFNVSEGKKLPDFKPEEVNSSIL |
| secretion signal | NINILNKDFKSFNWPYKKILSHIDPVKEQLGKDITIALIDSGIDRLHP |
| peptide | NLQDNNLRLKNYVNDIELDEYGHGTQVAGVIDTIAPRVNLNSYKV |
| (SEQ ID NO: 10) | MDGTDGNSINMLKAIVDATNDQVDIINVSLGSYKNMEIDDERFTV |
| | EAFRKVVNYARKNNILIVASAGNESRDISTGNEKHIPGGLESVITV |
| | GATKKSGDIADYSNYGSNVSIYGPAGGYGDNYKITGQIDAREMM |
| | MTYYPTSLVSPLGKAADFPDGYTLSFGTSLATPEVSAALAAIMSK |
| | NVDNSKDSNEVLNTLFENADSFIDKNSMLKYKEVRIK |
| LicP, full length | MKRIYIFLLCFAVLLPVGGKTAQAKEQAGEQYLLLEHVKDKSKLLD |
| (SEQ ID NO: 11) | TAEQFHIHADVIEEIGFAKVTGEKQKLAPFTKKLAEKVGADVIEKPI |
| | ANTAVNETESVISGSPAWGLDGILELKEYLWFAAKQTDSYRTYQI |
| | ERGHPDVKVALIDSGLDLDHPDLKASVNTNGGWNYIDGKPVSGD |
| | PTGHGTQTAGMINIIAPDVTITPYQVLDEKGGDSYNIMKAMVDAV |
| | NDGHEVINISTGSYTSLDREGKVLMKAYQRAANYAAKHQVLVFS |
| | SAGNKGVNLDEMRKTENKVHLPSALKHVVSVGSNMKSNNISPYS |
| | NQGREIEFTAPGGYLGETYDQDGMVRVTDLVLTTYPKGKDNTAL |
| | DQMLNIPKGYSLSYGTSLAAPQVAGTAALVISEYRERHHRKPSAK |
| | QVHHILRKSALDLGKPGKDVIYGYGEVRAYQALKMMNKE |
| LicP, with His₆-tag, | GSSHHHHHHSQDPKEQAGEQYLLLEHVKDKSKLLDTAEQFHIHA |
| without the | DVIEEIGFAKVTGEKQKLAPFTKKLAEKVGADVIEKPIANTAVNETE |
| secretion signal | SVISGSPAWGLDGILELKEYLWFAAKQTDSYRTYQIERGHPDVKV |
| peptide | ALIDSGLDLDHPDLKASVNTNGGWNYIDGKPVSGDPTGHGTQTA |
| (SEQ ID NO: 12) | GMINIIAPDVTITPYQVLDEKGGDSYNIMKAMVDAVNDGHEVINIS |
| | TGSYTSLDREGKVLMKAYQRAANYAAKHQVLVFSSAGNKGVNL |
| | DEMRKTENKVHLPSALKHVVSVGSNMKSNNISPYSNQGREIEFT |
| | APGGYLGETYDQDGMVRVTDLVLTTYPKGKDNTALDQMLNIPKG |
| | YSLSYGTSLAAPQVAGTAALVISEYRERHHRKPSAKQVHHILRKS |
| | ALDLGKPGKDVIYGYGEVRAYQALKMMNKE |
| LanP of B. | MYGRLGGKIMNIKRIHILLLCLLVVIIFALPGQASANDSQLDRYTIYL |
| licheniformis | ESPAHHDDWVKTLNERNIKLVYSVEEIGLYQIEGRQKEVQELAAEI |
| 9945A | NYINSHNQSVAMQPASLHNTAPAEATIFGGTPTLWDDFQWDMR |
| (SEQ ID NO: 14) | KATNNGQTYQITEGSKNTIVGIIDSGIDMNHPDLKENILSVRNFVP |
| | KGGLRGQEPYEKGDINDSTDYLGHGTFVAGQIAANGLMKGIAPE |
| | TGIRSYRVFGGKSADTVWIIDAIIQAAIDDVDIINVSFGTFLVKQNR |
| | KNKQMPDTNDLAEIKAFKKAVSFAHKKGSAVVASIGNEGLDLKDK |
| | NAVFEYWRSTKQEDLSADGKVILIPAQLPNVVTVSSIGPSGMRSV |
| | FSNYGKNVVDIAADGGDLRLLNEVGEDRYYGEGLFRQEYILGAA |
| | PGVTYTFSIGTSIAAPKVSGALALLIDQKQLHDKPDKAVKILLKNAD |
| | RNADKIDTGAGVLNVYRALTD |
| LanP of Bacillus | MKDWLKYRNTIAKIGAGITVGLLLNLPIVGVSTISAEEMKHNQIKEN |
| cereus Q1 (CerP) | SYVFTLRQGENTDEMIKEIRDKYPDLKLEVIKEIKMLNIEGDNLERV |
| (SEQ ID NO: 15) | QEAKQHLLKNYKGIIEKAGREDVVELKPPTIVKNPSNKIKYAPSQL |
| | EQEEADYSKWKWDVDKVTENGESYKIEQGNHSVKIGVIDSGIDV |
| | NHPALKGNIVSQGKILVPNVESIEDNIGHGTMIAGLIAADGKIKGVA |
| | PKIGIVPYKVFQGSSADSSWIIKAIVEAANDGMDVINLSLGTYKSIK |
| | DPEEGAVYLAYKRAIEYANSKGSLLVASSGTEGFDISNPFQLAKQ |
| | RGYENDLQLHMPGGLPGVVTVAGTNKEDRLSYYSNYGTNVDIAA |
| | PSGDYGSLWESEKVGDETAMVLTTYPTNLPQSQLSEWLGFDRG |
| | YELMIGTSLAVPKVSATAALVIAEYKEKFRLKPSNGFVKTRLYQGA |
| | KPALGDGKKYFGKGIVNAKGALDFRNTNFKKWER |
| LanP of Bacillus | MKRLKILMTILSVFLLSLVTQPVKAEKIETTDNYKFILLDNEIQKNNK |
| cereus FRI-35 | ENIINFLKENNAIEIKYTPEIQMISYKETNSNIQSDLDSKIRTKFKASI |
| (SEQ ID NO: 16) | ENVANSAKLTLQDPKILNPNEQFANNKLKYNLSSKRTLNSITINEP |
| | VSNVSPPWQIAKVTDNYKSHEITLGEKNVNIALVDSGVDYNHPEL |
| | KNSIKYLEGKSYVPSEPDLLDQNGHGTQVAGIIAAHDKLKGIVPNI |
| | TITPYKVIGKKDGESVWTIQAIIDAANNGADVINLSLGTYKAINLDD |
| | ENAIIKAYERAIQYAKEKNSIVVASAGNEGINLDKPFEKVDDEKGV |
| | LYKIHVPGGLSNVLTVSGTDKNDEFVNYSNFGPEVDFSAPSGSY |
| | GTIIDNQFKFDPSYLIGTTYPTYLEPTMLDAQLNTPKGYTLNLGTS |
| | LSAPSVSGTLGAIISRYYELHSTKPSADITIKYLKDGATDLGATGKD |
| | DQYGYGLVNSYKSTSSVK |
| LanP of Kyrpidia | MTGKGVRKRIQVVLMAGLAGGVAWGALDSVGVGRAAGLKGVDA |
| tusciae DSM 2912 | PAVAASVDRSGTGGARTAGEPGTAGQPVRGPADPEAGNRLLVV |
| (SEQ ID NO: 17) | FKSGQVPAGLVDALSAQPGVKVTPLPDIGAVAVKTATREGGDAV |
| | KRVLTARFGGDVDAVGADPILTLNTELNRPVGETLPLSAVDLTSV |
| | QVPPVPGAAPGATSPAENVSPASAKLSPAVDKAAPPATGAGSAA |
| | TAVTTGSSVDVDRAVLYQQWGWDIQEVTENGKSFAVQPGNHRV |
| | KVAVIDSGIDSNHPDLRNNIVSPGRSFVPGDPSTADDFGHGTMV |
| | AGTIAADGLLLGVGPHLGIVPYRVFQHGDAQSSWVIQAIVQAVKD |
| | GADVINLSLGTYKSLKNPADRADLLAYQKAVAYALVHGVTVVASS |
| | GTDGVDIGNPAQLANQLGHPGDLEVHAPGGLPGVLTTAATNRQ |
| | QARAYYSNYGRNVDLAAPGGDFGPLWDSEHIADVRNMCLVTYP |
| | TNLPQTPLSRMAGLPKGYEWMIGTSLAAPKVAAAAALVIAQERDK |
| | RGVRGVLGPWTNPANPVTVRNILERTAVDVGTPGRDPETGAGV |
| | VDAAKALQAVR |
| LanP of | MRRYLIHIILVSVLGAFAALSLGISAFGENQINLVEQTFLLNDKKEV |
| Enterococcus | TQLTQLVYDINPSIKQTVIEEIDMVHLENVTLEELETIENITQIEKLSE |
| caccae ATCC | KSGQMSKVETESIKITDVTTNFDDVKRIDNRQALFSRKSSSLLDLL |
| BAA-1240 | SWHVDDVTSNKQSYAIATGKNIKVAVIDSGIDTESAYFQKNLTLEN |
| (SEQ ID NO: 18) | AKSFVSNDESIIDENGHGTMVTGVLTQVAPDVKVTPYKVINATTG |
| | DSIWTIQAVIQAVKDKNQIINLSLGTYKKDNSKEERLTITAYERAIKF |
| | AKKNHVFVLASAGNDGLDLDALKLRERKIHLPGGMKNLFTIGATR |
| | RNDNKSSYSNYGNEIDFVAPGGDIYNDEGQLDLNEFIFTTYPVYL |
| | DNGLGALGIPQGYMFSGGTSLATPAVSGVVATIYEKYYQRYGIYP |
| | QNQVVNKLLKAGGVDIGTPGLDKYFGYGKVNGYQSLSKIN |
| LanP of Bacillus | MKKWQFKLCNITLIFTTIILFTFTFNKSSNAQTEHDQLIMFTDSKISN |
| cereus VPC1401 | EVNQFLEKKYPDLKKTVIPEIAVVKLEETNNKQYSKAISEIKRKFHN |
| (SEQ ID NO: 19) | EIESIGPESKIINPKESSLNAIISKEEIVPSSTQNIEEKAKLYEVLGW |
| | DIKKITEDGKSFKKQKGNHNVKIALIDSGIDFNHPDLKDNIISKGKS |
| | FVPNIDNTDDNMGHGTMAAGSIAANGNMLGVGPNLGIIPYKVMD |
| | NWQDGAESAWVTQAIIAAANDGVDVINLSLGTYKSLNKPEDQVVI |
| | ESYKRAVKYAHKRGVIVVASAGNESLDNTNPALIAEKRGFSGDM |
| | QVHLPGGGVPSLLTVSATDKDDLLSSYSNYGGISVAAPAGDYGP |
| | EWATQQKLDPFSMTLTTYPTNLPQPPISKALNLPEGYVLMAGTSV |
| | AAPKVSGAVGVLIAEQQKSGRKPLTLAKLEKILKNSSTDLGAYGK |
| | DPKYGYGLINVNKALEFIK |
| CrnP | MRILVKNILSIISSVLIFVVLSGNRVVAFAQEPSIDLLVSEGESIEQL |
| (SEQ ID NO: 20) | KEELTEVDNKLEFIEIPEINLLRIENPNEKVQEVIENSDEIDFSGKIS |
| | DKLILENNTITEKNVDFPKIFQYSAEEQNIETELLNRLTWYINTLTQ |
| | DKKAFEYSKGKNIKIGLIDSGVDTSHPLISPSLNLEKAKSFVKNDQ |
| | SIEDSNGHGTMVAGVISQVSPEAKITPYRVMSAIDGESIWTLQAII |
| | RAVNDKQDIINMSLGTYKYETKKDERITIEAFKRAACYAFIKKVLIIS |
| | SSGNQQLDSDVNYRENKIMHLPGDIKGVITVSAINKNNQLTNYSN |
| | TGTNVQYTAPGGEIIIDENGYLDARELIYTMYPLTLPNPMKDAGIP |
| | DGYTLTYGTSFSSAGVTAVFANYYSYYLQIMQKKPNSQNAHKFIA |
| | YNSTDFGVVGKDSQYGYGLPNLIRAYELISSNKNH |
| LanP of Bacillus | MKNWQSKLGTIIVILTTVILFTFTFNKSSNAESGNDQLFMFTDSNT |
| bombysepticus | SEEVNQFIKEKYPSIKSTIIPEIAMVKLEDTDNGQHIKASHEIKEKFH |
| (SEQ ID NO: 21) | NKIESTGPESKIIDPKEDNLNPSISENEIIPTVIPNIEEKAKLYQELG |
| | WDIKKITEDGKSFKKQKGNHNVKVAIIDSGIDFNHPDLKGNIISKGK |
| | SFVPNVDNTDDHMGHGTMAAGSIAANGNMLGVGPELGIIPYKVM |
| | DNWQDGAESAWVTQAIIAATKDGVDVINLSLGTYKSLEKPEDRVV |
| | VESYKRAVKYAHKHGVIVVASAGNESLDNNNPTLIAEKRGFSGDK |
| | QVHLPGGGLPSLITVSATDKNDLLASYSNYGGVSVAAPAGDYGP |
| | EWATQQKLDPFSMTLTTYPTNLPQPPISKALNLPEGYVLMAGTSV |
| | AAPKVSGAVGVLIAEQQKKGRKSLKLAQLKNILKKSSTDLGEPGK |
| | DDKYGYGLINVNKALELIK |
| LanP of Bacillus | MKDWPKYRNTIAKIGAGITVGLLLNLPIVGVSTISAEEMKHNQIKE |
| thuringiensis DB27 | NSYVFTLRQGENTDEMIKEIRDKYPDLKLEVIKEIKMLNIEGDNLE |
| (SEQ ID NO: 22) | RVQEAKQYLLKNYKGIIEKAGREDVVELKPPAIVKKPSNITKYAPS |
| | QLEQEEVDYSKWKWDVDKVTENGESYKIEQGNHSVKIGVIDSGI |
| | DVNHPALKGNIVSQGKILVPNVESIEDNIGHGTMIAGLIAADGKIKG |
| | VAPKIGIVPYKVFQGSSADSSWIIKAIVEAANDGMDVINLSLGTYK |
| | SIKNPEEGAVYLAYKRAIEYANSKGSLLVASSGTEGFDISNPFQLA |
| | KQRGYENDLQLHMPGGLPGVVTVAGTNKDDRLAYYSNYGTNVD |
| | IAAPSGDYGSLWESEKVGDETAMVLTTYPTNLPQSQLSEWLGFD |
| | RGYELMIGTSLAVPKVSATAALVIAEYKEKFRLKPSNGFVKTRLYQ |
| | GAKPALDDGKKYFGKGIVNAKGALDFRNTNFKKWER |
| LanP of | MNVQHKLPKALAGILALSVSMSCLTPSAQAAGFPKLPVQAEEEAK |
| Planomicrobium | MIYLFQEGTNLRMIAEDIQRIDPGASIDAVQEIETLTVKASNPAQSK |
| glaciei CHR43 | KIKKHVQNEFGTLITEKGEDQTVKAGDVRPSSMPPVSTLTSLAAV |
| (SEQ ID NO: 23) | SASQDTVNEEDSTYTKWLWDIDLVTQNGASRDIEIGNHGVKVGIV |
| | DSGLDFNHPDLKANIVSKGRSFVDGAADTQDYMGHGTMVAGSIA |
| | ANGHIKGIAPEIGIVPYKVFHTGNADSSDVVEAIVAAANDDMDVIN |
| | LSLGVYKSLRNKDEKAVYEAYKRALKYAEKENSFVVASSGTESA |
| | GFDIFNAKKLAAARGFSEDAQLHMPGGLDDVFTVAATNKDNALT |
| | FYSNYGENVSIGAPGGDYGPLADQGLYDVRHMTLTTYPTNLMQS |
| | ITSEYAGFEKGYEFMTGTSLAAPKVSATAALVIAEYEEVHGKKPK |
| | VHEVKKMLEKGALKGDKKNFGAGIVNAYNSLTLIK |
| LanP of Bacillus | MYRFKKYCLSIISFILIISFFPNNTNATQIAYYSILIRDNTDFNTVLDK |
| cereus VD045 | LSKDKQEVVYSIPEVNLIQVKGEKGKIISGIGIESIEEINPSTGEFKT |
| (SEQ ID NO: 24) | YTPNINDQKVLDNKAIWDVQWDIKRITNNGESYKLHSPSGKVSVA |
| | LIDSGYPENHPDIKSISIQKSKNLVPKGGYKGNEENETGNIHQLTD |
| | RTGHGTSVLSQVNADGLMKGVAPGMPVNMYRVFGESSAEGSWI |
| | IKGIIEAAKDKNDVINISAGSYLLKNGTYSDGSGNNRAEIKAYEKAI |
| | HYANKKGSIVVSALGNDSINININSELLSNLNSKIKDEGKSAKGIVQ |
| | DIPAQLAEVVSVASTGMDSKVSSFSNYGKNIIDFTAPGGDIKLLNK |
| | FGADVWMAEEMFKKEMILVAHPQGGYYFNYGNSLATPKVSGAL |
| | ALVIDKYGYKNKPNKAINHLKRNTNAENEIDLYKALQE |
| LanP from B. | MGSSHHHHHHSQDPDRYTIYLESPAHHDDWVKTLNERNIKLVY |
| licheniformis | SVEEIGLYQIEGRQKEVQELAAEINYINSHNQSVAMQPASLHNTA |
| 9945A (without | PAEATIFGGTPTLWDDFQWDMRKATNNGQTYQITEGSKNTIVGII |
| signal sequence, | DSGIDMNHPDLKENILSVRNFVPKGGLRGQEPYEKGDINDSTDYL |
| with His₆ tag) | GHGTFVAGQIAANGLMKGIAPETGIRSYRVFGGKSADTVWIIDAII |
| (SEQ ID NO: 29) | QAAIDDVDIINVSFGTFLVKQNRKNKQMPDTNDLAEIKAFKKAVSF |
| | AHKKGSAVVASIGNEGLDLKDKNAVFEYWRSTKQEDLSADGKVI |
| | LIPAQLPNVVTVSSIGPSGMRSVFSNYGKNVVDIAADGGDLRLLN |
| | EVGEDRYYGEGLFRQEYILGAAPGVTYTFSIGTSIAAPKVSGALAL |
| | LIDQKQLHDKPDKAVKILLKNADRNADKIDTGAGVLNVYRALTD |
| LanP of B. cereus | MGSSHHHHHHSQDPTQIAYYSILIRDNTDFNTVLDKLSKDNQEVV |
| VD156 (without | YSIPEVNLIQVKGEKGKIISGIGLESIEEINPSTGEFKTYTPNINDQK |
| signal sequence, | VLDNKAIWDVQWDIKRITNNGESYKLHSPSGKVSVALIDSGYPEN |
| with His₆ tag) | HPDIKSISIQKSKNLVPKGGYKGNEENETGNIHQLTDRTGHGTSV |
| (SEQ ID NO: 30) | LSQVNADGLMKGVAPGMPVNMYRVFGESSAEGSWIIKGIIEAAK |
| | DKNDVINISAGSYLLKNGTYSDGSGNNRAEIKAYEKAIHYANKKG |
| | SIVVSALGNDSININIYSELLSILNSKIKDEGKSATGIVQDIPAQLAQ |
| | VVSVASTGMDSKVSSFSNYGKNIIDFTAPGGDIKLLNKFGADVWM |
| | AEEMFKKEMILVAHPQGGYYFNYGNSLATPKVSGALALVIDKYGY |
| | KNKPNKAINHLKRNTNAENEIDLYKALQE |

### REFERENCES

Arnison, P. G., Bibb, M. J., Bierbaum, G., Bowers, A. A., Bugni, T. S., Bulaj, G., Camarero, J. A., Campopiano, D. J., Challis, G. L., Clardy, J., Cotter, P. D., Craik, D. J., Dawson, M., Dittmann, E., Donadio, S., Dorrestein, P. C., Entian, K.-D., Fischbach, M. A., Garavelli, J. S., Göransson, U., Gruber, C. W., Haft, D. H., Hemscheidt, T. K., Hertweck, C., Hill, C., Horswill, A. R., Jaspars, M., Kelly, W. L., Klinman, J. P., Kuipers, O. P., Link, A. J., Liu, W., Marahiel, M. A., Mitchell, D. A., Moll, G. N., Moore, B. S., Mdller, R., Nair, S. K., Nes, I. F., Norris, G. E., Olivera, B. M., Onaka, H., Patchett, M. L., Piel, J., Reaney, M. J. T., Rebuffat, S., Ross, R. P., Sahl, H.-G., Schmidt, E. W., Selsted, M. E., Severinov, K., Shen, B., Sivonen, K., Smith, L., Stein, T., Sissmuth, R. E., Tagg, J. R., Tang, G.-L., Truman, A. W., Vederas, J. C., Walsh, C. T., Walton, J. D., Wenzel, S. C., Willey, J. M., and van der Donk, W. A. (2013) Ribosomally Synthesized and Post-translationally Modified Peptide Natural Products: Overview and Recommendations for a Universal Nomenclature, Nat. Prod. Rep. 30, 108-160.
van der Meer, J. R., Rollema, H. S., Siezen, R. J., Beerthuyzen, M. M., Kuipers, O. P., and de Vos, W. M. (1994) Influence of amino acid substitutions in the nisin leader peptide on biosynthesis and secretion of nisin by Lactococcus lactis, J. Biol. Chem. 269, 3555-3562.
Li, B., Yu, J. P., Brunzelle, J. S., Moll, G. N., van der Donk, W. A., and Nair, S. K. (2006) Structure and mechanism of the lantibiotic cyclase involved in nisin biosynthesis, Science 311, 1464-1467.
McClerren, A. L., Cooper, L. E., Quan, C., Thomas, P. M., Kelleher, N. L., and van der Donk, W. A. (2006) Discovery and in vitro biosynthesis of haloduracin, a two-component lantibiotic, Proc. Natl. Acad. Sci. U.S.A. 103, 17243-17248.
Ekkelenkamp, M. B., Hanssen, M., Danny Hsu, S. T., de Jong, A., Milatovic, D., Verhoef, J., and van Nuland, N. A. (2005) Isolation and structural characterization of epilancin 15×, a novel lantibiotic from a clinical strain of Staphylococcus epidermidis, FEBS Lett. 579, 1917-1922.
Velasquez, J. E., Zhang, X., and van der Donk, W. A. (2011) Biosynthesis of the Antimicrobial Peptide Epilancin 15X and its Unusual N-terminal Lactate Moiety, Chem. Biol. 18, 857-867.
Knerr, P. J., and van der Donk, W. A. (2012) Discovery, biosynthesis, and engineering of lantipeptides, Annu. Rev. Biochem. 81, 479-505.
Zhang, Q., Yu, Y., Velasquez, J. E., and van der Donk, W. A. (2012) Evolution of lanthipeptide synthetases, Proc. Natl. Acad. Sci. U.S.A. 109, 18361-18366.
Hivarstein, L. S., Diep, D. B., and Nes, I. F. (1995) A family of bacteriocin ABC transporters carry out proteolytic processing of their substrates concomitant with export, Mol. Microbiol. 16, 229-240.
Oman, T. J., and van der Donk, W. A. (2010) Follow the leader: the use of leader peptides to guide natural product biosynthesis, Nat. Chem. Biol. 6, 9-18.
Dirix, G., Monsieurs, P., Dombrecht, B., Daniels, R., Marchal, K., Vanderleyden, J., and Michiels, J. (2004) Peptide signal molecules and bacteriocins in Gram-negative bacteria: a genome-wide in silico screening for peptides containing a double-glycine leader sequence and their cognate transporters, Peptides 25, 1425-1440.
Uguen, P., Hindré, T., Didelot, S., Marty, C., Haras, D., Le Pennec, J. P., Vallee-Rehel, K., and Dufour, A. (2005) Maturation by LctT is required for biosynthesis of full-length lantibiotic lacticin 481, Appl. Environ. Microbiol. 71, 562-565.
Ishii, S., Yano, T., and Hayashi, H. (2006) Expression and characterization of the peptidase domain of Streptococcus pneumoniae ComA, a bifunctional ATP-binding cassette transporter involved in quorum sensing pathway, J. Biol. Chem. 281, 4726-4731.
Furgerson Ihnken, L. A., Chatterjee, C., and van der Donk, W. A. (2008) In vitro Reconstitution and Substrate Specificity of a Lantibiotic Protease, Biochemistry 47, 7352-7363.
Nishie, M., Sasaki, M., Nagao, J., Zendo, T., Nakayama, J., and Sonomoto, K. (2011) Lantibiotic transporter requires cooperative functioning of the peptidase domain and the ATP binding domain, J. Biol. Chem. 286, 11163-11169.
Völler, G. H., Krawczyk, B., Ensle, P., and Sussmuth, R. D. (2013) Involvement and unusual substrate specificity of a prolyl oligopeptidase in class III lanthipeptide maturation, J. Am. Chem. Soc. 135, 7426-7429.
Siezen, R. J., Rollema, H. S., Kuipers, O. P., and de Vos, W. M. (1995) Homology modeling of the Lactococcus lactis leader peptidase NisP and its interaction with the precursor of the lantibiotic nisin, Prot. Eng. 8, 117-125.
Kuipers, A., de Boef, E., Rink, R., Fekken, S., Kluskens, L. D., Driessen, A. J., Leenhouts, K., Kuipers, O. P., and Moll, G. N. (2004) NisT, the transporter of the lantibiotic nisin, can transport fully modified, dehydrated, and unmodified prenisin and fusions of the leader peptide with non-lantibiotic peptides, J. Biol. Chem. 279, 22176-22182.
van der Meer, J. R., Polman, J., Beerthuyzen, M. M., Siezen, R. J., Kuipers, O. P., and de Vos, W. M. (1993) Characterization of the Lactococcus lactis nisin A operon genes nisP, encoding a subtilisin-like serine protease involved in precursor processing, and nisR, encoding a regulatory protein involved in nisin biosynthesis, J. Bacteriol. 175, 2578-2588.
Plat, A., Kluskens, L. D., Kuipers, A., Rink, R., and Moll, G. N. (2011) Requirements of the engineered leader peptide of nisin for inducing modification, export, and cleavage, Appl. Environ. Microbiol. 77, 604-611.
Kuipers, O. P., Rollema, H. S., de Vos, W. M., and Siezen, R. J. (1993) Biosynthesis and secretion of a precursor of nisin Z by Lactococcus lactis, directed by the leader peptide of the homologous lantibiotic subtilin from Bacillus subtilis, FEBS Lett. 330, 23-27.
Mavaro, A., Abts, A., Bakkes, P. J., Moll, G. N., Driessen, A. J., Smits, S. H., and Schmitt, L. (2011) Substrate recognition and specificity of the NisB protein, the lantibiotic dehydratase involved in nisin biosynthesis, J. Biol. Chem. 286, 30552-30560.
Abts, A., Montalban-Lopez, M., Kuipers, O. P., Smits, S. H., and Schmitt, L. (2013) NisC binds the FxLx motif of the nisin leader peptide, Biochemistry 52, 5387-5395.
Khusainov, R., Moll, G. N., and Kuipers, O. P. (2013) Identification of distinct nisin leader peptide regions that determine interactions with the modification enzymes NisB and NisC, FEBS Open Bio 3, 237-242.
Yang, X., and van der Donk, W. A. (2013) Ribosomally Synthesized and Post-Translationally Modified Peptide Natural Products: New Insights into the Role of Leader and Core Peptides during Biosynthesis, Chem. Eur. J. 19, 7662-7677.
Kaletta, C., Entian, K. D., Kellner, R., Jung, G., Reis, M., and Sahl, H. G. (1989) Pep5, a new lantibiotic: structural gene isolation and prepeptide sequence, Arch. Microbiol. 152, 16-19.
Meyer, C., Bierbaum, G., Heidrich, C., Reis, M., Siling, J., Iglesias-Wind, M. I., Kempter, C., Molitor, E., and Sahl, H.-G. (1995) Nucleotide Sequence of the Lantibiotic Pep5 Biosynthetic Gene Cluster and Functional Analysis of PepP and PepC, Eur. J. Biochem. 232, 478-489.
Ryan, M. P., Jack, R. W., Josten, M., Sahl, H. G., Jung, G., Ross, R. P., and Hill, C. (1999) Extensive post-translational modification, including serine to D-alanine conversion, in the two-component lantibiotic, lacticin 3147, J. Biol. Chem. 274, 37544-37550.
Begley, M., Cotter, P. D., Hill, C., and Ross, R. P. (2009) Identification of a novel two-peptide lantibiotic, lichenicidin, following rational genome mining for LanM proteins, Appl. Environ. Microbiol. 75, 5451-5460.
Dischinger, J., Josten, M., Szekat, C., Sahl, H. G., and Bierbaum, G. (2009) Production of the novel two-peptide lantibiotic lichenicidin by Bacillus licheniformis DSM 13, PLoS One 4, e6788.
Shenkarev, Z. O., Finkina, E. I., Nurmukhamedova, E. K., Balandin, S. V., Mineev, K. S., Nadezhdin, K. D., Yakimenko, Z. A., Tagaev, A. A., Temirov, Y. V., Arseniev, A. S., and Ovchinnikova, T. V. (2010) Isolation, structure elucidation, and synergistic antibacterial activity of a novel two-component lantibiotic lichenicidin from Bacillus licheniformis VK21, Biochemistry 49, 6462-6472.
Li, B., Sher, D., Kelly, L., Shi, Y., Huang, K., Knerr, P. J., Joewono, I., Rusch, D., Chisholm, S. W., and van der Donk, W. A. (2010) Catalytic promiscuity in the biosynthesis of cyclic peptide secondary metabolites in planktonic marine cyanobacteria, Proc. Natl. Acad. Sci. U.S.A. 107, 10430-10435.
Marceau, P., Bure, C., and Delmas, A. F. (2005) Efficient synthesis of C-terminal modified peptide ketones for chemical ligations, Bioorg. Med. Chem. Lett. 15, 5442-5445.
Mortvedt, C. I., Nissen-Meyer, J., Sletten, K., and Nes, I. F. (1991) Purification and amino acid sequence of lactocin S, a bacteriocin produced by Lactobacillus sake L45, Appl. Environ. Microbiol. 57, 1829-1834.
Ross, A. C., Liu, H., Pattabiraman, V. R., and Vederas, J. C. (2010) Synthesis of the lantibiotic lactocin S using peptide cyclizations on solid phase, J. Am. Chem. Soc. 132, 462-463.
Delgado, A., Brito, D., Fevereiro, P., Tenreiro, R., and Peres, C. (2005) Bioactivity quantification of crude bacteriocin solutions, J. Microbiol. Methods 62, 121-124.
Blaesse, M., Kupke, T., Huber, R., and Steinbacher, S. (2000) Crystal structure of the peptidyl-cysteine decarboxylase EpiD complexed with a pentapeptide substrate, Embo. J. 19, 6299-6310.
Blaesse, M., Kupke, T., Huber, R., and Steinbacher, S. (2003) Structure of MrsD, an FAD-binding protein of the HFCD family, Acta Cryst., Sect. D: Biol. Cryst. D59, 1414-1421.
Jornvall, H., Persson, B., Krook, M., Atrian, S., Gonzalez-Duarte, R., Jeffery, J., and Ghosh, D. (1995) Short-chain dehydrogenases/reductases (SDR), Biochemistry 34, 6003-6013.
Tanaka, N., Nonaka, T., Nakamura, K. T., and Hara, A. (2001) SDR: Structure, mechanism of action, and substrate recognition, Curr. Org. Chem. 5, 89-111.
Hall, B. G. (2005) Comparison of the accuracies of several phylogenetic methods using protein and DNA sequences, Mol. Biol. Evol. 22, 792-802.
Ronquist, F., Teslenko, M., van der Mark, P., Ayres, D. L., Darling, A., Hohna, S., Larget, B., Liu, L., Suchard, M. A., and Huelsenbeck, J. P. (2012) MrBayes 3.2: efficient Bayesian phylogenetic inference and model choice across a large model space, Systematic biology 61, 539-542.
Wolf, C. E., and Gibbons, W. R. (1996) Improved method for quantification of the bacteriocin nisin, J. Appl. Bacteriol. 80, 453-457.
Parente, E., Brienza, C., Moles, M., and Ricciardi, A. (1995) A comparison of methods for the measurement of bacteriocin activity, J. Microbiol. Methods 22, 95-108.
Velasquez, J. E., Zhang, X., and van der Donk, W. A. (2011) Biosynthesis of the Antimicrobial Peptide Epilancin 15X and its Unusual N-terminal Lactate Moiety, Chem. Biol. 18, 857-867.
Liu, H., and Naismith, J. H. (2008) An efficient one-step site-directed deletion, insertion, single and multiple-site plasmid mutagenesis protocol, BMC biotechnology 8, 91.
Garg, N., Salazar-Ocampo, L. M., and van der Donk, W. A. (2013) In vitro activity of the nisin dehydratase NisB, Proc. Natl. Acad. Sci. U.S.A. 110, 7258-7263.
Ishii, S., Yano, T., and Hayashi, H. (2006) Expression and characterization of the peptidase domain of Streptococcus pneumoniae ComA, a bifunctional ATP-binding cassette transporter involved in quorum sensing pathway, J. Biol. Chem. 281, 4726-4731.
Geoghegan, K. F., and Stroh, J. G. (1992) Site-Directed conjugation of non-peptide groups to peptides and proteins via periodate oxidation of a 2-amino alcohol. Application to modification at N-terminal serine, Bioconjugate Chem. 3, 138-146.
Otwinowski, Z., Borek, D., Majewski, W., and Minor, W. (2003) Multiparametric scaling of diffraction intensities, Acta crystallogr., Sect A: Found. Crystallogr. 59, 228-234.
McRee, D. E. (1999) XtalView/Xfit—A versatile program for manipulating atomic coordinates and electron density, J Struct Biol 125, 156-165.
Perrakis, A., Sixma, T. K., Wilson, K. S., and Lamzin, V. S. (1997) wARP: improvement and extension of crystallographic phases by weighted averaging of multiple-refined dummy atomic models, Acta Crystallogr., Sect. D: Biol. Crystallogr. 53, 448-455.
Murshudov, G. N., Vagin, A. A., and Dodson, E. J. (1997) Refinement of macromolecular structures by the maximum-likelihood method, Acta Crystallogr., Sect. D: Biol. Crystallogr. 53, 240-255.
Murshudov, G. N., Vagin, A. A., Lebedev, A., Wilson, K. S., and Dodson, E. J. (1999) Efficient anisotropic refinement of macromolecular structures using FFT, Acta Crystallogr., Sect. D: Biol. Crystallogr. 55, 247-255.
Kleywegt, G. J., and Brunger, A. T. (1996) Checking your imagination: applications of the free R value, Structure 4, 897-904.
Laskowski, R. A., Rullmannn, J. A., MacArthur, M. W., Kaptein, R., and Thornton, J. M. (1996) AQUA and PROCHECK-NMR: programs for checking the quality of protein structures solved by NMR, J. Biomol. NMR 8, 477-486.
Delgado, A., Brito, D., Fevereiro, P., Tenreiro, R., and Peres, C. (2005) Bioactivity quantification of crude bacteriocin solutions, J. Microbiol. Methods 62, 121-124.
Grant, S. G., Jessee, J., Bloom, F. R., and Hanahan, D. (1990) Differential plasmid rescue from transgenic mouse DNAs into Escherichia coli methylation-restriction mutants, Proc. Natl. Acad. Sci. U.S.A. 87, 4645-4649.
Mortvedt, C. I., Nissen-Meyer, J., Sletten, K., and Nes, I. F. (1991) Purification and amino acid sequence of lactocin S, a bacteriocin produced by Lactobacillus sake L45, Appl. Environ. Microbiol. 57, 1829-1834.
P. G. Arnison, M. J. Bibb, G. Bierbaum, A. A. Bowers, T. S. Bugni, G. Bulaj, J. A. Camarero, D. J. Campopiano, G. L. Challis, J. Clardy, P. D. Cotter, D. J. Craik, M. Dawson, E. Dittmann, S. Donadio, P. C. Dorrestein, K.-D. Entian, M. A. Fischbach, J. S. Garavelli, U. Göransson, C. W. Gruber, D. H. Haft, T. K. Hemscheidt, C. Hertweck, C. Hill, A. R. Horswill, M. Jaspars, W. L. Kelly, J. P. Klinman, O. P. Kuipers, A. J. Link, W. Liu, M. A. Marahiel, D. A. Mitchell, G. N. Moll, B. S. Moore, R. Müller, S. K. Nair, I. F. Nes, G. E. Norris, B. M. Olivera, H. Onaka, M. L. Patchett, J. Piel, M. J. T. Reaney, S. Rebuffat, R. P. Ross, H.-G. Sahl, E. W. Schmidt, M. E. Selsted, K. Severinov, B. Shen, K. Sivonen, L. Smith, T. Stein, R. E. Sissmuth, J. R. Tagg, G.-L. Tang, A. W. Truman, J. C. Vederas, C. T. Walsh, J. D. Walton, S. C. Wenzel, J. M. Willey, W. A. van der Donk, Nat. Prod. Rep. 2013, 30, 108.
X. Yang, W. A. van der Donk, Chem. Eur. J. 2013, 19, 7662.
Y. Yu, Q. Zhang, W. A. van der Donk, Protein Sci. 2013, 22, 1478.
P. J. Knerr, W. A. van der Donk, Annu. Rev. Biochem. 2012, 81, 479.
G. Bierbaum, H. G. Sahl, Curr. Pharm. Biotechnol. 2009, 10, 2
J. M. Willey, W. A. van der Donk, Annu. Rev. Microbiol. 2007, 61, 477.
A. Plat, A. Kuipers, R. Rink, G. N. Moll, Curr. Protein Pept. Sci. 2013, 14, 85.
M. Nishie, J. I. Nagao, K. Sonomoto, Biocontrol Sci. 2012, 17, 1.
M. M. Al-Mahrous, M. Upton, Expert Opinion on Drug Discovery 2011, 6, 155.
P. D. Cotter, C. Hill, R. P. Ross, Curr. Protein Pept. Sci. 2005, 6, 61.
D. Van Tyne, M. J. Martin, M. S. Gilmore, Toxins 2013, 5, 895.
K. M. Daly, P. D. Cotter, C. Hill, R. P. Ross, Curr. Protein Pept. Sci. 2012, 13, 509.
N. Garg, L. M. Salazar-Ocampo, W. A. van der Donk, Proc. Natl. Acad. Sci. U.S.A. 2013, 110, 7258.
B. Li, J. P. Yu, J. S. Brunzelle, G. N. Moll, W. A. van der Donk, S. K. Nair, Science 2006, 311, 1464.
L. Xie, L. M. Miller, C. Chatterjee, O. Averin, N. L. Kelleher, W. A. van der Donk, Science 2004, 303, 679.
W. M. Muller, T. Schmiederer, P. Ensle, R. D. Sussmuth, Angew. Chem., Int. Ed. 2010, 49, 2436.
Y. Goto, B. Li, J. Claesen, Y. Shi, M. J. Bibb, W. A. van der Donk, PLoS Biol. 2010, 8, e1000339.
J. R. van der Meer, J. Polman, M. M. Beerthuyzen, R. J. Siezen, O. P. Kuipers, W. M. de Vos, J. Bacteriol. 1993, 175, 2578.
S. Geissler, F. Götz, T. Kupke, J. Bacteriol. 1996, 178, 284; b) M. L. Kuhn, P. Prachi, G. Minasov, L. Shuvalova, J. Ruan, I. Dubrovska, J. Winsor, M. Giraldi, M. Biagini, S. Liberatori, S. Savino, F. Bagnoli, W. F. Anderson, G. Grandi, FASEB J. 2014, 4, 1780.
J. E. Velasquez, X. Zhang, W. A. van der Donk, Chem. Biol. 2011, 18, 857.
M. C. Booth, C. P. Bogie, H.-G. Sahl, R. J. Siezen, K. L. Hatter, M. S. Gilmore, Mol. Microbiol. 1996, 21, 1175.
T. Caetano, J. M. Krawczyk, E. Mosker, R. D. Sissmuth, S. Mendo, Chem. Biol. 2011, 18, 90.
J. Wang, L. Zhang, K. Teng, S. Sun, Z. Sun, J. Zhong, Appl. Environ. Microbiol. 2014, 80, 2633.
L. A. Furgerson Ihnken, C. Chatterjee, W. A. van der Donk, Biochemistry 2008, 47, 7352.
M. Nishie, K. Shioya, J. Nagao, H. Jikuya, K. Sonomoto, J. Biosci. Bioeng. 2009, 108, 460.
G. H. Völler, B. Krawczyk, P. Ensle, R. D. Sissmuth, J. Am. Chem. Soc. 2013, 135, 7426.
M. Nishie, M. Sasaki, J. Nagao, T. Zendo, J. Nakayama, K. Sonomoto, J. Biol. Chem. 2011, 286, 11163.
T. J. Oman, W. A. van der Donk, Nat. Chem. Biol. 2010, 6, 9.
C. R. Cox, P. S. Coburn, M. S. Gilmore, Curr. Protein Pept. Sci. 2005, 6, 77.
M. A. Ortega, J. E. Velasquez, N. Garg, Q. Zhang, R. Joyce, S. K. Nair, W. A. van der Donk, Submitted 2014.
C. L. Young, Z. T. Britton, A. S. Robinson, Biotechnol. J. 2012, 7, 620; b) Q. Li, L. Yi, P. Marek, B. L. Iverson, FEBS Lett. 2013, 587, 1155.
M. S. Gilmore, R. A. Segarra, M. C. Booth, Infect. Immun. 1990, 58, 3914.
W. Tang, W. A. van der Donk, Nat. Chem. Biol. 2013, 9, 157.
A. L. McClerren, L. E. Cooper, C. Quan, P. M. Thomas, N. L. Kelleher, W. A. van der Donk, Proc. Natl. Acad. Sci. U.S.A. 2006, 103, 17243.
Y. Shi, X. Yang, N. Garg, W. A. van der Donk, J. Am. Chem. Soc. 2011, 133, 2338.
H. Holo, Z. Jeknic, M. Daeschel, S. Stevanovic, I. F. Nes, Microbiology 2001, 147, 643.
A. Kuipers, E. De Boef, R. Rink, S. Fekken, L. D. Kluskens, A. J. Driessen, K. Leenhouts, O. P. Kuipers, G. N. Moll, J. Biol. Chem. 2004, 279, 22176.
S. D. Power, R. M. Adams, J. A. Wells, Proc. Natl. Acad. Sci. U.S.A. 1986, 83, 3096.
J. L. Silen, D. Frank, A. Fujishige, R. Bone, D. A. Agard, J. Bacteriol. 1989, 171, 1320.
C. Chatterjee, M. Paul, L. Xie, W. A. van der Donk, Chem. Rev. 2005, 105, 633.
D. Chen, L. Lei, R. Flores, Z. W. Huang, Z. M. Wu, J. J. Chai, G. M. Zhong, Microb. Pathog. 2010, 49, 164.
R. J. Siezen, W. M. Devos, J. A. M. Leunissen, B. W. Dijkstra, Protein Eng. 1991, 4, 719. J. J. Perona, C. S. Craik, Protein Sci. 1995, 4, 337; c) L. Yi, M. C. Gebhard, Q. Li, J. M. Taft, G. Georgiou, B. L. Iverson, Proc. Natl. Acad. Sci. U.S.A. 2013, 110, 7229.
B. Li, L. E. Cooper, W. A. van der Donk, Methods Enzymol. 2009, 458, 533.
Y. Shi, X. Yang, N. Garg, W. A. van der Donk, J. Am. Chem. Soc. 2011, 133, 2338
W. Tang, W. A. van der Donk, Nat. Chem. Biol. 2013, 9, 157

All references, including publications, patent applications, and patents, cited herein are hereby incorporated by reference to the same extent as if each reference were individually and specifically indicated to be incorporated by reference and were set forth in its entirety herein. Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated nucleic acid comprising an open reading frame encoding a lanthipeptide protease polypeptide for scarless tag removal from a polypeptide.

2. The isolated nucleic acid of claim 1, wherein the lanthipeptide protease is codon optimized for expression in an expression host.

3. The isolated nucleic acid of claim 2, wherein the expression host is selected from E. coli, S. cerevisiae, S. pombe, P. pastoris, an insect cell, a HeLa cell, a Jurkat cell, a 293 cell, a CHO cell and a COS cell.

4. The isolated nucleic acid of claim 1, wherein the lanthipeptide protease polypeptide is selected from SEQ ID NOS: 5, 7, 9-25, 29 and 30, including equivalents thereof and derivatives thereof.

5. The isolated nucleic acid of claim 1, wherein the lanthipeptide protease polypeptide recognizes a substrate recognition sequence selected from SEQ ID NOS: 1-3, 27, 31-46 and sequences of Table 3, including equivalents thereof and derivatives thereof.

6. The isolated nucleic acid of claim 1, wherein the polypeptide is a cognate lanthipeptide, a non-cognate lanthipeptide or a heterologous polypeptide.

7. The isolated nucleic acid of claim 1, further comprising a vector that includes a transcription controlling signal, wherein the isolated nucleic acid is operably linked to the transcriptional controlling signal to enable expression of the lanthipeptide protease polypeptide.

8. The isolated nucleic acid of claim 7, wherein the transcriptional controlling signal comprise a transcriptional initiation element.

9. The isolated nucleic acid of claim 8, wherein the transcriptional controlling signals further comprise a transcriptional termination element.

10. The isolated nucleic acid of claim 7, further comprising a translational controlling signal.

11. The isolated nucleic acid of claim 10, wherein the translational controlling signal comprises at least one selected from a translational enhancer and a post-translational processing element.

12. An expression cassette comprising an open reading frame for a polypeptide, wherein the open reading frame encodes a substrate recognition sequence for a lanthipeptide protease polypeptide.

13. The expression cassette of claim 12, wherein the substrate recognition sequence is selected from SEQ ID NOS: 1-3, 27, 31-46 and sequences of Table 3, including equivalents thereof and derivatives thereof.

14. The expression cassette of claim 12, wherein the lanthipeptide protease polypeptide is selected from SEQ ID NOS: 5, 7, 9-25, 29 and 30, including equivalents thereof and derivatives thereof.

15. The expression cassette of claim 12, wherein the polypeptide is selected from a cognate lanthipeptide, a non-cognate lanthipeptide or a heterologous polypeptide.

16. The expression cassette of claim 12, further comprising a transcription controlling signal, wherein the isolated nucleic acid is operably linked to the transcriptional controlling signal to enable expression of the polypeptide.

17. The expression cassette of claim 12, wherein the transcriptional controlling signal comprises a transcriptional initiation element.

18. The expression cassette of claim 17, wherein the transcriptional controlling signal further comprises a transcriptional termination element.

19. The expression cassette of claim 17, further comprising a translational controlling signal.

20. The expression cassette of claim 19, wherein the translational controlling signal comprises at least one selected from a translational enhancer and a post-translational processing element.

21. The expression cassette of claim 19, wherein the translational controlling signal comprises a post-translational processing element.

22. A method of scarless tag removal from a polypeptide, comprising;
providing the polypeptide, said polypeptide comprises the structure: T-R-P, wherein T comprises a tag motif, R comprises a lanthipeptide protease substrate recognition sequence and P comprises an open reading frame encoding a polypeptide without the tag motif and lanthipeptide protease substrate recognition sequence; and subjecting the polypeptide to a lanthipeptide protease having specificity for catalyzing proteolytic cleavage at the lanthipeptide protease substrate recognition sequence, thereby providing the polypeptide without a tag scar.

23. The method of claim 22, further comprising a step of purifying the polypeptide without a tag scar.

24. The method of claim 22, wherein the lanthipeptide protease is codon optimized for expression in an expression host.

25. The method of claim 24, wherein the expression host is selected from E. coli, S. cerevisiae, S. pombe, P. pastoris, an insect cell, a HeLa cell, a Jurkat cell, a 293 cell, a CHO cell and a COS cell.

26. The method of claim 22, wherein the lanthipeptide protease polypeptide is selected from SEQ ID NOS: 5, 7, 9-25, 29 and 30, including equivalents thereof and derivatives thereof.

27. The method of claim 22, wherein lanthipeptide protease substrate recognition sequence is selected from SEQ ID NOS: 1-3, 27, 31-46 and sequences of Table 3, including equivalents thereof and derivatives thereof.

28. The method of claim 22, wherein the tag motif comprises an affinity tag.

29. The method of claim 28, wherein the affinity tag is selected from polyhistine, maltose binding protein, glutathione-S-transferase, HaloTag®, AviTag, Calmodulin-tag, polyglutamate tag, FLAG-tag, HA-tag, Myc-tag, S-tag, SBP-tag, Softag 3, V5 tag and Xpress tag.

30. The method of claim 22, wherein the polypeptide is a cognate lanthipeptide, a non-cognate lanthipeptide or a heterologous polypeptide.

31. The method of claim 22, wherein the polypeptide is expressed in vivo or in vitro.

32. The method of claim 22, wherein the polypeptide is expressed in vivo from an expression cassette in an expression host.

33. The method of claim 31, wherein the expression host is selected from E. coli, S. cerevisiae, S. pombe, P. pastoris, an insect cell, a HeLa cell, a Jurkat cell, a 293 cell, a CHO cell and a COS cell.

34. The method of claim 22, where the polypeptide is expressed in vitro from an expression cassette in a coupled transcription-translation system or from a translation template in a translation system.

35. A kit for expressing a polypeptide without a tag scar, comprising: an expression vector comprising an expression cassette, said expression cassette encodes a polypeptide comprising the structure: T-R-P, wherein T comprises a tag motif, R comprises a lanthipeptide protease substrate recognition sequence and P comprises an open reading frame encoding a polypeptide without the tag motif and lanthipeptide protease substrate recognition sequence; and a lanthipeptide protease having specificity for catalyzing proteolytic cleavage at the lanthipeptide protease substrate recognition sequence, thereby providing the polypeptide without the tag scar.

36. The kit of claim 35, further comprising a reagent to purify the polypeptide without the tag scar.

37. The kit of claim 35, further comprising an expression host.

38. The kit of claim 37, wherein the wherein the expression host is selected from E. coli, S. cerevisiae, S. pombe, P. pastoris, an insect cell, a HeLa cell, a Jurkat cell, a 293 cell, a CHO cell and a COS cell.

39. The kit of claim 37, wherein the lanthipeptide protease is codon optimized for expression in the expression host.

40. The kit of claim 35, wherein the lanthipeptide protease is codon optimized for expression in an expression host.

41. The kit of claim 40, wherein the expression host is selected from E. coli, S. cerevisiae, S. pombe, P. pastoris, an insect cell, a HeLa cell, a Jurkat cell, a 293 cell, a CHO cell and a COS cell.

42. The kit of claim 35, wherein the lanthipeptide protease polypeptide is selected from SEQ ID NOS: 5, 7, 9-25, 29 and 30, including equivalents thereof and derivatives thereof.

43. The kit of claim 35, wherein lanthipeptide protease substrate recognition sequence is selected from SEQ ID NOS: 1-3, 27, 31-46 and sequences of Table 3, including equivalents thereof and derivatives thereof.

44. The kit of claim 35, wherein the tag motif comprises an affinity tag.

45. An isolated polypeptide comprising the structure:
T-R-P,
wherein T comprises a tag motif,
R comprises a lanthipeptide protease substrate recognition sequence and
P comprises an open reading frame encoding a polypeptide without the tag motif and lanthipeptide protease substrate recognition sequence.

46. The isolated polypeptide of claim 45, wherein the isolated polypeptide is codon optimized for expression in an expression host.

47. The isolated polypeptide of claim 45, wherein the expression host is selected from E. coli, S. cerevisiae, S. pombe, P. pastoris, an insect cell, a HeLa cell, a Jurkat cell, a 293 cell, a CHO cell and a COS cell.

48. The isolated polypeptide of claim 45, wherein lanthipeptide protease substrate recognition sequence is selected from SEQ ID NOS: 1-3, 27, 31-46 and sequences of Table 3, including equivalents thereof and derivatives thereof.

49. The isolated polypeptide of claim 45, wherein the tag motif comprises an affinity tag.

50. The isolated polypeptide of claim 45, wherein the affinity tag is selected from polyhistine, maltose binding protein, glutathione-S-transferase, HaloTag®, AviTag, Calmodulin-tag, poly glutamate tag, FLAG-tag, HA-tag, Myc-tag, S-tag, SBP-tag, Softag 3, V5 tag and Xpress tag.

51. The isolated polypeptide of claim 45, wherein the polypeptide is a cognate lanthipeptide, a non-cognate lanthipeptide or a heterologous polypeptide.
